(19) United States
(12) Patent Application Publication
(10) Pub. No.: US 2008/0305106 A1
(43) Pub. Date: Dec. 11, 2008

(54) Novel Gene Disruptions, Composition and Methods Relating Thereto

(76) Inventors: Jane Brennan, Lothian Region (GB); Frederic J. de Sauvage, Foster City, CA (US); Ellen Filvaroff, San Francisco, CA (US); Iqbal Grewal, Snohomish, WA (US); Bryan Irving, San Francisco, CA (US); Jagath Reddy Junutula, Foster City, CA (US); Daniel Kirchhofer, Los Altos, CA (US); Franklin Peale, San Carlos, CA (US); Heide Phillips, Palo Alto, CA (US); Tracy Tang, Redwood City, CA (US); Dineli Wickramasinghe, San Francisco, CA (US); Weilan Ye, Foster City, CA (US)

Correspondence Address: GENENTECH, INC., 1 DNA WAY, SOUTH SAN FRANCISCO, CA, 94080, US

(21) Appl. No.: 11/568,901

(22) PCT Filed: Apr. 12, 2005
(86) PCT No.: PCT/US05/12478

§ 371 Date: Nov. 9, 2006

Related U.S. Application Data
(60) Provisional application No. 60/571,347, filed on May 12, 2004.

(51) Int. Cl. A61K 39/395; C12Q 1/68; C12N 5/06; A61K 38/00; A61P 19/00; A61P 27/00; A01K 67/027; C07K 16/18

(52) U.S. Cl. 424/138.1; 435/6; 435/325; 435/352; 800/3; 530/387.1; 514/12

(57) ABSTRACT

The present invention relates to transgenic animals, as well as compositions and methods relating to the characterization of gene function. Specifically, the present invention provides transgenic mice comprising disruptions in PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 genes. Such in vivo studies and characterizations may provide valuable identification and discovery of therapeutics and/or treatments useful in the prevention, amelioration or correction of diseases or dysfunctions associated with gene disruptions such as neurological disorders; cardiovascular, endothelial or angiogenic disorders; eye abnormalities; immunological disorders; oncological disorders; bone metabolic abnormalities or disorders; lipid metabolic disorders; or developmental abnormalities.

### FIELD OF THE INVENTION

The present invention relates to compositions, including transgenic and knockout animals and methods of using such compositions for the diagnosis and treatment of diseases or disorders.

### BACKGROUND OF THE INVENTION

Extracellular proteins play important roles in, among other things, the formation, differentiation and maintenance of multicellular organisms. The fate of many individual cells, e.g., proliferation, migration, differentiation, or interaction with other cells, is typically governed by information received from other cells and/or the immediate environment. This information is often transmitted by secreted polypeptides (for instance, mitogenic factors, survival factors, cytotoxic factors, differentiation factors, neuropeptides, and hormones) which are, in turn, received and interpreted by diverse cell receptors or membrane-bound proteins. These secreted polypeptides or signaling molecules normally pass through the cellular secretory pathway to reach their site of action in the extracellular environment.

Secreted proteins have various industrial applications, including as pharmaceuticals, diagnostics, biosensors and bioreactors. Most protein drugs available at present, such as thrombolytic agents, interferons, interleukins, erythropoietines, colony stimulating factors, and various other cytokines, are secretory proteins. Their receptors, which are membrane proteins, also have potential as therapeutic or diagnostic agents. Efforts are being undertaken by both industry and academia to identify new, native secreted proteins. Many efforts are focused on the screening of mammalian recombinant DNA libraries to identify the coding sequences for novel secreted proteins. Examples of screening methods and techniques are described in the literature [see, for example, Klein et al., Proc. Natl. Acad. Sci. 93:7108-7113 (1996); U.S. Pat. No. 5,536,637)].

Membrane-bound proteins and receptors can play important roles in, among other things, the formation, differentiation and maintenance of multicellular organisms. The fate of many individual cells, e.g., proliferation, migration, differentiation, or interaction with other cells, is typically governed by information received from other cells and/or the immediate environment. This information is often transmitted by secreted polypeptides (for instance, mitogenic factors, survival factors, cytotoxic factors, differentiation factors, neuropeptides, and hormones) which are, in turn, received and interpreted by diverse cell receptors or membrane-bound proteins. Such membrane-bound proteins and cell receptors include, but are not limited to, cytokine receptors, receptor kinases, receptor phosphatases, receptors involved in cell-cell interactions, and cellular adhesion molecules like selectins and integrins. For instance, transduction of signals that regulate cell growth and differentiation is regulated in part by phosphorylation of various cellular proteins. Protein tyrosine kinases, enzymes that catalyze that process, can also act as growth factor receptors. Examples include fibroblast growth factor receptor and nerve growth factor receptor.

Membrane-bound proteins and receptor molecules have various industrial applications, including as pharmaceutical and diagnostic agents. Receptor immuno-adhesions, for instance, can be employed as therapeutic agents to block receptor-ligand interactions. The membrane-bound proteins can also be employed for screening of potential peptide or small molecule inhibitors of the relevant receptor/ligand interaction.

Efforts are being undertaken by both industry and academia to identify new, native receptor or membrane-bound proteins. Many efforts are focused on the screening of mammalian recombinant DNA libraries to identify the coding sequences for novel receptor or membrane-bound proteins.

Given the importance of secreted and membrane-bound proteins in biological and disease processes, in vivo studies and characterizations may provide valuable identification and discovery of therapeutics and/or treatments useful in the prevention, amelioration or correction of diseases or dysfunctions. In this regard, genetically engineered mice have proven to be invaluable tools for the functional dissection of biological processes relevant to human disease, including immunology, cancer, neuro-biology, cardiovascular biology, obesity and many others. Gene knockouts can be viewed as modeling the biological mechanism of drug action by presaging the activity of highly specific antagonists in vivo. Knockout mice have been shown to model drug activity; phenotypes of mice deficient for specific pharmaceutical target proteins can resemble the human clinical phenotype caused by the corresponding antagonist drug. Gene knockouts enable the discovery of the mechanism of action of the target, the predominant physiological role of the target, and mechanism-based side-effects that might result from inhibition of the target in mammals. Examples of this type include mice deficient in the angiotensin converting enzyme (ACE) [Esther, C. R. et al., Lab. Invest., 74:953-965 (1996)] and cyclooxygenase-1 (COX1) genes [Langenbach, R. et al., Cell, 83:483-492 (1995)]. Conversely, knocking the gene out in the mouse can have an opposite phenotypic effect to that observed in humans after administration of an agonist drug to the corresponding target. Examples include the erythropoietin knockout [Wu, C. S. et al., Cell, 83:59-67 (1996)], in which a consequence of the mutation is deficient red blood cell production, and the GABA(A)-R-β3 knockout [DeLorey, T. M., J. Neurosci., 18:8505-8514 (1998)], in which the mutant mice show hyperactivity and hyper-responsiveness. Both these phenotypes are opposite to the effects of erythropoietin and benzodiazepine administration in humans. A striking example of a target validated using mouse genetics is the ACC2 gene. Although the human ACC2 gene had been identified several years ago, interest in ACC2 as a target for drug development was stimulated only recently after analysis of ACC2 function using a knockout mouse. ACC2 mutant mice eat more than their wild-type littermates, yet burn more fat and store less fat in their adipocytes, making this enzyme a probable target for chemical antagonism in the treatment of obesity [Abu-Elheiga, L. et al., Science, 291:2613-2616 (2001)].

In the instant application, mutated gene disruptions have resulted in phenotypic observations related to various disease conditions or dysfunctions including: CNS/neurological disturbances or disorders such as anxiety; eye abnormalities and associated diseases; cardiovascular, endothelial or angiogenic disorders including atherosclerosis; abnormal metabolic disorders including diabetes and dyslipidemias associated with elevated serum triglycerides and cholesterol levels; immunological and inflammatory disorders; oncological disorders; bone metabolic abnormalities or disorders such as arthritis, osteoporosis and osteopetrosis; or a developmental disease such as embryonic lethality.

### SUMMARY OF THE INVENTION

### A. Embodiments

The invention provides an isolated nucleic acid molecule comprising a nucleotide sequence that encodes a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide.

In one aspect, the isolated nucleic acid molecule comprises a nucleotide sequence having at least about 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity to (a) a DNA molecule encoding a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide having a full-length amino acid sequence as disclosed herein, an amino acid sequence lacking the signal peptide as disclosed herein, an extracellular domain of a transmembrane protein, with or without the signal peptide, as disclosed herein or any other specifically defined fragment of the full-length amino acid sequence as disclosed herein, or (b) the complement of the DNA molecule of (a).

In other aspects, the isolated nucleic acid molecule comprises a nucleotide sequence having at least about 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity to (a) a DNA molecule comprising the coding sequence of a full-length PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide cDNA as disclosed herein, the coding sequence of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide lacking the signal peptide as disclosed herein, the coding sequence of an extracellular domain of a transmembrane PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, with or without the signal peptide, as disclosed herein or the coding sequence of any other specifically defined fragment of the full-length amino acid sequence as disclosed herein, or (b) the complement of the DNA molecule of (a).

In a further aspect, the invention concerns an isolated nucleic acid molecule comprising a nucleotide sequence having at least about 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity to (a) a DNA molecule that encodes the same mature polypeptide encoded by any of the human protein cDNAs deposited with the ATCC as disclosed herein, or (b) the complement of the DNA molecule of (a).

Another aspect of the invention provides an isolated nucleic acid molecule comprising a nucleotide sequence encoding a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide which is either transmembrane domain-deleted or transmembrane domain-inactivated, or is complementary to such encoding nucleotide sequence, wherein the transmembrane domain(s) of such polypeptide are disclosed herein. Therefore, soluble extracellular domains of the herein described PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides are contemplated.

The invention also provides fragments of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide coding sequence, or the complement thereof, that may find use as, for example, hybridization probes, for encoding fragments of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide that may optionally encode a polypeptide comprising a binding site for an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody or as antisense oligonucleotide probes. Such nucleic acid fragments usually are or are at least about 10 nucleotides in length, alternatively are or are at least about 15 nucleotides in length, alternatively are or are at least about 20 nucleotides in length, alternatively are or are at least about 30 nucleotides in length, alternatively are or are at least about 40 nucleotides in length, alternatively are or are at least about 50 nucleotides in length, alternatively are or are at least about 60 nucleotides in length, alternatively are or are at least about 70 nucleotides in length, alternatively are or are at least about 80 nucleotides in length, alternatively are or are at least about 90 nucleotides in length, alternatively are or are at least about 100 nucleotides in length, alternatively are or are at least about 110 nucleotides in length, alternatively are or are at least about 120 nucleotides in length, alternatively are or are at least about 130 nucleotides in length, alternatively are or are at least about 140 nucleotides in length, alternatively are or are at least about 150 nucleotides in length, alternatively are or are at least about 160 nucleotides in length, alternatively are or are at least about 170 nucleotides in length, alternatively are or are at least about 180 nucleotides in length, alternatively are or are at least about 190 nucleotides in length, alternatively are or are at least about 200 nucleotides in length, alternatively are or are at least about 250 nucleotides in length, alternatively are or are at least about 300 nucleotides in length, alternatively are or are at least about 350 nucleotides in length, alternatively are or are at least about 400 nucleotides in length, alternatively are or are at least about 450 nucleotides in length, alternatively are or are at least about 500 nucleotides in length, alternatively are or are at least about 600 nucleotides in length, alternatively are or are at least about 700 nucleotides in length, alternatively are or are at least about 800 nucleotides in length, alternatively are or are at least about 900 nucleotides in length and alternatively are or are at least about 1000 nucleotides in length, wherein in this context the term “about” means the referenced nucleotide sequence length plus or minus 10% of that referenced length. It is noted that novel fragments of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide-encoding nucleotide sequence may be determined in a routine manner by aligning the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide-encoding nucleotide sequence with other known nucleotide sequences using any of a number of well known sequence alignment programs and determining which PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide-encoding nucleotide sequence fragment(s) are novel. All of such PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide-encoding nucleotide sequences are contemplated herein. Also contemplated are the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide fragments encoded by these nucleotide molecule fragments, preferably those PRO256, PRO34421, PRO334, PRO770, PRO983, PRO1009, PRO107, PRO1158, PRO1250, PRO1317, PRO4334, PRO4395, PRO49192, PRO9799, PRO21175, PRO19837, PRO21331, PRO23949, PRO697 or PRO1480 polypeptide fragments that comprise a binding site for an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.

The invention provides isolated PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides encoded by any of the isolated nucleic acid sequences hereinabove identified.

In a certain aspect, the invention concerns an isolated PRO256, PRO34421, PRO334, PRO770, PRO983, PRO1009, PRO107, PRO1158, PRO1250, PRO1317, PRO4334, PRO4395, PRO49192, PRO9799, PRO21175, PRO19837, PRO21331, PRO23949, PRO697 or PRO1480 polypeptide, comprising an amino acid sequence having at least about 80% amino acid sequence identity, alternatively at least about 81% amino acid sequence identity, alternatively at least about 82% amino acid sequence identity, alternatively at least about 83% amino acid sequence identity, alternatively at least about 84% amino acid sequence identity, alternatively at least about 85% amino acid sequence identity, alternatively at least about 86% amino acid sequence identity, alternatively at least about 87% amino acid sequence identity, alternatively at least about 88% amino acid sequence identity, alternatively at least about 89% amino acid sequence identity, alternatively at least about 90% amino acid sequence identity, alternatively at least about 91% amino acid sequence identity, alternatively at least about 92% amino acid sequence identity, alternatively at least about 93% amino acid sequence identity, alternatively at least about 94% amino acid sequence identity, alternatively at least about 95% amino acid sequence identity, alternatively at least about 96% amino acid sequence identity, alternatively at least about 97% amino acid sequence identity, alternatively at least about 98% amino acid sequence identity and alternatively at least about 99% amino acid sequence identity to a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide having a full-length amino acid sequence as disclosed herein, an amino acid sequence lacking the signal peptide as disclosed herein, an extracellular domain of a transmembrane protein, with or without the signal peptide, as disclosed herein or any other specifically defined fragment of the full-length amino acid sequence as disclosed herein.

In a further aspect, the invention concerns an isolated PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide comprising an amino acid sequence having at least about 80% amino acid sequence identity, alternatively at least about 81% amino acid sequence identity, alternatively at least about 82% amino acid sequence identity, alternatively at least about 83% amino acid sequence identity, alternatively at least about 84% amino acid sequence identity, alternatively at least about 85% amino acid sequence identity, alternatively at least about 86% amino acid sequence identity, alternatively at least about 87% amino acid sequence identity, alternatively at least about 88% amino acid sequence identity, alternatively at least about 89% amino acid sequence identity, alternatively at least about 90% amino acid sequence identity, alternatively at least about 91% amino acid sequence identity, alternatively at least about 92% amino acid sequence identity, alternatively at least about 93% amino acid sequence identity, alternatively at least about 94% amino acid sequence identity, alternatively at least about 95% amino acid sequence identity, alternatively at least about 96% amino acid sequence identity, alternatively at least about 97% amino acid sequence identity, alternatively at least about 98% amino acid sequence identity and alternatively at least about 99% amino acid sequence identity to an amino acid sequence encoded by any of the human protein cDNAs deposited with the ATCC as disclosed herein.

In one aspect, the invention concerns PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 variant polypeptides which are or are at least about 10 amino acids in length, alternatively are or are at least about 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600 amino acids in length, or more. Optionally, PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 variant polypeptides will have or have no more than one conservative amino acid substitution as compared to the native PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide sequence, alternatively will have or will have no more than 2, 3, 4, 5, 6, 7, 8, 9, or 10 conservative amino acid substitution as compared to the native PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide sequence.

In a specific aspect, the invention provides an isolated PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide without the N-terminal signal sequence and/or the initiating methionine and is encoded by a nucleotide sequence that encodes such an amino acid sequence as hereinbefore described. Processes for producing the same are also herein described, wherein those processes comprise culturing a host cell comprising a vector which comprises the appropriate encoding nucleic acid molecule under conditions suitable for expression of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide and recovering the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide from the cell culture.

Another aspect the invention provides an isolated PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide which is either transmembrane domain-deleted or transmembrane domain-inactivated. Processes for producing the same are also herein described, wherein those processes comprise culturing a host cell comprising a vector which comprises the appropriate encoding nucleic acid molecule under conditions suitable for expression of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide and recovering the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide from the cell culture.

The invention provides agonists and antagonists of a native PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide as defined herein. In particular, the agonist or antagonist is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody or a small molecule.

The invention provides a method of identifying agonists or antagonists to a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide which comprise contacting the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide with a candidate molecule and monitoring a biological activity mediated by said PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. Preferably, the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide is a native PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide.

The invention provides a composition of matter comprising a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, or an agonist or antagonist of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide as herein described, or an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody, in combination with a carrier. Optionally, the carrier is a pharmaceutically acceptable carrier.

The invention provides the use of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, or an agonist or antagonist thereof as hereinbefore described, or an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody, for the preparation of a medicament useful in the treatment of a condition which is responsive to the anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.

The invention provides vectors comprising DNA encoding any of the herein described polypeptides. Host cell comprising any such vector are also provided. By way of example, the host cells may be CHO cells, E. coli, or yeast. A process for producing any of the herein described polypeptides is further provided and comprises culturing host cells under conditions suitable for expression of the desired polypeptide and recovering the desired polypeptide from the cell culture.

The invention provides chimeric molecules comprising any of the herein described polypeptides fused to a heterologous polypeptide or amino acid sequence. Example of such chimeric molecules comprise any of the herein described polypeptides fused to an epitope tag sequence or a Fc region of an immunoglobulin.

The invention provides an antibody which binds, preferably specifically, to any of the above or below described polypeptides. Optionally, the antibody is a monoclonal antibody, humanized antibody, antibody fragment or single-chain antibody.

The invention provides oligonucleotide probes which may be useful for isolating genomic and cDNA nucleotide sequences, measuring or detecting expression of an associated gene or as antisense probes, wherein those probes may be derived from any of the above or below described nucleotide sequences. Preferred probe lengths are described above.

The invention also provides a method of identifying a phenotype associated with a disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising:

(a) providing a non-human transgenic animal whose genome comprises a disruption of the gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide;

(b) measuring a physiological characteristic of the non-human transgenic animal; and

(c) comparing the measured physiological characteristic with that of a gender matched wild-type animal, wherein the physiological characteristic of the non-human transgenic animal that differs from the physiological characteristic of the wild-type animal is identified as a phenotype resulting from the gene disruption in the non-human transgenic animal. In one aspect, the non-human transgenic animal is a mammal. In another aspect, the mammal is a rodent. In still another aspect, the mammal is a rat or a mouse. In one aspect, the non-human transgenic animal is heterozygous for the disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. In another aspect, the phenotype exhibited by the non-human transgenic animal as compared with gender matched wild-type littermates is at least one of the following: a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality.

In yet another aspect, the neurological disorder is an increased anxiety-like response during open field activity testing. In yet another aspect, the neurological disorder is a decreased anxiety-like response during open field activity testing. In yet another aspect, the neurological disorder is an abnormal circadian rhythm during home-cage activity testing. In yet another aspect, the neurological disorder is an enhanced motor coordination during inverted screen testing. In yet another aspect, the neurological disorder is impaired motor coordination during inverted screen testing. In yet another aspect, the neurological disorder includes depression, generalized anxiety disorders, attention deficit disorder, sleep disorder, hyperactivity disorder, obsessive compulsive disorder, schizophrenia, cognitive disorders, hyperalgesia and sensory disorders. Such neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, social anxiety, autism, specific phobia, substance-induced anxiety disorder, acute alcohol withdrawal, obsessive compulsive disorder, agoraphobia, monopolar disorders, bipolar disorder I or II, bipolar disorder not otherwise specified, cyclothymic disorder, depressive disorder, major depressive disorder, mood disorder, substance-induced mood disorder, enhancement of cognitive function, loss of cognitive function associated with but not limited to Alzheimer's disease, stroke, or traumatic injury to the brain, seizures resulting from disease or injury including but not limited to epilepsy, learning disorders/disabilities, cerebral palsy. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

In another aspect, the eye abnormality is a retinal abnormality. In still another aspect, the eye abnormality is consistent with vision problems or blindness. In yet another aspect, the retinal abnormality is consistent with retinitis pigmentosa or is characterized by retinal degeneration or retinal dysplasia.

In still another aspect, the retinal abnormalities are consistent with retinal dysplasia, various retinopathies, including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstrom's syndrome, Cockayne's syndrome, dysplasia spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis.

In still another aspect, the eye abnormality is a cataract. In still yet another aspect, the cataract is a systemic disease such as human Down's syndrome, Hallerman-Streiff syndrome, Lowe syndrome, galactosemia, Marfan syndrome, Trismoy 13-15, Alport syndrome, myotonic dystrophy, Fabry disease, hypoparathyroidism or Conradi syndrome.

In still another aspect, the developmental abnormality comprises embryonic lethality or reduced viability.

In still yet another aspect, the cardiovascular, endothelial or angiogenic disorders are arterial diseases, such as diabetes mellitus; papilledema; optic atrophy; atherosclerosis; angina; myocardial infarctions such as acute myocardial infarctions, cardiac hypertrophy, and heart failure such as congestive heart failure; hypertension; inflammatory vasculitides; Reynaud's disease and Reynaud's phenomenon; aneurysms and arterial restenosis; venous and lymphatic disorders such as thrombophlebitis, lymphangitis, and lymphedema; peripheral vascular disease; cancer such as vascular tumors, e.g., hemangioma (capillary and cavernous), glomus tumors, telangiectasia, bacillary angiomatosis, hemangioendothelioma, angiosarcoma, hemangiopericytoma, Kaposi's sarcoma, lymphangioma, and lymphangiosarcoma; tumor angiogenesis; trauma such as wounds, burns, and other injured tissue, implant fixation, scarring; ischemia reperfusion injury; rheumatoid arthritis; cerebrovascular disease; renal diseases such as acute renal failure, or osteoporosis.

In still another aspect, the immunological disorders are consistent with systemic lupus erythematosis; rheumatoid arthritis; juvenile chronic arthritis; spondyloarthropathies; systemic sclerosis (scleroderma); idiopathic inflammatory myopathies (dermatomyositis, polymyositis); Sjögren's syndrome; systemic vasculitis; sarcoidosis; autoimmune hemolytic anemia (immune pancytopenia, paroxysmal nocturnal hemoglobinuria); autoimmune thrombocytopenia (idiopathic thrombocytopenic purpura, immune-mediated thrombocytopenia); thyroiditis (Grave's disease, Hashimoto's thyroiditis, juvenile lymphocytic thyroiditis, atrophic thyroiditis); diabetes mellitus; immune-mediated renal disease (glomerulonephritis, tubulointerstitial nephritis); demyelinating diseases of the central and peripheral nervous systems such as multiple sclerosis, idiopathic demyelinating polyneuropathy or Guillain-Barré syndrome, and chronic inflammatory demyelinating polyneuropathy; hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis; inflammatory bowel disease (ulcerative colitis: Crohn's disease); gluten-sensitive enteropathy, and Whipple's disease; autoimmune or immune-mediated skin diseases including bullous skin diseases, erythema multiform and contact dermatitis, psoriasis; allergic diseases such as asthma, allergic rhinitis, atopic dermatitis, food hypersensitivity and urticaria; immunologic diseases of the lung such as eosinophilic pneumonia, idiopathic pulmonary fibrosis and hypersensitivity pneumonitis; or transplantation associated diseases including graft rejection and graft-versus-host disease.

In still another aspect, the bone metabolic abnormality or disorder is arthritis, osteoporosis, osteopenia or osteopetrosis.

In another aspect, the non-human transgenic animal exhibits at least one of the following physiological characteristics compared with gender matched wild-type littermates: a decreased anxiety-like response during open field activity testing; an abnormal circadian rhythm during home-cage activity testing; an enhanced motor coordination during inverted screen testing; exophthalamus in functional observation testing; severe retinal degeneration marked by attenuated retinal vessels; retinal microaneurisms; decreased mean artery-to-vein ratio; decreased lens size; mature cataracts; an increased mean serum cholesterol level; an increased mean serum triglyceride level; a decreased mean serum cholesterol level; an enhanced glucose tolerance; a decreased glucose tolerance; an increased mean serum insulin level; a decreased mean serum insulin level; a decreased mean serum IgG1 and IgG2a responses to an ovalbumin challenge; an increased mean serum IgG2a response to an ovalbumin challenge; an impaired IgG2a response to an ovalbumin challenge; a decreased mean absolute blood neutrophil count; an increased mean serum levels of IgG1, IgG3, IgA, IgG2a and IgG2b; an increased mean serum TNF-alpha and IL6 response to a LPS challenge; a decreased mean platelet count; a reduced level of RBC's, platelets, hemoglobin and hematocrit; an increased mean percent body fat; a decreased skin fibroblast proliferation; an increased skin fibroblast proliferation; an increased total tissue mass (TTM); an increased lean body mass (LBM); an increased bone mineral density (BMD); an increased bone mineral content (BMC), an increased bone mineral content index (BMC/LBM); an increased midshaft femur total area; a decrease in trabecular bone volume and connectivity density; a decreased volumetric bone mineral density; a decreased bone mineral content index (BMC/LBM); a decreased mean bone mineral density in total body, femur and vertebrate; a decreased mean bone mineral density (BMD), a decreased mean trabecular bone volume, decreased thickness, and decreased connectivity density; a decreased body weight and length; a decreased total tissue mass (TTM); a decreased lean body mass (LBM); a decreased total fat mass; a decreased bone mineral content (BMC); a decreased mean volumetric bone mineral density (vBMD) in total body and femur; a decreased femoral midshaft cross-sectional area and thickness; growth retardation with decreased mean body weight and length, decreased mean percent of total body fat, decreased total tissue mass and decreased bone mineral density; a decreased femoral midshaft cortical thickness; cardiomegaly; an impaired renal function; renal mesonephric duct development abnormalities; seminiferous tubular degeneration; greatly reduced viability [only three (−/−) mutant mice survived showing severe growth retardation as compared to the expected 14 (−/−) mutants]; a significant reduction in expected numbers of homozygotes; and embryonic lethality.

The invention also provides an isolated cell derived from a non-human transgenic animal whose genome comprises a disruption of the gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. In one aspect, the isolated cell is a murine cell. In yet another aspect, the murine cell is an embryonic stem cell. In still another aspect, the isolated cell is derived from a non-human transgenic animal which exhibits at least one of the following phenotypes compared with gender matched wild-type littermates: a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality. The invention also provides a method of identifying an agent that modulates a phenotype associated with a disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising:

(a) providing a non-human transgenic animal whose genome comprises a disruption of the gene which encodes for the PRO256, PRO34421, PRO334, PRO770, PRO983, PRO1009, PRO107, PRO1158, PRO1250, PRO1317, PRO4334, PRO4395, PRO49192, PRO9799, PRO21175, PRO19837, PRO21331, PRO23949, PRO697 or PRO1480 polypeptide;

(b) measuring a physiological characteristic of the non-human transgenic animal of (a);

(c) comparing the measured physiological characteristic of (b) with that of a gender matched wild-type animal, wherein the physiological characteristic of the non-human transgenic animal that differs from the physiological characteristic of the wild-type animal is identified as a phenotype resulting from the gene disruption in the non-human transgenic animal;

(d) administering a test agent to the non-human transgenic animal of (a); and

(e) determining whether the test agent modulates the identified phenotype associated with gene disruption in the non-human transgenic animal.

In one aspect, the phenotype associated with the gene disruption comprises a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality.

In yet another aspect, the neurological disorder is an increased anxiety-like response during open field activity testing. In yet another aspect, the neurological disorder is a decreased anxiety-like response during open field activity testing. In yet another aspect, the neurological disorder is an abnormal circadian rhythm during home-cage activity testing. In yet another aspect, the neurological disorder is an enhanced motor coordination during inverted screen testing. In yet another aspect, the neurological disorder is impaired motor coordination during inverted screen testing. In yet another aspect, the neurological disorder includes depression, generalized anxiety disorders, attention deficit disorder, sleep disorder, hyperactivity disorder, obsessive compulsive disorder, schizophrenia, cognitive disorders, hyperalgesia and sensory disorders. Such neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, social anxiety, autism, specific phobia, substance-induced anxiety disorder, acute alcohol withdrawal, obsessive compulsive disorder, agoraphobia, monopolar disorders, bipolar disorder I or II, bipolar disorder not otherwise specified, cyclothymic disorder, depressive disorder, major depressive disorder, mood disorder, substance-induced mood disorder, enhancement of cognitive function, loss of cognitive function associated with but not limited to Alzheimer's disease, stroke, or traumatic injury to the brain, seizures resulting from disease or injury including but not limited to epilepsy, learning disorders/disabilities, cerebral palsy. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

In yet another aspect, the eye abnormality is a retinal abnormality. In still another aspect, the eye abnormality is consistent with vision problems or blindness. In yet another aspect, the retinal abnormality is consistent with retinitis pigmentosa or is characterized by retinal degeneration or retinal dysplasia.

In still another aspect, the retinal abnormalities are consistent with retinal dysplasia, various retinopathies, including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstrom's syndrome, Cockayne's syndrome, dysplasia spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis.

In still another aspect, the eye abnormality is a cataract. In still yet another aspect, the cataract is a systemic disease such as human Down's syndrome, Hallerman-Streiff syndrome, Lowe syndrome, galactosemia, Marfan syndrome, Trismoy 13-15, Alport syndrome, myotonic dystrophy, Fabry disease, hypoparathyroidism, or Conradi syndrome.

In still another aspect, the developmental abnormality comprises embryonic lethality or reduced viability.

In still another aspect, the cardiovascular, endothelial or angiogenic disorders are arterial diseases, such as diabetes mellitus; papilledema; optic atrophy; atherosclerosis; angina; myocardial infarctions such as acute myocardial infarctions, cardiac hypertrophy, and heart failure such as congestive heart failure; hypertension; inflammatory vasculitides; Reynaud's disease and Reynaud's phenomenon; aneurysms and arterial restenosis; venous and lymphatic disorders such as thrombophlebitis, lymphangitis, and lymphedema; peripheral vascular disease; cancer such as vascular tumors, e.g., hemangioma (capillary and cavernous), glomus tumors, telangiectasia, bacillary angiomatosis, hemangioendothelioma, angiosarcoma, hemangiopericytoma, Kaposi's sarcoma, lymphangioma, and lymphangiosarcoma; tumor angiogenesis; trauma such as wounds, burns, and other injured tissue, implant fixation, scarring; ischemia reperfusion injury; rheumatoid arthritis; cerebrovascular disease; renal diseases such as acute renal failure, or osteoporosis.

In still another aspect, the immunological disorders are consistent with systemic lupus erythematosis; rheumatoid arthritis; juvenile chronic arthritis; spondyloarthropathies; systemic sclerosis (scleroderma); idiopathic inflammatory myopathies (dermatomyositis, polymyositis); Sjögren's syndrome; systemic vasculitis; sarcoidosis; autoimmune hemolytic anemia (immune pancytopenia, paroxysmal nocturnal hemoglobinuria); autoimmune thrombocytopenia (idiopathic thrombocytopenic purpura, immune-mediated thrombocytopenia); thyroiditis (Grave's disease, Hashimoto's thyroiditis, juvenile lymphocytic thyroiditis, atrophic thyroiditis); diabetes mellitus; immune-mediated renal disease (glomerulonephritis, tubulointerstitial nephritis); demyelinating diseases of the central and peripheral nervous systems such as multiple sclerosis, idiopathic demyelinating polyneuropathy or Guillain-Barré syndrome, and chronic inflammatory demyelinating polyneuropathy; hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis; inflammatory bowel disease (ulcerative colitis: Crohn's disease); gluten-sensitive enteropathy, and Whipple's disease; autoimmune or immune-mediated skin diseases including bullous skin diseases, erythema multiform and contact dermatitis, psoriasis; allergic diseases such as asthma, allergic rhinitis, atopic dermatitis, food hypersensitivity and urticaria; immunologic diseases of the lung such as eosinophilic pneumonia, idiopathic pulmonary fibrosis and hypersensitivity pneumonitis; or transplantation associated diseases including graft rejection and graft-versus-host disease.

In yet another aspect, the bone metabolic abnormality or disorder is arthritis, osteoporosis, osteopenia or osteopetrosis.

In another aspect, the non-human transgenic animal exhibits at least one of the following physiological characteristics compared with gender matched wild-type littermates: a decreased anxiety-like response during open field activity testing; an abnormal circadian rhythm during home-cage activity testing; an enhanced motor coordination during inverted screen testing; exophthalamus in functional observation testing; severe retinal degeneration marked by attenuated retinal vessels; retinal microaneurisms; decreased mean artery-to-vein ratio; decreased lens size; mature cataracts; an increased mean serum cholesterol level; an increased mean serum triglyceride level; a decreased mean serum cholesterol level; an enhanced glucose tolerance; a decreased glucose tolerance; an increased mean serum insulin level; a decreased mean serum insulin level; a decreased mean serum IgG1 and IgG2a responses to an ovalbumin challenge; an increased mean serum IgG2a response to an ovalbumin challenge; an impaired IgG2a response to an ovalbumin challenge; a decreased mean absolute blood neutrophil count; an increased mean serum levels of IgG1, IgG3, IgA, IgG2a and IgG2b; an increased mean serum TNF-alpha and IL6 response to a LPS challenge; a decreased mean platelet count; a reduced level of RBC's, platelets, hemoglobin and hematocrit; an increased mean percent body fat; a decreased skin fibroblast proliferation; an increased skin fibroblast proliferation; an increased total tissue mass (TTM); an increased lean body mass (LBM); an increased bone mineral density (BMD); an increased bone mineral content (BMC), an increased bone mineral content index (BMC/LBM); an increased midshaft femur total area; a decrease in trabecular bone volume and connectivity density; a decreased volumetric bone mineral density; a decreased bone mineral content index (BMC/LBM); a decreased mean bone mineral density in total body, femur and vertebrate; a decreased mean bone mineral density (BMD), a decreased mean trabecular bone volume, decreased thickness, and decreased connectivity density; a decreased body weight and length; a decreased total tissue mass (TTM); a decreased lean body mass (LBM); a decreased total fat mass; a decreased bone mineral content (BMC); a decreased mean volumetric bone mineral density (vBMD) in total body and femur; a decreased femoral midshaft cross-sectional area and thickness; growth retardation with decreased mean body weight and length, decreased mean percent of total body fat, decreased total tissue mass and decreased bone mineral density; a decreased femoral midshaft cortical thickness; cardiomegaly; an impaired renal function; renal mesonephric duct development abnormalities; seminiferous tubular degeneration; greatly reduced viability [only three (−/−) mutant mice survived showing severe growth retardation as compared to the expected 14 (−/−) mutants]; a significant reduction in expected numbers of homozygotes; and embryonic lethality.

The invention also provides an agent which modulates the phenotype associated with gene disruption. In one aspect, the agent is an agonist or antagonist of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. In yet another aspect, the agonist agent is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody. In still another aspect, the antagonist agent is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.

The invention also provides a method of identifying an agent that modulates a physiological characteristic associated with a disruption of the gene which encodes for a PRO256, PRO34421, PRO334, PRO770, PRO983, PRO1009, PRO107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising:

(a) providing a non-human transgenic animal whose genome comprises a disruption of the gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide;

(b) measuring a physiological characteristic exhibited by the non-human transgenic animal of (a);

(c) comparing the measured physiological characteristic of (b) with that of a gender matched wild-type animal, wherein the physiological characteristic exhibited by the non-human transgenic animal that differs from the physiological characteristic exhibited by the wild-type animal is identified as a physiological characteristic associated with gene disruption;

(d) administering a test agent to the non-human transgenic animal of (a); and

(e) determining whether the physiological characteristic associated with gene disruption is modulated.

In one aspect, the non-human transgenic animal exhibits at least one of the following physiological characteristics compared with gender matched wild-type littermates:

In another aspect, the non-human transgenic animal exhibits at least one of the following physiological characteristics compared with gender matched wild-type littermates: a decreased anxiety-like response during open field activity testing; an abnormal circadian rhythm during home-cage activity testing; an enhanced motor coordination during inverted screen testing; exophthalamus in functional observation testing; severe retinal degeneration marked by attenuated retinal vessels; retinal microaneurisms; decreased mean artery-to-vein ratio; decreased lens size; mature cataracts; an increased mean serum cholesterol level; an increased mean serum triglyceride level; a decreased mean serum cholesterol level; an enhanced glucose tolerance; a decreased glucose tolerance; an increased mean serum insulin level; a decreased mean serum insulin level; a decreased mean serum IgG1 and IgG2a responses to an ovalbumin challenge; an increased mean serum IgG2a response to an ovalbumin challenge; an impaired IgG2a response to an ovalbumin challenge; a decreased mean absolute blood neutrophil count; an increased mean serum levels of IgG1, IgG3, IgA, IgG2a and IgG2b; an increased mean serum TNF-alpha and IL6 response to a LPS challenge; a decreased mean platelet count; a reduced level of RBC's, platelets, hemoglobin and hematocrit; an increased mean percent body fat; a decreased, skin fibroblast proliferation; an increased skin fibroblast proliferation; an increased total tissue mass (TTM); an increased lean body mass (LBM); an increased bone mineral density (BMD); an increased bone mineral content (BMC), an increased bone mineral content index (BMC/LBM); an increased midshaft femur total area; a decrease in trabecular bone volume and connectivity density; a decreased volumetric bone mineral density; a decreased bone mineral content index (BMC/LBM); a decreased mean bone mineral density in total body, femur and vertebrate; a decreased mean bone mineral density (BMD), a decreased mean trabecular bone volume, decreased thickness, and decreased connectivity density; a decreased body weight and length; a decreased total tissue mass (TTM); a decreased lean body mass (LBM); a decreased total fat mass; a decreased bone mineral content (BMC); a decreased mean volumetric bone mineral density (vBMD) in total body and femur; a decreased femoral midshaft cross-sectional area and thickness; growth retardation with decreased mean body weight and length, decreased mean percent of total body fat, decreased total tissue mass and decreased bone mineral density; a decreased femoral midshaft cortical thickness; cardiomegaly; an impaired renal function; renal mesonephric duct development abnormalities; seminiferous tubular degeneration; greatly reduced viability [only three (−/−) mutant mice survived showing severe growth retardation as compared to the expected 14 (−/−) mutants]; a significant reduction in expected numbers of homozygotes; and embryonic lethality.

The invention also provides an agent that modulates a physiological characteristic which is associated with gene disruption. In one aspect, the agent is an agonist or antagonist of the phenotype associated with a disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. In yet another aspect, the agent is an agonist or antagonist of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. In yet another aspect, the agonist agent is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody. In still another aspect, the antagonist agent is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.

The invention also provides a method of identifying an agent which modulates a behavior associated with a disruption of the gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising:

(a) providing a non-human transgenic animal whose genome comprises a disruption of the gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide;

(b) observing the behavior exhibited by the non-human transgenic animal of (a);

(c) comparing the observed behavior of (b) with that of a gender matched wild-type animal, wherein the observed behavior exhibited by the non-human transgenic animal that differs from the observed behavior exhibited by the wild-type animal is identified as a behavior associated with gene disruption;

(d) administering a test agent to the non-human transgenic animal of (a); and

(e) determining whether the agent modulates the behavior associated with gene disruption.

In one aspect, the observed behavior is an increased anxiety-like response during open field activity testing. In yet another aspect, the observed behavior is a decreased anxiety-like response during open field activity testing. In yet another aspect, the observed behavior is an abnormal circadian rhythm during home-cage activity testing. In yet another aspect, the observed behavior is an enhanced motor coordination during inverted screen testing. In yet another aspect, the observed behavior is impaired motor coordination during inverted screen testing. In yet another aspect, the observed behavior includes depression, generalized anxiety disorders, attention deficit disorder, sleep disorder, hyperactivity disorder, obsessive compulsive disorder, schizophrenia, cognitive disorders, hyperalgesia and sensory disorders. Such disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, social anxiety, autism, specific phobia, substance-induced anxiety disorder, acute alcohol withdrawal, obsessive compulsive disorder, agoraphobia, monopolar disorders, bipolar disorder I or II, bipolar disorder not otherwise specified, cyclothymic disorder, depressive disorder, major depressive disorder, mood disorder, substance-induced mood disorder, enhancement of cognitive function, loss of cognitive function associated with but not limited to Alzheimer's disease, stroke, or traumatic injury to the brain, seizures resulting from disease or injury including but not limited to epilepsy, learning disorders/disabilities, cerebral palsy. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

The invention also provides an agent that modulates a behavior which is associated with gene disruption. In one aspect, the agent is an agonist or antagonist of the phenotype associated with a disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. In yet another aspect, the agent is an agonist or antagonist of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. In yet another aspect, the agonist agent is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody. In still another aspect, the antagonist agent is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.

The invention also provides a method of identifying an agent that ameliorates or modulates a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality associated with a disruption in the gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising:

(a) providing a non-human transgenic animal whose genome comprises a disruption of the gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide;

(b) administering a test agent to said non-human transgenic animal; and

(c) determining whether the test agent ameliorates or modulates the neurological disorder; cardiovascular, endothelial or angiogenic disorder; eye abnormality; immunological disorder; oncological disorder; bone metabolic abnormality or disorder; lipid metabolic disorder; or developmental abnormality associated with the gene disruption in the non-human transgenic animal.

In yet another aspect, the neurological disorder is an increased anxiety-like response during open field activity testing. In yet another aspect, the neurological disorder is a decreased anxiety-like response during open field activity testing. In yet another aspect, the neurological disorder is an abnormal circadian rhythm during home-cage activity testing. In yet another aspect, the neurological disorder is an enhanced motor coordination during inverted screen testing. In yet another aspect, the neurological disorder is impaired motor coordination during inverted screen testing. In yet another aspect, the neurological disorder includes depression, generalized anxiety disorders, attention deficit disorder, sleep disorder, hyperactivity disorder, obsessive compulsive disorder, schizophrenia, cognitive disorders, hyperalgesia and sensory disorders. Such neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, social anxiety, autism, specific phobia, substance-induced anxiety disorder, acute alcohol withdrawal, obsessive compulsive disorder, agoraphobia, monopolar disorders, bipolar disorder I or II, bipolar disorder not otherwise specified, cyclothymic disorder, depressive disorder, major depressive disorder, mood disorder, substance-induced mood disorder, enhancement of cognitive function, loss of cognitive function associated with but not limited to Alzheimer's disease, stroke, or traumatic injury to the brain, seizures resulting from disease or injury including but not limited to epilepsy, learning disorders/disabilities, cerebral palsy. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

In another aspect, the eye abnormality is a retinal abnormality. In still another aspect, the eye abnormality is consistent with vision problems or blindness. In yet another aspect, the retinal abnormality is consistent with retinitis pigmentosa or is characterized by retinal degeneration or retinal dysplasia.

In still another aspect, the retinal abnormalities the retinal abnormalities are consistent with retinal dysplasia, various retinopathies, including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedi syndrome, Alport's syndrome, Alstrom's syndrome, Cockayne's syndrome, dysplasia spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis.

In still another aspect, the eye abnormality is a cataract. In still yet another aspect, the cataract is a systemic disease such as human Down's syndrome, Hallerman-Streiff syndrome, Lowe syndrome, galactosemia, Marfan syndrome, Trismoy 13-15, Alport syndrome, myotonic dystrophy, Fabry disease, hypoparathyroidism, or Conradi syndrome.

In still another aspect, the developmental abnormality comprises embryonic lethality or reduced viability.

In yet another aspect, the cardiovascular, endothelial or angiogenic disorders are arterial diseases, such as diabetes mellitus; papilledema; optic atrophy; atherosclerosis; angina; myocardial infarctions such as acute myocardial infarctions, cardiac hypertrophy, and heart failure such as congestive heart failure; hypertension; inflammatory vasculitides; Reynaud's disease and Reynaud's phenomenon; aneurysms and arterial restenosis; venous and lymphatic disorders such as thrombophlebitis, lymphangitis, and lymphedema; peripheral vascular disease; cancer such as vascular tumors, e.g., hemangioma (capillary and cavernous), glomus tumors, telangiectasia, bacillary angiomatosis, hemangioendothelioma, angiosarcoma, hemangiopericytoma, Kaposi's sarcoma, lymphangioma, and lymphangiosarcoma; tumor angiogenesis; trauma such as wounds, burns, and other injured tissue, implant fixation, scarring; ischemia reperfusion injury; rheumatoid arthritis; cerebrovascular disease; renal diseases such as acute renal failure, or osteoporosis.

In still yet another aspect, the immunological disorders are consistent with systemic lupus erythematosis; rheumatoid arthritis; juvenile chronic arthritis; spondyloarthropathies; systemic sclerosis (scleroderma); idiopathic inflammatory myopathies (dermatomyositis, polymyositis); Sjögren's syndrome; systemic vasculitis; sarcoidosis; autoimmune hemolytic anemia (immune pancytopenia, paroxysmal nocturnal hemoglobinuria); autoimmune thrombocytopenia (idiopathic thrombocytopenic purpura, immune-mediated thrombocytopenia); thyroiditis (Grave's disease, Hashimoto's thyroiditis, juvenile lymphocytic thyroiditis, atrophic thyroiditis); diabetes mellitus; immune-mediated renal disease (glomerulonephritis, tubulointerstitial nephritis); demyelinating diseases of the central and peripheral nervous systems such as multiple sclerosis, idiopathic demyelinating polyneuropathy or Guillain-Barré syndrome, and chronic inflammatory demyelinating polyneuropathy; hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis; inflammatory bowel disease (ulcerative colitis: Crohn's disease); gluten-sensitive enteropathy, and Whipple's disease; autoimmune or immune-mediated skin diseases including bullous skin diseases, erythema multiform and contact dermatitis, psoriasis; allergic diseases such as asthma, allergic rhinitis, atopic dermatitis, food hypersensitivity and urticaria; immunologic diseases of the lung such as eosinophilic pneumonia, idiopathic pulmonary fibrosis and hypersensitivity pneumonitis; or transplantation associated diseases including graft rejection and graft-versus-host disease.

In yet another aspect, the bone metabolic abnormality or disorder is arthritis, osteoporosis, osteopenia or osteopetrosis.

In another aspect, the non-human transgenic animal exhibits at least one of the following physiological characteristics compared with gender matched wild-type littermates: a decreased anxiety-like response during open field activity testing; an abnormal circadian rhythm during home-cage activity testing; an enhanced motor coordination during inverted screen testing; exophthalamus in functional observation testing; severe retinal degeneration marked by attenuated retinal vessels; retinal microaneurisms; decreased mean artery-to-vein ratio; decreased lens size; mature cataracts; an increased mean serum cholesterol level; an increased mean serum triglyceride level; a decreased mean serum cholesterol level; an enhanced glucose tolerance; a decreased glucose tolerance; an increased mean serum insulin level; a decreased mean serum insulin level; a decreased mean serum IgG1 and IgG2a responses to an ovalbumin challenge; an increased mean serum IgG2a response to an ovalbumin challenge; an impaired IgG2a response to an ovalbumin challenge; a decreased mean absolute blood neutrophil count; an increased mean serum levels of IgG1, IgG3, IgA, IgG2a and IgG2b; an increased mean serum TNF-alpha and IL6 response to a LPS challenge; a decreased mean platelet count; a reduced level of RBC's, platelets, hemoglobin and hematocrit; an increased mean percent body fat; a decreased skin fibroblast proliferation; an increased skin fibroblast proliferation; an increased total tissue mass (TTM); an increased lean body mass (LBM); an increased bone mineral density (BMD); an increased bone mineral content (BMC), an increased bone mineral content index (BMC/LBM); an increased midshaft femur total area; a decrease in trabecular bone volume and connectivity density; a decreased volumetric bone mineral density; a decreased bone mineral content index (BMC/LBM); a decreased mean bone mineral density in total body, femur and vertebrate; a decreased mean bone mineral density (BMD), a decreased mean trabecular bone volume, decreased thickness, and decreased connectivity density; a decreased body weight and length; a decreased total tissue mass (TTM); a decreased lean body mass (LBM); a decreased total fat mass; a decreased bone mineral content (BMC); a decreased mean volumetric bone mineral density (vBMD) in total body and femur; a decreased femoral midshaft cross-sectional area and thickness; growth retardation with decreased mean body weight and length, decreased mean percent of total body fat, decreased total tissue mass and decreased bone mineral density; a decreased femoral midshaft cortical thickness; cardiomegaly; an impaired renal function; renal mesonephric duct development abnormalities; seminiferous tubular degeneration; greatly reduced viability [only three (−/−) mutant mice survived showing severe growth retardation as compared to the expected 14 (−/−) mutants]; a significant reduction in expected numbers of homozygotes; and embryonic lethality.

The invention also provides an agent that ameliorates or modulates a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality which is associated with gene disruption. In one aspect, the agent is an agonist or antagonist of the phenotype associated with a disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. In yet another aspect, the agent is an agonist or antagonist of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. In yet another aspect, the agonist agent is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody. In still another aspect, the antagonist agent is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.

The invention also provides a therapeutic agent for the treatment of a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality.

The invention also provides a method of identifying an agent that modulates the expression of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising:

(a) contacting a test agent with a host cell expressing a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide; and

(b) determining whether the test agent modulates the expression of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide by the host cell.

The invention also provides an agent that modulates the expression of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. In one aspect, the agent is an agonist or antagonist of the phenotype associated with a disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. In yet another aspect, the agent is an agonist or antagonist of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. In yet another aspect, the agonist agent is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody. In still another aspect, the antagonist agent is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.

The invention also provides a method of evaluating a therapeutic agent capable of affecting a condition associated with a disruption of a gene which encodes for a PRO256, PRO34421, PRO334, PRO770, PRO983, PRO1009, PRO107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising:

(a) providing a non-human transgenic animal whose genome comprises a disruption of the gene which encodes for the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide;

(b) measuring a physiological characteristic of the non-human transgenic animal of (a);

(c) comparing the measured physiological characteristic of (b) with that of a gender matched wild-type animal, wherein the physiological characteristic of the non-human transgenic animal that differs from the physiological characteristic of the wild-type animal is identified as a condition resulting from the gene disruption in the non-human transgenic animal;

(d) administering a test agent to the non-human transgenic animal of (a); and

(e) evaluating the effects of the test agent on the identified condition associated with gene disruption in the non-human transgenic animal.

In one aspect, the condition is a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality.

The invention also provides a therapeutic agent which is capable of affecting a condition associated with gene disruption. In one aspect, the agent is an agonist or antagonist of the phenotype associated with a disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. In yet another aspect, the agent is an agonist or antagonist of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. In yet another aspect, the agonist agent is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody. In still another aspect, the antagonist agent is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.

The invention also provides a pharmaceutical composition comprising a therapeutic agent capable of affecting the condition associated with gene disruption.

The invention also provides a method of treating or preventing or ameliorating a neurological disorder; cardiovascular, endothelial or angiogenic disorder; immunological disorder; oncological disorder; bone metabolic abnormality or disorder, or embryonic lethality associated with the disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising administering to a subject in need of such treatment whom may already have the disorder, or may be prone to have the disorder or may be in whom the disorder is to be prevented, a therapeutically effective amount of a therapeutic agent, or agonists or antagonists thereof, thereby effectively treating or preventing or ameliorating said disorder or disease.

In yet another aspect, the neurological disorder is an increased anxiety-like response during open field activity testing. In yet another aspect, the neurological disorder is a decreased anxiety-like response during open field activity testing. In yet another aspect, the neurological disorder is an abnormal circadian rhythm during home-cage activity testing. In yet another aspect, the neurological disorder is an enhanced motor coordination during inverted screen testing. In yet another aspect, the neurological disorder is impaired motor coordination during inverted screen testing. In yet another aspect, the neurological disorder includes depression, generalized anxiety disorders, attention deficit disorder, sleep disorder, hyperactivity disorder, obsessive compulsive disorder, schizophrenia, cognitive disorders, hyperalgesia and sensory disorders. Such neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, social anxiety, autism, specific phobia, substance-induced anxiety disorder, acute alcohol withdrawal, obsessive compulsive disorder, agoraphobia, monopolar disorders, bipolar disorder I or II, bipolar disorder not otherwise specified, cyclothymic disorder, depressive disorder, major depressive disorder, mood disorder, substance-induced mood disorder, enhancement of cognitive function, loss of cognitive function associated with but not limited to Alzheimer's disease, stroke, or traumatic injury to the brain, seizures resulting from disease or injury including but not limited to epilepsy, learning disorders/disabilities, cerebral palsy. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

In another aspect, the eye abnormality is a retinal abnormality. In still another aspect, the eye abnormality is consistent with vision problems or blindness. In yet another aspect, the retinal abnormality is consistent with retinitis pigmentosa or is characterized by retinal degeneration or retinal dysplasia.

In still another aspect, the retinal abnormalities are consistent with retinal dysplasia, various retinopathies, including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstrom's syndrome, Cockayne's syndrome, dysplasia spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis.

In still another aspect, the eye abnormality is a cataract. In still yet another aspect, the cataract is a systemic disease such as human Down's syndrome, Hallerman-Streiff syndrome, Lowe syndrome, galactosemia, Marfan syndrome, Trismoy 13-15, Alport syndrome, myotonic dystrophy, Fabry disease, hypoparathyroidism or Conradi syndrome.

In still another aspect, the developmental abnormality comprises embryonic lethality or reduced viability.

In yet another aspect, the cardiovascular, endothelial or angiogenic disorders are arterial diseases, such as diabetes mellitus; papilledema; optic atrophy; atherosclerosis; angina; myocardial infarctions such as acute myocardial infarctions, cardiac hypertrophy, and heart failure such as congestive heart failure; hypertension; inflammatory vasculitides; Reynaud's disease and Reynaud's phenomenon; aneurysms and arterial restenosis; venous and lymphatic disorders such as thrombophlebitis, lymphangitis, and lymphedema; peripheral vascular disease; cancer such as vascular tumors, e.g., hemangioma (capillary and cavernous), glomus tumors, telangiectasia, bacillary angiomatosis, hemangioendothelioma, angiosarcoma, hemangiopericytoma, Kaposi's sarcoma, lymphangioma, and lymphangiosarcoma; tumor angiogenesis; trauma such as wounds, burns, and other injured tissue, implant fixation, scarring; ischemia reperfusion injury; rheumatoid arthritis; cerebrovascular disease; renal diseases such as acute renal failure, or osteoporosis.

In still yet another aspect, the immunological disorders are consistent with systemic lupus erythematosis; rheumatoid arthritis; juvenile chronic arthritis; spondyloarthropathies; systemic sclerosis (scleroderma); idiopathic inflammatory myopathies (dermatomyositis, polymyositis); Sjögren's syndrome; systemic vasculitis; sarcoidosis; autoimmune hemolytic anemia (immune pancytopenia, paroxysmal nocturnal hemoglobinuria); autoimmune thrombocytopenia (idiopathic thrombocytopenic purpura, immune-mediated thrombocytopenia); thyroiditis (Grave's disease, Hashimoto's thyroiditis, juvenile lymphocytic thyroiditis, atrophic thyroiditis); diabetes mellitus; immune-mediated renal disease (glomerulonephritis, tubulointerstitial nephritis); demyelinating diseases of the central and peripheral nervous systems such as multiple sclerosis, idiopathic demyelinating polyneuropathy or Guillain-Barré syndrome, and chronic inflammatory demyelinating polyneuropathy; hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis; inflammatory bowel disease (ulcerative colitis: Crohn's disease); gluten-sensitive enteropathy, and Whipple's disease; autoimmune or immune-mediated skin diseases including bullous skin diseases, erythema multiform and contact dermatitis, psoriasis; allergic diseases such as asthma, allergic rhinitis, atopic dermatitis, food hypersensitivity and urticaria; immunologic diseases of the lung such as eosinophilic pneumonia, idiopathic pulmonary fibrosis and hypersensitivity pneumonitis; or transplantation associated diseases including graft rejection and graft-versus-host disease.

In yet another aspect, the bone metabolic abnormality or disorder is arthritis, osteoporosis, osteopenia or osteopetrosis.

In another aspect the therapeutic agent is an agonist or antagonist of the phenotype associated with a disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. In yet another aspect, the agent is an agonist or antagonist of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. In yet another aspect, the agonist agent is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody. Instill another aspect, the antagonist agent is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.

The invention also provides a method of identifying an agent that ameliorates or modulates a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality associated with a disruption in the gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising:

(a) providing a non-human transgenic animal cell culture, each cell of said culture comprising a disruption of the gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide;

(b) administering a test agent to said cell culture; and

(c) determining whether the test agent ameliorates or modulates the neurological disorder; cardiovascular, endothelial or angiogenic disorder; eye abnormality; immunological disorder; oncological disorder; bone metabolic abnormality or disorder; lipid metabolic disorder; or developmental abnormality in said culture. In yet another aspect, the neurological disorder is an increased anxiety-like response during open field activity testing. In yet another aspect, the neurological disorder is a decreased anxiety-like response during open field activity testing. In yet another aspect, the neurological disorder is an abnormal circadian rhythm during home-cage activity testing.

In yet another aspect, the neurological disorder is an enhanced motor coordination during inverted screen testing. In yet another aspect, the neurological disorder is impaired motor coordination during inverted screen testing. In yet another aspect, the neurological disorder includes depression, generalized anxiety disorders, attention deficit disorder, sleep disorder, hyperactivity disorder, obsessive compulsive disorder, schizophrenia, cognitive disorders, hyperalgesia and sensory disorders. Such neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, social anxiety, autism, specific phobia, substance-induced anxiety disorder, acute alcohol withdrawal, obsessive compulsive disorder, agoraphobia, monopolar disorders, bipolar disorder I or II, bipolar disorder not otherwise specified, cyclothymic disorder, depressive disorder, major depressive disorder, mood disorder, substance-induced mood disorder, enhancement of cognitive function, loss of cognitive function associated with but not limited to Alzheimer's disease, stroke, or traumatic injury to the brain, seizures resulting from disease or injury including but not limited to epilepsy, learning disorders/disabilities, cerebral palsy. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

In another aspect, the eye abnormality is a retinal abnormality. In still another aspect, the eye abnormality is consistent with vision problems or blindness. In yet another aspect, the retinal abnormality is consistent with retinitis pigmentosa or is characterized by retinal degeneration or retinal dysplasia.

In still another aspect, the retinal abnormalities are consistent with retinal dysplasia, various retinopathies, including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including-Grave's disease), corneal and other tissue transplantation, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstrom's syndrome, Cockayne's syndrome, dysplasia spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis.

In still another aspect, the eye abnormality is a cataract. In still yet another aspect, the cataract is a systemic disease such as human Down's syndrome, Hallerman-Streiff syndrome, Lowe syndrome, galactosemia, Marfan syndrome, Trismoy 13-15, Alport syndrome, myotonic dystrophy, Fabry disease, hypoparathyroidism or Conradi syndrome.

In still another aspect, the developmental abnormality comprises embryonic lethality or reduced viability.

In yet another aspect, the cardiovascular, endothelial or angiogenic disorders are arterial diseases, such as diabetes mellitus; papilledema; optic atrophy; atherosclerosis; angina; myocardial infarctions such as acute myocardial infarctions, cardiac hypertrophy, and heart failure such as congestive heart failure; hypertension; inflammatory vasculitides; Reynaud's disease and Reynaud's phenomenon; aneurysms and arterial restenosis; venous and lymphatic disorders such as thrombophlebitis, lymphangitis, and lymphedema; peripheral vascular disease; cancer such as vascular tumors, e.g., hemangioma (capillary and cavernous), glomus tumors, telangiectasia, bacillary angiomatosis, hemangioendothelioma, angiosarcoma, hemangiopericytoma, Kaposi's sarcoma, lymphangioma, and lymphangiosarcoma; tumor angiogenesis; trauma such as wounds, burns, and other injured tissue, implant fixation, scarring; ischemia reperfusion injury; rheumatoid arthritis; cerebrovascular disease; renal diseases such as acute renal failure, or osteoporosis.

In still yet another aspect, the immunological disorders are consistent with systemic lupus erythematosis; rheumatoid arthritis; juvenile chronic arthritis; spondyloarthropathies; systemic sclerosis (scleroderma); idiopathic inflammatory myopathies (dermatomyositis, polymyositis); Sjögren's syndrome; systemic vasculitis; sarcoidosis; autoimmune hemolytic anemia (immune pancytopenia, paroxysmal nocturnal hemoglobinuria); autoimmune thrombocytopenia (idiopathic thrombocytopenic purpura, immune-mediated thrombocytopenia); thyroiditis (Grave's disease, Hashimoto's thyroiditis, juvenile lymphocytic thyroiditis, atrophic thyroiditis); diabetes mellitus; immune-mediated renal disease (glomerulonephritis, tubulointerstitial nephritis); demyelinating diseases of the central and peripheral nervous systems such as multiple sclerosis, idiopathic demyelinating polyneuropathy or Guillain-Barré syndrome, and chronic inflammatory demyelinating polyneuropathy; hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis; inflammatory bowel disease (ulcerative colitis: Crohn's disease); gluten-sensitive enteropathy, and Whipple's disease; autoimmune or immune-mediated skin diseases including bullous skin diseases, erythema multiform and contact dermatitis, psoriasis; allergic diseases such as asthma, allergic rhinitis, atopic dermatitis, food hypersensitivity and urticaria; immunologic diseases of the lung such as eosinophilic pneumonia, idiopathic pulmonary fibrosis and hypersensitivity pneumonitis; or transplantation associated diseases including graft rejection and graft-versus-host disease.

In yet another aspect, the bone metabolic abnormality or disorder is arthritis, osteoporosis, osteopenia or osteopetrosis.

The invention also provides an agent that ameliorates or modulates a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality which is associated with gene disruption in said culture. In one aspect, the agent is an agonist or antagonist of the phenotype associated with a disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. In yet another aspect, the agent is an agonist or antagonist of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. In yet another aspect, the agonist agent is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody. In still another aspect, the antagonist agent is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.

The invention also provides a method of modulating a phenotype associated with a disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising administering to a subject whom may already have the phenotype, or may be prone to have the phenotype or may be in whom the phenotype is to be prevented, an effective amount of an agent identified as modulating said phenotype, or agonists or antagonists thereof, thereby effectively modulating the phenotype.

The invention also provides a method of modulating a physiological characteristic associated with a disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising administering to a subject whom may already exhibit the physiological characteristic, or may be prone to exhibit the physiological characteristic or may be in whom the physiological characteristic is to be prevented, an effective amount of an agent identified as modulating said physiological characteristic, or agonists or antagonists thereof, thereby effectively modulating the physiological characteristic.

The invention also provides a method of modulating a behavior associated with a disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising administering to a subject whom may already exhibit the behavior, or may be prone to exhibit the behavior or may be in whom the exhibited behavior is to be prevented, an effective amount of an agent identified as modulating said behavior, or agonists or antagonists thereof, thereby effectively modulating the behavior.

The invention also provides a method of modulating the expression of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising administering to a host cell expressing said PRO256, PRO34421, PRO334, PRO770, PRO983, PRO1009, PRO107, PRO1158, PRO1250, PRO1317, PRO4334, PRO4395, PRO49192, PRO9799, PRO21175, PRO19837, PRO21331, PRO23949, PRO697 or PRO1480 polypeptide, an effective amount of an agent identified as modulating said expression, or agonists or antagonists thereof, thereby effectively modulating the expression of said polypeptide.

The invention also provides a method of modulating a condition associated with a disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising administering to a subject whom may have the condition, or may be prone to have the condition or may be in whom the condition is to be prevented, a therapeutically effective amount of a therapeutic agent identified as modulating said condition, or agonists or antagonists thereof, thereby effectively modulating the condition.

The invention also provides a method of treating or preventing or ameliorating a neurological disorder; cardiovascular, endothelial or angiogenic disorder; immunological disorder; oncological disorder; bone metabolic abnormality or disorder, or embryonic lethality associated with the disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising administering to a non-human transgenic animal cell culture, each cell of said culture comprising a disruption of the gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, an effective amount of an agent identified as treating or preventing or ameliorating said disorder, or agonists or antagonists thereof, thereby effectively treating or preventing or ameliorating said disorder.

### B. Further Embodiments

In yet further embodiments, the invention is directed to the following set of potential claims for this application:

1. A method of identifying a phenotype associated with a disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising:

(a) providing a non-human transgenic animal whose genome comprises a disruption of the gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide;

(b) measuring a physiological characteristic of the non-human transgenic animal; and

(c) comparing the measured physiological characteristic with that of a gender matched wild-type animal, wherein the physiological characteristic of the non-human transgenic animal that differs from the physiological characteristic of the wild-type animal is identified as a phenotype resulting from the gene disruption in the non-human transgenic animal.

2. The method of Claim 1, wherein the non-human transgenic animal is heterozygous for the disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide.
3. The method of Claim 1, wherein the phenotype exhibited by the non-human transgenic animal as compared with gender matched wild-type littermates is at least one of the following: a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality.
4. The method of Claim 3, wherein the neurological disorder is an increased anxiety-like response during open field activity testing.
5. The method of Claim 3, wherein the neurological disorder is a decreased anxiety-like response during open field activity testing.
6. The method of Claim 3, wherein the neurological disorder is an abnormal circadian rhythm during home-cage activity testing.
7. The method of Claim 3, wherein the neurological disorder is an enhanced motor coordination during inverted screen testing.
8. The method of Claim 3, wherein the neurological disorder is an impaired motor coordination during inverted screen testing.
9. The method of Claim 3, wherein the neurological disorder is depression, generalized anxiety disorders, attention deficit disorder, sleep disorder, hyperactivity disorder, obsessive compulsive disorder, schizophrenia, cognitive disorders, hyperalgesia or sensory disorders.
10. The method of Claim 3, wherein the eye abnormality is a retinal abnormality.
11. The method of Claim 3, wherein the eye abnormality is consistent with vision problems or blindness.
12. The method of Claim 10, wherein the retinal abnormality is consistent with retinitis pigmentosa.
13. The method of Claim 10, wherein the retinal abnormality is characterized by retinal degeneration or retinal dysplasia.
14. The method of Claim 10, wherein the retinal abnormality is consistent with retinal dysplasia, various retinopathies, including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstrom's syndrome, Cockayne's syndrome, dysplasia spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis.
15. The method of Claim 3, wherein the eye abnormality is a cataract.
16. The method of Claim 15, wherein the cataract is consistent with systemic diseases such as human Down's syndrome, Hallerman-Streiff syndrome, Lowe syndrome, galactosemia, Marfan syndrome, Trismoy 13-15, Alport syndrome, myotonic dystrophy, Fabry disease, hypoparathyroidism or Conradi syndrome.
17. The method of Claim 3, wherein the developmental abnormality comprises embryonic lethality or reduced viability.
18. The method of Claim 3, wherein the cardiovascular, endothelial or angiogenic disorders are arterial diseases, such as diabetes mellitus; papilledema; optic atrophy; atherosclerosis; angina; myocardial infarctions such as acute myocardial infarctions, cardiac hypertrophy, and heart failure such as congestive heart failure; hypertension; inflammatory vasculitides; Reynaud's disease and Reynaud's phenomenon; aneurysms and arterial restenosis; venous and lymphatic disorders such as thrombophlebitis, lymphangitis, and lymphedema; peripheral vascular disease; cancer such as vascular tumors, e.g., hemangioma (capillary and cavernous), glomus tumors, telangiectasia, bacillary angiomatosis, hemangioendothelioma, angiosarcoma, hemangiopericytoma, Kaposi's sarcoma, lymphangioma, and lymphangiosarcoma; tumor angiogenesis; trauma such as wounds, burns, and other injured tissue, implant fixation, scarring; ischemia reperfusion injury; rheumatoid arthritis; cerebrovascular disease; renal diseases such as acute renal failure, or osteoporosis.
19. The method of Claim 3, wherein the immunological disorders are systemic lupus erythematosis; rheumatoid arthritis; juvenile chronic arthritis; spondyloarthropathies; systemic sclerosis (scleroderma); idiopathic inflammatory myopathies (dermatomyositis, polymyositis); Sjögren's syndrome; systemic vasculitis; sarcoidosis; autoimmune hemolytic anemia (immune pancytopenia, paroxysmal nocturnal hemoglobinuria); autoimmune thrombocytopenia (idiopathic thrombocytopenic purpura, immune-mediated thrombocytopenia); thyroiditis (Grave's disease, Hashimoto's thyroiditis, juvenile lymphocytic thyroiditis, atrophic thyroiditis); diabetes mellitus; immune-mediated renal disease (glomerulonephritis, tubulointerstitial nephritis); demyelinating diseases of the central and peripheral nervous systems such as multiple sclerosis, idiopathic demyelinating polyneuropathy or Guillain-Barré syndrome, and chronic inflammatory demyelinating polyneuropathy; hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis; inflammatory bowel disease (ulcerative colitis: Crohn's disease); gluten-sensitive enteropathy, and Whipple's disease; autoimmune or immune-mediated skin diseases including bullous skin diseases, erythema multiform and contact dermatitis, psoriasis; allergic diseases such as asthma, allergic rhinitis, atopic dermatitis, food hypersensitivity and urticaria; immunologic diseases of the lung such as eosinophilic pneumonia, idiopathic pulmonary fibrosis and hypersensitivity pneumonitis; or transplantation associated diseases including graft rejection and graft-versus-host disease.
20. The method of Claim 3, wherein the bone metabolic abnormality or disorder is arthritis, osteoporosis or osteopetrosis.
21. The method of Claim 1, wherein the non-human transgenic animal exhibits at least one of the following physiological characteristics compared with gender matched wild-type littermates: a decreased anxiety-like response during open field activity testing; an abnormal circadian rhythm during home-cage activity testing; an enhanced motor coordination during inverted screen testing; exophthalamus in functional observation testing; severe retinal degeneration marked by attenuated retinal vessels; retinal microaneurisms; decreased mean artery-to-vein ratio; decreased lens size; mature cataracts; an increased mean serum cholesterol level; an increased mean serum triglyceride level; a decreased mean serum cholesterol level; an enhanced glucose tolerance; a decreased glucose tolerance; an increased mean serum insulin level; a decreased mean serum insulin level; a decreased mean serum IgG1 and IgG2a responses to an ovalbumin challenge; an increased mean serum IgG2a response to an ovalbumin challenge; an impaired IgG2a response to an ovalbumin challenge; a decreased mean absolute blood neutrophil count; an increased mean serum levels of IgG1, IgG3, IgA, IgG2a and IgG2b; an increased mean serum TNF-alpha and IL6 response to a LPS challenge; a decreased mean platelet count; a reduced level of RBC's, platelets, hemoglobin and hematocrit; an increased mean percent body fat; a decreased skin fibroblast proliferation; an increased skin fibroblast proliferation; an increased total tissue mass (TTM); an increased lean body mass (LBM); an increased bone mineral density (BMD); an increased bone mineral content (BMC), an increased bone mineral content index (BMC/LBM); an increased midshaft femur total area; a decrease in trabecular bone volume and connectivity density; a decreased volumetric bone mineral density; a decreased bone mineral content index (BMC/LBM); a decreased mean bone mineral density in total body, femur and vertebrate; a decreased mean bone mineral density (BMD), a decreased mean trabecular bone volume, decreased thickness, and decreased connectivity density; a decreased body weight and length; a decreased total tissue mass (TTM); a decreased lean body mass (LBM); a decreased total fat mass; a decreased bone mineral content (BMC); a decreased mean volumetric bone mineral density (vBMD) in total body and femur; a decreased femoral midshaft cross-sectional area and thickness; growth retardation with decreased mean body weight and length, decreased mean percent of total body fat, decreased total tissue mass and decreased bone mineral density; a decreased femoral midshaft cortical thickness; cardiomegaly; an impaired renal function; renal mesonephric duct development abnormalities; seminiferous tubular degeneration; greatly reduced viability [only three (−/−) mutant mice survived showing severe growth retardation as compared to the expected 14 (−/−) mutants]; a significant reduction in expected numbers of homozygotes; and embryonic lethality.
22. An isolated cell derived from a non-human transgenic animal whose genome comprises a disruption of the gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide.
23. The isolated cell of Claim 22 which is a murine cell.
24. The isolated cell of Claim 23, wherein the murine cell is an embryonic stem cell.
25. The isolated cell of Claim 22, wherein the non-human transgenic animal exhibits at least one of the following phenotypes compared with gender matched wild-type littermates: a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality.
26. A method of identifying an agent that modulates a phenotype associated with a disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising:

(a) providing a non-human transgenic animal whose genome comprises a disruption of the gene which encodes for the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide;

(b) measuring a physiological characteristic of the non-human transgenic animal of (a);

(c) comparing the measured physiological characteristic of (b) with that of a gender matched wild-type animal, wherein the physiological characteristic of the non-human transgenic animal that differs from the physiological characteristic of the wild-type animal is identified as a phenotype resulting from the gene disruption in the non-human transgenic animal;

(d) administering a test agent to the non-human transgenic animal of (a); and

(e) determining whether the test agent modulates the identified phenotype associated with gene disruption in the non-human transgenic animal.

27. The method of Claim 26, wherein the phenotype associated with the gene disruption comprises a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality.
28. The method of Claim 27, wherein the neurological disorder is an increased anxiety-like response during open field activity testing.
29. The method of Claim 27, wherein the neurological disorder is a decreased anxiety-like response during open field activity testing.
30. The method of Claim 27, wherein the neurological disorder is an abnormal circadian rhythm during home-cage activity testing.
31. The method of Claim 27, wherein the neurological disorder is an enhanced motor coordination during inverted screen testing.
32. The method of Claim 27, wherein the neurological disorder is an impaired motor coordination during inverted screen testing.
33. The method of Claim 27, wherein the neurological disorder is depression, generalized anxiety disorders, attention deficit disorder, sleep disorder, hyperactivity disorder, obsessive compulsive disorder, schizophrenia, cognitive disorders, hyperalgesia or sensory disorders.
34. The method of Claim 27, wherein the eye abnormality is a retinal abnormality.
35. The method of Claim 27, wherein the eye abnormality is consistent with vision problems or blindness.
36. The method of Claim 34, wherein the retinal abnormality is consistent with retinitis pigmentosa.
37. The method of Claim 34, wherein the retinal abnormality is characterized by retinal degeneration or retinal dysplasia.
38. The method of Claim 34, wherein the retinal abnormality is consistent with retinal dysplasia, various retinopathies, including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstrom's syndrome, Cockayne's syndrome, dysplasia spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis.
39. The method of Claim 27, wherein the eye abnormality is a cataract.
40. The method of Claim 39, wherein the cataract is consistent with systemic diseases such as human Down's syndrome, Hallerman-Streiff syndrome, Lowe syndrome, galactosemia, Marfan syndrome, Trismoy 13-15, Alport syndrome, myotonic dystrophy, Fabry disease, hypoparathyroidism or Conradi syndrome.
41. The method of Claim 27, wherein the developmental abnormality comprises embryonic lethality or reduced viability.
42. The method of Claim 27, wherein the cardiovascular, endothelial or angiogenic disorders are arterial diseases, such as diabetes mellitus; papilledema; optic atrophy; atherosclerosis; angina; myocardial infarctions such as acute myocardial infarctions, cardiac hypertrophy, and heart failure such as congestive heart failure; hypertension; inflammatory vasculitides; Reynaud's disease and Reynaud's phenomenon; aneurysms and arterial restenosis; venous and lymphatic disorders such as thrombophlebitis, lymphangitis, and lymphedema; peripheral vascular disease; cancer such as vascular tumors, e.g., hemangioma (capillary and cavernous), glomus tumors, telangiectasia, bacillary angiomatosis, hemangioendothelioma, angiosarcoma, hemangiopericytoma, Kaposi's sarcoma, lymphangioma, and lymphangiosarcoma; tumor angiogenesis; trauma such as wounds, burns, and other injured tissue, implant fixation, scarring; ischemia reperfusion injury; rheumatoid arthritis; cerebrovascular disease; renal diseases such as acute renal failure, or osteoporosis.
43. The method of Claim 27, wherein the immunological disorders are systemic lupus erythematosis; rheumatoid arthritis; juvenile chronic arthritis; spondyloarthropathies; systemic sclerosis (scleroderma); idiopathic inflammatory myopathies (dermatomyositis, polymyositis); Sjögren's syndrome; systemic vasculitis; sarcoidosis; autoimmune hemolytic anemia (immune pancytopenia, paroxysmal nocturnal hemoglobinuria); autoimmune thrombocytopenia (idiopathic thrombocytopenic purpura, immune-mediated thrombocytopenia); thyroiditis (Grave's disease, Hashimoto's thyroiditis, juvenile lymphocytic thyroiditis, atrophic thyroiditis); diabetes mellitus; immune-mediated renal disease (glomerulonephritis, tubulointerstitial nephritis); demyelinating diseases of the central and peripheral nervous systems such as multiple sclerosis, idiopathic demyelinating polyneuropathy or Guillain-Barré syndrome, and chronic inflammatory demyelinating polyneuropathy; hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis; inflammatory bowel disease (ulcerative colitis: Crohn's disease); gluten-sensitive enteropathy, and Whipple's disease; autoimmune or immune-mediated skin diseases including bullous skin diseases, erythema multiform and contact dermatitis, psoriasis; allergic diseases such as asthma, allergic rhinitis, atopic dermatitis, food hypersensitivity and urticaria; immunologic diseases of the lung such as eosinophilic pneumonia, idiopathic pulmonary fibrosis and hypersensitivity pneumonitis; or transplantation-associated diseases including graft rejection and graft-versus-host disease.
44. The method of Claim 27, wherein said bone metabolic abnormality or disorder is arthritis, osteoporosis or osteopetrosis.
45. The method of Claim 26, wherein the non-human transgenic animal exhibits at least one of the following physiological characteristics compared with gender matched wild-type littermates: a decreased anxiety-like response during open field activity testing; an abnormal circadian rhythm during home-cage activity testing; an enhanced motor coordination during inverted screen testing; exophthalamus in functional observation testing; severe retinal degeneration marked by attenuated retinal vessels; retinal microaneurisms; decreased mean artery-to-vein ratio; decreased lens size; mature cataracts; an increased mean serum cholesterol level; an increased mean serum triglyceride level; a decreased mean serum cholesterol level; an enhanced glucose tolerance; a decreased glucose tolerance; an increased mean serum insulin level; a decreased mean serum insulin level; a decreased mean serum IgG1 and IgG2a responses to an ovalbumin challenge; an increased mean serum IgG2a response to an ovalbumin challenge; an impaired IgG2a response to an ovalbumin challenge; a decreased mean absolute blood neutrophil count; an increased mean serum levels of IgG1, IgG3, IgA, IgG2a and IgG2b; an increased mean serum TNF-alpha and IL6 response to a LPS challenge; a decreased mean platelet count; a reduced level of RBC's, platelets, hemoglobin and hematocrit; an increased mean percent body fat; a decreased skin fibroblast proliferation; an increased skin fibroblast proliferation; an increased total tissue mass (TTM); an increased lean body mass (LBM); an increased bone mineral density (BMD); an increased bone mineral content (BMC), an increased bone mineral content index (BMC/LBM); an increased midshaft femur total area; a decrease in trabecular bone volume and connectivity density; a decreased volumetric bone mineral density; a decreased bone mineral content index (BMC/LBM); a decreased mean bone mineral density in total body, femur and vertebrate; a decreased mean bone mineral density (BMD), a decreased mean trabecular bone volume, decreased thickness, and decreased connectivity density; a decreased body weight and length; a decreased total tissue mass (TTM); a decreased lean body mass (LBM); a decreased total fat mass; a decreased bone mineral content (BMC); a decreased mean volumetric bone mineral density (vBMD) in total body and femur; a decreased femoral midshaft cross-sectional area and thickness; growth retardation with decreased mean body weight and length, decreased mean percent of total body fat, decreased total tissue mass and decreased bone mineral density; a decreased femoral midshaft cortical thickness; cardiomegaly; an impaired renal function; renal mesonephric duct development abnormalities; seminiferous tubular degeneration; greatly reduced viability [only three (−/−) mutant mice survived showing severe growth retardation as compared to the expected 14 (−/−) mutants]; a significant reduction in expected numbers of homozygotes; and embryonic lethality.
46. An agent identified by the method of Claim 26.
47. The agent of Claim 46 which is an agonist or antagonist of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide.
48. The agent of Claim 47, wherein the agonist is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.
49. The agent of Claim 47, wherein the antagonist is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.
50. A method of identifying an agent that modulates a physiological characteristic associated with a disruption of the gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising:

(a) providing a non-human transgenic animal whose genome comprises a disruption of the gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide;

(b) measuring a physiological characteristic exhibited by the non-human transgenic animal of (a);

(c) comparing the measured physiological characteristic of (b) with that of a gender matched wild-type animal, wherein the physiological characteristic exhibited by the non-human transgenic animal that differs from the physiological characteristic exhibited by the wild-type animal is identified as a physiological characteristic associated with gene disruption;

(d) administering a test agent to the non-human transgenic animal of (a); and

(e) determining whether the physiological characteristic associated with gene disruption is modulated.

51. The method of Claim 50, wherein the non-human transgenic animal exhibits at least one of the following physiological characteristics compared with gender matched wild-type littermates: a decreased anxiety-like response during open field activity testing; an abnormal circadian rhythm during home-cage activity testing; an enhanced motor coordination during inverted screen testing; exophthalamus in functional observation testing; severe retinal degeneration marked by attenuated retinal vessels; retinal microaneurisms; decreased mean artery-to-vein ratio; decreased lens size; mature cataracts; an increased mean serum cholesterol level; an increased mean serum triglyceride level; a decreased mean serum cholesterol level; an enhanced glucose tolerance; a decreased glucose tolerance; an increased mean serum insulin level; a decreased mean serum insulin level; a decreased mean serum IgG1 and IgG2a responses to an ovalbumin challenge; an increased mean serum IgG2a response to an ovalbumin challenge; an impaired IgG2a response to an ovalbumin challenge; a decreased mean absolute blood neutrophil count; an increased mean serum levels of IgG1, IgG3, IgA, IgG2a and IgG2b; an increased mean serum TNF-alpha and IL6 response to a LPS challenge; a decreased mean platelet count; a reduced level of RBC's, platelets, hemoglobin and hematocrit; an increased mean percent body fat; a decreased skin fibroblast proliferation; an increased skin fibroblast proliferation; an increased total tissue mass (TTM); an increased lean body mass (LBM); an increased bone mineral density (BMD); an increased bone mineral content (BMC), an increased bone mineral content index (BMC/LBM); an increased midshaft femur total area; a decrease in trabecular bone volume and connectivity density; a decreased volumetric bone mineral density; a decreased bone mineral content index (BMC/LBM); a decreased mean bone mineral density in total body, femur and vertebrate; a decreased mean bone mineral density (BMD), a decreased mean trabecular bone volume, decreased thickness, and decreased connectivity density; a decreased body weight and length; a decreased total tissue mass (TTM); a decreased lean body mass (LBM); a decreased total fat mass; a decreased bone mineral content (BMC); a decreased mean volumetric bone mineral density (vBMD) in total body and femur; a decreased femoral midshaft cross-sectional area and thickness; growth retardation with decreased mean body weight and length, decreased mean percent of total body fat, decreased total tissue mass and decreased bone mineral density; a decreased femoral midshaft cortical thickness; cardiomegaly; an impaired renal function; renal mesonephric duct development abnormalities; seminiferous tubular degeneration; greatly reduced viability [only three (−/−) mutant mice survived showing severe growth retardation as compared to the expected 14 (−/−) mutants]; a significant reduction in expected numbers of homozygotes; and embryonic lethality.
52. An agent identified by the method of Claim 50.
53. The agent of Claim 52 which is an agonist or antagonist of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide.
54. The agent of Claim 53, wherein the agonist is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.
55. The agent of Claim 53, wherein the antagonist is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.
56. A method of identifying an agent which modulates a behavior associated with a disruption of the gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising:

(a) providing a non-human transgenic animal whose genome comprises a disruption of the gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide;

(b) observing the behavior exhibited by the non-human transgenic animal of (a);

(c) comparing the observed behavior of (b) with that of a gender matched wild-type animal, wherein the observed behavior exhibited by the non-human transgenic animal that differs from the observed behavior exhibited by the wild-type animal is identified as a behavior associated with gene disruption;

(d) administering a test agent to the non-human transgenic animal of (a); and

(e) determining whether the agent modulates the behavior associated with gene disruption.

57. The method of Claim 56, wherein the behavior is an increased anxiety-like response during open field activity testing.
58. The method of Claim 56, wherein the behavior is a decreased anxiety-like response during open field activity testing.
59. The method of Claim 56, wherein the behavior is an abnormal circadian rhythm during home-cage activity testing.
60. The method of Claim 56, wherein the behavior is an enhanced motor coordination during inverted screen testing.
61. The method of Claim 56, wherein the behavior is an impaired motor coordination during inverted screen testing.
62. The method of Claim 56, wherein the behavior is depression, generalized anxiety disorders, attention deficit disorder, sleep disorder, hyperactivity disorder, obsessive compulsive disorder, schizophrenia, cognitive disorders, hyperalgesia or sensory disorders.
63. An agent identified by the method of Claim 56.
64. The agent of Claim 63 which is an agonist or antagonist of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide.
65. The agent of Claim 64, wherein the agonist is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.
66. The agent of Claim 64, wherein the antagonist is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.
67. A method of identifying an agent that ameliorates or modulates a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality associated with a disruption in the gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising:

(a) providing a non-human transgenic animal whose genome comprises a disruption of the gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide;

(b) administering a test agent to said non-human transgenic animal; and

(c) determining whether said test agent ameliorates or modulates the neurological disorder; cardiovascular, endothelial or angiogenic disorder; eye abnormality; immunological disorder; oncological disorder; bone metabolic abnormality or disorder; lipid metabolic disorder; or developmental abnormality in the non-human transgenic animal.

68. The method of Claim 67, wherein the neurological disorder is an increased anxiety-like response during open field activity testing.
69. The method of Claim 67, wherein the neurological disorder is a decreased anxiety-like response during open field activity testing.
70. The method of Claim 67, wherein the neurological disorder is an abnormal circadian rhythm during home-cage activity testing.
71. The method of Claim 67, wherein the neurological disorder is an enhanced motor coordination during inverted screen testing.
72. The method of Claim 67, wherein the neurological disorder is an impaired motor coordination during inverted screen testing.
73. The method of Claim 73, wherein the neurological disorder is depression, generalized anxiety disorders, attention deficit disorder, sleep disorder, hyperactivity disorder, obsessive compulsive disorder, schizophrenia, cognitive disorders, hyperalgesia or sensory disorders.
74. The method of Claim 67, wherein the eye abnormality is a retinal abnormality.
75. The method of Claim 67, wherein the eye abnormality is consistent with vision problems or blindness.
76. The method of Claim 74, wherein the retinal abnormality is consistent with retinitis pigmentosa.
77. The method of Claim 74, wherein the retinal abnormality is characterized by retinal degeneration or retinal dysplasia.
78. The method of Claim 74, wherein the retinal abnormality is consistent with retinal dysplasia, various retinopathies, including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstrom's syndrome, Cockayne's syndrome, dysplasia spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis.
79. The method of Claim 67, wherein the eye abnormality is a cataract.
80. The method of Claim 79, wherein the cataract is a systemic disease such as human Down's syndrome, Hallerman-Streiff syndrome, Lowe syndrome, galactosemia, Marfan syndrome, Trismoy 13-15, Alport syndrome, myotonic dystrophy, Fabry disease, hypoparathyroidism or Conradi syndrome.
81. The method of Claim 67, wherein the developmental abnormality comprises embryonic lethality or reduced viability.
82. The method of Claim 67, wherein the cardiovascular, endothelial or angiogenic disorders are arterial diseases, such as diabetes mellitus; papilledema; optic atrophy; atherosclerosis; angina; myocardial infarctions such as acute myocardial infarctions, cardiac hypertrophy, and heart failure such as congestive heart failure; hypertension; inflammatory vasculitides; Reynaud's disease and Reynaud's phenomenon; aneurysms and arterial restenosis; venous and lymphatic disorders such as thrombophlebitis, lymphangitis, and lymphedema; peripheral vascular disease; cancer such as vascular tumors, e.g., hemangioma (capillary and cavernous), glomus tumors, telangiectasia, bacillary angiomatosis, hemangioendothelioma, angiosarcoma, hemangiopericytoma, Kaposi's sarcoma, lymphangioma, and lymphangiosarcoma; tumor angiogenesis; trauma such as wounds, burns, and other injured tissue, implant fixation, scarring; ischemia reperfusion injury; rheumatoid arthritis; cerebrovascular disease; renal diseases such as acute renal failure, or osteoporosis.
83. The method of Claim 67, wherein the immunological disorders are systemic lupus erythematosis; rheumatoid arthritis; juvenile chronic arthritis; spondyloarthropathies; systemic sclerosis (scleroderma); idiopathic inflammatory myopathies (dermatomyositis, polymyositis); Sjögren's syndrome; systemic vasculitis; sarcoidosis; autoimmune hemolytic anemia (immune pancytopenia, paroxysmal nocturnal hemoglobinuria); autoimmune thrombocytopenia (idiopathic thrombocytopenic purpura, immune-mediated thrombocytopenia); thyroiditis (Grave's disease, Hashimoto's thyroiditis, juvenile lymphocytic thyroiditis, atrophic thyroiditis); diabetes mellitus; immune-mediated renal disease (glomerulonephritis, tubulointerstitial nephritis); demyelinating diseases of the central and peripheral nervous systems such as multiple sclerosis, idiopathic demyelinating polyneuropathy or Guillain-Barré syndrome, and chronic inflammatory demyelinating polyneuropathy; hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis; inflammatory bowel disease (ulcerative colitis: Crohn's disease); gluten-sensitive enteropathy, and Whipple's disease; autoimmune or immune-mediated skin diseases including bullous skin diseases, erythema multiform and contact dermatitis, psoriasis; allergic diseases such as asthma, allergic rhinitis, atopic dermatitis, food hypersensitivity and urticaria; immunologic diseases of the lung such as eosinophilic pneumonia, idiopathic pulmonary fibrosis and hypersensitivity pneumonitis; or transplantation associated diseases including graft rejection and graft-versus-host disease.
84. The method of Claim 67, wherein said bone metabolic abnormality or disorder is arthritis, osteoporosis or osteopetrosis.
85. The method of Claim 67, wherein the non-human transgenic animal exhibits at least one of the following physiological characteristics compared with gender matched wild-type littermates: a decreased anxiety-like response during open field activity testing; an abnormal circadian rhythm during home-cage activity testing; an enhanced motor coordination during inverted screen testing; exophthalamus in functional observation testing; severe retinal degeneration marked by attenuated retinal vessels; retinal microaneurisms; decreased mean artery-to-vein ratio; decreased lens size; mature cataracts; an increased mean serum cholesterol level; an increased mean serum triglyceride level; a decreased mean serum cholesterol level; an enhanced glucose tolerance; a decreased glucose tolerance; an increased mean serum insulin level; a decreased mean serum insulin level; a decreased mean serum IgG1 and IgG2a responses to an ovalbumin challenge; an increased mean serum IgG2a response to an ovalbumin challenge; an impaired IgG2a response to an ovalbumin challenge; a decreased mean absolute blood neutrophil count; an increased mean serum levels of IgG1, IgG3, IgA, IgG2a and IgG2b; an increased mean serum TNF-alpha and IL6 response to a LPS challenge; a decreased mean platelet count; a reduced level of RBC's, platelets, hemoglobin and hematocrit; an increased mean percent body fat; a decreased skin fibroblast proliferation; an increased skin fibroblast proliferation; an increased total tissue mass (TTM); an increased lean body mass (LBM); an increased bone mineral density (BMD); an increased bone mineral content (BMC), an increased bone mineral content index (BMC/LBM); an increased midshaft femur total area; a decrease in trabecular bone volume and connectivity density; a decreased volumetric bone mineral density; a decreased bone mineral content index (BMC/LBM); a decreased mean bone mineral density in total body, femur and vertebrate; a decreased mean bone mineral density (BMD), a decreased mean trabecular bone volume, decreased thickness, and decreased connectivity density; a decreased body weight and length; a decreased total tissue mass (TTM); a decreased lean body mass (LBM); a decreased total fat mass; a decreased bone mineral content (BMC); a decreased mean volumetric bone mineral density (vBMD) in total body and femur; a decreased femoral midshaft cross-sectional area and thickness; growth retardation with decreased mean body weight and length, decreased mean percent of total body fat, decreased total tissue mass and decreased bone mineral density; a decreased femoral midshaft cortical thickness; cardiomegaly; an impaired renal function; renal mesonephric duct development abnormalities; seminiferous tubular degeneration; greatly reduced viability [only three (−/−) mutant mice survived showing severe growth retardation as compared to the expected 14 (−/−) mutants]; a significant reduction in expected numbers of homozygotes; and embryonic lethality.
86. An agent identified by the method of Claim 67.
87. The agent of Claim 86 which is an agonist or antagonist of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide.
88. The agent of Claim 87, wherein the agonist is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.
89. The agent of Claim 87, wherein the antagonist is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.
90. A therapeutic agent identified by the method of Claim 67.
91. A method of identifying an agent that modulates the expression of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising:

(a) contacting a test agent with a host cell expressing a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide; and

(b) determining whether the test agent modulates the expression of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide by the host cell.

92. An agent identified by the method of Claim 91.
93. The agent of Claim 92 which is an agonist or antagonist of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide.
94. The agent of Claim 93, wherein the agonist is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.
95. The agent of Claim 93, wherein the antagonist is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.
96. A method of evaluating a therapeutic agent capable of affecting a condition associated with a disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising:

(a) providing a non-human transgenic animal whose genome comprises a disruption of the gene which encodes for the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide;

(b) measuring a physiological characteristic of the non-human transgenic animal of (a);

(c) comparing the measured physiological characteristic of (b) with that of a gender matched wild-type animal, wherein the physiological characteristic of the non-human transgenic animal that differs from the physiological characteristic of the wild-type animal is identified as a condition resulting from the gene disruption in the non-human transgenic animal;

(d) administering a test agent to the non-human transgenic animal of (a); and

(e) evaluating the effects of the test agent on the identified condition associated with gene disruption in the non-human transgenic animal.

97. The method of Claim 96, wherein the condition is a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality.
98. A therapeutic agent identified by the method of Claim 96.
99. The therapeutic agent of Claim 98 which is an agonist or antagonist of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide.
100. The therapeutic agent of Claim 99, wherein the agonist is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.
101. The therapeutic agent of Claim 99, wherein the antagonist is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.
102. A pharmaceutical composition comprising the therapeutic agent of Claim 98.
103. A method of treating or preventing or ameliorating a neurological disorder; cardiovascular, endothelial or angiogenic disorder; immunological disorder; oncological disorder; bone metabolic abnormality or disorder, or embryonic lethality associated with the disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising administering to a subject in need of such treatment whom may already have the disorder, or may be prone to have the disorder or may be in whom the disorder is to be prevented, a therapeutically effective amount of the therapeutic agent of Claim 94, or agonists or antagonists thereof, thereby effectively treating or preventing or ameliorating said disorder.
104. The method of Claim 103, wherein the neurological disorder is an increased anxiety-like response during open field activity testing.
105. The method of Claim 103, wherein the neurological disorder is a decreased anxiety-like response during open field activity testing.
106. The method of Claim 103, wherein the neurological disorder is an abnormal circadian rhythm during home-cage activity testing.
107. The method of Claim 103, wherein the neurological disorder is an enhanced motor coordination during inverted screen testing.
108. The method of Claim 103, wherein the neurological disorder is an impaired motor coordination during inverted screen testing.
109. The method of Claim 103, wherein the neurological disorder is depression, generalized anxiety disorders, attention deficit disorder, sleep disorder, hyperactivity disorder, obsessive compulsive disorder, schizophrenia, cognitive disorders, hyperalgesia or sensory disorders.
110. The method of Claim 103, wherein the eye abnormality is a retinal abnormality.
111. The method of Claim 103, wherein the eye abnormality is consistent with vision problems or blindness.
112. The method of Claim 110, wherein the retinal abnormality is consistent with retinitis pigmentosa.
113. The method of Claim 110, wherein the retinal abnormality is characterized by retinal degeneration or retinal dysplasia.
114. The method of Claim 110, wherein the retinal abnormality is consistent with retinal dysplasia, various retinopathies, including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstrom's syndrome, Cockayne's syndrome, dysplasia spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis.
115. The method of Claim 103, wherein the eye abnormality is a cataract.
116. The method of Claim 115, wherein the cataract is a systemic disease such as human Down's syndrome, Hallerman-Streiff syndrome, Lowe syndrome, galactosemia, Marfan syndrome, Trismoy 13-15, Alport syndrome, myotonic dystrophy, Fabry disease, hypoparathyroidism or Conradi syndrome.
117. The method of Claim 103, wherein the developmental abnormality comprises embryonic lethality or reduced viability.
118. The method of Claim 103, wherein the cardiovascular, endothelial or angiogenic disorders are arterial diseases, such as diabetes mellitus; papilledema; optic atrophy; atherosclerosis; angina; myocardial infarctions such as acute myocardial infarctions, cardiac hypertrophy, and heart failure such as congestive heart failure; hypertension; inflammatory vasculitides; Reynaud's disease and Reynaud's phenomenon; aneurysms and arterial restenosis; venous and lymphatic disorders such as thrombophlebitis, lymphangitis, and lymphedema; peripheral vascular disease; cancer such as vascular tumors, e.g., hemangioma (capillary and cavernous), glomus tumors, telangiectasia, bacillary angiomatosis, hemangioendothelioma, angiosarcoma, hemangiopericytoma, Kaposi's sarcoma, lymphangioma, and lymphangiosarcoma; tumor angiogenesis; trauma such as wounds, burns, and other injured tissue, implant fixation, scarring; ischemia reperfusion injury; rheumatoid arthritis; cerebrovascular disease; renal diseases such as acute renal failure, or osteoporosis.
119. The method of Claim 103, wherein the immunological disorders are systemic lupus erythematosis; rheumatoid arthritis; juvenile chronic arthritis; spondyloarthropathies; systemic sclerosis (scleroderma); idiopathic inflammatory myopathies (dermatomyositis, polymyositis); Sjögren's syndrome; systemic vasculitis; sarcoidosis; autoimmune hemolytic anemia (immune pancytopenia, paroxysmal nocturnal hemoglobinuria); autoimmune thrombocytopenia (idiopathic thrombocytopenic purpura, immune-mediated thrombocytopenia); thyroiditis (Grave's disease, Hashimoto's thyroiditis, juvenile lymphocytic thyroiditis, atrophic thyroiditis); diabetes mellitus; immune-mediated renal disease (glomerulonephritis, tubulointerstitial nephritis); demyelinating diseases of the central and peripheral nervous systems such as multiple sclerosis, idiopathic demyelinating polyneuropathy or Guillain-Barré syndrome, and chronic inflammatory demyelinating polyneuropathy; hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis; inflammatory bowel disease (ulcerative colitis: Crohn's disease); gluten-sensitive enteropathy, and Whipple's disease; autoimmune or immune-mediated skin diseases including bullous skin diseases, erythema multiform and contact dermatitis, psoriasis; allergic diseases such as asthma, allergic rhinitis, atopic dermatitis, food hypersensitivity and urticaria; immunologic diseases of the lung such as eosinophilic pneumonia, idiopathic pulmonary fibrosis and hypersensitivity pneumonitis; or transplantation associated diseases including graft rejection and graft-versus-host disease.
120. The method of Claim 103, wherein said bone metabolic abnormality or disorder is arthritis, osteoporosis or osteopetrosis.
121. A method of identifying an agent that ameliorates or modulates a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality associated with a disruption in the gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising:

(a) providing a non-human transgenic animal cell culture, each cell of said culture comprising a disruption of the gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide;

(b) administering a test agent to said cell culture; and

(c) determining whether said test agent ameliorates or modulates the neurological disorder; cardiovascular, endothelial or angiogenic disorder; eye abnormality; immunological disorder; oncological disorder; bone metabolic abnormality or disorder; lipid metabolic disorder; or developmental abnormality in said cell culture.

122. The method of Claim 121, wherein the neurological disorder is an increased anxiety-like response during open field activity testing.
123. The method of Claim 121, wherein the neurological disorder is a decreased anxiety-like response during open field activity testing.
124. The method of Claim 121, wherein the neurological disorder is an abnormal circadian rhythm during home-cage activity testing.
125. The method of Claim 121, wherein the neurological disorder is an enhanced motor coordination during inverted screen testing.
126. The method of Claim 121, wherein the neurological disorder is an impaired motor coordination during inverted screen testing.
127. The method of Claim 121, wherein the neurological disorder is depression, generalized anxiety disorders, attention deficit disorder, sleep disorder, hyperactivity disorder, obsessive compulsive disorder, schizophrenia, cognitive disorders, hyperalgesia or sensory disorders.
128. The method of Claim 121, wherein the eye abnormality is a retinal abnormality.
129. The method of Claim 121, wherein the eye abnormality is consistent with vision problems or blindness.
130. The method of Claim 128, wherein the retinal abnormality is consistent with retinitis pigmentosa.
131. The method of Claim 128, wherein the retinal abnormality is characterized by retinal degeneration or retinal dysplasia.
132. The method of Claim 128, wherein the retinal abnormality is consistent with retinal dysplasia, various retinopathies, including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstrom's syndrome, Cockayne's syndrome, dysplasia spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis.
133. The method of Claim 121, wherein the eye abnormality is a cataract.
134. The method of Claim 133, wherein the cataract is a systemic disease such as human Down's syndrome, Hallerman-Streiff syndrome, Lowe syndrome, galactosemia, Marfan syndrome, Trismoy 13-15, Alport syndrome, myotonic dystrophy, Fabry disease, hypoparathyroidism or Conradi syndrome.
135. The method of Claim 121, wherein the developmental abnormality comprises embryonic lethality or reduced viability.
136. The method of Claim 121, wherein the cardiovascular, endothelial or angiogenic disorders are arterial diseases, such as diabetes mellitus; papilledema; optic atrophy; atherosclerosis; angina; myocardial infarctions such as acute myocardial infarctions, cardiac hypertrophy, and heart failure such as congestive heart failure; hypertension; inflammatory vasculitides; Reynaud's disease and Reynaud's phenomenon; aneurysms and arterial restenosis; venous and lymphatic disorders such as thrombophlebitis, lymphangitis, and lymphedema; peripheral vascular disease; cancer such as vascular tumors, e.g., hemangioma (capillary and cavernous), glomus tumors, telangiectasia, bacillary angiomatosis, hemangioendothelioma, angiosarcoma, hemangiopericytoma, Kaposi's sarcoma, lymphangioma, and lymphangiosarcoma; tumor angiogenesis; trauma such as wounds, burns, and other injured tissue, implant fixation, scarring; ischemia reperfusion injury; rheumatoid arthritis; cerebrovascular disease; renal diseases such as acute renal failure, or osteoporosis.
137. The method of Claim 121, wherein the immunological disorders are systemic lupus erythematosis; rheumatoid arthritis; juvenile chronic arthritis; spondyloarthropathies; systemic sclerosis (scleroderma); idiopathic inflammatory myopathies (dermatomyositis, polymyositis); Sjögren's syndrome; systemic vasculitis; sarcoidosis; autoimmune hemolytic anemia (immune pancytopenia, paroxysmal nocturnal hemoglobinuria); autoimmune thrombocytopenia (idiopathic thrombocytopenic purpura, immune-mediated thrombocytopenia); thyroiditis (Grave's disease, Hashimoto's thyroiditis, juvenile lymphocytic thyroiditis, atrophic thyroiditis); diabetes mellitus; immune-mediated renal disease (glomerulonephritis, tubulointerstitial nephritis); demyelinating diseases of the central and peripheral nervous systems such as multiple sclerosis, idiopathic demyelinating polyneuropathy or Guillain-Barré syndrome, and chronic inflammatory demyelinating polyneuropathy; hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis; inflammatory bowel disease (ulcerative colitis: Crohn's disease); gluten-sensitive enteropathy, and Whipple's disease; autoimmune or immune-mediated skin diseases including bullous skin diseases, erythema multiform and contact dermatitis, psoriasis; allergic diseases such as asthma, allergic rhinitis, atopic dermatitis, food hypersensitivity and urticaria; immunologic diseases of the lung such as eosinophilic pneumonia, idiopathic pulmonary fibrosis and hypersensitivity pneumonitis; or transplantation associated diseases including graft rejection and graft-versus-host disease.
138. The method of Claim 121, wherein said bone metabolic abnormality or disorder is arthritis, osteoporosis or osteopetrosis.
139. An agent identified by the method of Claim 121.
140. The agent of Claim 139 which is an agonist or antagonist of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide.
141. The agent of Claim 140, wherein the agonist is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.
142. The agent of Claim 140, wherein the antagonist is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.
143. A therapeutic agent identified by the method of Claim 121.
144. A method of modulating a phenotype associated with a disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising administering to a subject whom may already have the phenotype, or may be prone to have the phenotype or may be in whom the phenotype is to be prevented, an effective amount of the agent of Claim 46, or agonists or antagonists thereof, thereby effectively modulating the phenotype.
145. A method of modulating a physiological characteristic associated with a disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising administering to a subject whom may already exhibit the physiological characteristic, or may be prone to exhibit the physiological characteristic or may be in whom the physiological characteristic is to be prevented, an effective amount of the agent of Claim 52, or agonists or antagonists thereof, thereby effectively modulating the physiological characteristic.
146. A method of modulating a behavior associated with a disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising administering to a subject whom may already exhibit the behavior, or may be prone to exhibit the behavior or may be in whom the exhibited behavior is to be prevented, an effective amount of the agent of Claim 63, or agonists or antagonists thereof, thereby effectively modulating the behavior.
147. A method of modulating the expression of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising administering to a host cell expressing said PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, an effective amount of the agent of Claim 92, or agonists or antagonists thereof, thereby effectively modulating the expression of said polypeptide.
148. A method of modulating a condition associated with a disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising administering to a subject whom may have the condition, or may be prone to have the condition or may be in whom the condition is to be prevented, a therapeutically effective amount of the therapeutic agent of Claim 98, or agonists or antagonists thereof, thereby effectively modulating the condition.
149. A method of treating or preventing or ameliorating a neurological disorder; cardiovascular, endothelial or angiogenic disorder; immunological disorder; oncological disorder; bone metabolic abnormality or disorder, or embryonic lethality associated with the disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising administering to a non-human transgenic animal cell culture, each cell of said culture comprising a disruption of the gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, a therapeutically effective amount of the agent of Claim 139, or agonists or antagonists thereof, thereby effectively treating or preventing or ameliorating said disorder.

### BRIEF DESCRIPTION OF THE DRAWINGS

FIG. 1 shows a nucleotide sequence (SEQ ID NO:1) of a native sequence PRO256 cDNA, wherein SEQ ID NO:1 is a clone designated herein as “DNA35880-1160” (UNQ223).

FIG. 2 shows the amino acid sequence (SEQ ID NO:2) derived from the coding sequence of SEQ ID NO:1 shown in FIG. 1.

FIG. 3 shows a nucleotide sequence (SEQ ID NO:3) of a native sequence PRO34421 cDNA, wherein SEQ ID NO:3 is a clone designated herein as “DNA212937” (UNQ281).

FIG. 4 shows the amino acid sequence (SEQ ID NO:4) derived from the coding sequence of SEQ ID NO:3 shown in FIG. 3.

FIG. 5 shows a nucleotide sequence (SEQ ID NO:5) of a native sequence PRO334 cDNA, wherein SEQ ID NO:5 is a clone designated herein as “DNA41379-1236” (UNQ295).

FIG. 6 shows the amino acid sequence (SEQ ID NO:6) derived from the coding sequence of SEQ ID NO:5 shown in FIG. 5.

FIG. 7 shows a nucleotide sequence (SEQ ID NO:7) of a native sequence PRO770 cDNA, wherein SEQ ID NO:7 is a clone designated herein as “DNA54228-1366-1” (UNQ408).

FIG. 8 shows the amino acid sequence (SEQ ID NO:8) derived from the coding sequence of SEQ ID NO:7 shown in FIG. 7.

FIG. 9 shows a nucleotide sequence (SEQ ID NO:9) of a native sequence PRO983 cDNA, wherein SEQ ID NO:9 is a clone designated herein as “DNA53977-1371” (UNQ484).

FIG. 10 shows the amino acid sequence (SEQ ID NO:10) derived from the coding sequence of SEQ ID NO:9 shown in FIG. 9.

FIG. 11 shows a nucleotide sequence (SEQ ID NO:11) of a native sequence PRO1009 cDNA, wherein SEQ ID NO:11 is a clone designated herein as “DNA57129-1413” (UNQ493).

FIG. 12 shows the amino acid sequence (SEQ ID NO:12) derived from the coding sequence of SEQ ID NO:11 shown in FIG. 11.

FIG. 13 shows a nucleotide sequence (SEQ ID NO:13) of a native sequence PRO1107 cDNA, wherein SEQ ID NO:13 is a clone designated herein as “DNA59606-1471” (UNQ550).

FIG. 14 shows the amino acid sequence (SEQ ID NO:14) derived from the coding sequence of SEQ ID NO:13 shown in FIG. 13.

FIG. 15 shows a nucleotide sequence (SEQ ID NO:15) of a native sequence PRO1158 cDNA, wherein SEQ ID NO:15 is a clone designated herein as “DNA60625-1507” (UNQ588).

FIG. 16 shows the amino acid sequence (SEQ ID NO:16) derived from the coding sequence of SEQ ID NO:15 shown in FIG. 15.

FIG. 17 shows a nucleotide sequence (SEQ ID NO:17) of a native sequence PRO1250 cDNA, wherein SEQ ID NO:17 is a clone designated herein as “DNA60775-1532” (UNQ633).

FIG. 18 shows the amino acid sequence (SEQ ID NO:18) derived from the coding sequence of SEQ ID NO:17 shown in FIG. 17.

FIG. 19 shows a nucleotide sequence (SEQ ID NO:19) of a native sequence PRO1317 cDNA, wherein SEQ ID NO:19 is a clone designated herein as “DNA71166-1685” (UNQ783).

FIG. 20 shows the amino acid sequence (SEQ ID NO:20) derived from the coding sequence of SEQ ID NO:19 shown in FIG. 19.

FIG. 21 shows a nucleotide sequence (SEQ ID NO:21) of a native sequence PRO4334 cDNA, wherein SEQ ID NO:21 is a clone designated herein as “DNA59608-2577” (UNQ1889).

FIG. 22 shows the amino acid sequence (SEQ ID NO:22) derived from the coding sequence of SEQ ID NO:21 shown in FIG. 21.

FIG. 23 shows a nucleotide sequence (SEQ ID NO:23) of a native sequence PRO4395 cDNA, wherein SEQ ID NO:23 is a clone designated herein as “DNA80840-2605” (UNQ1921).

FIG. 24 shows the amino acid sequence (SEQ ID NO:24) derived from the coding sequence of SEQ ID NO:23 shown in FIG. 23.

FIG. 25 shows a nucleotide sequence (SEQ ID NO:25) of a native sequence PRO49192 cDNA, wherein SEQ ID NO:25 is a clone designated herein as “DNA237637” (UNQ2239).

FIG. 26 shows the amino acid sequence (SEQ ID NO:26) derived from the coding sequence of SEQ ID NO:25 shown in FIG. 25.

FIG. 27 shows a nucleotide sequence (SEQ ID NO:27) of a native sequence PRO9799 cDNA, wherein SEQ ID NO:27 is a clone designated herein as “DNA108696-2966” (UNQ3018).

FIG. 28 shows the amino acid sequence (SEQ ID NO:28) derived from the coding sequence of SEQ ID NO:27 shown in FIG. 27.

FIG. 29 shows a nucleotide sequence (SEQ ID NO:29) of a native sequence PRO21175 cDNA, wherein SEQ ID NO:29 is a clone designated herein as “DNA173894-2947” (UNQ3096).

FIG. 30 shows the amino acid sequence (SEQ ID NO:30) derived from the coding sequence of SEQ ID NO:29 shown in FIG. 29.

FIG. 31 shows a nucleotide sequence (SEQ ID NO:31) of a native sequence PRO19837 cDNA, wherein SEQ ID NO:31 is a clone designated herein as “DNA148009-2889” (UNQ5931).

FIG. 32 shows the amino acid sequence (SEQ ID NO:32) derived from the coding sequence of SEQ ID NO:31 shown in FIG. 31.

FIG. 33 shows a nucleotide sequence (SEQ ID NO:33) of a native sequence PRO21331 cDNA, wherein SEQ ID NO:33 is a clone designated herein as “DNA175959-2948” (UNQ6427).

FIG. 34 shows the amino acid sequence (SEQ ID NO:34) derived from the coding sequence of SEQ ID NO:33 shown in FIG. 33.

FIG. 35 shows a nucleotide sequence (SEQ ID NO:35) of a native sequence PRO23949 cDNA, wherein SEQ ID NO:35 is a clone designated herein as “DNA194607” (UNQ8923).

FIG. 36 shows the amino acid sequence (SEQ ID NO:36) derived from the coding sequence of SEQ ID NO:35 shown in FIG. 35.

FIG. 37 shows a nucleotide sequence (SEQ ID NO:37) of a native sequence PRO697 cDNA, wherein SEQ ID NO:37 is a clone designated herein as “DNA50920-1325” (UNQ361).

FIG. 38 shows the amino acid sequence (SEQ ID NO:38) derived from the coding sequence of SEQ ID NO:37 shown in FIG. 37.

FIG. 39 shows a nucleotide sequence (SEQ ID NO:39) of a native sequence PRO1480 cDNA, wherein SEQ ID NO:39 is a clone designated herein as “DNA67962-1649” (UNQ749).

FIG. 40 shows the amino acid sequence (SEQ ID NO:40) derived from the coding sequence of SEQ ID NO:39 shown in FIG. 39.

### DETAILED DESCRIPTION OF THE PREFERRED EMBODIMENTS

### I. Definitions

The terms “PRO polypeptide” and “PRO” as used herein and when immediately followed by a numerical designation refer to various polypeptides, wherein the complete designation (i.e., PRO/number) refers to specific polypeptide sequences as described herein. The terms “PRO/number polypeptide” and “PRO/number” wherein the term “number” is provided as an actual numerical designation as used herein encompass native sequence polypeptides and polypeptide variants (which are further defined herein). The PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides described herein may be isolated from a variety of sources, such as from human tissue types or from another source, or prepared by recombinant or synthetic methods. The term “PRO polypeptide” refers to each individual PRO/number polypeptide disclosed herein. All disclosures in this specification which refer to the “PRO polypeptide” refer to each of the polypeptides individually as well as jointly. For example, descriptions of the preparation of, purification of, derivation of, formation of antibodies to or against, administration of, compositions containing, treatment of a disease with, etc., pertain to each polypeptide of the invention individually. The term “PRO polypeptide” also includes variants of the PRO/number polypeptides disclosed herein.

A “native sequence PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide” comprises a polypeptide having the same amino acid sequence as the corresponding PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide derived from nature. Such native sequence PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides can be isolated from nature or can be produced by recombinant or synthetic means. The term “native sequence PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide” specifically encompasses naturally-occurring truncated or secreted forms of the specific PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide (e.g., an extracellular domain sequence), naturally-occurring variant forms (e.g., alternatively spliced forms) and naturally-occurring allelic variants of the polypeptide. The invention provides native sequence PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides disclosed herein which are mature or full-length native sequence polypeptides comprising the full-length amino acids sequences shown in the accompanying figures. Start and stop codons are shown in bold font and underlined in the figures. However, while the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide disclosed in the accompanying figures are shown to begin with methionine residues designated herein as amino acid position 1 in the figures, it is conceivable and possible that other methionine residues located either upstream or downstream from the amino acid position 1 in the figures may be employed as the starting amino acid residue for the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides.

The PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide “extracellular domain” or “ECD” refers to a form of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide which is essentially free of the transmembrane and cytoplasmic domains. Ordinarily, a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide ECD will have less than 1% of such transmembrane and/or cytoplasmic domains and preferably, will have less than 0.5% of such domains. It will be understood that any transmembrane domains identified for the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides of the present invention are identified pursuant to criteria routinely employed in the art for identifying that type of hydrophobic domain. The exact boundaries of a transmembrane domain may vary but most likely by no more than about 5 amino acids at either end of the domain as initially identified herein. Optionally, therefore, an extracellular domain of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide may contain from about 5 or fewer amino acids on either side of the transmembrane domain/extracellular domain boundary as identified in the Examples or specification and such polypeptides, with or without the associated signal peptide, and nucleic acid encoding them, are contemplated by the present invention.

The approximate location of the “signal peptides” of the various PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides disclosed herein are shown in the present specification and/or the accompanying figures. It is noted, however, that the C-terminal boundary of a signal peptide may vary, but most likely by no more than about 5 amino acids on either side of the signal peptide C-terminal boundary as initially identified herein, wherein the C-terminal boundary of the signal peptide may be identified pursuant to criteria routinely employed in the art for identifying that type of amino acid sequence element (e.g., Nielsen et al., Prot. Eng. 10:1-6 (1997) and von Heinje et al., Nucl. Acids. Res. 14:4683-4690 (1986)). Moreover, it is also recognized that, in some cases, cleavage of a signal sequence from a secreted polypeptide is not entirely uniform, resulting in more than one secreted species. These mature polypeptides, where the signal peptide is cleaved within no more than about 5 amino acids on either side of the C-terminal boundary of the signal peptide as identified herein, and the polynucleotides encoding them, are contemplated by the present invention.

“PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide variant” means a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, preferably an active PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, as defined herein having at least about 80% amino acid sequence identity with a full-length native sequence PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide sequence as disclosed herein, a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide sequence lacking the signal peptide as disclosed herein, an extracellular domain of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, with or without the signal peptide, as disclosed herein or any other fragment of a full-length PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide sequence as disclosed herein (such as those encoded by a nucleic acid that represents only a portion of the complete coding sequence for a full-length PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide). Such PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide variants include, for instance, PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides wherein one or more amino acid residues are added, or deleted, at the N- or C-terminus of the full-length native amino acid sequence. Ordinarily, a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide variant will have or will have at least about 80% amino acid sequence identity, alternatively will have or will have at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to a full-length native sequence PRO256, PRO34421. PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide sequence as disclosed herein, a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide sequence lacking the signal peptide as disclosed herein, an extracellular domain of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, with or without the signal peptide, as disclosed herein or any other specifically defined fragment of a full-length PRO256, PRO34421, PRO334, PRO770, PRO983, PRO1009, PRO107, PRO1158, PRO1250, PRO1317, PRO4334, PRO4395, PRO49192, PRO9799, PRO21175, PRO19837, PRO21331, PRO23949, PRO697 or PRO1480 polypeptide sequence as disclosed herein. Ordinarily, PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 variant polypeptides are or are at least about 10 amino acids in length, alternatively are or are at least about 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600 amino acids in length, or more. Optionally, PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 variant polypeptides will have no more than one conservative amino acid substitution as compared to the native PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide sequence, alternatively will have or will have no more than 2, 3, 4, 5, 6, 7, 8, 9, or 10 conservative amino acid substitution as compared to the native PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide sequence.

“Percent (%) amino acid sequence identity” with respect to the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide sequences identified herein is defined as the percentage of amino acid residues in a candidate sequence that are identical with the amino acid residues in the specific PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2, wherein the complete source code for the ALIGN-2 program is provided in Table 1 below. The ALIGN-2 sequence comparison computer program was authored by Genentech, Inc. and the source code shown in Table 1 below has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1 below. The ALIGN-2 program should be compiled for use on a UNIX operating system, preferably digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. As examples of % amino acid sequence identity calculations using this method, Tables 2 and 3 demonstrate how to calculate the % amino acid sequence identity of the amino acid sequence designated “Comparison Protein” to the amino acid sequence designated “PRO”, wherein “PRO” represents the amino acid sequence of a hypothetical PRO polypeptide of interest, “Comparison Protein” represents the amino acid sequence of a polypeptide against which the “PRO” polypeptide of interest is being compared, and “X, “Y” and “Z” each represent different hypothetical amino acid residues. Unless specifically stated otherwise, all % amino acid sequence identity values used herein are obtained as described in the immediately preceding paragraph using the ALIGN-2 computer program.

“PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 variant polynucleotide” or “PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 variant nucleic acid sequence” means a nucleic acid molecule which encodes a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, preferably an active PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, as defined herein and which has at least about 80% nucleic acid sequence identity with a nucleotide acid sequence encoding a full-length native sequence PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide sequence as disclosed herein, a full-length native sequence PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide sequence lacking the signal peptide as disclosed herein, an extracellular domain of a PRO256, PRO34421, PRO334, PRO770, PRO983, PRO1009, PRO107, PRO1158, PRO1250, PRO1317, PRO4334, PRO4395, PRO49192, PRO9799, PRO21175, PRO19837, PRO21331, PRO23949, PRO697 or PRO1480 polypeptide, with or without the signal peptide, as disclosed herein or any other fragment of a full-length PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide sequence as disclosed herein (such as those encoded by a nucleic acid that represents only a portion of the complete coding sequence for a full-length PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide). Ordinarily, a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 variant polynucleotide will have or will have at least about 80% nucleic acid sequence identity, alternatively will have or will have at least about 81%, 82%, 83%, 84%, 85%, 86%, 87%, 88%, 89%, 90%, 91%, 92%, 93%, 94%, 95%, 96%, 97%, 98%, or 99% nucleic acid sequence identity with a nucleic acid sequence encoding a full-length native sequence PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide sequence as disclosed herein, a full-length native sequence PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide sequence lacking the signal peptide as disclosed herein, an extracellular domain of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, with or without the signal sequence, as disclosed herein or any other fragment of a full-length PRO256, PRO34421, PRO334, PRO770, PRO983, PRO1009, PRO107, PRO1158, PRO1250, PRO1317, PRO4334, PRO4395, PRO49192, PRO9799, PRO21175, PRO19837, PRO21331, PRO23949, PRO697 or PRO1480 polypeptide sequence as disclosed herein. Variants do not encompass the native nucleotide sequence.

Ordinarily, PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 variant polynucleotides are or are at least about 5 nucleotides in length, alternatively are or are at least about 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides in length, wherein in this context the term “about” means the referenced nucleotide sequence length plus or minus 10% of that referenced length.

“Percent (%) nucleic acid sequence identity” with respect to PRO256-, PRO344-, PRO334-, PRO770-, PRO983-, PRO1009-, PRO1107-, PRO1158-, PRO1250-, PRO1317-, PRO4334-, PRO4395-, PRO49192-, PRO9799-, PRO21175-, PRO19837-, PRO21331-, PRO23949-, PRO697- or PRO1480-encoding nucleic acid sequences identified herein is defined as the percentage of nucleotides in a candidate sequence that are identical with the nucleotides in the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 nucleic acid sequence of interest, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN or Megalign (DNASTAR) software. For purposes herein, however, % nucleic acid sequence identity values are generated using the sequence comparison computer program ALIGN-2, wherein the complete source code for the ALIGN-2 program is provided in Table 1 below. The ALIGN-2 sequence comparison computer program was authored by Genentech, Inc. and the source code shown in Table 1 below has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1 below. The ALIGN-2 program should be compiled for use on a UNIX operating system, preferably digital UNIX V4.0D. All sequence comparison parameters are set by the ALIGN-2 program and do not vary.

In situations where ALIGN-2 is employed for nucleic acid sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:

100 times the fraction W/Z

where W is the number of nucleotides scored as identical matches by the sequence alignment program ALIGN-2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C. As examples of % nucleic acid sequence identity calculations, Tables 4 and 5, demonstrate how to calculate the % nucleic acid sequence identity of the nucleic acid sequence designated “Comparison DNA” to the nucleic acid sequence designated “PRO-DNA”, wherein “PRO-DNA” represents a hypothetical PRO-encoding nucleic acid sequence of interest, “Comparison DNA” represents the nucleotide sequence of a nucleic acid molecule against which the “PRO-DNA” nucleic acid molecule of interest is being compared, and “N”, “L” and “V” each represent different hypothetical nucleotides. Unless specifically stated otherwise, all % nucleic acid sequence identity values used herein are obtained as described in the immediately preceding paragraph using the ALIGN-2 computer program.

The invention also provides PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 variant polynucleotides which are nucleic acid molecules that encode a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide and which are capable of hybridizing, preferably under stringent hybridization and wash conditions, to nucleotide sequences encoding a full-length PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide as disclosed herein. PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 variant polypeptides may be those that are encoded by a PRO256, PRO34421, PRO334, PRO770, PRO983, PRO1009, PRO107, PRO1158, PRO1250, PRO1317, PRO4334, PRO4395, PRO49192, PRO9799, PRO21175, PRO19837, PRO21331, PRO23949, PRO697 or PRO1480 variant polynucleotide.

The term “full-length coding region” when used in reference to a nucleic acid encoding a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide refers to the sequence of nucleotides which encode the full-length PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide of the invention (which is often shown between start and stop codons, inclusive thereof, in the accompanying figures). The term “full-length coding region” when used in reference to an ATCC deposited nucleic acid refers to the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide-encoding portion of the cDNA that is inserted into the vector deposited with the ATCC (which is often shown between start and stop codons, inclusive thereof, in the accompanying figures).

“Isolated,” when used to describe the various polypeptides disclosed herein, means polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. The invention provides that the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated polypeptide includes polypeptide in situ within recombinant cells, since at least one component of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide natural environment will not be present. Ordinarily, however, isolated polypeptide will be prepared by at least one purification step.

An “isolated” PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide-encoding nucleic acid or other polypeptide-encoding nucleic acid is a nucleic acid molecule that is identified and separated from at least one contaminant nucleic acid molecule with which it is ordinarily associated in the natural source of the polypeptide-encoding nucleic acid. An isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal location different from that of natural cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The term “epitope tagged” when used herein refers to a chimeric polypeptide comprising a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide fused to a “tag polypeptide”. The tag polypeptide has enough residues to provide an epitope against which an antibody can be made, yet is short enough such that it does not interfere with activity of the polypeptide to which it is fused. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues).

“Active” or “activity” for the purposes herein refers to form(s) of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide which retain a biological and/or an immunological activity of native or naturally-occurring PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, wherein “biological” activity refers to a biological function (either inhibitory or stimulatory) caused by a native or naturally-occurring PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide other than the ability to induce the production of an antibody against an antigenic epitope possessed by a native or naturally-occurring PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide and an “immunological” activity refers to the ability to induce the production of an antibody against an antigenic epitope possessed by a native or naturally-occurring PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide.

The term “antagonist” is used in the broadest sense [unless otherwise qualified], and includes any molecule that partially or fully blocks, inhibits, or neutralizes a biological activity of a native PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide disclosed herein. In a similar manner, the term “agonist” is used in the broadest sense [unless otherwise qualified] and includes any molecule that mimics a biological activity of a native PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide disclosed herein. Suitable agonist or antagonist molecules specifically include agonist or antagonist antibodies or antibody fragments, fragments or amino acid sequence variants of native PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides, peptides, antisense oligonucleotides, small organic molecules, etc. Methods for identifying agonists or antagonists of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide may comprise contacting a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide with a candidate agonist or antagonist molecule and measuring a detectable change in one or more biological activities normally associated with the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide.

“Treating” or “treatment” or “alleviation” refers to both therapeutic treatment and prophylactic or preventative measures, wherein the object is to prevent or slow down (lessen) the targeted pathologic condition or disorder. A subject in need of treatment may already have the disorder, or may be prone to have the disorder or may be in whom the disorder is to be prevented.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

“Mammal” for purposes of treatment refers to any animal classified as a mammal, including humans, rodents such as rats or mice, domestic and farm animals, and zoo, sports, or pet animals, such as dogs, cats, cattle, horses, sheep, pigs, goats, rabbits, etc. Preferably, the mammal is human.

Administration “in combination with” one or more further therapeutic agents includes simultaneous (concurrent) and consecutive administration in any order.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

By “solid phase” is meant a non-aqueous matrix to which the antibody of the present invention can adhere. Examples of solid phases encompassed herein include those formed partially or entirely of glass (e.g., controlled pore glass), polysaccharides (e.g., agarose), polyacrylamides, polystyrene, polyvinyl alcohol and silicones. Depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

A “liposome” is a small vesicle composed of various types of lipids, phospholipids and/or surfactant which is useful for delivery of a drug (such as a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide or antibody thereto) to a mammal. The components of the liposome are commonly arranged in a bilayer formation, similar to the lipid arrangement of biological membranes.

A “small molecule” is defined herein to have a molecular weight below about 500 Daltons.

An “effective amount” of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody, a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 binding oligopeptide, a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 binding organic molecule or an agonist or antagonist thereof as disclosed herein is an amount sufficient to carry out a specifically stated purpose. An “effective amount” may be determined empirically and in a routine manner, in relation to the stated purpose.

The term “therapeutically effective amount” refers to an amount of an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody, a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 binding oligopeptide, a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 binding organic molecule or other drug effective to “treat” a disease or disorder in a subject or mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. See the definition herein of “treating”. To the extent the drug may prevent growth and/or kill existing cancer cells, it may be cytostatic and/or cytotoxic.

The phrases “cardiovascular, endothelial and angiogenic disorder”, “cardiovascular, endothelial and angiogenic dysfunction”, “cardiovascular, endothelial or angiogenic disorder” and “cardiovascular, endothelial or angiogenic dysfunction” are used interchangeably and refer in part to systemic disorders that affect vessels, such as diabetes mellitus, as well as diseases of the vessels themselves, such as of the arteries, capillaries, veins, and/or lymphatics. This would include indications that stimulate angiogenesis and/or cardiovascularization, and those that inhibit angiogenesis and/or cardiovascularization. Such disorders include, for example, arterial disease, such as atherosclerosis, hypertension, inflammatory vasculitides, Reynaud's disease and Reynaud's phenomenon, aneurysms, and arterial restenosis; venous and lymphatic disorders such as thrombophlebitis, lymphangitis, and lymphedema; and other vascular disorders such as peripheral vascular disease, cancer such as vascular tumors, e.g., hemangioma (capillary and cavernous), glomus tumors, telangiectasia, bacillary angiomatosis, hemangioendothelioma, angiosarcoma, hemangiopericytoma, Kaposi's sarcoma, lymphangioma, and lymphangiosarcoma, tumor angiogenesis, trauma such as wounds, burns, and other injured tissue, implant fixation, scarring, ischemia reperfusion injury, rheumatoid arthritis, cerebrovascular disease, renal diseases such as acute renal failure, or osteoporosis. This would also include angina, myocardial infarctions such as acute myocardial infarctions, cardiac hypertrophy, and heart failure such as CHF.

“Hypertrophy”, as used herein, is defined as an increase in mass of an organ or structure independent of natural growth that does not involve tumor formation. Hypertrophy of an organ or tissue is due either to an increase in the mass of the individual cells (true hypertrophy), or to an increase in the number of cells making up the tissue (hyperplasia), or both. Certain organs, such as the heart, lose the ability to divide shortly after birth. Accordingly, “cardiac hypertrophy” is defined as an increase in mass of the heart, which, in adults, is characterized by an increase in myocyte cell size and contractile protein content without concomitant cell division. The character of the stress responsible for inciting the hypertrophy, (e.g., increased preload, increased afterload, loss of myocytes, as in myocardial infarction, or primary depression of contractility), appears to play a critical role in determining the nature of the response. The early stage of cardiac hypertrophy is usually characterized morphologically by increases in the size of myofibrils and mitochondria, as well as by enlargement of mitochondria and nuclei. At this stage, while muscle cells are larger than normal, cellular organization is largely preserved. At a more advanced stage of cardiac hypertrophy, there are preferential increases in the size or number of specific organelles, such as mitochondria, and new contractile elements are added in localized areas of the cells, in an irregular manner. Cells subjected to long-standing hypertrophy show more obvious disruptions in cellular organization, including markedly enlarged nuclei with highly lobulated membranes, which displace adjacent myofibrils and cause breakdown of normal Z-band registration. The phrase “cardiac hypertrophy” is used to include all stages of the progression of this condition, characterized by various degrees of structural damage of the heart muscle, regardless of the underlying cardiac disorder. Hence, the term also includes physiological conditions instrumental in the development of cardiac hypertrophy, such as elevated blood pressure, aortic stenosis, or myocardial infarction.

“Heart failure” refers to an abnormality of cardiac function where the heart does not pump blood at the rate needed for the requirements of metabolizing tissues. The heart failure can be caused by a number of factors, including ischemic, congenital, rheumatic, or idiopathic forms.

“Congestive heart failure” (CHF) is a progressive pathologic state where the heart is increasingly unable to supply adequate cardiac output (the volume of blood pumped by the heart over time) to deliver the oxygenated blood to peripheral tissues. As CHF progresses, structural and hemodynamic damages occur. While these damages have a variety of manifestations, one characteristic symptom is ventricular hypertrophy. CHF is a common end result of a number of various cardiac disorders.

“Myocardial infarction” generally results from atherosclerosis of the coronary arteries, often with superimposed coronary thrombosis. It may be divided into two major types: transmural infarcts, in which myocardial necrosis involves the full thickness of the ventricular wall, and subendocardial (nontransmural) infarcts, in which the necrosis involves the subendocardium, the intramural myocardium, or both, without extending all the way through the ventricular wall to the epicardium. Myocardial infarction is known to cause both a change in hemodynamic effects and an alteration in structure in the damaged and healthy zones of the heart. Thus, for example, myocardial infarction reduces the maximum cardiac output and the stroke volume of the heart. Also associated with myocardial infarction is a stimulation of the DNA synthesis occurring in the interstice as well as an increase in the formation of collagen in the areas of the heart not affected.

As a result of the increased stress or strain placed on the heart in prolonged hypertension due, for example, to the increased total peripheral resistance, cardiac hypertrophy has long been associated with “hypertension”. A characteristic of the ventricle that becomes hypertrophic as a result of chronic pressure overload is an impaired diastolic performance. Fouad et al., J. Am. Coll. Cardiol., 4:1500-1506 (1984); Smith et al., J. Am. Coll. Cardiol., 5: 869-874 (1985). A prolonged left ventricular relaxation has been detected in early essential hypertension, in spite of normal or supranormal systolic function. Hartford et al., Hypertension, 6: 329-338 (1984). However, there is no close parallelism between blood pressure levels and cardiac hypertrophy. Although improvement in left ventricular function in response to antihypertensive therapy has been reported in humans, patients variously treated with a diuretic (hydrochlorothiazide), a β-blocker (propranolol), or a calcium channel blocker (diltiazem), have shown reversal of left ventricular hypertrophy, without improvement in diastolic function. Inouye et al., Am. J. Cardiol, 53: 1583-7 (1984).

Another complex cardiac disease associated with cardiac hypertrophy is “hypertrophic cardiomyopathy”. This condition is characterized by a great diversity of morphologic, functional, and clinical features (Maron et al., N. Engl. J. Med., 316: 780-789 (1987); Spirito et al., N. Engl. J. Med., 320: 749-755 (1989); Louie and Edwards, Prog. Cardiovasc. Dis., 36: 275-308 (1994); Wigle et al., Circulation 92: 1680-1692 (1995)), the heterogeneity of which is accentuated by the fact that it afflicts patients of all ages. Spirito et al., N. Engl. J. Med. 1336:775-785 (1997). The causative factors of hypertrophic cardiomyopathy are also diverse and little understood. In general, mutations in genes encoding sarcomeric proteins are associated with hypertrophic cardiomyopathy. Recent data suggest that β-myosin heavy chain mutations may account for approximately 30 to 40 percent of cases of familial hypertrophic cardiomyopathy. Watkins et al., N. Engl. J. Med., 326: 1108-1114 (1992); Schwartz et al, Circulation, 91: 532-540 (1995); Marian and Roberts, Circulation, 92: 1336-1347 (1995); Thierfelder et al., Cell, 77: 701-712 (1994); Watkins et al., Nat. Gen., 11: 434-437 (1995). Besides β-myosin heavy chain, other locations of genetic mutations include cardiac troponin T, alpha tropomyosin, cardiac myosin binding protein C, essential myosin light chain, and regulatory myosin light chain. See, Malik and Watkins, Curr. Opin. Cardiol., 12: 295-302 (1997).

Supravalvular “aortic stenosis” is an inherited vascular disorder characterized by narrowing of the ascending aorta, but other arteries, including the pulmonary arteries, may also be affected. Untreated aortic stenosis may lead to increased intracardiac pressure resulting in myocardial hypertrophy and eventually heart failure and death. The pathogenesis of this disorder is not fully understood, but hypertrophy and possibly hyperplasia of medial smooth muscle are prominent features of this disorder. It has been reported that molecular variants of the elastin gene are involved in the development and pathogenesis of aortic stenosis. U.S. Pat. No. 5,650,282 issued Jul. 22, 1997.

“Valvular regurgitation” occurs as a result of heart diseases resulting in disorders of the cardiac valves. Various diseases, like rheumatic fever, can cause the shrinking or pulling apart of the valve orifice, while other diseases may result in endocarditis, an inflammation of the endocardium or lining membrane of the atrioventricular orifices and operation of the heart. Defects such as the narrowing of the valve stenosis or the defective closing of the valve result in an accumulation of blood in the heart cavity or regurgitation of blood past the valve. If uncorrected, prolonged valvular stenosis or insufficiency may result in cardiac hypertrophy and associated damage to the heart muscle, which may eventually necessitate valve replacement.

The term “immune related disease” means a disease in which a component of the immune system of a mammal causes, mediates or otherwise contributes to a morbidity in the mammal. Also included are diseases in which stimulation or intervention of the immune response has an ameliorative effect on progression of the disease. Included within this term are immune-mediated inflammatory diseases, non-immune-mediated inflammatory diseases, infectious diseases, immunodeficiency diseases, neoplasia, etc.

The term “T cell mediated disease” means a disease in which T cells directly or indirectly mediate or otherwise contribute to a morbidity in a mammal. The T cell mediated disease may be associated with cell mediated effects, lymphokine mediated effects, etc., and even effects associated with B cells if the B cells are stimulated, for example, by the lymphokines secreted by T cells.

Examples of immune-related and inflammatory diseases, some of which are immune or T cell mediated, include systemic lupus erythematosis, rheumatoid arthritis, juvenile chronic arthritis, spondyloarthropathies, systemic sclerosis (scleroderma), idiopathic inflammatory myopathies (dermatomyositis, polymyositis), Sjögren's syndrome, systemic vasculitis, sarcoidosis, autoimmune hemolytic anemia (immune pancytopenia, paroxysmal nocturnal hemoglobinuria), autoimmune thrombocytopenia (idiopathic thrombocytopenic purpura, immune-mediated thrombocytopenia), thyroiditis (Grave's disease, Hashimoto's thyroiditis, juvenile lymphocytic thyroiditis, atrophic thyroiditis), diabetes mellitus, immune-mediated renal disease (glomerulonephritis, tubulointerstitial nephritis), demyelinating diseases of the central and peripheral nervous systems such as multiple sclerosis, idiopathic demyelinating polyneuropathy or Guillain-Barré syndrome, and chronic inflammatory demyelinating polyneuropathy, hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis, inflammatory bowel disease (ulcerative colitis: Crohn's disease), gluten-sensitive enteropathy, and Whipple's disease, autoimmune or immune-mediated skin diseases including bullous skin-diseases, erythema multiform and contact dermatitis, psoriasis, allergic diseases such as asthma, allergic rhinitis, atopic dermatitis, food hypersensitivity and urticaria, immunologic diseases of the lung such as eosinophilic pneumonia, idiopathic pulmonary fibrosis and hypersensitivity pneumonitis, or transplantation associated diseases including graft rejection and graft-versus-host-disease. Infectious diseases including viral diseases such as AIDS (HIV infection), hepatitis A, B, C, D, and E, herpes, etc., bacterial infections, fungal infections, protozoal infections and parasitic infections.

An “autoimmune disease” herein is a disease or disorder arising from and directed against an individual's own tissues or a co-segregate or manifestation thereof or resulting condition therefrom. Examples of autoimmune diseases or disorders include, but are not limited to arthritis (rheumatoid arthritis, juvenile rheumatoid arthritis, osteoarthritis, psoriatic arthritis, and ankylosing spondylitis), psoriasis, dermatitis including atopic dermatitis; chronic idiopathic urticaria, including chronic autoimmune urticaria, polymyositis/dermatomyositis, toxic epidermal necrolysis, systemic scleroderma and sclerosis, responses associated with inflammatory bowel disease (IBD) (Crohn's disease, ulcerative colitis), and IBD with co-segregate of pyoderma gangrenosum, erythema nodosum, primary sclerosing cholangitis, and/or episcleritis), respiratory distress syndrome, including adult respiratory distress syndrome (ARDS), meningitis, IgE-mediated diseases such as anaphylaxis and allergic rhinitis, encephalitis such as Rasmussen's encephalitis, uveitis, colitis such as microscopic colitis and collagenous colitis, glomerulonephritis (GN) such as membranous GN, idiopathic membranous GN, membranous proliferative GN (MPGN), including Type I and Type II, and rapidly progressive GN, allergic conditions, eczema, asthma, conditions involving infiltration of T cells and chronic inflammatory responses, atherosclerosis, autoimmune myocarditis, leukocyte adhesion deficiency, systemic lupus erythematosus (SLE) such as cutaneous SLE, lupus (including nephritis, cerebritis, pediatric, non-renal, discoid, alopecia), juvenile onset diabetes, multiple sclerosis (MS) such as spino-optical MS, allergic encephalomyelitis, immune responses associated with acute and delayed hypersensitivity mediated by cytokines and T-lymphocytes, tuberculosis, sarcoidosis, granulomatosis including Wegener's granulomatosis, agranulocytosis, vasculitis (including Large Vessel vasculitis (including Polymyalgia Rheumatica and Giant Cell (Takayasu's) Arteritis), Medium Vessel vasculitis (including Kawasaki's Disease and Polyarteritis Nodosa), CNS vasculitis, and ANCA-associated vasculitis, such as Churg-Strauss vasculitis or syndrome (CSS)), aplastic anemia, Coombs positive anemia, Diamond Blackfan anemia, immune hemolytic anemia including autoimmune hemolytic anemia (AIHA), pernicious anemia, pure red cell aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome, myasthenia gravis, antigen-antibody complex mediated diseases, anti-glomerular basement membrane disease, anti-phospholipid antibody syndrome, allergic neuritis, Bechet disease, Castleman's syndrome, Goodpasture's Syndrome, Lambert-Eaton Myasthenic Syndrome, Reynaud's syndrome, Sjorgen's syndrome, Stevens-Johnson syndrome, solid organ transplant rejection (including pretreatment for high panel reactive antibody titers, IgA deposit in tissues, and rejection arising from renal transplantation, liver transplantation, intestinal transplantation, cardiac transplantation, etc.), graft versus host disease (GVHD), pemphigoid bullous, pemphigus (including vulgaris, foliaceus, and pemphigus mucus-membrane pemphigoid), autoimmune polyendocrinopathies, Reiter's disease, stiff-man syndrome, immune complex nephritis, IgM polyneuropathies or IgM mediated neuropathy, idiopathic thrombocytopenic purpura (ITP), thrombotic thrombocytopenic purpura (TTP), thrombocytopenia (as developed by myocardial infarction patients, for example), including autoimmune thrombocytopenia, autoimmune disease of the testis and ovary including autoimmune orchitis and oophoritis, primary hypothyroidism; autoimmune endocrine diseases including autoimmune thyroiditis, chronic thyroiditis (Hashimoto's Thyroiditis), subacute thyroiditis, idiopathic hypothyroidism, Addison's disease, Grave's disease, autoimmune polyglandular syndromes (or polyglandular endocrinopathy syndromes), Type I diabetes also referred to as insulin-dependent diabetes mellitus (IDDM), including pediatric IDDM, and Sheehan's syndrome; autoimmune hepatitis, Lymphoid interstitial pneumonitis (HIV), bronchiolitis obliterans (non-transplant) vs NSIP, Guillain-Barré Syndrome, Berger's Disease (IgA nephropathy), primary biliary cirrhosis, celiac sprue (gluten enteropathy), refractory sprue with co-segregate dermatitis herpetiformis, cryoglobulinemia, amylotrophic lateral sclerosis (ALS; Lou Gehrig's disease), coronary artery disease, autoimmune inner ear disease (AIED), autoimmune hearing loss, opsoclonus myoclonus syndrome (OMS), polychondritis such as refractory polychondritis, pulmonary alveolar proteinosis, amyloidosis, giant cell hepatitis, scleritis, monoclonal gammopathy of uncertain/unknown significance (MGUS), peripheral neuropathy, paraneoplastic syndrome, channelopathies such as epilepsy, migraine, arrhythmia, muscular disorders, deafness, blindness, periodic paralysis, and channelopathies of the CNS; autism, inflammatory myopathy, and focal segmental glomerulosclerosis (FSGS).

The phrase “anxiety related disorders” refers to disorders of anxiety, mood, and substance abuse, including but not limited to: depression, generalized anxiety disorders, attention deficit disorder, sleep disorder, hyperactivity disorder, obsessive compulsive disorder, schizophrenia, cognitive disorders, hyperalgesia and sensory disorders. Such disorders include the mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, social anxiety, autism, specific phobia, substance-induced anxiety disorder, acute alcohol withdrawal, obsessive compulsive disorder, agoraphobia, monopolar disorders, bipolar disorder I or II, bipolar disorder not otherwise specified, cyclothymic disorder, depressive disorder, major depressive disorder, mood disorder, substance-induced mood disorder, enhancement of cognitive function, loss of cognitive function associated with but not limited to Alzheimer's disease, stroke, or traumatic injury to the brain, seizures resulting from disease or injury including but not limited to epilepsy, learning disorders/disabilities, cerebral palsy. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

The term “lipid metabolic disorder” refers to abnormal clinical chemistry levels of cholesterol and triglycerides, wherein elevated levels of these lipids is an indication for atherosclerosis. Additionally, abnormal serum lipid levels may be an indication of various cardiovascular diseases including hypertension, stroke, coronary artery diseases, diabetes and/or obesity.

The phrase “eye abnormality” refers to such potential disorders of the eye as they may be related to atherosclerosis or various opthalmological abnormalities. Such disorders include but are not limited to the following: retinal dysplasia, various retinopathies, restenosis, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstrom's syndrome, Cockayne's syndrome, dysplasia spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis. Cataracts are also considered an eye abnormality and are associated with such systemic diseases as: Human Down's syndrome, Hallerman-Streiff syndrome, Lowe syndrome, galactosemia, Marfan syndrome, Trismoy 13-15 condition, Alport syndrome, myotonic dystrophy, Fabry disease, hypothroidisms, or Conradi syndrome. Other ocular developmental anomalies include: Aniridia, anterior segment and dysgenesis syndrome. Cataracts may also occur as a result of an intraocular infection or inflammation (uveitis).

A “growth inhibitory amount” of an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody, PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 binding oligopeptide or PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 binding organic molecule is an amount capable of inhibiting the growth of a cell, especially tumor, e.g., cancer cell, either in vitro or in vivo. A “growth inhibitory amount” of an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody, PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 binding oligopeptide or PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 binding organic molecule for purposes of inhibiting neoplastic cell growth may be determined empirically and in a routine manner.

A “cytotoxic amount” of an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody, PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 binding oligopeptide or PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 binding organic molecule is an amount capable of causing the destruction of a cell, especially tumor, e.g., cancer cell, either in vitro or in vivo. A “cytotoxic amount” of an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody, PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 binding oligopeptide or PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 binding organic molecule for purposes of inhibiting neoplastic cell growth may be determined empirically and in a routine manner.

The term “antibody” is used in the broadest sense and specifically covers, for example, single anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody monoclonal antibodies (including agonist, antagonist, and neutralizing antibodies), anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody compositions with polyepitopic specificity, polyclonal antibodies, single chain anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibodies, and fragments of anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibodies (see below) as long as they exhibit the desired biological or immunological activity. The term “immunoglobulin” (Ig) is used interchangeable with antibody herein.

An “isolated antibody” is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. The invention provides that the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The basic 4-chain antibody unit is a heterotetrameric glycoprotein composed of two identical light (L) chains and two identical heavy (H) chains (an IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain). In the case of IgGs, the 4-chain unit is generally about 150,000 daltons. Each L chain is linked to a H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain (C_{L}) at its other end. The V_{L} is aligned with the V_{H} and the C_{L} is aligned with the first constant domain of the heavy chain (C_{H} 1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated α, δ, ε, γ, and μ, respectively. The γ and α classes are further divided into subclasses on the basis of relatively minor differences in C_{H} sequence and function, e.g., humans express the following subclasses: IgG1, IgG2, IgG3, IgG4, IgA1, and IgA2.

The term “variable” refers to the fact that certain segments of the variable domains differ extensively in sequence among antibodies. The V domain mediates antigen binding and define specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g. around about residues 24-34 (L1), 50-56 (L2) and 89-97 (L3) in the V_{L}, and around about 1-35 (H1), 50-65 (H2) and 95-102 (H3) in the V_{H}; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the V_{L}, and 26-32 (H1), 53-55 (H2) and 96-101 (H3) in the V_{H}; Chothia and Lesk J. Mol. Biol. 196:901-917 (1987)).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, Ape etc), and human constant region sequences.

An “intact” antibody is one which comprises an antigen-binding site as well as a C_{L} and at least heavy chain constant domains, C_{H} 1, C_{H} 2 and C_{H} 3. The constant domains may be native sequence constant domains (e.g. human native sequence constant domains) or amino acid sequence variant thereof. Preferably, the intact antibody has one or more effector functions.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (see U.S. Pat. No. 5,641,870, Example 2; Zapata et al., Protein Eng. 8(10): 1057-1062 [1995]); single-chain antibody molecules; and multispecific antibodies formed from antibody fragments.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H} 1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having divalent antigen-binding activity and is still capable of cross-linking antigen. Fab′ fragments differ from Fab fragments by having additional few residues at the carboxy terminus of the C_{H} 1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, which region is also the part recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

The term “diabodies” refers to small antibody fragments prepared by constructing sFv fragments (see preceding paragraph) with short linkers (about 5-10 residues) between the V_{H} and V_{L} domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the V_{H} and V_{L} domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

A “species-dependent antibody,” e.g., a mammalian anti-human IgE antibody, is an antibody which has a stronger binding affinity for an antigen from a first mammalian species than it has for a homologue of that antigen from a second mammalian species. Normally, the species-dependent antibody “bind specifically” to a human antigen (i.e., has a binding affinity (Kd) value of no more than about 1×10⁻⁷ M, preferably no more than about 1×10⁻⁸ and most preferably no more than about 1×10⁻⁹ M) but has a binding affinity for a homologue of the antigen from a second non-human mammalian species which is at least about 50 fold, or at least about 500 fold, or at least about 1000 fold, weaker than its binding affinity for the human antigen. The species-dependent antibody can be of any of the various types of antibodies as defined above, but preferably is a humanized or human antibody.

A “PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 binding oligopeptide” is an oligopeptide that binds, preferably specifically, to a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide as described herein. PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 binding oligopeptides may be chemically synthesized using known oligopeptide synthesis methodology or may be prepared and purified using recombinant technology. PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 binding oligopeptides usually are or are at least about 5 amino acids in length, alternatively are or are at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length or more, wherein such oligopeptides that are capable of binding, preferably specifically, to a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide as described herein. PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 binding oligopeptides may be identified without undue experimentation using well known techniques. In this regard, it is noted that techniques for screening oligopeptide libraries for oligopeptides that are capable of specifically binding to a polypeptide target are well known in the art (see, e.g., U.S. Pat. Nos. 5,556,762, 5,750,373, 4,708,871, 4,833,092, 5,223,409, 5,403,484, 5,571,689, 5,663,143; PCT Publication Nos. WO 84/03506 and WO84/03564; Geysen et al., Proc. Natl. Acad. Sci. U.S.A., 81:3998-4002 (1984); Geysen et al., Proc. Natl. Acad. Sci. U.S.A., 82:178-182 (1985); Geysen et al., in Synthetic Peptides as Antigens, 130-149 (1986); Geysen et al., J. Immunol. Meth., 102:259-274 (1987); Schoofs et al., J. Immunol., 140:611-616 (1988), Cwirla, S. E. et al. (1990) Proc. Natl. Acad. Sci. USA, 87:6378; Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363, and Smith, G. P. (1991) Current Opin. Biotechnol., 2:668).

A “PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 binding organic molecule” is an organic molecule other than an oligopeptide or antibody as defined herein that binds, preferably specifically, to a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide as described herein. PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 binding organic molecules may be identified and chemically synthesized using known methodology (see, e.g., PCT Publication Nos. WO00/00823 and WO00/39585). PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 binding organic molecules are usually less than about 2000 daltons in size, alternatively less than about 1500, 750, 500, 250 or 200 daltons in size, wherein such organic molecules that are capable of binding, preferably specifically, to a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide as described herein may be identified without undue experimentation using well known techniques. In this regard, it is noted that techniques for screening organic molecule libraries for molecules that are capable of binding to a polypeptide target are well known in the art (see, e.g., PCT Publication Nos. WO00/00823 and WO00/39585).

An antibody, oligopeptide or other organic molecule “which binds” an antigen of interest, e.g. a tumor-associated polypeptide antigen target, is one that binds the antigen with sufficient affinity such that the antibody, oligopeptide or other organic molecule is preferably useful as a diagnostic and/or therapeutic agent in targeting a cell or tissue expressing the antigen, and does not significantly cross-react with other proteins. The extent of binding of the antibody, oligopeptide or other organic molecule to a “non-target” protein will be less than about 10% of the binding of the antibody, oligopeptide or other organic molecule to its particular target protein as determined by fluorescence activated cell sorting (FACS) analysis or radioimmunoprecipitation (RIA). With regard to the binding of an antibody, oligopeptide or other organic molecule to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a Kd for the target of at least about 10⁻⁴ M, alternatively at least about 10⁻⁵ M, alternatively at least about 10⁻⁶ M, alternatively at least about 10⁻⁷ M, alternatively at least about 10⁻⁸ M, alternatively at least about 10⁻⁹ M, alternatively at least about 10⁻¹⁰ M, alternatively at least about 10⁻¹¹ M, alternatively at least about 10⁻¹² M, or greater. The term “specific binding” refers to binding where a molecule binds to a particular polypeptide or epitope on a particular polypeptide without substantially binding to any other polypeptide or polypeptide epitope.

An antibody, oligopeptide or other organic molecule that “inhibits the growth of tumor cells expressing a “PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480” or a “growth inhibitory” antibody, oligopeptide or other organic molecule is one which results in measurable growth inhibition of cancer cells expressing or overexpressing the appropriate PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. The PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide may be a transmembrane polypeptide expressed on the surface of a cancer cell or may be a polypeptide that is produced and secreted by a cancer cell. Preferred growth inhibitory anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibodies, oligopeptides or organic molecules inhibit growth of PRO256-, PRO34421-, PRO334-, PRO770-, PRO983-, PRO1009-, PRO1107-, PRO1158-, PRO1250-, PRO1317-, PRO4334-, PRO4395-, PRO49192-, PRO9799-, PRO21175-, PRO19837-, PRO21331-, PRO23949-, PRO697- or PRO1480-expressing tumor cells by or by greater than 20%, preferably from about 20% to about 50%, and even more preferably, by or by greater than 50% (e.g., from about 50% to about 100%) as compared to the appropriate control, the control typically being tumor cells not treated with the antibody, oligopeptide or other organic molecule being tested. Growth inhibition can be measured at an antibody concentration of about 0.1 to 30 μg/ml or about 0.5 nM to 200 nM in cell culture, where the growth inhibition is determined 1-10 days after exposure of the tumor cells to the antibody. Growth inhibition of tumor cells in vivo can be determined in various ways. The antibody is growth inhibitory in vivo if administration of the anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody at about 1 μg/kg to about 100 mg/kg body weight results in reduction in tumor size or tumor cell proliferation within about 5 days to 3 months from the first administration of the antibody, preferably within about 5 to 30 days.

An antibody, oligopeptide or other organic molecule which “induces apoptosis” is one which induces programmed cell death as determined by binding of annexin V, fragmentation of DNA, cell shrinkage, dilation of endoplasmic reticulum, cell fragmentation, and/or formation of membrane vesicles (called apoptotic bodies). The cell is usually one which overexpresses a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. Preferably the cell is a tumor cell, e.g., a prostate, breast, ovarian, stomach, endometrial, lung, kidney, colon, bladder cell. Various methods are available for evaluating the cellular events associated with apoptosis. For example, phosphatidyl serine (PS) translocation can be measured by annexin binding; DNA fragmentation can be evaluated through DNA laddering; and nuclear/chromatin condensation along with DNA fragmentation can be evaluated by any increase in hypodiploid cells. Preferably, the antibody, oligopeptide or other organic molecule which induces apoptosis is one which results in or in about 2 to 50 fold, preferably in or in about 5 to 50 fold, and most preferably in or in about 10 to 50 fold, induction of annexin binding relative to untreated cell in an annexin binding assay.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Natl. Acad. Sci. U.S.A. 95:652-656 (1998).

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see review M. in Daëron, Annu. Rev. Immunol. 15:203-234 (1997)). FcRs are reviewed in Ravetch and Kinet, Annu. Rev. Immunol. 9:457-492 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source, e.g., from blood.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g., as described in Gazzano-Santoro et al., J. Immunol. Methods 202:163 (1996), may be performed.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD). Preferably, the cancer comprises a tumor that expresses an IGF receptor, more preferably breast cancer, lung cancer, colorectal cancer, or prostate cancer, and most preferably breast or prostate cancer.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodepa, carboquone, meturedepa, and uredepa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhône-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE™ vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

The terms “cell proliferative disorder” and “proliferative disorder” refer to disorders that are associated with some degree of abnormal cell proliferation. In one aspect of the invention, the cell proliferative disorder is cancer.

“Tumor”, as used herein, refers to all neoplastic cell growth and proliferation, whether malignant or benign, and all pre-cancerous and cancerous cells and tissues.

An antibody, oligopeptide or other organic molecule which “induces cell death” is one which causes a viable cell to become nonviable. The cell is one which expresses a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, preferably a cell that overexpresses a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide as compared to a normal cell of the same tissue type. The PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide may be a transmembrane polypeptide expressed on the surface of a cancer cell or may be a polypeptide that is produced and secreted by a cancer cell. Preferably, the cell is a cancer cell, e.g., a breast, ovarian, stomach, endometrial, salivary gland, lung, kidney, colon, thyroid, pancreatic or bladder cell. Cell death in vitro may be determined in the absence of complement and immune effector cells to distinguish cell death induced by antibody-dependent cell-mediated cytotoxicity (ADCC) or complement dependent cytotoxicity (CDC). Thus, the assay for cell death may be performed using heat inactivated serum (i.e., in the absence of complement) and in the absence of immune effector cells. To determine whether the antibody, oligopeptide or other organic molecule is able to induce cell death, loss of membrane integrity as evaluated by uptake of propidium iodide (PI), trypan blue (see Moore et al. Cytotechnology 17:1-11 (1995)) or 7AAD can be assessed relative to untreated cells. Preferred cell death-inducing antibodies, oligopeptides or other organic molecules are those which induce PI uptake in the PI uptake assay in BT474 cells.

As used herein, the term “immunoadhesion” designates antibody-like molecules which combine the binding specificity of a heterologous protein (an “adhesion”) with the effector functions of immunoglobulin constant domains. Structurally, the immunoadhesions comprise a fusion of an amino acid sequence with the desired binding specificity which is other than the antigen recognition and binding site of an antibody (i.e., is “heterologous”), and an immunoglobulin constant domain sequence. The adhesion part of an immunoadhesion molecule typically is a contiguous amino acid sequence comprising at least the binding site of a receptor or a ligand. The immunoglobulin constant domain sequence in the immunoadhesion may be obtained from any immunoglobulin, such as IgG-1, IgG-2, IgG-3, or IgG-4 subtypes, IgA (including IgA-1 and IgA-2), IgE, IgD or IgM.

The word “label” when used herein refers to a detectable compound or composition which is conjugated directly or indirectly to the antibody so as to generate a “labeled” antibody. The label may be detectable by itself (e.g. radioisotope labels or fluorescent labels) or, in the case of an enzymatic label, may catalyze chemical alteration of a substrate compound or composition which is detectable.

“Replication-preventing agent” is an agent wherein replication, function, and/or growth of the cells is inhibited or prevented, or cells are destroyed, no matter what the mechanism, such as by apoptosis, angiostasis, cytosis, tumoricide, mytosis inhibition, blocking cell cycle progression, arresting cell growth, binding to tumors, acting as cellular mediators, etc. Such agents include a chemotherapeutic agent, cytotoxic agent, cytokine, growth-inhibitory agent, or anti-hormonal agent, e.g., an anti-estrogen compound such as tamoxifen, an anti-progesterone such as onapristone (see, EP 616 812); or an anti-androgen such as flutamide, as well as aromidase inhibitors, or a hormonal agent such as an androgen.

The term “cytotoxic agent” as used herein refers to a substance that inhibits or prevents the function of cells and/or causes destruction of cells. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents e.g. methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A tumoricidal agent causes destruction of tumor cells.

Preferred cytotoxic agents herein for the specific tumor types to use in combination with the antagonists herein are as follows:

1. Prostate cancer: androgens, docetaxel, paclitaxel, estramustine, doxorubicin, mitoxantrone, antibodies to ErbB2 domain(s) such as 2C4 (WO 01/00245; hybridoma ATCC HB-12697), which binds to a region in the extracellular domain of ErbB2 (e.g., any one or more residues in the region from about residue 22 to about residue 584 of ErbB2, inclusive), AVASTIN™ anti-vascular endothelial growth factor (VEGF), TARCEVA™ OSI-774 (erlotinib) (Genenetech and OSI Pharmaceuticals), or other epidermal growth factor receptor tyrosine kinase inhibitors (EGFR TKI's).
2. Stomach cancer: 5-fluorouracil (5FU), XELODA™ capecitabine, methotrexate, etoposide, cisplatin/carboplatin, paclitaxel, docetaxel, gemcitabine, doxorubicin, and CPT-11 (camptothcin-11; irinotecan, USA Brand Name: CAMPTOSAR®).
3. Pancreatic cancer: gemcitabine, 5FU, XELODA™ capecitabine, CPT-11, docetaxel, paclitaxel, cisplatin, carboplatin, TARCEVA™ erlotinib, and other EGFR TKI's.
4. Colorectal cancer: 5FU, XELODA™ capecitabine, CPT-11, oxaliplatin, AVASTIN™ anti-VEGF, TARCEVA™ erlotinib and other EGFR TKI's, and ERBITUX™ (formerly known as IMC-C225) human:murine-chimerized monoclonal antibody that binds to EGFR and blocks the ability of EGF to initiate receptor activation and signaling to the tumor.
5. Renal cancer: IL-2, interferon alpha, AVASTIN™ anti-VEGF, MEGACE™ (Megestrol acetate) progestin, vinblastine, TARCEVA™ erlotinib, and other EGFR TKI's.

A “growth inhibitory agent” when used herein refers to a compound or composition which inhibits growth of a cell, especially a PRO256-, PRO34421-, PRO334-, PRO770-, PRO983-, PRO1009-, PRO1107-, PRO1158-, PRO1250-, PRO1317-, PRO4334-, PRO4395-, PRO49192-, PRO9799-, PRO21175-, PRO19837-, PRO21331-, PRO23949-, PRO697- or PRO1480-expressing cancer cell, either in vitro or in vivo. Thus, the growth inhibitory agent may be one which significantly reduces the percentage of PRO256-, PRO34421-, PRO334-, PRO770-, PRO983-, PRO1009-, PRO1107-, PRO1158-, PRO1250-, PRO1317-, PRO4334-, PRO4395-, PRO49192-, PRO9799-, PRO21175-, PRO19837-, PRO21331-, PRO23949-, PRO697- or PRO1480-expressing cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (W B Saunders: Philadelphia, 1995), especially p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

“Doxorubicin” is an anthracycline antibiotic. The full chemical name of doxorubicin is (8S-cis)-10-[(3-amino-2,3,6-trideoxy-α-L-lyxo-hexapyranosyl)oxy]-7,8,9,10-tetrahydro-6,8,11-trihydroxy-8-(hydroxyacetyl)-1-methoxy-5,12-naphthacenedione.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1a, IL-2, IL-3, IL-4, IL-5, IL-6, IL-7, IL-8, IL-9, IL-11, IL-12; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native sequence cytokines.

The term “package insert” is used to refer to instructions customarily included in commercial packages of therapeutic products, that contain information about the indications, usage, dosage, administration, contraindications and/or warnings concerning the use of such therapeutic products.

The term “gene” refers to (a) a gene containing at least one of the DNA sequences disclosed herein; (b) any DNA sequence that encodes the amino acid sequence encoded by the DNA sequences disclosed herein and/or; ©) any DNA sequence that hybridizes to the complement of the coding sequences disclosed herein. Preferably, the term includes coding as well as noncoding regions, and preferably includes all sequences necessary for normal gene expression.

The term “gene targeting” refers to a type of homologous recombination that occurs when a fragment of genomic DNA is introduced into a mammalian cell and that fragment locates and recombines with endogenous homologous sequences. Gene targeting by homologous recombination employs recombinant DNA technologies to replace specific genomic sequences with exogenous DNA of particular design.

The term “homologous recombination” refers to the exchange of DNA fragments between two DNA molecules or chromatids at the site of homologous nucleotide sequences.

The term “target gene” (alternatively referred to as “target gene sequence” or “target DNA sequence”) refers to any nucleic acid molecule, polynucleotide, or gene to be modified by homologous recombination. The target sequence includes an intact gene, an exon or intron, a regulatory sequence or any region between genes. The target gene my comprise a portion of a particular gene or genetic locus in the individual's genomic DNA.

“Disruption” of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 gene occurs when a fragment of genomic DNA locates and recombines with an endogenous homologous sequence wherein the disruption is a deletion of the native gene or a portion thereof, or a mutation in the native gene or wherein the disruption is the functional inactivation of the native gene. Alternatively, sequence disruptions may be generated by nonspecific insertional inactivation using a gene trap vector (i.e. non-human transgenic animals containing and expressing a randomly inserted transgene; see for example U.S. Pat. No. 6,436,707 issued Aug. 20, 2002). These sequence disruptions or modifications may include insertions, missense, frameshift, deletion, or substitutions, or replacements of DNA sequence, or any combination thereof. Insertions include the insertion of entire genes, which may be of animal, plant, fungal, insect, prokaryotic, or viral origin. Disruption, for example, can alter the normal gene product by inhibiting its production partially or completely or by enhancing the normal gene product's activity. Preferably, the disruption is a null disruption, wherein there is no significant expression of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 gene.

The term “native expression” refers to the expression of the full-length polypeptide encoded by the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 gene, at expression levels present in the wild-type mouse. Thus, a disruption in which there is “no native expression” of the endogenous PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 gene refers to a partial or complete reduction of the expression of at least a portion of a polypeptide encoded by an endogenous PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 gene of a single cell, selected cells, or all of the cells of a mammal.

The term “knockout” refers to the disruption of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 gene wherein the disruption results in: the functional inactivation of the native gene; the deletion of the native gene or a portion thereof; or a mutation in the native gene.

The term “knock-in” refers to the replacement of the mouse ortholog (or other mouse gene) with a human cDNA encoding any of the specific human PRO256-, PRO34421-, PRO334-, PRO770-, PRO983-, PRO1009-, PRO1107-, PRO1158-, PRO1250-, PRO1317-, PRO4334-, PRO4395-, PRO49192-, PRO9799-, PRO21175-, PRO19837-, PRO21331-, PRO23949-, PRO697- or PRO1480-encoding genes or variants thereof (i.e. the disruption results in a replacement of a native mouse gene with a native human gene).

The term “construct” or “targeting construct” refers to an artificially assembled DNA segment to be transferred into a target tissue, cell line or animal. Typically, the targeting construct will include a gene or a nucleic acid sequence of particular interest, a marker gene and appropriate control sequences. As provided herein, the targeting construct comprises a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 targeting construct. A “PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 targeting construct” includes a DNA sequence homologous to at least one portion of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 gene and is capable of producing a disruption in a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 gene in a host cell.

The term “transgenic cell” refers to a cell containing within its genome a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 gene that has been disrupted, modified, altered, or replaced completely or partially by the method of gene targeting.

The term “transgenic animal” refers to an animal that contains within its genome a specific gene that has been disrupted or otherwise modified or mutated by the methods described herein or methods otherwise well known in the art. Preferably the non-human transgenic animal is a mammal. More preferably, the mammal is a rodent such as a rat or mouse. In addition, a “transgenic animal” may be a heterozygous animal (i.e., one defective allele and one wild-type allele) or a homozygous animal (i.e., two defective alleles). An embryo is considered to fall within the definition of an animal. The provision of an animal includes the provision of an embryo or foetus in utero, whether by mating or otherwise, and whether or not the embryo goes to term.

As used herein, the terms “selective marker” and position selection marker” refer to a gene encoding a product that enables only the cells that carry the gene to survive and/or grow under certain conditions. For example, plant and animal cells that express the introduced neomycin resistance (Neo^{r}) gene are resistant to the compound G418. Cells that do not carry the Neo^{r} gene marker are killed by G418. Other positive selection markers are known to, or are within the purview of, those of ordinary skill in the art.

The term “modulates” or “modulation” as used herein refers to the decrease, inhibition, reduction, amelioration, increase or enhancement of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 gene function, expression, activity, or alternatively a phenotype associated with PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 gene.

The term “ameliorates” or “amelioration” as used herein refers to a decrease, reduction or elimination of a condition, disease, disorder, or phenotype, including an abnormality or symptom.

The term “abnormality” refers to any disease, disorder, condition, or phenotype in which PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 is implicated, including pathological conditions and behavioral observations.

TABLE 2

| | | |
|---|---|---|
| PRO | XXXXXXXXXXXXXXX | (Length = 15 amino acids) |
| Comparison | XXXXXYYYYYYY | (Length = 12 amino acids) |
| Protein | | |

% amino acid sequence identity = (the number of identically matching amino acid residues between the two polypeptide sequences as determined by ALIGN-2) divided by (the total number of amino acid residues of the PRO polypeptide) = 5 divided by 15 = 33.3%

TABLE 3

| | | |
|---|---|---|
| PRO | XXXXXXXXXX | (Length = 10 amino acids) |
| Comparison | XXXXXYYYYYYZZYZ | (Length = 15 amino acids) |
| Protein | | |

% amino acid sequence identity = (the number of identically matching amino acid residues between the two polypeptide sequences as determined by ALIGN-2) divided by (the total number of amino acid residues of the PRO polypeptide) = 5 divided by 10 = 50%

TABLE 4

| | | |
|---|---|---|
| PRO-DNA | NNNNNNNNNNNNNN | (Length = 14 nucleotides) |
| Comparison | NNNNNNLLLLLLLLLL | (Length = 16 nucleotides) |
| DNA | | |

% nucleic acid sequence identity = (the number of identically matching nucleotides between the two nucleic acid sequences as determined by ALIGN-2) divided by (the total number of nucleotides of the PRO-DNA nucleic acid sequence) = 6 divided by 14 = 42.9%

TABLE 5

| | | |
|---|---|---|
| PRO-DNA | NNNNNNNNNNNN | (Length = 12 nucleotides) |
| Comparison DNA | NNNNLLLVV | (Length = 9 nucleotides) |

% nucleic acid sequence identity = (the number of identically matching nucleotides between the two nucleic acid sequences as determined by ALIGN-2) divided by (the total number of nucleotides of the PRO-DNA nucleic acid sequence) = 4 divided by 12 = 33.3%

### II. Compositions and Methods of the Invention

### A. Full-Length PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 Polypeptides

The present invention provides newly identified and isolated nucleotide sequences encoding polypeptides referred to in the present application as PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides. In particular, cDNAs encoding various PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides have been identified and isolated, as disclosed in further detail in the Examples below. It is noted that proteins produced in separate expression rounds may be given different PRO numbers but the UNQ number is unique for any given DNA and the encoded protein, and will not be changed. However, for sake of simplicity, in the present specification the protein encoded by the full length native nucleic acid molecules disclosed herein as well as all further native homologues and variants included in the foregoing definition of PRO, will be referred to as “PRO/number”, regardless of their origin or mode of preparation.

As disclosed in the Examples below, various cDNA clones have been deposited with the ATCC. The actual nucleotide sequences of those clones can readily be determined by the skilled artisan by sequencing of the deposited clone using routine methods in the art. The predicted amino acid sequence can be determined from the nucleotide sequence using routine skill. For the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides and encoding nucleic acids described herein, Applicants have identified what is believed to be the reading frame best identifiable with the sequence information available at the time.

### B. PRO256, PRO34421, PRO334, PRO770, PRO983. PRO1009, PRO1107, PRO1158, PRO1250, PRO1317, PRO4334, PRO4395, PRO49192, PRO9799, PRO21175, PRO19837. PRO21331, PRO23949, PRO697 or PRO1480 Polypeptide Variants

In addition to the full-length native sequence PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides described herein, it is contemplated that PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 variants can be prepared. PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 variants can be prepared by introducing appropriate nucleotide changes into the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 DNA, and/or by synthesis of the desired PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. Those skilled in the art will appreciate that amino acid changes may alter post-translational processes of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, such as changing the number or position of glycosylation sites or altering the membrane anchoring characteristics.

Variations in the native full-length sequence PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide or in various domains of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide described herein, can be made, for example, using any of the techniques and guidelines for conservative and non-conservative mutations set forth, for instance, in U.S. Pat. No. 5,364,934. Variations may be a substitution, deletion or insertion of one or more codons encoding the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide that results in a change in the amino acid sequence of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide as compared with the native sequence PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. Optionally the variation is by substitution of at least one amino acid with any other amino acid in one or more of the domains of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. Guidance in determining which amino acid residue may be inserted, substituted or deleted without adversely affecting the desired activity may be found by comparing the sequence of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence.

PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide fragments are provided herein. Such fragments may be truncated at the N-terminus or C-terminus, or may lack internal residues, for example, when compared with a full length native protein. Certain fragments lack amino acid residues that are not essential for a desired biological activity of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide.

PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 fragments may be prepared by any of a number of conventional techniques. Desired peptide fragments may be chemically synthesized. An alternative approach involves generating PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 fragments by enzymatic digestion, e.g., by treating the protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding a desired polypeptide fragment, by polymerase chain reaction (PCR). Oligonucleotides that define the desired termini of the DNA fragment are employed at the 5′ and 3′ primers in the PCR. Preferably, PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide fragments share at least one biological and/or immunological activity with the native PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide disclosed herein.

Conservative substitutions of interest are shown in Table 6 under the heading of preferred substitutions. If such substitutions result in a change in biological activity, then more substantial changes, denominated exemplary substitutions in Table 6, or as further described below in reference to amino acid classes, are preferably introduced and the products screened.

TABLE 6

| Original | Exemplary | Preferred |
|---|---|---|
| Residue | Substitutions | Substitutions |
| Ala (A) | Val; Leu; Ile | Val |
| Arg ®) | Lys; Gln; Asn | Lys |
| Asn (N) | Gln; His; Asp, Lys; Arg | Gln |
| Asp (D) | Glu; Asn | Glu |
| Cys ©) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; | Leu |
| | Phe; Norleucine | |
| Leu (L) | Norleucine; Ile; Val; | Ile |
| | Met; Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; | Leu |
| | Ala; Norleucine | |

Substantial modifications in function or immunological identity of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

Amino acids may be grouped according to similarities in the properties of their side chains (in A. L. Lehninger, in Biochemistry, second ed., pp. 73-75, Worth Publishers, New York (1975)):
(1) non-polar: Ala (A), Val (V), Leu (L), Ile (I), Pro (P), Phe (F), Trp (W), Met (M)
(2) uncharged polar: Gly (G), Ser (S), Thr (T), Cys (C), Tyr (Y), Asn (N), Gln (Q)
(3) acidic: Asp (D), Glu (E)
(4) basic: Lys (K), Arg (R), His (H)
Alternatively, naturally occurring residues may be divided into groups based on common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: H is, Lys, Arg;
(5) residues that influence chain orientation: Gly, Pro;
(6) aromatic: Trp, Tyr, Phe.

Non-conservative substitutions will entail exchanging a member of one of these classes for another class. Such substituted residues also may be introduced into the conservative substitution sites or, more preferably, into the remaining (non-conserved) sites.

The variations can be made using methods known in the art such as oligonucleotide-mediated (site-directed) mutagenesis, alanine scanning, and PCR mutagenesis. Site-directed mutagenesis [Carter et al., Nucl. Acids Res., 13:4331 (1986); Zoller et al., Nucl. Acids Res., 10:6487 (1987)], cassette mutagenesis [Wells et al., Gene, 34:315 (1985)], restriction selection mutagenesis [Wells et al., Philos. Trans. R. Soc. London SerA, 317:415 (1986)] or other known techniques can be performed on the cloned DNA to produce the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant [Cunningham and Wells, Science, 244: 1081-1085 (1989)]. Alanine is also typically preferred because it is the most common amino acid. Further, it is frequently found in both buried and exposed positions [Creighton, The Proteins, (W.H. Freeman & Co., N.Y.); Chothia, J. Mol. Biol., 150:1 (1976)]. If alanine substitution does not yield adequate amounts of variant, an isoteric amino acid can be used.

### C. Modifications of PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 Polypeptides

Covalent modifications of PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides are included within the scope of this invention. One type of covalent modification includes reacting targeted amino acid residues of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide with an organic derivatizing agent that is capable of reacting with selected side chains or the N- or C-terminal residues of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. Derivatization with bifunctional agents is useful, for instance, for crosslinking PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides to a water-insoluble support matrix or surface for use in the method for purifying anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibodies, and vice-versa. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains [T. E. Creighton, Proteins: Structure and Molecular Properties, W.H. Freeman & Co., San Francisco, pp. 79-86 (1983)], acetylation of the N-terminal amine, and amidation of any C-terminal carboxyl group.

Another type of covalent modification of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide included within the scope of this invention comprises altering the native glycosylation pattern of the polypeptide. “Altering the native glycosylation pattern” is intended for purposes herein to mean deleting one or more carbohydrate moieties found in native sequence PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides (either by removing the underlying glycosylation site or by deleting the glycosylation by chemical and/or enzymatic means), and/or adding one or more glycosylation sites that are not present in the native sequence PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. In addition, the phrase includes qualitative changes in the glycosylation of the native proteins, involving a change in the nature and proportions of the various carbohydrate moieties present.

Addition of glycosylation sites to the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide may be accomplished by altering the amino acid sequence. The alteration may be made, for example, by the addition of, or substitution by, one or more serine or threonine residues to the native sequence PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 (for O-linked glycosylation sites). The PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 amino acid sequence may optionally be altered through changes at the DNA level, particularly by mutating the DNA encoding the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide at preselected bases such that codons are generated that will translate into the desired amino acids.

Another means of increasing the number of carbohydrate moieties on the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide is by chemical or enzymatic coupling of glycosides to the polypeptide. Such methods are described in the art, e.g., in WO 87/05330 published 11 Sep. 1987, and in Aplin and Wriston, CRC Crit. Rev. Biochem., pp. 259-306 (1981).

Removal of carbohydrate moieties present on the PRO256, PRO34421, PRO334, PRO770, PRO983, PRO1009, PRO107, PRO1158, PRO1250, PRO1317, PRO4334, PRO4395, PRO49192, PRO9799, PRO21175, PRO19837, PRO21331, PRO23949, PRO697 or PRO1480 polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides comprises linking the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide to one of a variety of nonproteinaceous polymers, e.g., polyethylene glycol (PEG), polypropylene glycol, or polyoxyalkylenes, in the manner set forth in U.S. Pat. No. 4,640,835; 4,496,689; 4,301,144; 4,670,417; 4,791,192 or 4,179,337.

The PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides of the present invention may also be modified in a way to form a chimeric molecule comprising the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide fused to another, heterologous polypeptide or amino acid sequence.

Such a chimeric molecule comprises a fusion of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide with a tag polypeptide which provides an epitope to which an anti-tag antibody can selectively bind. The epitope tag is generally placed at the amino- or carboxyl-terminus of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. The presence of such epitope-tagged forms of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide can be detected using an antibody against the tag polypeptide. Also, provision of the epitope tag enables the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide to be readily purified by affinity purification using an anti-tag antibody or another type of affinity matrix that binds to the epitope tag. Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; an α-tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

The chimeric molecule may comprise a fusion of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide with an immunoglobulin or a particular region of an immunoglobulin. For a bivalent form of the chimeric molecule (also referred to as an “immunoadhesin”), such a fusion could be to the Fc region of an IgG molecule. The Ig fusions preferably include the substitution of a soluble (transmembrane domain deleted or inactivated) form of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide in place of at least one variable region within an Ig molecule. In a particularly preferred aspect of the invention, the immunoglobulin fusion includes the hinge, CH2 and CH3, or the hinge, CH1, CH2 and C3 regions of an IgG1 molecule. For the production of immunoglobulin fusions see also U.S. Pat. No. 5,428,130 issued Jun. 27, 1995.

### D. Preparation of PRO256, PRO34421, PRO334, PRO770, PRO983, PRO1009, PRO1107, PRO1158, PRO1250, PRO1317, PRO4334, PRO4395, PRO49192, PRO9799. PRO21175, PRO19837, PRO21331, PRO23949, PRO697 or PRO1480 Polypeptides

The description below relates primarily to production of PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides by culturing cells transformed or transfected with a vector containing PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 nucleic acid. It is, of course, contemplated that alternative methods, which are well known in the art, may be employed to prepare PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides. For instance, the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 sequence, or portions thereof, may be produced by direct peptide synthesis using solid-phase techniques [see, e.g., Stewart et al., Solid-Phase Peptide Synthesis, W.H. Freeman Co., San Francisco, Calif. (1969); Merrifield, J. Am. Chem. Soc., 85:2149-2154 (1963)]. In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide may be chemically synthesized separately and combined using chemical or enzymatic methods to produce the full-length PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide.

1. Isolation of DNA Encoding PRO256, PRO34421, PRO334, PRO770, PRO983, PRO1009 PRO1107, PRO1158, PRO1250, PRO1317, PRO4334, PRO4395, PRO49192, PRO9799, PRO21175, PRO19837, PRO21331, PRO23949, PRO697 or PRO1480 Polypeptides

DNA encoding PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides may be obtained from a cDNA library prepared from tissue believed to possess the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 mRNA and to express it at a detectable level. Accordingly, human PRO256-, PRO34421-, PRO334-, PRO770-, PRO983-, PRO1009-, PRO1107-, PRO1158-, PRO1250-, PRO1317-, PRO4334-, PRO4395-, PRO49192-, PRO9799-, PRO21175-, PRO19837-, PRO21331-, PRO23949-, PRO697- or PRO1480-DNA can be conveniently obtained from a cDNA library prepared from human tissue, such as described in the Examples. The PRO256-, PRO34421-, PRO334-, PRO770-, PRO983-, PRO1009-, PRO1107-, PRO1158-, PRO1250-, PRO1317-, PRO4334-, PRO4395-, PRO49192-, PRO9799-, PRO21175-, PRO19837-, PRO21331-, PRO23949-, PRO697- or PRO1480-encoding gene may also be obtained from a genomic library or by known synthetic procedures (e.g., automated nucleic acid synthesis).

Libraries can be screened with probes (such as antibodies to the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide or oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 is to use PCR methodology [Sambrook et al., supra; Dieffenbach et al., PCR Primer: A Laboratory Manual (Cold Spring Harbor Laboratory Press, 1995)].

The Examples below describe techniques for screening a cDNA library. The oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like ³²P-labeled ATP, biotinylation or enzyme labeling. Hybridization conditions, including moderate stringency and high stringency, are provided in Sambrook et al., supra.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined using methods known in the art and as described herein.

Nucleic acid having protein coding sequence may be obtained by screening selected cDNA or genomic libraries using the deduced amino acid sequence disclosed herein for the first time, and, if necessary, using conventional primer extension procedures as described in Sambrook et al., supra, to detect precursors and processing intermediates of mRNA that may not have been reverse-transcribed into cDNA.

2. Selection and Transformation of Host Cells

Host cells are transfected or transformed with expression or cloning vectors described herein for PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

Methods of eukaryotic cell transfection and prokaryotic cell transformation are known to the ordinarily skilled artisan, for example, CaCl₂, CaPO₄, liposome-mediated and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene, 23:315 (1983) and WO 89/05859 published 29 Jun. 1989. For mammalian cells without such cell walls, the calcium phosphate precipitation method of Graham and van der Eb, Virology, 52:456-457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT kan^{r}; E. coli W3110 strain 37D6, which has the complete genotype tonA ptr3 phoA E15 (argF-lac)169 degP ompT rbs7 ilvG kan^{r}; E. coli W3110 strain 40B4, which is strain 37D6 with a non-kanamycin resistant degP deletion mutation; and an E. coli strain having mutant periplasmic protease disclosed in U.S. Pat. No. 4,946,783 issued 7 Aug. 1990. Alternatively, in vitro methods of cloning, e.g., PCR or other nucleic acid polymerase reactions, are suitable.

In addition to prokaryotes, eukaryotic microbes such as filamentous fungi or yeast are suitable cloning or expression hosts for PRO256-, PRO34421-, PRO334-, PRO770-, PRO983-, PRO1009-, PRO1107-, PRO1158-, PRO1250-, PRO1317-, PRO4334-, PRO4395-, PRO49192-, PRO9799-, PRO21175-, PRO19837-, PRO21331, PRO23949-, PRO697- or PRO1480-encoding vectors. Saccharomyces cerevisiae is a commonly used lower eukaryotic host microorganism. Others include Schizosaccharomyces pombe (Beach and Nurse, Nature, 290: 140 [1981]; EP 139,383 published 2 May 1985); Kluyveromyces hosts (U.S. Pat. No. 4,943,529; Fleer et al., Bio/Technology, 9:968-975 (1991)) such as, e.g., K. lactis (MW98-8C, CBS683, CBS4574; Louvencourt et al., J. Bacteriol., 154(2):737-742 [1983]), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28:265-278 [1988]); Candida; Trichoderma reesia (EP 244,234); Neurospora crassa (Case et al., Proc. Natl. Acad. Sci. USA, 76:5259-5263 [1979]); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., Biochem. Biophys. Res. Commun., 112:284-289 [1983]; Tilburn et al., Gene, 26:205-221 [1983]; Yelton et al., Proc. Natl. Acad. Sci. USA, 81: 1470-1474 [1984]) and A. niger (Kelly and Hynes, EMBO J., 4:475-479 [1985]). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs, 269 (1982).

Suitable host cells for the expression of glycosylated PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides are derived from multicellular organisms. Examples of invertebrate cells include insect cells such as Drosophila S2 and Spodoptera Sf9, as well as plant cells. Examples of useful mammalian host cell lines include Chinese hamster ovary (CHO) and COS cells. More specific examples include monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); Chinese hamster ovary cells/−DHFR(CHO, Urlaub and Chasin, Proc. Natl. Acad. Sci. USA, 77:4216 (1980)); mouse sertoli cells (TM4, Mather, Biol. Reprod., 23:243-251 (1980)); human lung cells (W138, ATCC CCL 75); human liver cells (Hep G2, HB 8065); and mouse mammary tumor (MMT 060562, ATCC CCL51). The selection of the appropriate host cell is deemed to be within the skill in the art.

3. Selection and Use of a Replicable Vector

The nucleic acid (e.g., cDNA or genomic DNA) encoding PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the PRO256-, PRO34421-, PRO334-, PRO770-, PRO983-, PRO1009-, PRO1107-, PRO1158-, PRO1250-, PRO1317-, PRO4334-, PRO4395-, PRO49192-, PRO9799-, PRO21175-, PRO19837-, PRO21331-, PRO23949-, PRO697- or PRO1480-encoding DNA that is inserted into the vector. The signal sequence may be a prokaryotic signal sequence selected, for example, from the group of the alkaline phosphatase, penicillinase, Ipp, or heat-stable enterotoxin II leaders. For yeast secretion the signal sequence may be, e.g., the yeast invertase leader, alpha factor leader (including Saccharomyces and Kluyveromyces α-factor leaders, the latter described in U.S. Pat. No. 5,010,182), or acid phosphatase leader, the C. albicans glucoamylase leader (EP 362,179 published 4 Apr. 1990), or the signal described in WO 90/13646 published 15 Nov. 1990. In mammalian cell expression, mammalian signal sequences may be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the PRO256-, PRO34421-, PRO334-, PRO770-, PRO983-, PRO1009-, PRO1107-, PRO1158-, PRO1250-, PRO1317-, PRO4334-, PRO4395-, PRO49192-, PRO9799-, PRO21175-, PRO19837-, PRO21331-, PRO23949-, PRO697- or PRO1480-encoding nucleic acid, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77:4216 (1980). A suitable selection gene for use in yeast is the trp1 gene present in the yeast plasmid YRp7 [Stinchcomb et al., Nature, 282:39 (1979); Kingsman et al., Gene, 7:141 (1979); Tschemper et al., Gene, 10:157 (1980)]. The trp1 gene provides a selection marker for a mutant strain of yeast lacking the ability to grow in tryptophan, for example, ATCC No. 44076 or PEP4-1 [Jones, Genetics, 85:12 (1977)].

Expression and cloning vectors usually contain a promoter operably linked to the PRO256-, PRO34421-, PRO334-, PRO770-, PRO983-, PRO1009-, PRO1107-, PRO1158-, PRO1250-, PRO1317-, PRO4334-, PRO4395-, PRO49192-, PRO9799-, PRO21175-, PRO19837-, PRO21331-, PRO23949-, PRO697- or PRO1480-encoding nucleic acid sequence to direct mRNA synthesis. Promoters recognized by a variety of potential host cells are well known. Promoters suitable for use with prokaryotic hosts include the β-lactamase and lactose promoter systems [Chang et al., Nature, 275:615 (1978); Goeddel et al., Nature, 281:544 (1979)], alkaline phosphatase, a tryptophan (trp) promoter system [Goeddel, Nucleic Acids Res., 8:4057 (1980); EP 36,776], and hybrid promoters such as the tac promoter [deBoer et al., Proc. Natl. Acad. Sci. USA, 80:21-25 (1983)]. Promoters for use in bacterial systems also will contain a Shine-Dalgarno (S.D.) sequence operably linked to the DNA encoding PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides.

Examples of suitable promoting sequences for use with yeast hosts include the promoters for 3-phosphoglycerate kinase [Hitzeman et al., J. Biol. Chem., 255:2073 (1980)] or other glycolytic enzymes [Hess et al., J. Adv. Enzyme Reg., 7:149 (1968); Holland, Biochemistry, 17:4900 (1978)], such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 transcription from vectors in mammalian host cells is controlled, for example, by promoters obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides.

Still other methods, vectors, and host cells suitable for adaptation to the synthesis of PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides in recombinant vertebrate cell culture are described in Gething et al., Nature, 293:620-625 (1981); Mantei et al., Nature, 281:40-46 (1979); EP 117,060; and EP 117,058.

4. Detecting Gene Amplification/Expression

Gene amplification and/or expression may be measured in a sample directly, for example, by conventional Southern blotting, Northern blotting to quantitate the transcription of mRNA [Thomas, Proc. Natl. Acad. Sci. USA, 77:5201-5205 (1980)], dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide or against a synthetic peptide based on the DNA sequences provided herein or against exogenous sequence fused to PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 DNA and encoding a specific antibody epitope.

5. Purification of Polypeptide

Forms of PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides may be recovered from culture medium or from host cell lysates. Ifinembrane-bound, it can be released from the membrane using a suitable detergent solution (e.g. Triton-X 100) or by enzymatic cleavage. Cells employed in expression of PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides can be disrupted by various physical or chemical means, such as freeze-thaw cycling, sonication, mechanical disruption, or cell lysing agents.

It may be desired to purify PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide produced.

### E. Uses for PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 Polypeptides

Nucleotide sequences (or their complement) encoding PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides have various applications in the art of molecular biology, including uses as hybridization probes, in chromosome and gene mapping and in the generation of anti-sense RNA and DNA. PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 nucleic acid will also be useful for the preparation of PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides by the recombinant techniques described herein.

The full-length native sequence PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 gene, or portions thereof, may be used as hybridization probes for a cDNA library to isolate the full-length PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 cDNA or to isolate still other cDNAs (for instance, those encoding naturally-occurring variants of PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides or PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides from other species) which have a desired sequence identity to the native PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 sequence disclosed herein. Optionally, the length of the probes will be about 20 to about 50 bases. The hybridization probes may be derived from at least partially novel regions of the full length native nucleotide sequence wherein those regions may be determined without undue experimentation or from genomic sequences including promoters, enhancer elements and introns of native sequence PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480. By way of example, a screening method will comprise isolating the coding region of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 gene using the known DNA sequence to synthesize a selected probe of about 40 bases. Hybridization probes may be labeled by a variety of labels, including radionucleotides such as ³²P or ³⁵S, or enzymatic labels such as alkaline phosphatase coupled to the probe via avidin/biotin coupling systems. Labeled probes having a sequence complementary to that of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 gene of the present invention can be used to screen libraries of human cDNA, genomic DNA or mRNA to determine which members of such libraries the probe hybridizes to. Hybridization techniques are described in further detail in the Examples below.

Any EST sequences disclosed in the present application may similarly be employed as probes, using the methods disclosed herein.

Other useful fragments of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 nucleic acids include antisense or sense oligonucleotides comprising a singe-stranded nucleic acid sequence (either RNA or DNA) capable of binding to target PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 mRNA (sense) or PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 DNA (antisense) sequences. Antisense or sense oligonucleotides, according to the present invention, comprise a fragment of the coding region of PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 DNA. Such a fragment generally comprises at least about 14 nucleotides, preferably from about 14 to 30 nucleotides. The ability to derive an antisense or a sense oligonucleotide, based upon a cDNA sequence encoding a given protein is described in, for example, Stein and Cohen (Cancer Res. 48:2659, 1988) and van der Krol et al. (BioTechniques 6:958, 1988).

Binding of antisense or sense oligonucleotides to target nucleic acid sequences results in the formation of duplexes that block transcription or translation of the target sequence by one of several means, including enhanced degradation of the duplexes, premature termination of transcription or translation, or by other means. The antisense oligonucleotides thus may be used to block expression of PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480. Antisense or sense oligonucleotides further comprise oligonucleotides having modified sugar-phosphodiester backbones (or other sugar linkages, such as those described in WO 91/06629) and wherein such sugar linkages are resistant to endogenous nucleases. Such oligonucleotides with resistant sugar linkages are stable in vivo (i.e., capable of resisting enzymatic degradation) but retain sequence specificity to be able to bind to target nucleotide sequences.

Other examples of sense or antisense oligonucleotides include those oligonucleotides which are covalently linked to organic moieties, such as those described in WO 90/10048, and other moieties that increases affinity of the oligonucleotide for a target nucleic acid sequence, such as poly-(L-lysine). Further still, intercalating agents, such as ellipticine, and alkylating agents or metal complexes may be attached to sense or antisense oligonucleotides to modify binding specificities of the antisense or sense oligonucleotide for the target nucleotide sequence.

Antisense or sense oligonucleotides may be introduced into a cell containing the target nucleic acid sequence by any gene transfer method, including, for example, CaPO₄-mediated DNA transfection, electroporation, or by using gene transfer vectors such as Epstein-Barr virus. In a preferred procedure, an antisense or sense oligonucleotide is inserted into a suitable retroviral vector. A cell containing the target nucleic acid sequence is contacted with the recombinant retroviral vector, either in vivo or ex vivo. Suitable retroviral vectors include, but are not limited to, those derived from the murine retrovirus M-MuLV, N2 (a retrovirus derived from M-MuLV), or the double copy vectors designated DCT5A, DCT5B and DCT5C (see WO 90/13641).

Sense or antisense oligonucleotides also may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell.

Alternatively, a sense or an antisense oligonucleotide may be introduced into a cell containing the target nucleic acid sequence by formation of an oligonucleotide-lipid complex, as described in WO 90/10448. The sense or antisense oligonucleotide-lipid complex is preferably dissociated within the cell by an endogenous lipase.

Antisense or sense RNA or DNA molecules are generally at least about 5 bases in length, about 10 bases in length, about 15 bases in length, about 20 bases in length, about 25 bases in length, about 30 bases in length, about 35 bases in length, about 40 bases in length, about 45 bases in length, about 50 bases in length, about 55 bases in length, about 60 bases in length, about 65 bases in length, about 70 bases in length, about 75 bases in length, about 80 bases in length, about 85 bases in length, about 90 bases in length, about 95 bases in length, about 100 bases in length, or more.

The probes may also be employed in PCR techniques to generate a pool of sequences for identification of closely related PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 coding sequences.

Nucleotide sequences encoding a PRO256, PRO34421, PRO334, PRO770, PRO983, PRO1009, PRO107, PRO1158, PRO1250, PRO1317, PRO4334, PRO4395, PRO49192, PRO9799, PRO21175, PRO19837, PRO21331, PRO23949, PRO697 or PRO1480 polypeptide can also be used to construct hybridization probes for mapping the gene which encodes that PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide and for the genetic analysis of individuals with genetic disorders. The nucleotide sequences provided herein may be mapped to a chromosome and specific regions of a chromosome using known techniques, such as in situ hybridization, linkage analysis against known chromosomal markers, and hybridization screening with libraries.

When the coding sequences for PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 encode a protein which binds to another protein (for example, where the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 is a receptor), the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide can be used in assays to identify the other proteins or molecules involved in the binding interaction. By such methods, inhibitors of the receptor/ligand binding interaction can be identified. Proteins involved in such binding interactions can also be used to screen for peptide or small molecule inhibitors or agonists of the binding interaction. Also, the receptor PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 can be used to isolate correlative ligand(s). Screening assays can be designed to find lead compounds that mimic the biological activity of a native PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide or a receptor for PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides. Such screening assays will include assays amenable to high-throughput screening of chemical libraries, making them particularly suitable for identifying small molecule drug candidates. Small molecules contemplated include synthetic organic or inorganic compounds. The assays can be performed in a variety of formats, including protein-protein binding assays, biochemical screening assays, immunoassays and cell based assays, which are well characterized in the art.

Nucleic acids which encode PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides or its modified forms can also be used to generate either transgenic animals or “knock out” animals which, in turn, are useful in the development and screening of therapeutically useful reagents. A transgenic animal (e.g., a mouse or rat) is an animal having cells that contain a transgene, which transgene was introduced into the animal or an ancestor of the animal at a prenatal, e.g., an embryonic stage. A transgene is a DNA which is integrated into the genome of a cell from which a transgenic animal develops. The invention provides cDNA encoding a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide which can be used to clone genomic DNA encoding a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide in accordance with established techniques and the genomic sequences used to generate transgenic animals that contain cells which express DNA encoding PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides. Any technique known in the art may be used to introduce a target gene transgene into animals to produce the founder lines of transgenic animals. Such techniques include, but are not limited to pronuclear microinjection (U.S. Pat. Nos. 4,873,191, 4,736,866 and 4,870,009); retrovirus mediated gene transfer into germ lines (Van der Putten, et al., Proc. Natl. Acad. Sci., USA, 82:6148-6152 (1985)); gene targeting in embryonic stem cells (Thompson, et al., Cell, 56:313-321 (1989)); nonspecific insertional inactivation using a gene trap vector (U.S. Pat. No. 6,436,707); electroporation of embryos (Lo, Mol. Cell. Biol., 3:1803-1814 (1983)); and sperm-mediated gene transfer (Lavitrano, et al., Cell, 57:717-723 (1989)); etc. Typically, particular cells would be targeted for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 transgene incorporation with tissue-specific enhancers. Transgenic animals that include a copy of a transgene encoding a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide introduced into the germ line of the animal at an embryonic stage can be used to examine the effect of increased expression of DNA encoding PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides. Such animals can be used as tester animals for reagents thought to confer protection from, for example, pathological conditions associated with its overexpression. In accordance with this facet of the invention, an animal is treated with the reagent and a reduced incidence of the pathological condition, compared to untreated animals bearing the transgene, would indicate a potential therapeutic intervention for the pathological condition. Alternatively, non-human homologues of PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides can be used to construct a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 “knockout” animal whichhas adefective or altered gene encoding PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 proteins as a result of homologous recombination between the endogenous gene encoding PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides and altered genomic DNA encoding PRO256, PRO34421, PRO334, PRO770, PRO983, PRO1009, PRO107, PRO1158, PRO1250, PRO1317, PRO4334, PRO4395, PRO49192, PRO9799, PRO21175, PRO19837, PRO21331, PRO23949, PRO697 or PRO1480 polypeptides introduced into an embryonic stem cell of the animal. Preferably the knock out animal is a mammal. More preferably, the mammal is a rodent such as a rat or mouse. For example, cDNA encoding PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides can be used to clone genomic DNA encoding PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides in accordance with established techniques. A portion of the genomic DNA encoding the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide can be deleted or replaced with another gene, such as a gene encoding a selectable marker which can be used to monitor integration. Typically, several kilobases of unaltered flanking DNA (both at the 5′ and 3′ ends) are included in the vector [see e.g., Thomas and Capecchi, Cell, 51:503 (1987) for a description of homologous recombination vectors]. The vector is introduced into an embryonic stem cell line (e.g., by electroporation) and cells in which the introduced DNA has homologously recombined with the endogenous DNA are selected [see e.g., Li et al., Cell, 69:915 (1992)]. The selected cells are then injected into a blastocyst of an animal (e.g., a mouse or rat) to form aggregation chimeras [see e.g., Bradley, in Teratocarcinomas and Embryonic Stem Cells: A Practical Approach, E. J. Robertson, ed. (IRL, Oxford, 1987), pp. 113-152]. A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of the gene encoding the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide.

In addition, knockout mice can be highly informative in the discovery of gene function and pharmaceutical utility for a drug target, as well as in the determination of the potential on-target side effects associated with a given target. Gene function and physiology are so well conserved between mice and humans., since they are both mammals and contain similar numbers of genes, which are highly conserved between the species. It has recently been well documented, for example, that 98% of genes on mouse chromosome 16 have a human ortholog (Mural et al., Science 296:1661-71 (2002)).

Although gene targeting in embryonic stem (ES) cells has enabled the construction of mice with null mutations in many genes associated with human disease, not all genetic diseases are attributable to null mutations.

One can design valuable mouse models of human diseases by establishing a method for gene replacement (knock-in) which will disrupt the mouse locus and introduce a human counterpart with mutation, Subsequently one can conduct in vivo drug studies targeting the human protein (Kitamoto et. Al., Biochemical and Biophysical Res. Commun., 222:742-47 (1996)).

Nucleic acid encoding the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides may also be used in gene therapy. In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. (Zamecnik et al., Proc. Natl. Acad. Sci. USA 83:4143-4146 [1986]). The oligonucleotides can be modified to enhance their uptake, e.g. by substituting their negatively charged phosphodiester groups by uncharged groups.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection (Dzau et al., Trends in Biotechnology 11, 205-210 [1993]). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262, 4429-4432 (1987); and Wagner et al., Proc. Natl. Acad. Sci. USA 87, 3410-3414 (1990). For review of gene marking and gene therapy protocols see Anderson et al., Science 256, 808-813 (1992).

The PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides described herein may also be employed as molecular weight markers for protein electrophoresis purposes and the isolated nucleic acid sequences may be used for recombinantly expressing those markers.

The nucleic acid molecules encoding the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides or fragments thereof described herein are useful for chromosome identification. In this regard, there exists an ongoing need to identify new chromosome markers, since relatively few chromosome marking reagents, based upon actual sequence data are presently available. Each PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 nucleic acid molecule of the present invention can be used as a chromosome marker.

The PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides and nucleic acid molecules of the present invention may also be used diagnostically for tissue typing, wherein the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides of the present invention may be differentially expressed in one tissue as compared to another, preferably in a diseased tissue as compared to a normal tissue of the same tissue type. PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 nucleic acid molecules will find use for generating probes for PCR, Northern analysis, Southern analysis and Western analysis.

The PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides described herein may also be employed as therapeutic agents. The PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides of the present invention can be formulated according to known methods to prepare pharmaceutically useful compositions, whereby the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 product hereof is combined in admixture with a pharmaceutically acceptable carrier vehicle. Therapeutic formulations are prepared for storage by mixing the active ingredient having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™ or PEG.

The formulations to be used for in vivo administration must be sterile. This is readily accomplished by filtration through sterile filtration membranes, prior to or following lyophilization and reconstitution.

Therapeutic compositions herein generally are placed into a container having a sterile access port, for example, an intravenous solution bag or vial having a stopper pierceable by a hypodermic injection needle.

The route of administration is in accord with known methods, e.g. injection or infusion by intravenous, intraperitoneal, intracerebral, intramuscular, intraocular, intraarterial or intralesional routes, topical administration, or by sustained release systems.

Dosages and desired drug concentrations of pharmaceutical compositions of the present invention may vary depending on the particular use envisioned. The determination of the appropriate dosage or route of administration is well within the skill of an ordinary physician. Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” In Toxicokinetics and New Drug Development, Yacobi et al., Eds., Pergamon Press, New York 1989, pp. 42-96.

When in vivo administration of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide or agonist or antagonist thereof is employed, normal dosage amounts may vary from about 10 ng/kg to up to 100 mg/kg of mammal body weight or more per day, preferably about 1 μg/kg/day to 10 mg/kg/day, depending upon the route of administration. Guidance as to particular dosages and methods of delivery is provided in the literature; see, for example, U.S. Pat. Nos. 4,657,760; 5,206,344; or 5,225,212. It is anticipated that different formulations will be effective for different treatment compounds and different disorders, that administration targeting one organ or tissue, for example, may necessitate delivery in a manner different from that to another organ or tissue.

Where sustained-release administration of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide is desired in a formulation with release characteristics suitable for the treatment of any disease or disorder requiring administration of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, microencapsulation of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide is contemplated. Microencapsulation of recombinant proteins for sustained release has been successfully performed with human growth hormone (rhGH), interferon-(rhIFN—), interleukin-2, and MN rgp120. Johnson et al., Nat. Med., 2:795-799 (1996); Yasuda, Biomed. Ther., 27:1221-1223 (1993); Hora et al., Bio/Technology, 8:755-758 (1990); Cleland, “Design and Production of Single Immunization Vaccines Using Polylactide Polyglycolide Microsphere Systems,” in Vaccine Design: The Subunit and Adjuvant Approach, Powell and Newman, eds, (Plenum Press: New York, 1995), pp. 439-462; WO 97/03692, WO 96/40072, WO 96/07399; and U.S. Pat. No. 5,654,010.

The sustained-release formulations of these proteins were developed using poly-lactic-coglycolic acid (PLGA) polymer due to its biocompatibility and wide range of biodegradable properties. The degradation products of PLGA, lactic and glycolic acids, can be cleared quickly within the human body. Moreover, the degradability of this polymer can be adjusted from months to years depending on its molecular weight and composition. Lewis, “Controlled release of bioactive agents from lactide/glycolide polymer,” in: M. Chasin and R. Langer (Eds.), Biodegradable Polymers as Drug Delivery Systems (Marcel Dekker: New York, 1990), pp. 1-41.

This invention encompasses methods of screening compounds to identify those that mimic the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide (agonists) or prevent the effect of the PRO256, PRO34421, PRO334, PRO770, PRO983, PRO1009, PRO107, PRO1158, PRO1250, PRO1317, PRO4334, PRO4395, PRO49192, PRO9799, PRO21175, PRO19837, PRO21331, PRO23949, PRO697 or PRO1480 polypeptide (antagonists). Agonists that mimic a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide would be especially valuable therapeutically in those instances where a negative phenotype is observed based on findings with the non-human transgenic animal whose genome comprises a disruption of the gene which encodes for the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. Antagonists that prevent the effects of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide would be especially valuable therapeutically in those instances where a positive phenotype is observed based upon observations with the non-human transgenic knockout animal. Screening assays for antagonist drug candidates are designed to identify compounds that bind or complex with the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide encoded by the genes identified herein, or otherwise interfere with the interaction of the encoded polypeptide with other cellular proteins.

Such screening assays will include assays amenable to high-throughput screening of chemical libraries, making them particularly suitable for identifying small molecule drug candidates.

The assays can be performed in a variety of formats, including protein-protein binding assays, biochemical screening assays, immunoassays, and cell-based assays, which are well characterized in the art.

All assays for antagonists are common in that they call for contacting the drug candidate with a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide encoded by a nucleic acid identified herein under conditions and for a time sufficient to allow these two components to interact.

In binding assays, the interaction is binding and the complex formed can be isolated or detected in the reaction mixture. The PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide encoded by the gene identified herein or the drug candidate is immobilized on a solid phase, e.g., on a microtiter plate, by covalent or non-covalent attachments. Non-covalent attachment generally is accomplished by coating the solid surface with a solution of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide and drying. Alternatively, an immobilized antibody, e.g., a monoclonal antibody, specific for the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

If the candidate compound interacts with but does not bind to a particular PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide encoded by a gene identified herein, its interaction with that polypeptide can be assayed by methods well known for detecting protein-protein interactions. Such assays include traditional approaches, such as, e.g., cross-linking, co-immunoprecipitation, and co-purification through gradients or chromatographic columns. In addition, protein-protein interactions can be monitored by using a yeast-based genetic system described by Fields and co-workers (Fields and Song, Nature (London), 340:245-246 (1989); Chien et al., Proc. Natl. Acad. Sci. USA, 88:9578-9582 (1991)) as disclosed by Chevray and Nathans, Proc. Natl. Acad. Sci. USA, 89: 5789-5793 (1991). Many transcriptional activators, such as yeast GAL4, consist of two physically discrete modular domains, one acting as the DNA-binding domain, the other one functioning as the transcription-activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL1-lacZ reporter gene under control of a GAL4-activated promoter depends on reconstitution of GAL4 activity via protein-protein interaction. Colonies containing interacting polypeptides are detected with a chromogenic substrate for β-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

Compounds that interfere with the interaction of a gene encoding a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide identified herein and other intra- or extracellular components can be tested as follows: usually a reaction mixture is prepared containing the product of the gene and the intra- or extracellular component under conditions and for a time allowing for the interaction and binding of the two products. To test the ability of a candidate compound to inhibit binding, the reaction is run in the absence and in the presence of the test compound. In addition, a placebo may be added to a third reaction mixture, to serve as positive control. The binding (complex formation) between the test compound and the intra- or extracellular component present in the mixture is monitored as described hereinabove. The formation of a complex in the control reaction(s) but not in the reaction mixture containing the test compound indicates that the test compound interferes with the interaction of the test compound and its reaction partner.

To assay for antagonists, the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide may be added to a cell along with the compound to be screened for a particular activity and the ability of the compound to inhibit the activity of interest in the presence of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide indicates that the compound is an antagonist to the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. Alternatively, antagonists may be detected by combining the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide and a potential antagonist with membrane-bound PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide receptors or recombinant receptors under appropriate conditions for a competitive inhibition assay. The PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide can be labeled, such as by radioactivity, such that the number of PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide molecules bound to the receptor can be used to determine the effectiveness of the potential antagonist. The gene encoding the receptor can be identified by numerous methods known to those of skill in the art, for example, ligand panning and FACS sorting. Coligan et al., Current Protocols in Immun., 1(2): Chapter 5 (1991). Preferably, expression cloning is employed wherein polyadenylated RNA is prepared from a cell responsive to the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide and a cDNA library created from this RNA is divided into pools and used to transfect COS cells or other cells that are not responsive to the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. Transfected cells that are grown on glass slides are exposed to labeled PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. The PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide can be labeled by a variety of means including iodination or inclusion of a recognition site for a site-specific protein kinase. Following fixation and incubation, the slides are subjected to autoradiographic analysis. Positive pools are identified and sub-pools are prepared and re-transfected using an interactive sub-pooling and re-screening process, eventually yielding a single clone that encodes the putative receptor.

As an alternative approach for receptor identification, the labeled PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide can be photoaffinity-linked with cell membrane or extract preparations that express the receptor molecule. Cross-linked material is resolved by PAGE and exposed to X-ray film. The labeled complex containing the receptor can be excised, resolved into peptide fragments, and subjected to protein micro-sequencing. The amino acid sequence obtained from micro-sequencing would be used to design a set of degenerate oligonucleotide probes to screen a cDNA library to identify the gene encoding the putative receptor.

Another approach in assessing the effect of an antagonist to a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, would be administering a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 antagonist to a wild-type mouse in order to mimic a known knockout phenotype. Thus, one would initially knockout the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 gene of interest and observe the resultant phenotype as a consequence of knocking out or disrupting the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 gene. Subsequently, one could then assess the effectiveness of an antagonist to the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide by administering an antagonist to the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide to a wild-type mouse. An effective antagonist would be expected to mimic the phenotypic effect that was initially observed in the knockout animal.

Likewise, one could assess the effect of an agonist to a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, by administering a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 agonist to a non-human transgenic mouse in order to ameliorate a known negative knockout phenotype. Thus, one would initially knockout the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 gene of interest and observe the resultant phenotype as a consequence of knocking out or disrupting the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 gene. Subsequently, one could then assess the effectiveness of an agonist to the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide by administering an agonist to the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide to athe non-human transgenic mouse. An effective agonist would be expected to ameliorate the negative phenotypic effect that was initially observed in the knockout animal.

In another assay for antagonists, mammalian cells or a membrane preparation expressing the receptor would be incubated with a labeled PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide in the presence of the candidate compound. The ability of the compound to enhance or block this interaction could then be measured.

More specific examples of potential antagonists include an oligonucleotide that binds to the fusions of immunoglobulin with the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, and, in particular, antibodies including, without limitation, poly- and monoclonal antibodies and antibody fragments, single-chain antibodies, anti-idiotypic antibodies, and chimeric or humanized versions of such antibodies or fragments, as well as human antibodies and antibody fragments. Alternatively, a potential antagonist may be a closely related protein, for example, a mutated form of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide that recognizes the receptor but imparts no effect, thereby competitively inhibiting the action of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide.

Another potential PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide antagonist is an antisense RNA or DNA construct prepared using antisense technology, where, e.g., an antisense RNA or DNA molecule acts to block directly the translation of mRNA by hybridizing to targeted mRNA and preventing protein translation. Antisense technology can be used to control gene expression through triple-helix formation or antisense DNA or RNA, both of which methods are based on binding of a polynucleotide to DNA or RNA. For example, the 5′ coding portion of the polynucleotide sequence, which encodes the mature PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides herein, is used to design an antisense RNA oligonucleotide of from about 10 to 40 base pairs in length. A DNA oligonucleotide is designed to be complementary to a region of the gene involved in transcription (triple helix—see Lee et al., Nucl. Acids Res., 6:3073 (1979); Cooney et al., Science, 241: 456 (1988); Dervan et al., Science, 251:1360 (1991)), thereby preventing transcription and the production of the PRO256, PRO34421, PRO334, PRO770, PRO983, PRO1009, PRO107, PRO1158, PRO1250, PRO1317, PRO4334, PRO4395, PRO49192, PRO9799, PRO21175, PRO19837, PRO21331, PRO23949, PRO697 or PRO1480 polypeptide. The antisense RNA oligonucleotide hybridizes to the 330 mRNA in vivo and blocks translation of the mRNA molecule into the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide (antisense—Okano, Neurochem., 56:560 (1991); Oligodeoxynucleotides as Antisense Inhibitors of Gene Expression (CRC Press: Boca Raton, Fla., 1988). The oligonucleotides described above can also be delivered to cells such that the antisense RNA or DNA may be expressed in vivo to inhibit production of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. When antisense DNA is used, oligodeoxyribonucleotides derived from the translation-initiation site, e.g., between about −10 and +10 positions of the target gene nucleotide sequence, are preferred.

Potential antagonists include small molecules that bind to the active site, the receptor binding site, or growth factor or other relevant binding site of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, thereby blocking the normal biological activity of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. Examples of small molecules include, but are not limited to, small peptides or peptide-like molecules, preferably soluble peptides, and synthetic non-peptidyl organic or inorganic compounds.

Ribozymes are enzymatic RNA molecules capable of catalyzing the specific cleavage of RNA. Ribozymes act by sequence-specific hybridization to the complementary target RNA, followed by endonucleolytic cleavage. Specific ribozyme cleavage sites within a potential RNA target can be identified by known techniques. For further details see, e.g., Rossi, Current Biology, 4:469-471 (1994), and PCT publication No. WO 97/33551 (published Sep. 18, 1997).

Nucleic acid molecules in triple-helix formation used to inhibit transcription should be single-stranded and composed of deoxynucleotides. The base composition of these oligonucleotides is designed such that it promotes triple-helix formation via Hoogsteen base-pairing rules, which generally require sizeable stretches of purines or pyrimidines on one strand of a duplex. For further details see, e.g., PCT publication No. WO 97/33551, supra.

These small molecules can be identified by any one or more of the screening assays discussed hereinabove and/or by any other screening techniques well known for those skilled in the art.

Diagnostic and therapeutic uses of the herein disclosed molecules may also be based upon the positive functional assay hits disclosed and described below.

### F. Anti-PRO256, Anti-PRO34421, Anti-PRO334, Anti-PRO770, Anti-PRO983, Anti-PRO1009, Anti-PRO1107, Anti-PRO1158, Anti-PRO1250, Anti-PRO1317, Anti-PRO4334, Anti-PRO4395, Anti-PRO49192, Anti-PRO9799, Anti-PRO21175, Anti-PRO19837, Anti-PRO21331, Anti-PRO23949, Anti-PRO697 or Anti-PRO1480 Antibodies

The present invention provides anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibodies which may find use herein as therapeutic and/or diagnostic agents. Exemplary antibodies include polyclonal, monoclonal, humanized, bispecific, and heteroconjugate antibodies.

1. Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen (especially when synthetic peptides are used) to a protein that is immunogenic in the species to be immunized. For example, the antigen can be conjugated to keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R¹ and R′ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. Seven to 14 days later, the animals are bled and the serum is assayed for antibody titer. Animals are boosted until the titer plateaus. Conjugates also can be made in recombinant cell culture as protein fusions. Also, aggregating agents such as alum are suitably used to enhance the immune response.

2. Monoclonal Antibodies

Monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium which medium preferably contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which substances prevent the growth of HGPRT-deficient cells.

Preferred fusion partner myeloma cells are those that fuse efficiently, support stable high-level production of antibody by the selected antibody-producing cells, and are sensitive to a selective medium that selects against the unfused parental cells. Preferred myeloma cell lines are murine myeloma lines, such as those derived from MOPC-21 and MPC-11 mouse tumors available from the Salk Institute Cell Distribution Center, San Diego, Calif. USA, and SP-2 and derivatives e.g., X63—Ag8-653 cells available from the American Type Culture Collection, Manassas, Va., USA. Human myeloma and mouse-human heteromyeloma cell lines also have been described for the production of human monoclonal antibodies (Kozbor, J. Immunol., 133:3001 (1984); and Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The binding affinity of the monoclonal antibody can, for example, be determined by the Scatchard analysis described in Munson et al., Anal. Biochem., 107:220 (1980).

Once hybridoma cells that produce antibodies of the desired specificity, affinity, and/or activity are identified, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal e.g., by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Plückthun, Immunol. Revs. 130:151-188 (1992).

Monoclonal antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res. 21:2265-2266 (1993)). Thus, these techniques are viable alternatives to traditional monoclonal antibody hybridoma techniques for isolation of monoclonal antibodies.

The DNA that encodes the antibody may be modified to produce chimeric or fusion antibody polypeptides, for example, by substituting human heavy chain and light chain constant domain (C_{H} and C_{L}) sequences for the homologous murine sequences (U.S. Pat. No. 4,816,567; and Morrison, et al., Proc. Natl. Acad. Sci. USA, 81:6851 (1984)), or by fusing the immunoglobulin coding sequence with all or part of the coding sequence for a non-immunoglobulin polypeptide (heterologous polypeptide). The non-immunoglobulin polypeptide sequences can substitute for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

3. Human and Humanized Antibodies

The anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibodies of the invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol. 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity and HAMA response (human anti-mouse antibody) when the antibody is intended for human therapeutic use. According to the so-called “best-fit” method, the sequence of the variable domain of a rodent antibody is screened against the entire library of known human variable domain sequences. The human V domain sequence which is closest to that of the rodent is identified and the human framework region (FR) within it accepted for the humanized antibody (Sims et al., J. Immunol. 151:2296 (1993); Chothia et al., J. Mol. Biol., 196:901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol. 151:2623 (1993)).

It is further important that antibodies be humanized with retention of high binding affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Various forms of a humanized anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody are contemplated. For example, the humanized antibody may be an antibody fragment, such as a Fab, which is optionally conjugated with one or more cytotoxic agent(s) in order to generate an immunoconjugate. Alternatively, the humanized antibody may be an intact antibody, such as an intact IgG1 antibody.

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germline mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggemann et al., Year in Immuno. 7:33 (1993); U.S. Pat. Nos. 5,545,806, 5,569,825, 5,591,669 (all of GenPharm); 5,545,807; and WO 97/17852.

Alternatively, phage display technology (McCafferty et al., Nature 348:552-553 [1990]) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S, and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

As discussed above, human antibodies may also be generated by in vitro activated B cells (see U.S. Pat. Nos. 5,567,610 and 5,229,275).

4. Antibody fragments

In certain circumstances there are advantages of using antibody fragments, rather than whole antibodies. The smaller size of the fragments allows for rapid clearance, and may lead to improved access to solid tumors.

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Fab and F(ab′)₂ fragment with increased in vivo half-life comprising a salvage receptor binding epitope residues are described in U.S. Pat. No. 5,869,046. Other techniques for the production of antibody fragments will be apparent to the skilled practitioner. The antibody of choice is a single chain Fv fragment (scFv). See WO 93/16185; U.S. Pat. No. 5,571,894; and U.S. Pat. No. 5,587,458. Fv and sFv are the only species with intact combining sites that are devoid of constant regions; thus, they are suitable for reduced nonspecific binding during in vivo use. sFv fusion proteins may be constructed to yield fusion of an effector protein at either the amino or the carboxy terminus of an sFv. See Antibody Engineering, ed. Borrebaeck, supra. The antibody fragment may also be a “linear antibody”, e.g., as described in U.S. Pat. No. 5,641,870 for example. Such linear antibody fragments may be monospecific or bispecific.

5. Bispecific Antibodies

Bispecific antibodies are antibodies that have binding specificities for at least two different epitopes. Exemplary bispecific antibodies may bind to two different epitopes of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 protein as described herein. Other such antibodies may combine a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 binding site with a binding site for another protein. Alternatively, an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 arm may be combined with an arm which binds to a triggering molecule on a leukocyte such as a T-cell receptor molecule (e.g. CD3), or Fc receptors for IgG (FcγR), such as FcγRI (CD64), FcγRII (CD32) and FcγRIII (CD16), so as to focus and localize cellular defense mechanisms to the PRO256-, PRO34421-, PRO334-, PRO770-, PRO983-, PRO1009-, PRO1107-, PRO1158-, PRO1250-, PRO1317-, PRO4334-, PRO4395-, PRO49192-, PRO9799-, PRO21175-, PRO19837-, PRO21331-, PRO23949-, PRO697- or PRO1480-expressing cell. Bispecific antibodies may also be used to localize cytotoxic agents to cells which express a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide. These antibodies possess a PRO256-, PRO34421-, PRO334-, PRO770-, PRO983-, PRO1009-, PRO1107-, PRO1158-, PRO1250-, PRO1317-, PRO4334-, PRO4395-, PRO49192-, PRO9799-, PRO21175-, PRO19837-, PRO21331-, PRO23949-, PRO697- or PRO1480-binding arm and an arm which binds the cytotoxic agent (e.g., saporin, anti-interferon-α, vinca alkaloid, ricin A chain, methotrexate or radioactive isotope hapten). Bispecific antibodies can be prepared as full length antibodies or antibody fragments (e.g., F(ab′)₂ bispecific antibodies).

WO 96/16673 describes a bispecific anti-ErbB2/anti-FcγRIII antibody and U.S. Pat. No. 5,837,234 discloses a bispecific anti-ErbB2/anti-FcγRI antibody. A bispecific anti-ErbB2/Fcα antibody is shown in WO98/02463. U.S. Pat. No. 5,821,337 teaches a bispecific anti-ErbB2/anti-CD3 antibody.

Methods for making bispecific antibodies are known in the art. Traditional production of full length bispecific antibodies is based on the co-expression of two immunoglobulin heavy chain-light chain pairs, where the two chains have different specificities (Millstein et al., Nature 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J. 10:3655-3659 (1991).

According to a different approach, antibody variable domains with the desired binding specificity (antibody-antigen combining sites) are fused to immunoglobulin constant domain sequences. Preferably, the fusion is with an Ig heavy chain constant domain, comprising at least part of the hinge, C_{H}2, and C_{H}3 regions. It is preferred to have the first heavy-chain constant region (C_{H}1) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant affect on the yield of the desired chain combination.

The invention provides bispecific antibodies which are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology 121:210 (1986).

According to another approach described in U.S. Pat. No. 5,731,168, the interface between a pair of antibody molecules can be engineered to maximize the percentage of heterodimers which are recovered from recombinant cell culture. The preferred interface comprises at least a part of the C_{H}3 domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med. 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets. Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a V_{H} connected to a V_{L} by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al., J. Immunol. 147:60 (1991).

6. Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/200373; EP 03089]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

7. Multivalent Antibodies

A multivalent antibody may be internalized (and/or catabolized) faster than a bivalent antibody by a cell expressing an antigen to which the antibodies bind. The antibodies of the present invention can be multivalent antibodies (which are other than of the IgM class) with three or more antigen binding sites (e.g. tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable domains. For instance, the polypeptide chain(s) may comprise VD1-(X1)ₙ—VD2-(X2)ₙ—Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH—CH1-flexible linker-VH—CH1-Fc region chain; or VH—CH1-VH—CH1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

8. Effector Function Engineering

It may be desirable to modify the antibody of the invention with respect to effector function, e.g., so as to enhance antigen-dependent cell-mediated cytotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al., Cancer Research 53:2560-2565 (1993). Alternatively, an antibody can be engineered which has dual Fc regions and may thereby have enhanced complement lysis and ADCC capabilities. See Stevenson et al., Anti-Cancer Drug Design 3:219-230 (1989). To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

9. Immunoconjugates

The invention also pertains to immunoconjugates comprising an antibody conjugated to a cytotoxic agent such as a chemotherapeutic agent, a growth inhibitory agent, a toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re. Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

Conjugates of an antibody and one or more small molecule toxins, such as a calicheamicin, maytansinoids, a trichothene, and CC1065, and the derivatives of these toxins that have toxin activity, are also contemplated herein.

### Maytansine and Maytansinoids

The invention provides an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody (full length or fragments) which is conjugated to one or more maytansinoid molecules.

Maytansinoids are mitototic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533, the disclosures of which are hereby expressly incorporated by reference.

### Maytansinoid-Antibody Conjugates

In an attempt to improve their therapeutic index, maytansine and maytansinoids have been conjugated to antibodies specifically binding to tumor cell antigens. Immunoconjugates containing maytansinoids and their therapeutic use are disclosed, for example, in U.S. Pat. Nos. 5,208,020, 5,416,064 and European Patent EP 0 425 235 B1, the disclosures of which are hereby expressly incorporated by reference. Liu et al., Proc. Natl. Acad. Sci. USA 93:8618-8623 (1996) described immunoconjugates comprising a maytansinoid designated DM1 linked to the monoclonal antibody C242 directed against human colorectal cancer. The conjugate was found to be highly cytotoxic towards cultured colon cancer cells, and showed antitumor activity in an in vivo tumor growth assay. Chari et al., Cancer Research 52:127-131 (1992) describe immunoconjugates in which a maytansinoid was conjugated via a disulfide linker to the murine antibody A7 binding to an antigen on human colon cancer cell lines, or to another murine monoclonal antibody TA. 1 that binds the HER-2/neu oncogene. The cytotoxicity of the TA. 1-maytansonoid conjugate was tested in vitro on the human breast cancer cell line SK—BR-3, which expresses 3×10⁵ HER-2 surface antigens per cell. The drug conjugate achieved a degree of cytotoxicity similar to the free maytansonid drug, which could be increased by increasing the number of maytansinoid molecules per antibody molecule. The A7-maytansinoid conjugate showed low systemic cytotoxicity in mice.

Anti-PRO256, Anti-PRO34421, Anti-PRO334, Anti-PRO770, Anti-PRO983, Anti-PRO1009, Anti-PRO1107, Anti-PRO1158, Anti-PRO1250, Anti-PRO1317, Anti-PRO4334, Anti-PRO4395, Anti-PRO49192, Anti-PRO9799, Anti-PRO21175, Anti-PRO19837, Anti-PRO21331, Anti-PRO23949, Anti-PRO697 or Anti-PRO1480 Antibody-Maytansinoid Conjugates (Immunoconjugates)

Anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody-maytansinoid conjugates are prepared by chemically linking an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody to a maytansinoid molecule without significantly diminishing the biological activity of either the antibody or the maytansinoid molecule. An average of 3-4 maytansinoid molecules conjugated per antibody molecule has shown efficacy in enhancing cytotoxicity of target cells without negatively affecting the function or solubility of the antibody, although even one molecule of toxin/antibody would be expected to enhance cytotoxicity over the use of naked antibody. Maytansinoids are well known in the art and can be synthesized by known techniques or isolated from natural sources. Suitable maytansinoids are disclosed, for example, in U.S. Pat. No. 5,208,020 and in the other patents and nonpatent publications referred to hereinabove. Preferred maytansinoids are maytansinol and maytansinol analogues modified in the aromatic ring or at other positions of the maytansinol molecule, such as various maytansinol esters.

There are many linking groups known in the art for making antibody-maytansinoid conjugates, including, for example, those disclosed in U.S. Pat. No. 5,208,020 or EP Patent 0 425 235 B1, and Chari et al., Cancer Research 52:127-131 (1992). The linking groups include disulfide groups, thioether groups, acid labile groups, photolabile groups, peptidase labile groups, or esterase labile groups, as disclosed in the above-identified patents, disulfide and thioether groups being preferred.

Conjugates of the antibody and maytansinoid may be made using a variety of bifunctional protein coupling agents such as N-succinimidyl-3-(2-pyridyldithio) propionate (SPDP), succinimidyl-4-(N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as his (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Particularly preferred coupling agents include N-succinimidyl-3-(2-pyridyldithio) propionate (SPDP) (Carlsson et al., Biochem. J. 173:723-737 [1978]) and N-succinimidyl-4-(2-pyridylthio)pentanoate (SPP) to provide for a disulfide linkage.

The linker may be attached to the maytansinoid molecule at various positions, depending on the type of the link. For example, an ester linkage may be formed by reaction with a hydroxyl group using conventional coupling techniques. The reaction may occur at the C-3 position having a hydroxyl group, the C-14 position modified with hydroxymethyl, the C-15 position modified with a hydroxyl group, and the C-20 position having a hydroxyl group. The linkage is formed at the C-3 position of maytansinol or a maytansinol analogue.

### Calicheamicin

Another immunoconjugate of interest comprises an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody conjugated to one or more calicheamicin molecules. The calicheamicin family of antibiotics are capable of producing double-stranded DNA breaks at sub-picomolar concentrations. For the preparation of conjugates of the calicheamicin family, see U.S. Pat. Nos. 5,712,374, 5,714,586, 5,739,116, 5,767,285, 5,770,701, 5,770,710, 5,773,001, 5,877,296 (all to American Cyanamid Company). Structural analogues of calicheamicin which may be used include, but are not limited to, γ₁^{I}, α₁^{I}, α₁^{I}, N-acetyl-γ₁^{I}, PSAG and θ^{I}₁, (Hinman et al., Cancer Research 53:3336-3342 (1993), Lode et al., Cancer Research 58:2925-2928 (1998) and the aforementioned U.S. patents to American Cyanamid). Another anti-tumor drug that the antibody can be conjugated is QFA which is an antifolate. Both calicheamicin and QFA have intracellular sites of action and do not readily cross the plasma membrane. Therefore, cellular uptake of these agents through antibody mediated internalization greatly enhances their cytotoxic effects.

### Other Cytotoxic Agents

Other antitumor agents that can be conjugated to the anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibodies of the invention include BCNU, streptozoicin, vincristine and 5-fluorouracil, the family of agents known collectively LL-E33288 complex described in U.S. Pat. Nos. 5,053,394, 5,770,710, as well as esperamicins (U.S. Pat. No. 5,877,296).

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO 93/21232 published Oct. 28, 1993.

The present invention further contemplates an immunoconjugate formed between an antibody and a compound with nucleolytic-activity (e.g., a ribonuclease or a DNA endonuclease such as a deoxyribonuclease; DNase).

For selective destruction of the tumor, the antibody may comprise a highly radioactive atom. A variety of radioactive isotopes are available for the production of radioconjugated anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibodies. Examples include At²¹¹, I¹³¹, I¹²⁵, Y⁹⁰, R¹⁸⁶, Re¹⁸⁸, Sm¹⁵³, Bi²¹², P³², Pb²¹² and radioactive isotopes of Lu. When the conjugate is used for diagnosis, it may comprise a radioactive atom for scintigraphic studies, for example tc^{99m} or I¹²³, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

The radio- or other labels may be incorporated in the conjugate in known ways. For example, the peptide may be biosynthesized or may be synthesized by chemical amino acid synthesis using suitable amino acid precursors involving, for example, fluorine-19 in place of hydrogen. Labels such as tc^{99m} or I¹²³, Re¹⁸⁶, Re¹⁸⁸ and In¹¹¹ can be attached via a cysteine residue in the peptide. Yttrium-90 can be attached via a lysine residue. The IODOGEN method (Fraker et al (1978) Biochem. Biophys. Res. Commun. 80: 49-57 can be used to incorporate iodine-123. “Monoclonal Antibodies in Immunoscintigraphy” (Chatal, CRC Press 1989) describes other methods in detail.

Conjugates of the antibody and cytotoxic agent may be made using a variety of bifunctional protein coupling agents such as N-succinimidyl-3-(2-pyridyldithio) propionate (SPDP), succinimidyl-4-(N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of the cytotoxic drug in the cell. For example, an acid-labile linker, peptidase-sensitive linker, photolabile linker, dimethyl linker or disulfide-containing linker (Chari et al., Cancer Research 52:127-131 (1992); U.S. Pat. No. 5,208,020) may be used.

Alternatively, a fusion protein comprising the anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

The invention provides that the antibody may be conjugated to a “receptor” (such streptavidin) for utilization in tumor pre-targeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a radionucleotide).

10. Immunoliposomes

The anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibodies disclosed herein may also be formulated as immunoliposomes. A “liposome” is a small vesicle composed of various types of lipids, phospholipids and/or surfactant which is useful for delivery of a drug to a mammal. The components of the liposome are commonly arranged in a bilayer formation, similar to the lipid arrangement of biological membranes. Liposomes containing the antibody are prepared by methods known in the art, such as described in Epstein et al., Proc. Natl. Acad. Sci. USA 82:3688 (1985); Hwang et al., Proc. Natl. Acad. Sci. USA 77:4030 (1980); U.S. Pat. Nos. 4,485,045 and 4,544,545; and WO97/38731 published Oct. 23, 1997. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem. 257:286-288 (1982) via a disulfide interchange reaction. A chemotherapeutic agent is optionally contained within the liposome. See Gabizon et al., J. National Cancer Inst. 81(19):1484 (1989).

11. Pharmaceutical Compositions of Antibodies

Antibodies specifically binding a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide identified herein, as well as other molecules identified by the screening assays disclosed hereinbefore, can be administered for the treatment of various disorders in the form of pharmaceutical compositions.

If the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide is intracellular and whole antibodies are used as inhibitors, internalizing antibodies are preferred. However, lipofections or liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein may also contain more than one active compound as necessary for the particular indication being treated, preferably those with complementary activities that do not adversely affect each other. Alternatively, or in addition, the composition may comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles, and nanocapsules) or in macroemulsions. Such techniques are disclosed in Remington's Pharmaceutical Sciences, supra.

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

### G. Uses for Anti-PRO256, Anti-PRO34421, Anti-PRO334, Anti-PRO770, Anti-PRO983, Anti-PRO1009, Anti-PRO1107, Anti-PRO1158, Anti-PRO1250, Anti-PRO1317, Anti-PRO4334, Anti-PRO4395, Anti-PRO49192, Anti-PRO9799, Anti-PRO21175, Anti-PRO19837, Anti-PRO21331, Anti-PRO23949, Anti-PRO697 or Anti-PRO1480 Antibodies

The anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibodies of the invention have various therapeutic and/or diagnostic utilities for a neurological disorder; a cardiovascular, endothelial or angiogenic disorder; an immunological disorder; an oncological disorder; an embryonic developmental disorder or lethality, or a metabolic abnormality. For example, anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibodies may be used in diagnostic assays for PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480, e.g., detecting its expression (and in some cases, differential expression) in specific cells, tissues, or serum. Various diagnostic assay techniques known in the art may be used, such as competitive binding assays, direct or indirect sandwich assays and immunoprecipitation assays conducted in either heterogeneous or homogeneous phases [Zola, Monoclonal Antibodies: A Manual of Techniques, CRC Press, Inc. (1987) pp. 147-158]. The antibodies used in the diagnostic assays can be labeled with a detectable moiety. The detectable moiety should be capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the detectable moiety may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982).

Anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibodies also are useful for the affinity purification of PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides from recombinant cell culture or natural sources. In this process, the antibodies against PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides are immobilized on a suitable support, such a Sephadex resin or filter paper, using methods well known in the art. The immobilized antibody then is contacted with a sample containing the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide to be purified, and thereafter the support is washed with a suitable solvent that will remove substantially all the material in the sample except the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, which is bound to the immobilized antibody. Finally, the support is washed with another suitable solvent that will release the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide from the antibody.

The following examples are offered for illustrative purposes only, and are not intended to limit the scope of the present invention in any way.

All patent and literature references cited in the present specification are hereby incorporated by reference in their entirety.

### EXAMPLES

Commercially available reagents referred to in the examples were used according to manufacturer's instructions unless otherwise indicated. The source of those cells identified in the following examples, and throughout the specification, by ATCC accession numbers is the American Type Culture Collection, Manassas, Va.

### Example 1

### Extracellular Domain Homology Screening to Identify Novel Polypeptides and cDNA Encoding Therefor

The extracellular domain (ECD) sequences (including the secretion signal sequence, if any) from about 950 known secreted proteins from the Swiss-Prot public database were used to search EST databases. The EST databases included public databases (e.g., Dayhoff, GenBank), and proprietary databases (e.g. LIFESEQ™, Incyte Pharmaceuticals, Palo Alto, Calif.). The search was performed using the computer program BLAST or BLAST-2 (Altschul et al., Methods in Enzymology, 266:460-480 (1996)) as a comparison of the ECD protein sequences to a 6 frame translation of the EST sequences. Those comparisons with a BLAST score of 70 (or in some cases 90) or greater that did not encode known proteins were clustered and assembled into consensus DNA sequences with the program “phrap” (Phil Green, University of Washington, Seattle, Wash.).

Using this extracellular domain homology screen, consensus DNA sequences were assembled relative to the other identified EST sequences using phrap. In addition, the consensus DNA sequences obtained were often (but not always) extended using repeated cycles of BLAST or BLAST-2 and phrap to extend the consensus sequence as far as possible using the sources of EST sequences discussed above.

Based upon the consensus sequences obtained as described above, oligonucleotides were then synthesized and used to identify by PCR a cDNA library that contained the sequence of interest and for use as probes to isolate a clone of the full-length coding sequence for a PRO polypeptide. Forward and reverse PCR primers generally range from 20 to 30 nucleotides and are often designed to give a PCR product of about 100-1000 bp in length. The probe sequences are typically 40-55 bp in length. In some cases, additional oligonucleotides are synthesized when the consensus sequence is greater than about 1-1.5 kbp. In order to screen several libraries for a full-length clone, DNA from the libraries was screened by PCR amplification, as per Ausubel et al., Current Protocols in Molecular Biology, with the PCR primer pair. A positive library was then used to isolate clones encoding the gene of interest using the probe oligonucleotide and one of the primer pairs.

The cDNA libraries used to isolate the cDNA clones were constructed by standard methods using commercially available reagents such as those from Invitrogen, San Diego, Calif. The cDNA was primed with oligo dT containing a NotI site, linked with blunt to SalI hemikinased adaptors, cleaved with NotI, sized appropriately by gel electrophoresis, and cloned in a defined orientation into a suitable cloning vector (such as pRKB or pRKD; pRK5B is a precursor of pRK5D that does not contain the SfiI site; see, Holmes et al., Science, 253:1278-1280 (1991)) in the unique XhoI and NotI sites.

### Example 2

### Isolation of cDNA Clones by Amylase Screening

1. Preparation of Oligo dT Primed cDNA Library

mRNA was isolated from a human tissue of interest using reagents and protocols from Invitrogen, San Diego, Calif. (Fast Track 2). This RNA was used to generate an oligo dT primed cDNA library in the vector pRK5D using reagents and protocols from Life Technologies, Gaithersburg, Md. (Super Script Plasmid System). In this procedure, the double stranded cDNA was sized to greater than 1000 bp and the SalI/NotI linkered cDNA was cloned into XhoI/NotI cleaved vector. pRK5D is a cloning vector that has an sp6 transcription initiation site followed by an SfiI restriction enzyme site preceding the XhoI/NotI cDNA cloning sites.

2. Preparation of Random Primed cDNA Library

A secondary cDNA library was generated in order to preferentially represent the 5′ ends of the primary cDNA clones. Sp6 RNA was generated from the primary library (described above), and this RNA was used to generate a random primed cDNA library in the vector pSST-AMY.0 using reagents and protocols from Life Technologies (Super Script Plasmid System, referenced above). In this procedure the double stranded cDNA was sized to 500-1000 bp, linkered with blunt to NotI adaptors, cleaved with SfiI, and cloned into SfiI/NotI cleaved vector. pSST-AMY.0 is a cloning vector that has a yeast alcohol dehydrogenase promoter preceding the cDNA cloning sites and the mouse amylase sequence (the mature sequence without the secretion signal) followed by the yeast alcohol dehydrogenase terminator, after the cloning sites. Thus, cDNAs cloned into this vector that are fused in frame with amylase sequence will lead to the secretion of amylase from appropriately transfected yeast colonies.

3. Transformation and Detection

DNA from the library described in paragraph 2 above was chilled on ice to which was added electrocompetent DH10B bacteria (Life Technologies, 20 ml). The bacteria and vector mixture was then electroporated as recommended by the manufacturer. Subsequently, SOC media (Life Technologies, 1 ml) was added and the mixture was incubated at 37° C. for 30 minutes. The transformants were then plated onto 20 standard 150 mm LB plates containing ampicillin and incubated for 16 hours (37° C.). Positive colonies were scraped off the plates and the DNA was isolated from the bacterial pellet using standard protocols, e.g. CsCl-gradient. The purified DNA was then carried on to the yeast protocols below.

The yeast methods were divided into three categories: (1) Transformation of yeast with the plasmid/cDNA combined vector; (2) Detection and isolation of yeast clones secreting amylase; and (3) PCR amplification of the insert directly from the yeast colony and purification of the DNA for sequencing and further analysis.

The yeast strain used was HD56-5A (ATCC-90785). This strain has the following genotype: MAT alpha, ura3-52, leu2-3, leu2-112, his3-11, his3-15, MAL⁺, SUC⁺, GAL⁺. Preferably, yeast mutants can be employed that have deficient post-translational pathways. Such mutants may have translocation deficient alleles in sec71, sec72, sec62, with truncated sec71 being most preferred. Alternatively, antagonists (including antisense nucleotides and/or ligands) which interfere with the normal operation of these genes, other proteins implicated in this post translation pathway (e.g., SEC61p, SEC72p, SEC62p, SEC63p, TDJ1p or SSA1p-4-p) or the complex formation of these proteins may also be preferably employed in combination with the amylase-expressing yeast. Transformation was performed based on the protocol outlined by Gietz et al., Nucl. Acid. Res., 20:1425 (1992). Transformed cells were then inoculated from agar into YEPD complex media broth (100 ml) and grown overnight at 30° C. The YEPD broth was prepared as described in Kaiser et al., Methods in Yeast Genetics, Cold Spring Harbor Press, Cold Spring Harbor, N.Y., p. 207 (1994). The overnight culture was then diluted to about 2×10⁶ cells/ml (approx. OD₆₀₀=0.1) into fresh YEPD broth (500 ml) and regrown to 1×10⁷ cells/ml (approx. OD₆₀₀=0.4-0.5).

The cells were then harvested and prepared for transformation by transfer into GS3 rotor bottles in a Sorval GS3 rotor at 5,000 rpm for 5 minutes, the supernatant discarded, and then resuspended into sterile water, and centrifuged again in 50 ml falcon tubes at 3,500 rpm in a Beckman GS-6KR centrifuge. The supernatant was discarded and the cells were subsequently washed with LiAc/TE (10 ml, 10 mM Tris-HCl, 1 mM EDTA pH 7.5, 100 mM Li₂OOCCH₃), and resuspended into LiAc/TE (2.5 ml).

Transformation took place by mixing the prepared cells (100 μl) with freshly denatured single stranded salmon testes DNA (Lofstrand Labs, Gaithersburg, Md.) and transforming DNA (1 μg, vol. <10 μl) in microfuge tubes. The mixture was mixed briefly by vortexing, then 40% PEG/TE (600 μl, 40% polyethylene glycol-4000, 10 mM Tris-HCl, 1 mM EDTA, 100 mM Li₂OOCCH₃, pH 7.5) was added. This mixture was gently mixed and incubated at 30° C. while agitating for 30 minutes. The cells were then heat shocked at 42° C. for 15 minutes, and the reaction vessel centrifuged in a microfuge at 12,000 rpm for 5-10 seconds, decanted and resuspended into TE (500 μl, 10 mM Tris-HCl, 1 mM EDTA pH 7.5) followed by recentrifugation. The cells were then diluted into TE (1 ml) and aliquots (200 μl) were spread onto the selective media previously prepared in 150 mm growth plates (VWR).

Alternatively, instead of multiple small reactions, the transformation was performed using a single, large scale reaction, wherein reagent amounts were scaled up accordingly.

The selective media used was a synthetic complete dextrose agar lacking uracil (SCD-Ura) prepared as described in Kaiser et al., Methods in Yeast Genetics, Cold Spring Harbor Press, Cold Spring Harbor, N.Y., p. 208-210 (1994). Transformants were grown at 30° C. for 2-3 days.

The detection of colonies secreting amylase was performed by including red starch in the selective growth media. Starch was coupled to the red dye (Reactive Red-120, Sigma) as per the procedure described by Biely et al., Anal. Biochem., 172:176-179 (1988). The coupled starch was incorporated into the SCD-Ura agar plates at a final concentration of 0.15% (w/v), and was buffered with potassium phosphate to a pH of 7.0 (50-100 mM final concentration).

The positive colonies were picked and streaked across fresh selective media (onto 150 mm plates) in order to obtain well isolated and identifiable single colonies. Well isolated single colonies positive for amylase secretion were detected by direct incorporation of red starch into buffered SCD-Ura agar. Positive colonies were determined by their ability to break down starch resulting in a clear halo around the positive colony visualized directly.

4. Isolation of DNA by PCR Amplification

When a positive colony was isolated, a portion of it was picked by a toothpick and diluted into sterile water (30 μl) in a 96 well plate. At this time, the positive colonies were either frozen and stored for subsequent analysis or immediately amplified. An aliquot of cells (5 μl) was used as a template for the PCR reaction in a 25 μl volume containing: 0.5 μl Klentaq (Clontech, Palo Alto, Calif.); 4.0 μl 10 mM dNTP's (Perkin Elmer-Cetus); 2.5 μl Kentaq buffer (Clontech); 0.25 μl forward oligo 1; 0.25 μl reverse oligo 2; 12.5 μl distilled water. The sequence of the forward oligonucleotide 1 was:

(SEQ ID NO:41)

5′-TGTAAAACGACGGCCAGTTAAATAGACCTGCAATTATTAATCT-3′

The sequence of reverse oligonucleotide 2 was:

(SEQ ID NO:42)

5′-CAGGAAACAGCTATGACCACCTGCACACCTGCAAATCCATT-3′

PCR was then performed as follows:

| | | | |
|---|---|---|---|
| a. | | Denature | 92° C., 5 minutes |
| b. | 3 cycles of: | Denature | 92° C., 30 seconds |
| | | Anneal | 59° C., 30 seconds |
| | | Extend | 72° C., 60 seconds |
| c. | 3 cycles of: | Denature | 92° C., 30 seconds |
| | | Anneal | 57° C., 30 seconds |
| | | Extend | 72° C., 60 seconds |
| d. | 25 cycles of: | Denature | 92° C., 30 seconds |
| | | Anneal | 55° C., 30 seconds |
| | | Extend | 72° C., 60 seconds |
| e. | | Hold | 4° C. |

The underlined regions of the oligonucleotides annealed to the ADH promoter region and the amylase region, respectively, and amplified a 307 bp region from vector pSST-AMY.0 when no insert was present. Typically, the first 18 nucleotides of the 5′ end of these oligonucleotides contained annealing sites for the sequencing primers. Thus, the total product of the PCR reaction from an empty vector was 343 bp. However, signal sequence-fused cDNA resulted in considerably longer nucleotide sequences.

Following the PCR, an aliquot of the reaction (5 μl) was examined by agarose gel electrophoresis in a 1% agarose gel using a Tris-Borate-EDTA (TBE) buffering system as described by Sambrook et al., supra. Clones resulting in a single strong PCR product larger than 400 bp were further analyzed by DNA sequencing after purification with a 96 Qiaquick PCR clean-up column (Qiagen Inc., Chatsworth, Calif.).

### Example 3

### Isolation of cDNA Clones Using Signal Algorithm Analysis

Various polypeptide-encoding nucleic acid sequences were identified by applying a proprietary signal sequence finding algorithm developed by Genentech, Inc. (South San Francisco, Calif.) upon ESTs as well as clustered and assembled EST fragments from public (e.g., GenBank) and/or private (LIFESEQ®, Incyte Pharmaceuticals, Inc., Palo Alto, Calif.) databases. The signal sequence algorithm computes a secretion signal score based on the character of the DNA nucleotides surrounding the first and optionally the second methionine codon(s) (ATG) at the 5′-end of the sequence or sequence fragment under consideration. The nucleotides following the first ATG must code for at least 35 unambiguous amino acids without any stop codons. If the first ATG has the required amino acids, the second is not examined. If neither meets the requirement, the candidate sequence is not scored. In order to determine whether the EST sequence contains an authentic signal sequence, the DNA and corresponding amino acid sequences surrounding the ATG codon are scored using a set of seven sensors (evaluation parameters) known to be associated with secretion signals. Use of this algorithm resulted in the identification of numerous polypeptide-encoding nucleic acid sequences.

Using the techniques described in Examples 1 to 3 above, numerous full-length cDNA clones were identified as encoding PRO256, PRO334, PRO770, PRO983, PRO1009, PRO1107, PRO1158, PRO1250, PRO1317, PRO4334, PRO4395, PRO9799, PRO21175, PRO19837, PRO21331, PRO697 and PRO1480 polypeptides as disclosed herein. These cDNAs were then deposited under the terms of the Budapest Treaty with the American Type Culture Collection, 10801 University Blvd., Manassas, Va. 20110-2209, USA (ATCC) as shown in Table 7 below. In addition, the sequence of DNA212937 encoding PRO34421 polypeptides, also known as EGFL6 (human EGF-like-domain, multiple 6), was identified from GenBank accession no.: AF186084. The sequence of DNA237637 encoding PRO49192 polypeptides, also known as SLC7A5 (ORTHOLOG OF HUMAN SOLUTE CARRIER FAMILY 7, MEMBER 5), was identified from GenBank accession no.: AB017908. The sequence of DNA194607 encoding PRO23949 polypeptides, also known as TMPRSS2 (human transmembrane protease, serine 2), was identified from GenBank accession no.: AF123453.

TABLE 7

| Material | ATCC Dep. No. | Deposit Date |
|---|---|---|
| DNA35880-1160 | 209379 | Oct. 16, 1997 |
| DNA41379-1236 | 209488 | Nov. 21, 1997 |
| DNA54228-1366-1 | 209801 | Apr. 23, 1998 |
| DNA53977-1371 | 209862 | May 14, 1998 |
| DNA57129-1413 | 209977 | Jun. 16, 1998 |
| DNA59606-1471 | 209945 | Jun. 9, 1998 |
| DNA60625-1507 | 209975 | Jun. 16, 1998 |
| DNA60775-1532 | 203173 | Sep. 1, 1998 |
| DNA71166-1685 | 203355 | Oct. 20, 1998 |
| DNA59608-2577 | 203870 | Mar. 23, 1999 |
| DNA80840-2605 | 203949 | Apr. 20, 1999 |
| DNA108696-2966 | PTA-2315 | Aug. 1, 2000 |
| DNA173894-2947 | PTA-2108 | Jun. 20, 2000 |
| DNA148009-2889 | PTA-1839 | May 9, 2000 |
| DNA175959-2948 | PTA-2248 | Jul. 18, 2000 |
| DNA50920-1325 | 209700 | Mar. 26, 1998 |
| DNA67962-1649 | 203291 | Sep. 29, 1998 |

These deposits were made under the provisions of the Budapest Treaty on the International Recognition of the Deposit of Microorganisms for the Purpose of Patent Procedure and the Regulations thereunder (Budapest Treaty). This assures maintenance of a viable culture of the deposit for 30 years from the date of deposit. The deposits will be made available by ATCC under the terms of the Budapest Treaty, and subject to an agreement between Genentech, Inc. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 USC § 122 and the Commissioner's rules pursuant thereto (including 37 CFR § 1.14 with particular reference to 886 OG 638).

The assignee of the present application has agreed that if a culture of the materials on deposit should die or be lost or destroyed when cultivated under suitable conditions, the materials will be promptly replaced on notification with another of the same. Availability of the deposited material is not to be construed as a license to practice the invention in contravention of the rights granted under the authority of any government in accordance with its patent laws.

### Example 4

### Isolation of cDNA Clones Encoding Human PRO256 Polypeptides [UNQ223]

A consensus DNA sequence was assembled relative to other EST sequences using phrap as described in Example 1 above. This consensus sequence is herein designated DNA28725. Based on the DNA28725 consensus sequence, oligonucleotides were synthesized: 1) to identify by PCR a cDNA library that contained the sequence of interest, and 2) for use as probes to isolate a clone of the full-length coding sequence for PRO256. Forward and reverse PCR primers generally range from 20 to 30 nucleotides and are often designed to give a PCR product of about 100-1000 bp in length. The probe sequences are typically 40-55 bp in length. In some cases, additional oligonucleotides are synthesized when the consensus sequence is greater than about 1-1.5 kbp. In order to screen several libraries for a full-length clone, DNA from the libraries was screened by PCR amplification, as per Ausubel et al., Current Protocols in Molecular Biology, with the PCR primer pair. A positive library was then used to isolate clones encoding the gene of interest using the probe oligonucleotide and one of the primer pairs.

A pair of PCR primers (forward and reverse) were synthesized:

(SEQ ID NO:43)

forward PCR primer: 5′-TGTCCACCAAGCAGACAGAAG-3′

(SEQ ID NO:44)

reverse PCR primer: 5′-ACTGGATGGCGCCTTTCCATG-3′

Additionally, two synthetic oligonucleotide hybridization probes were constructed from the consensus DNA28725 sequence which had the following nucleotide sequence:

hybridization probes:

(SEQ ID NO:45)

5′-CTGACAGTGACTAGCTCAGACCACCCAGAGGACACGGCCAACGTCAC
AGT-3′

(SEQ ID NO:46)
5′-GGGCTCTTTCCCACGCTGGTACTATGACCCCACGGAGCAGATCT
G-3′

In order to screen several libraries for a source of a full-length clone, DNA from the libraries was screened by PCR amplification with the PCR primer pair identified above. A positive library was then used to isolate clones encoding the PRO256 gene using one of the probe oligonucleotides and one of the PCR primers.

RNA for construction of the cDNA libraries was isolated from human placenta tissue. The cDNA libraries used to isolate the cDNA clones were constructed by standard methods using commercially available reagents such as those from Invitrogen, San Diego, Calif. The cDNA was primed with oligo dT containing a NotI site, linked with blunt to SalI hemikinased adaptors, cleaved with NotI, sized appropriately by gel electrophoresis, and cloned in a defined orientation into a suitable cloning vector (such as pRKB or pRKD; pRK5B is a precursor of pRK5D that does not contain the SfiI site; see, Holmes et al., Science, 253:1278-1280 (1991)) in the unique XhoI and NotI sites.

DNA sequencing of the clones isolated as described above gave the full-length DNA sequence for PRO256 [herein designated as DNA35880-1160] (SEQ ID NO:1) and the derived protein sequence for PRO256.

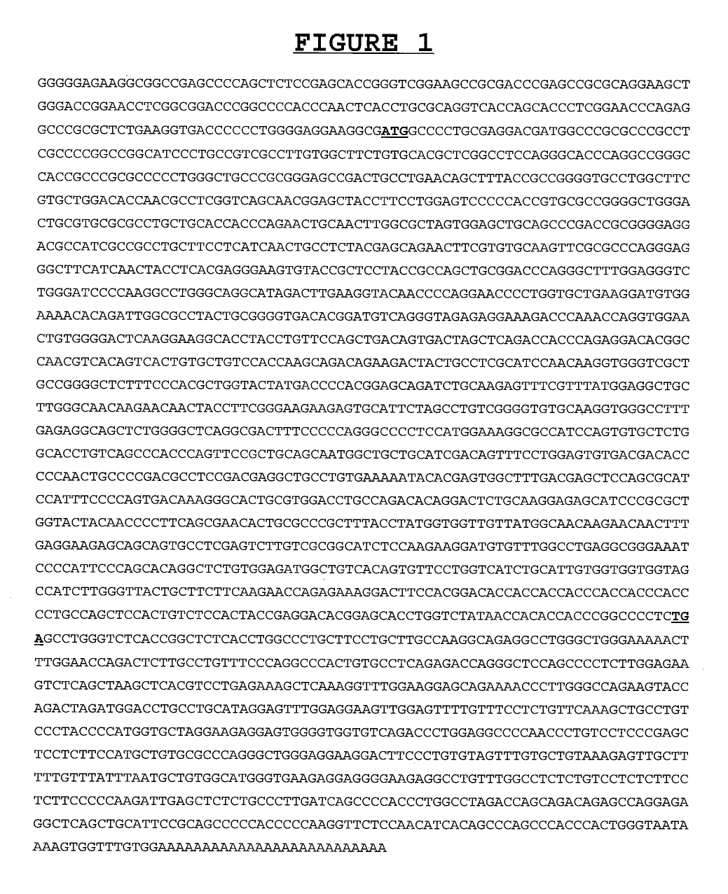

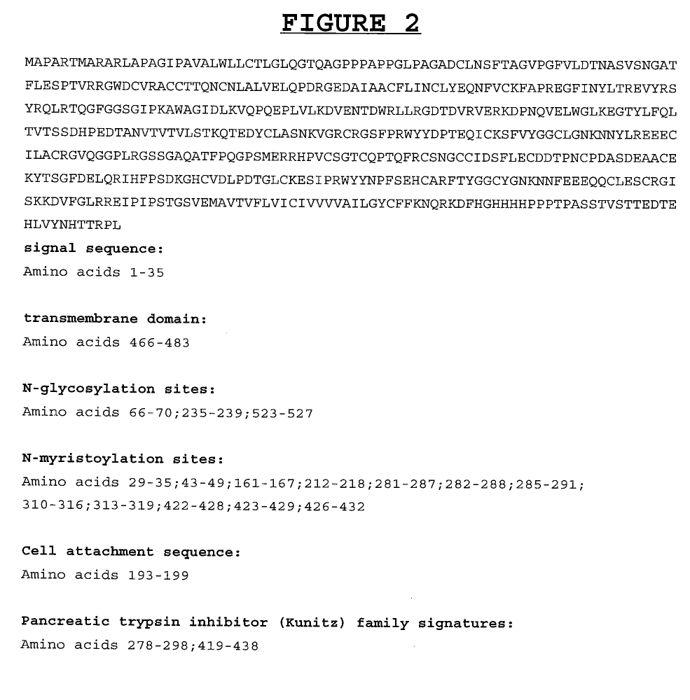

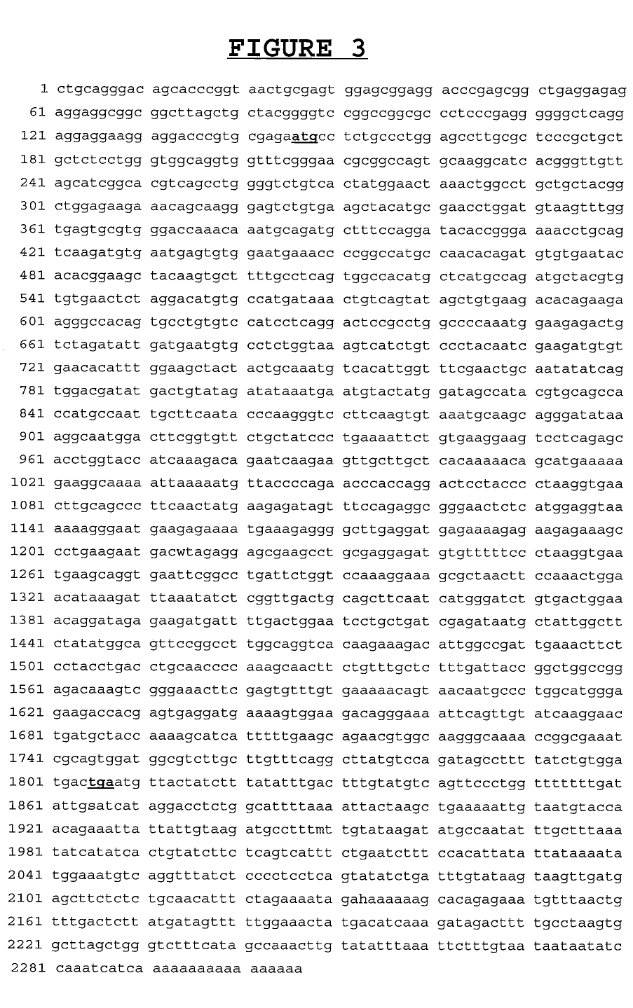

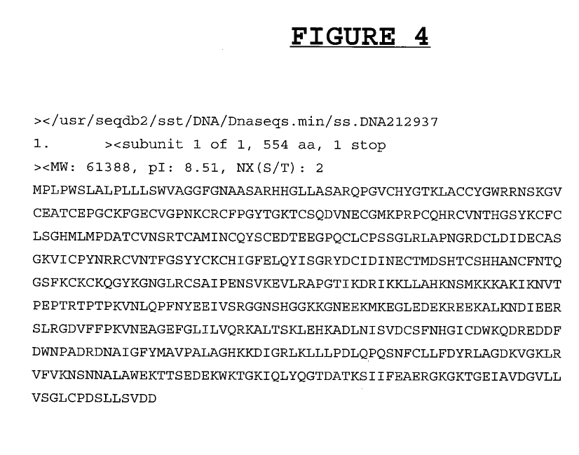

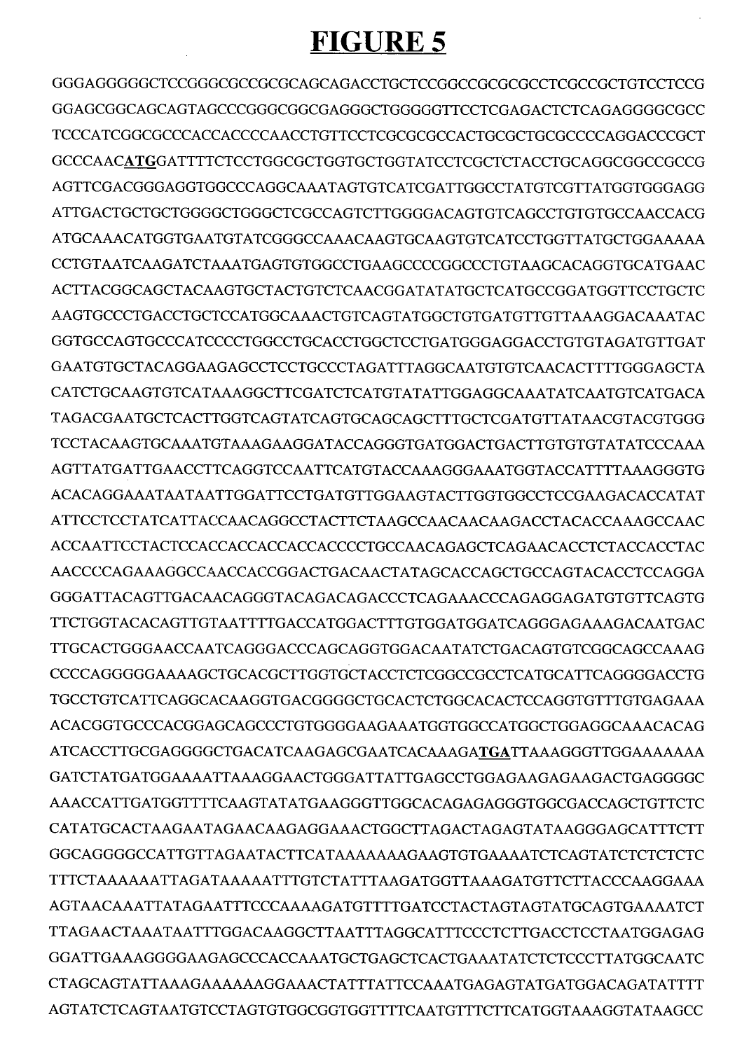

The entire nucleotide sequence of DNA35880-1160 is shown in FIG. 1 (SEQ ID NO:1), Clone DNA35880-1160 contains a single open reading frame with an apparent translational initiation site at nucleotide positions 188-190 and ending at the stop codon at nucleotide positions 1775-1777. The predicted polypeptide precursor is 529 amino acids long (FIG. 2; SEQ ID NO:2). Clone DNA35880-1160 has been deposited with ATCC on Oct. 16, 1997 and is assigned ATCC deposit no. 209379.

Analysis of the amino acid sequence of the full-length PRO256 polypeptide suggests that portions of it possess significant homology to the human bikunin protein, thereby indicating that PRO256 may be a novel proteinase inhibitor.

### Example 5

### Isolation of cDNA Clones Encoding Human PRO334 Polypeptides [UNQ295]

A consensus DNA sequence was assembled relative to other EST sequences using phrap as described in Example 1 above. Based on the consensus sequence, oligonucleotides were synthesized: 1) to identify by PCR a cDNA library that contained the sequence of interest, and 2) for use as probes to isolate a clone of the full-length coding sequence for PRO334.

Forward and reverse PCR primers were synthesized for the determination of PRO334:

(SEQ ID NO:47)

forward PCR primer 5′-GATGGTTCCTGCTCAAGTGCCCTG-3′

(SEQ ID NO:48)

reverse PCR primer 5′-TTGCACTTGTAGGACCCACGTACG-3′

Additionally, a synthetic oligonucleotide hybridization probe was constructed for the determination of PRO334 which had the following nucleotide sequence

hybridization probe

(SEQ ID NO:49)

5′-CTGATGGGAGGACCTGTGTAGATGTTGATGAATGTGCTACAGGAAGA
GCC-3′

In order to screen several libraries for a source of a full-length clone, DNA from the libraries was screened by PCR amplification with the PCR primer pair identified above. A positive library was then used to isolate clones encoding the PRO334 gene using the probe oligonucleotide and one of the PCR primers.

Human fetal kidney cDNA libraries used to isolate the cDNA clones were constructed by standard methods using commercially available reagents such as those from Invitrogen, San Diego, Calif.

DNA sequencing of the clones isolated as described above gave the full-length DNA sequence for PRO334 [herein designated as DNA41379-1236] (SEQ ID NO:5) and the derived protein sequence for PRO334.

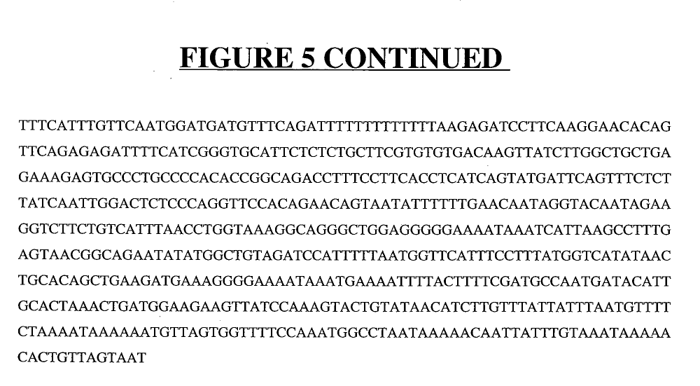

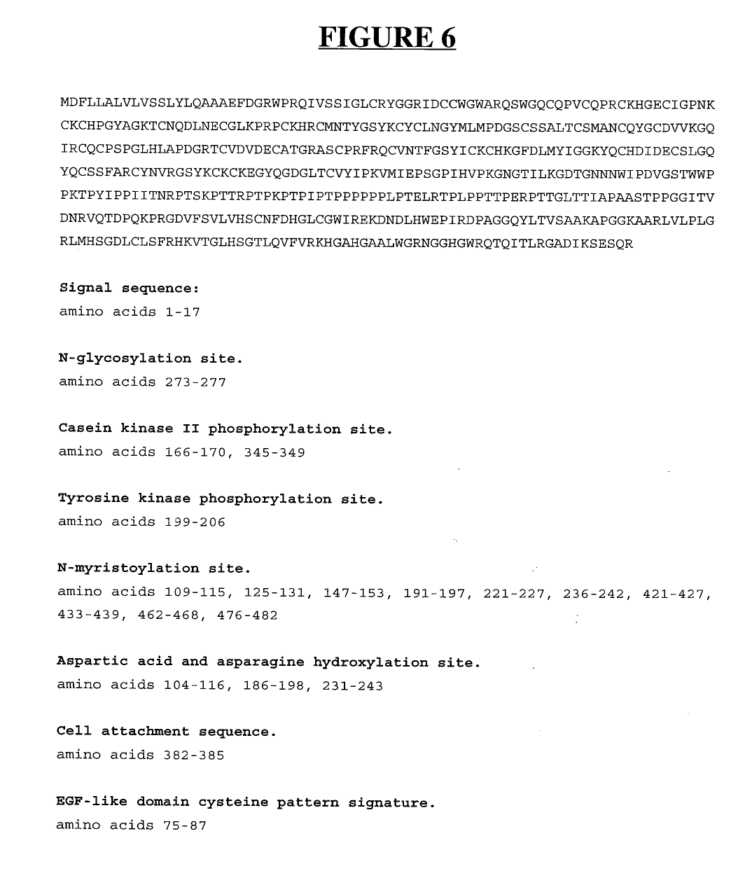

The entire nucleotide sequence of DNA41379-1236 (also referred to as UNQ295) is shown in FIG. 5 (SEQ ID NO:5). Clone DNA41379-1236 contains a single open reading frame with an apparent translational initiation site at nucleotide positions 203-205 and ending at the stop codon at nucleotide positions 1730-1732 (FIG. 5). The predicted polypeptide precursor is 509 amino acids long (FIG. 6; SEQ ID NO:6). Clone DNA41379-1236 has been deposited with ATCC on Nov. 21, 1997 and is assigned ATCC deposit no. 209488.

Analysis of the amino acid sequence of the full-length PRO334 polypeptide suggests that portions of it possess significant homology to the fibulin and fibrillin proteins, thereby indicating that PRO334 may be a novel member of the EGF protein family.

### Example 6

### Isolation of cDNA Clones Encoding Human PRO770 Polypeptides [UNQ408]

A public expressed sequence tag (EST) DNA database (Merck/Washington University) was searched with the full-length murine m-FIZZ1 DNA, and an EST, designated AA524300 was identified, which showed homology with the m-FIZZ1 DNA.

The full-length clone corresponding to the EST AA524300 was purchased from Incyte (Incyte Pharmaceuticals, Palo Alto, Calif.) and sequenced in entirety.

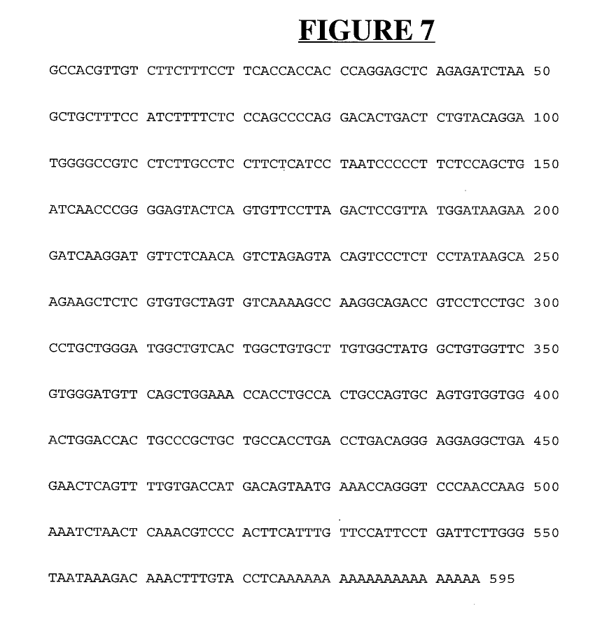

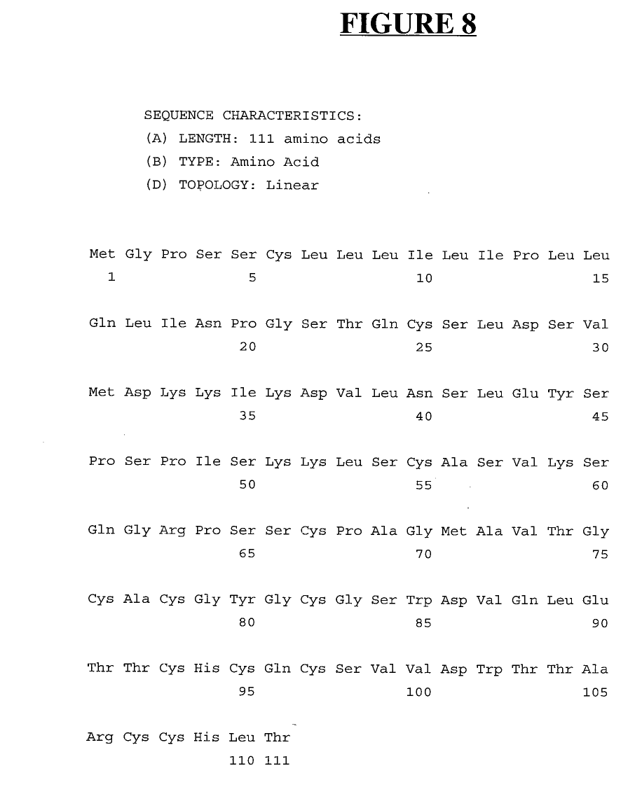

The entire nucleotide sequence of the resulting PRO770-encoding full-length clone is shown in FIG. 7; SEQ ID NO:7. This full-length clone, designated DNA54228-1366-1 (SEQ ID NO:7), contains a single open reading frame with an apparent translation initiation site at nucleotide positions 100-102 (FIG. 7; SEQ ID NO:7) and ending at the stop codon (TGA) at residues 433-435, as indicated by bolded underline. The predicted PRO770 polypeptide precursor (including a putative signal sequence of 20 amino acids) (i.e., UNQ408, FIG. 8; SEQ ID NO:8) is 111 amino acids long, has a calculated molecular weight of 11,730 daltons and a pI of 7.82. Based upon its homology to m-FIZZ1 (50%, using the ALIGN software), the protein is believed to be the human homolog of m-FIZZ1, and has been designated h-FIZZ1. A cDNA clone containing DNA54228-1366-1 (SEQ ID NO:7) has been deposited with ATCC on Apr. 23, 1998 and is assigned ATCC deposit no. 209801.

### Example 7

### Isolation of cDNA Clones Encoding Human PRO983 Polypeptides [UNQ484]

A consensus sequence was obtained relative to a variety of EST sequences as described in Example 1 above, wherein the consensus sequence obtained is herein designated DNA47473. Various proprietary Genentech EST sequences were employed in the assembly. Based on the DNA47473 consensus sequence, oligonucleotides were synthesized: 1) to identify by PCR a cDNA library that contained the sequence of interest, and 2) for use as probes to isolate a clone of the full-length coding sequence for PRO983.

A pair of PCR primers (forward and reverse) were synthesized:

forward PCR primer

(SEQ ID NO:50)

5′-GCACCACCGTAGGTACTTGTGTGAGGC-3′
reverse PCR primer

(SEQ ID NO:51)

5′-AACCACCAGAGCCAAGAGCCGGG-3′

Additionally, a synthetic oligonucleotide hybridization probe was constructed from the consensus DNA47473 sequence which had the following nucleotide sequence

hybridization probe

(SEQ ID NO:52)

5′-CAGCGGAATCATCGATGCAGGGGCCTCAATTAATGTATCTGTGATGT
TAC-3′

In order to screen several libraries for a source of a full-length clone, DNA from the libraries was screened by PCR amplification with the PCR primer pair identified above. A positive library was then used to isolate clones encoding the PRO983 gene using the probe oligonucleotide and one of the PCR primers. RNA for construction of the cDNA libraries was isolated from human bone marrow (LIB256).

DNA sequencing of the clones isolated as described above gave the full-length DNA sequence for PRO983 [herein designated as UNQ484 (DNA53977-1371)] (SEQ ID NO:9) and the derived protein sequence for PRO983.

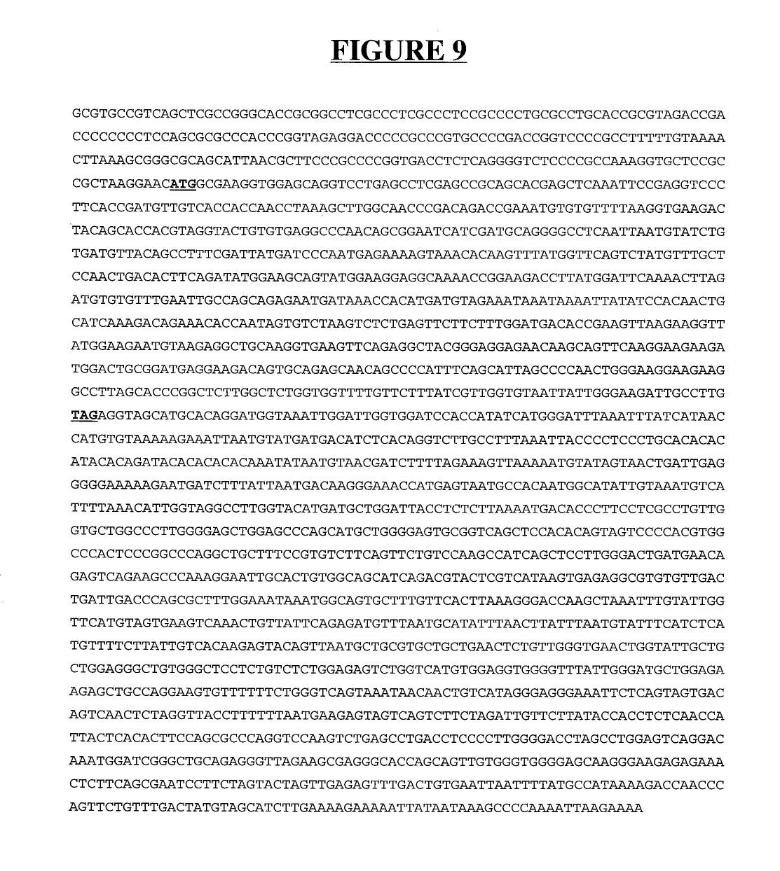

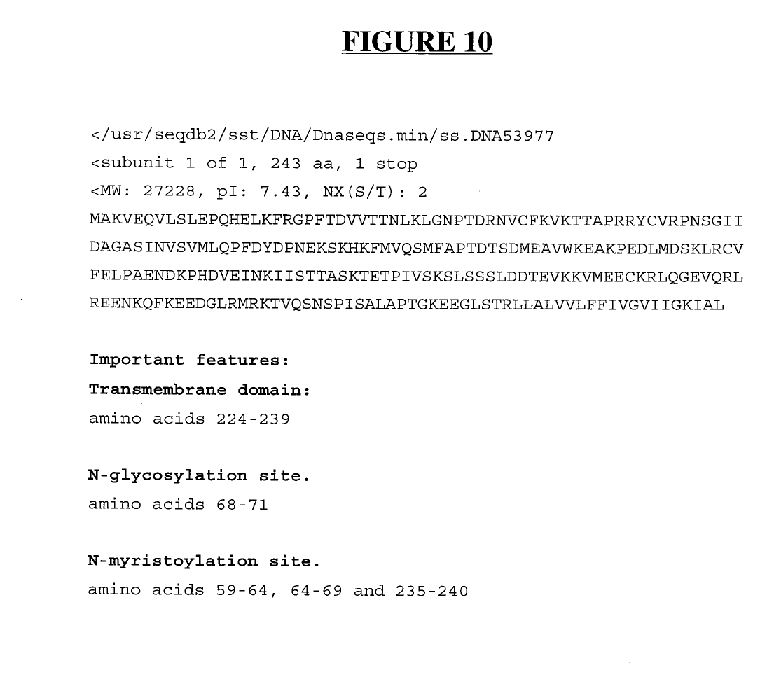

The entire nucleotide sequence of UNQ484 (DNA53977-1371) is shown in FIG. 9 (SEQ ID NO:9). Clone UNQ484 (DNA53977-1371) contains a single open reading frame with an apparent translational initiation site at nucleotide positions 234-236 and ending at the stop codon at nucleotide positions 963-965 (FIG. 9). The predicted polypeptide precursor is 243 amino acids long (FIG. 10; SEQ ID NO:10). The full-length PRO983 protein shown in FIG. 10 has an estimated molecular weight of about 27,228 daltons and a pI of about 7.43. Analysis of the full-length PRO983 sequence shown in FIG. 10 (SEQ ID NO:10) evidences the presence of the following features: a putative transmembrane domain from about amino acid 224 to about amino acid 239; a potential N-glycosylation site from about amino acid 68 to about amino acid 71; and three potential N-myristoylation sites from about amino acid 59 to about amino acid 64, from about amino acid 64 to about amino acid 69, and from about amino acid 235 to about amino acid 240. Clone UNQ484 (DNA53977-1371) has been deposited with ATCC on May 14, 1998 and is assigned ATCC deposit no. 209862.

Analysis of the amino acid sequence of the full-length PRO983 polypeptide suggests that it possesses significant sequence similarity to the vesicle-associated protein, VAP-33, thereby indicating that PRO983 may be a novel vesicle associated membrane protein. More specifically, an analysis of the Dayhoff database (version 35.45 SwissProt 35) evidenced significant homology between the PRO983 amino acid sequence and the following Dayhoff sequences: VP33_APLCA, CELF33D11_{—}12, CELF42G2_{—}2, S50623, YDFC_SCHPO, CELF54H5_{—}2, CELZC196_{—}8, CEF57A10_{—}3, MSP3_GLORO, CEC15H11_{—}1.

### Example 8

### Isolation of cDNA Clones Encoding Human PRO1009 Polypeptides [UNQ493]

A cDNA clone (DNA57129-1413) encoding a native human PRO1009 polypeptide was identified by the use of a yeast screen, in a human SK—Lu-1 adenocarcinoma cell line cDNA library that preferentially represents the 5′ ends of the primary cDNA clones. First a pre-consensus sequence was identified, which was extended by alignments to other EST sequences to form a consensus sequence. Oligonucleotide probes based upon the consensus sequence were synthesized and used to screen the cDNA library which gave rise to the full-length DNA57129-1413 clone.

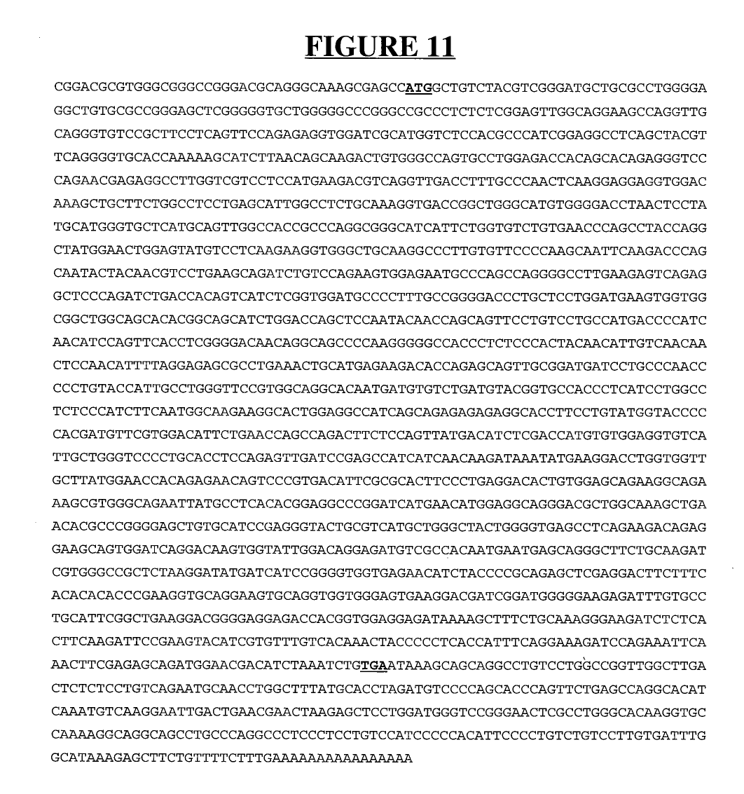

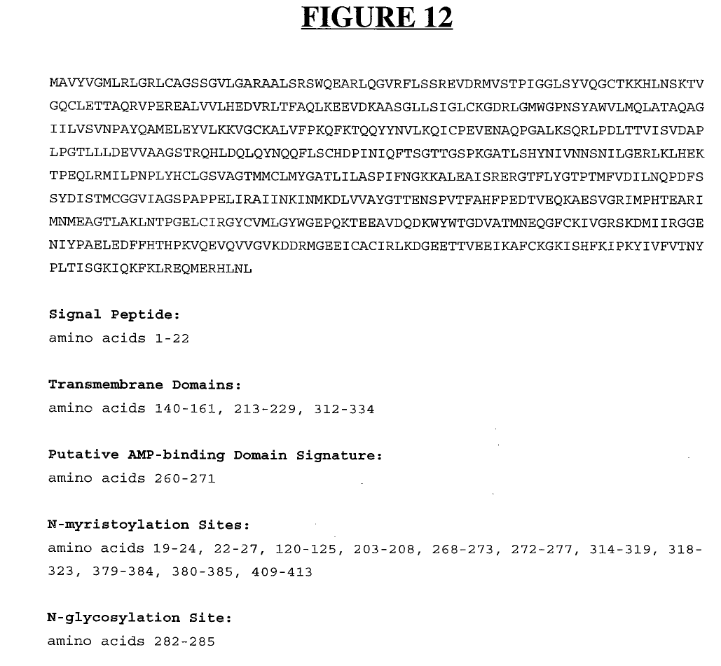

The full length DNA57129-1413 clone shown in FIG. 11 contained a single open reading frame with an apparent translational initiation site at nucleotide positions 41-43 and ending at the stop codon found at nucleotide positions 1886-1888 (FIG. 11; SEQ ID NO:11). The predicted polypeptide precursor (FIG. 12, SEQ ID NO:12) is 615 amino acids long. FIG. 12 also shows the approximate locations of the signal sequence, transmembrane domains, myristoylation sites, a glycosylation site and an AMP-binding domain. PRO1009 has a calculated molecular weight of approximately 68,125 daltons and an estimated pI of approximately 7.82. Clone DNA57129-1413 has been deposited with ATCC on Jun. 16, 1998 and is assigned ATCC deposit no. 209977. It is understood that the deposited clone has the actual and correct sequence and that the representations herein may have minor, normal sequencing errors.

Based on a WU-BLAST-2 sequence alignment analysis (using the ALIGN computer program) of the full-length sequence, PRO1009 shows amino acid sequence identity to at least the following proteins which were designated in a Dayhoff database as follows: F69893, CEF28F8_{—}2, BSY13917_{—}7, BSY13917_{—}7, D69187, D69649, XCRPFB_{—}1, E64928, YDID_ECOLI, BNACSF8_{—}1 and RPU75363_{—}2.

### Example 9

### Isolation of cDNA Clones Encoding Human PRO1107 Polypeptides [UNQ550]

Use of the signal sequence algorithm described in Example 3 above allowed identification of a certain EST cluster sequence from the Incyte database. This EST cluster sequence was then compared to a variety of expressed sequence tag (EST) databases which included public EST databases (e.g., GenBank) and a proprietary EST DNA database (LIFESEQ®, Incyte Pharmaceuticals, Palo Alto, Calif.) to identify existing homologies. The homology search was performed using the computer program BLAST or BLAST2 (Altshul et al., Methods in Enzymology 266:460-480 (1996)). Those comparisons resulting in a BLAST score of 70 (or in some cases 90) or greater that did not encode known proteins were clustered and assembled into a consensus. DNA sequence with the program “phrap” (Phil Green, University of Washington, Seattle, Wash.). The consensus sequence obtained therefrom is herein designated DNA56402.

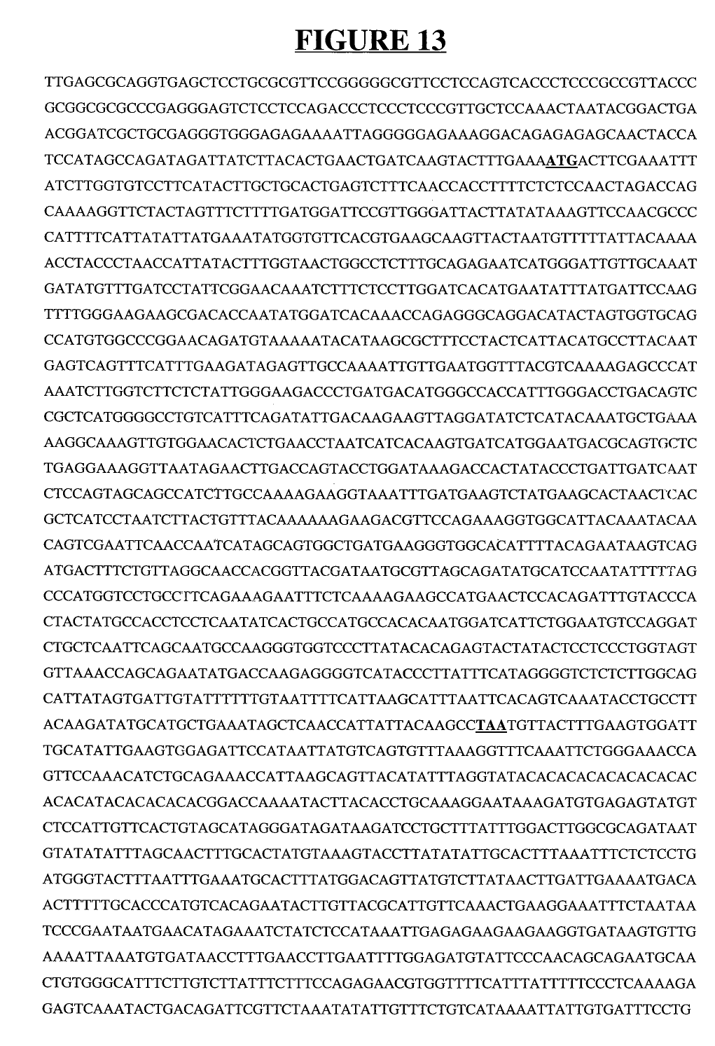

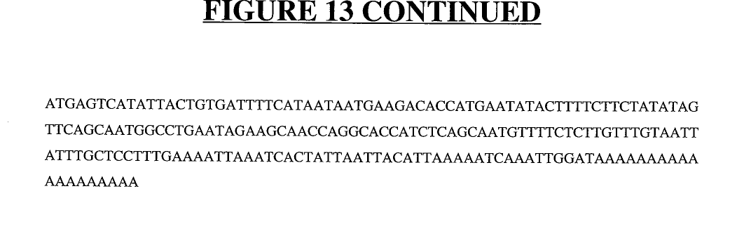

In light of an observed sequence homology between the DNA56402 sequence and an EST sequence contained within Incyte EST clone no. 3203694, the Incyte EST clone no. 3203694 was purchased and the cDNA insert was obtained and sequenced. It was found that the insert encoded a full-length protein. The sequence of this cDNA insert is shown in FIG. 13 and is herein designated as DNA59606-1471.

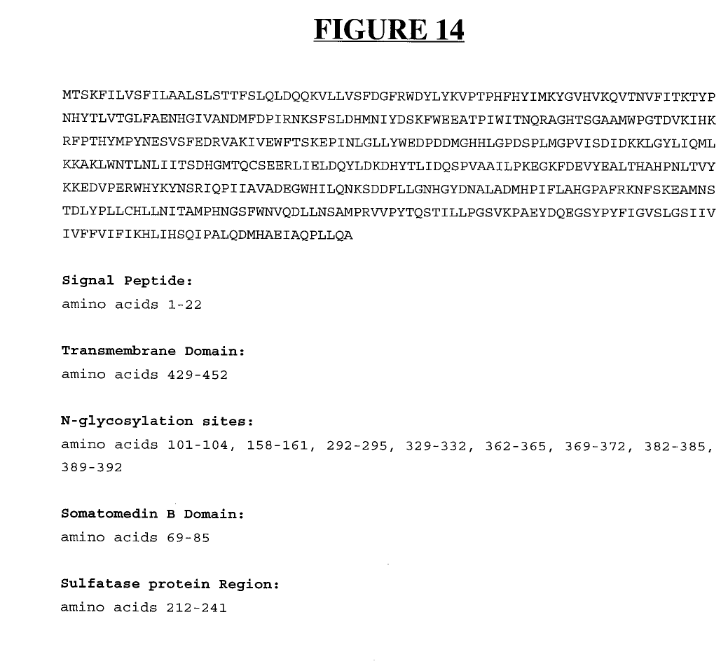

The entire nucleotide sequence of DNA59606-1471 is shown in FIG. 13 (SEQ ID NO:13). Clone DNA59606-1471 contains a single open reading frame with an apparent translational initiation site at nucleotide positions 244-246 and ending at the stop codon at nucleotide positions 1675-1677 of SEQ ID NO:13 (FIG. 13). The predicted polypeptide precursor is 477 amino acids long (FIG. 14; SEQ ID NO:14). The full-length PRO1107 protein shown in FIG. 14 has an estimated molecular weight of about 54,668 daltons and a pI of about 6.33. Clone DNA59606-1471 has been deposited with ATCC on Jun. 9, 1998 as ATCC accession number 209945. It is understood that the deposited clone has the actual nucleic acid sequence and that the sequences provided herein are based on known sequencing techniques.

Analysis of the amino acid sequence of the full-length PRO1107 polypeptide suggests that it possesses significant sequence similarity to phosphodiesterase I/nucleotide pyrophosphatase, human insulin receptor tyrosine kinase inhibitor, alkaline phosphodiesterase and autotoxin, thereby indicating that PRO1107 may have at least one or all of the activities of these proteins, and that PRO1107 is a novel phosphodiesterase. More specifically, an analysis of the Dayhoff database (version 35.45 SwissProt 35) evidenced sequence identity between the PRO1107 amino acid sequence and at least the following Dayhoff sequences: AF005632_{—}1, P_R79148, RNU78787_{—}1, AF060218_{—}4, A57080 and HUMATXT_{—}1.

### Example 10

### Isolation of cDNA Clones Encoding Human PRO1158 Polypeptides [UNQ588]

Use of the signal sequence algorithm described in Example 3 above allowed identification of a single EST cluster sequence from the Incyte database. This EST cluster sequence was then compared to a variety of expressed sequence tag (EST) databases which included public EST databases (e.g., GenBank) and a proprietary EST DNA database (LIFESEQ®, Incyte Pharmaceuticals, Palo Alto, Calif.) to identify existing homologies. The homology search was performed using the computer program BLAST or BLAST2 (Altshul et al., Methods in Enzymology 266:460-480 (1996)). Those comparisons resulting in a BLAST score of 70 (or in some cases 90) or greater that did not encode known proteins were clustered and assembled into a consensus DNA sequence with the program “phrap” (Phil Green, University of Washington, Seattle, Wash.). The consensus sequence obtained therefrom is herein designated DNA57248.

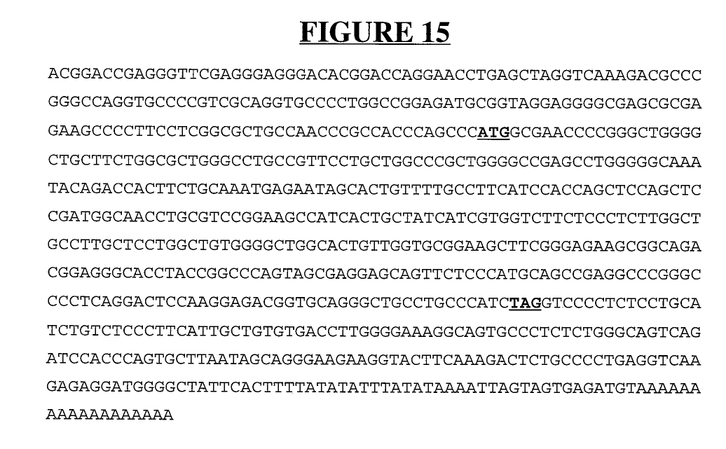

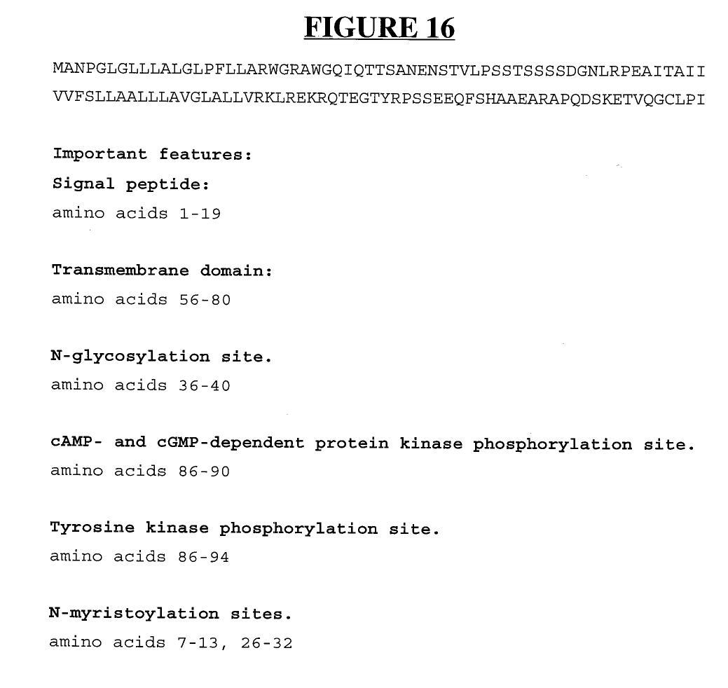

In light of an observed sequence homology between the DNA57248 consensus sequence and an EST sequence encompassed within the Incyte EST clone no. 2640776, the Incyte EST clone 2640776 was purchased and the cDNA insert was obtained and sequenced. It was found that this insert encoded a full-length protein. The sequence of this cDNA insert is shown in FIG. 15 and is herein designated as DNA60625-1507 (SEQ ID NO:15).

The full length clone shown in FIG. 15 contained a single open reading frame with an apparent translational initiation site at nucleotide positions 163 to 165 and ending at the stop codon found at nucleotide positions 532 to 534 (FIG. 15; SEQ ID NO:15). The predicted polypeptide precursor (FIG. 16, SEQ ID NO:16) is 123 amino acids long. PRO1158 has a calculated molecular weight of approximately 13,113 daltons and an estimated pI of approximately 8.53. Additional features include a signal peptide sequence at about amino acids 1-19, a transmembrane domain at about amino acids 56-80, and a potential N-glycosylation site at about amino acids 36-39. Clone DNA60625-1507 was deposited with the ATCC on Jun. 16, 1998 and is assigned ATCC deposit no. 209975.

An analysis of the Dayhoff database (version 35.45 SwissProt 35), using a WU-BLAST2 sequence alignment analysis of the full-length sequence shown in FIG. 16 (SEQ ID NO:16), revealed some homology between the PRO1158 amino acid sequence and the following Dayhoff sequences: ATAC00310510F18A8.10, P_R85151, PHS2_SOLTU, RNMHCIBAC_{—}1, RNA1FMHC_{—}1, I68771, RNRT1A10G_{—}1, PTPA_HUMAN, HUMGACA_{—}1, and CHKPTPA_{—}1.

### Example 11

### Isolation of cDNA Clones Encoding Human PRO1250 Polypeptides [UNQ633]

Use of the signal sequence algorithm described in Example 3 above allowed identification of an EST cluster sequence from the Incyte database, designated Incyte EST cluster sequence no. 56523. This EST cluster sequence was then compared to a variety of expressed sequence tag (EST) databases which included public EST databases (e.g., GenBank) and a proprietary EST DNA database (Lifeseq®, Incyte Pharmaceuticals, Palo Alto, Calif.) to identify existing homologies. The homology search was performed using the computer program BLAST or BLAST2 (Altshul et al., Methods in Enzymology 266:460-480 (1996)). Those comparisons resulting in a BLAST score of 70 (or in some cases 90) or greater that did not encode known proteins were clustered and assembled into a consensus DNA sequence with the program “phrap” (Phil Green, University of Washington, Seattle, Wash.). The consensus sequence obtained therefrom is herein designated DNA56103.

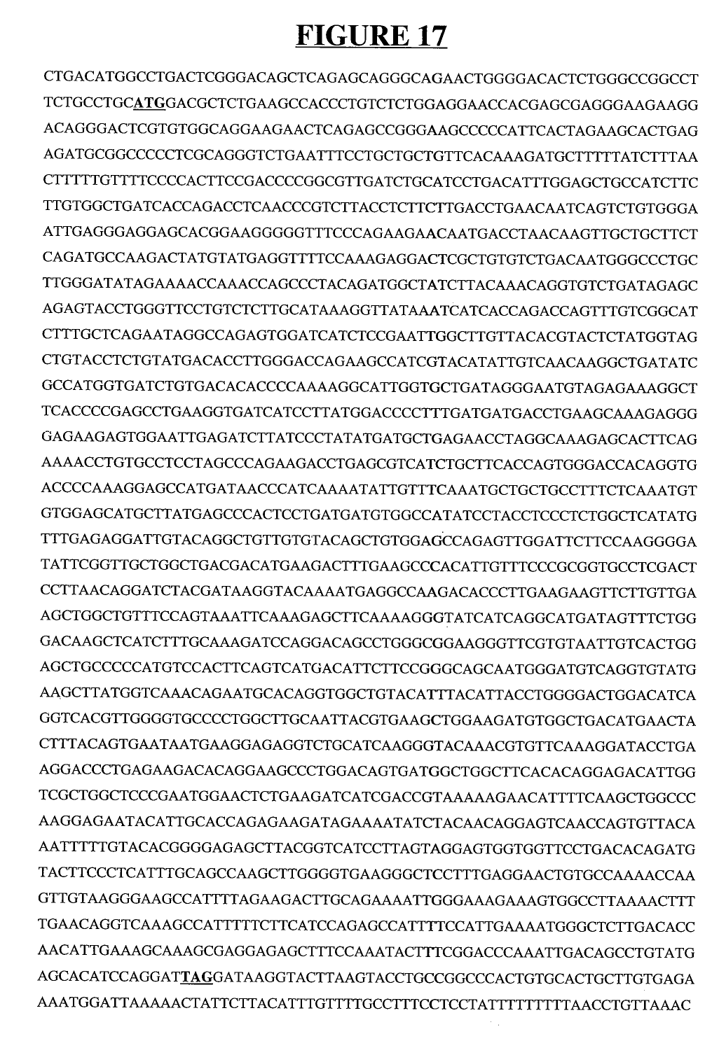

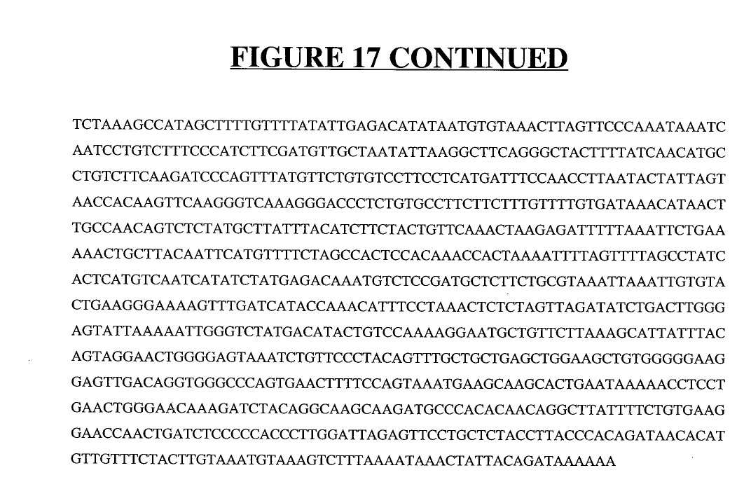

In light of the sequence homology between the DNA56103 sequence and an EST sequence contained within the Incyte EST clone no. 3371784, the Incyte EST clone no. 3371784 was purchased and the cDNA insert was obtained and sequenced. The sequence of this cDNA insert is shown in FIG. 17 and is herein designated as DNA60775-1532.

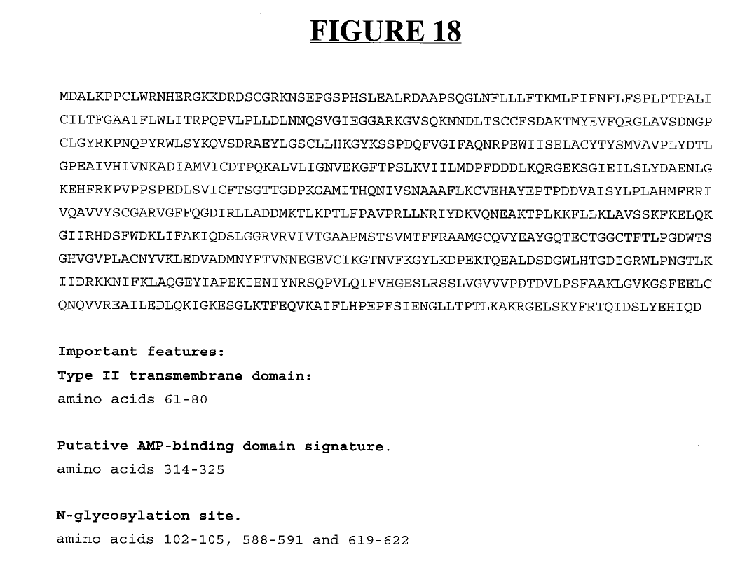

Clone DNA60775-1532 contains a single open reading frame with an apparent translational initiation site at nucleotide positions 74-76 and ending at the stop codon at nucleotide positions 2291-2293 (FIG. 17; SEQ ID NO:17). The predicted polypeptide precursor is 739 amino acids long (FIG. 18; SEQ ID NO:18). The full-length PRO1250 protein shown in FIG. 18 has an estimated molecular weight of about 82,263 daltons and a pI of about 7.55. Analysis of the full-length PRO1250 sequence shown in FIG. 18 (SEQ ID NO:18) evidences the presence of the following: a type II transmembrane domain from about amino acid 61 to about amino acid 80, a putative AMP-binding domain signature sequence from about amino acid 314 to about amino acid 325, and potential N-glycosylation sites from about amino acid 102 to about amino acid 105, from about amino acid 588 to about amino acid 591 and from about amino acid 619 to about amino acid 622. Clone DNA60775-1532 has been deposited with ATCC on Sep. 1, 1998 and is assigned ATCC deposit no. 203173.

An analysis of the Dayhoff database (version. 35.45 SwissProt 35), using a WU-BLAST2 sequence alignment analysis of the full-length sequence shown in FIG. 18 (SEQ ID NO:18), evidenced significant homology between the PRO1250 amino acid sequence and the following Dayhoff sequences: LCFB_HUMAN, S56508_{—}1, BNAMPBP2_{—}1, BNACS7_{—}1, CELT08B1_{—}6, CELC46F4_{—}2, AF0082066 CELR07C3_{—}11, LMU70253_{—}2 and AF008206_{—}7.

### Example 12

### Isolation of cDNA Clones Encoding Human PRO1317 Polypeptides [UNQ783]

A consensus DNA sequence was assembled relative to other EST sequences using phrap as described in Example 1 above. This consensus sequence is designated herein “Consen8865”. In addition, the Consen8865 consensus sequence was extended using repeated cycles of BLAST and phrap to extend the consensus sequence as far as possible using the sources of EST sequences discussed above. The extended consensus sequence is designated herein as “DNA63334”. Based on the DNA63334 consensus sequence, oligonucleotides were synthesized: 1) to identify by PCR a cDNA library that contained the sequence of interest, and 2) for use as probes to isolate a clone of the full-length coding sequence for PRO1317.

PCR primers (forward and reverse) were synthesized:

forward PCR primer: CTGCTGGTGAAATCTGGCGTGGAG
(63334.f1; SEQ ID NO:53);
and
reverse PCR primer: GTCTGGTCCTGGCTGTCCACCCAG
(63334.r1; SEQ ID NO:54).

Additionally, a synthetic oligonucleotide hybridization probe was constructed from the consensus DNA63334 sequence which had the following nucleotide sequence:

hybridization probe

(63334.p1; SEQ ID NO:55)

CATCTTGTCATGTACCTGGGAACCACCACAGGGTCGCTCCACAAG.

In order to screen several libraries for a source of a full-length clone, DNA from the libraries was screened by PCR amplification with the PCR primer pair identified above. A positive library was then used to isolate clones encoding the PRO1317 gene using the probe oligonucleotide and one of the PCR primers. RNA for construction of the cDNA libraries was isolated from human hippocampal tissue.

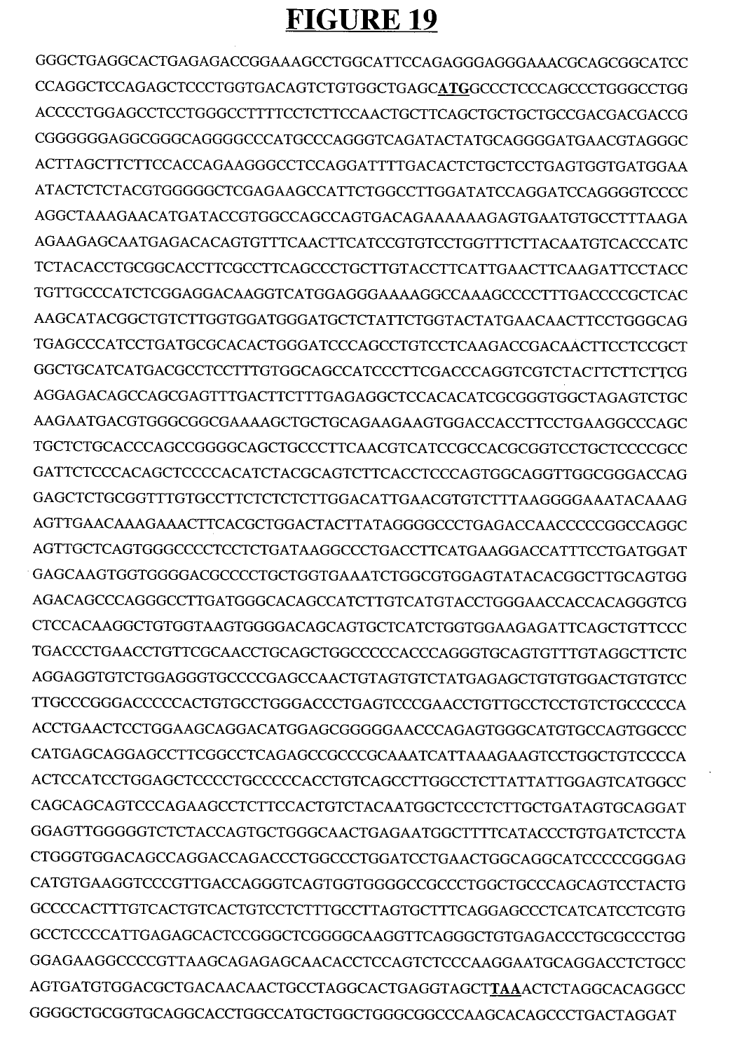

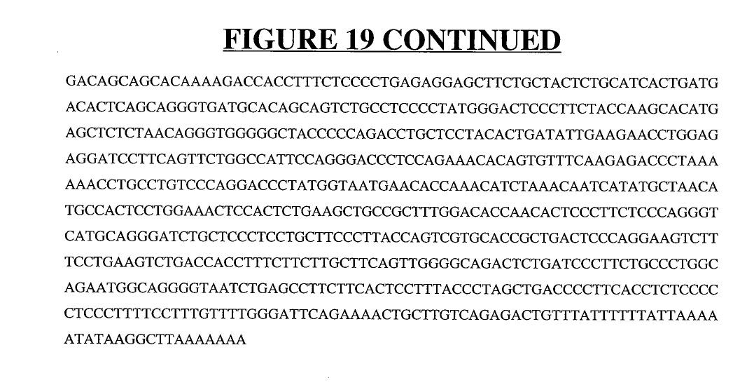

DNA sequencing of the clones isolated as described above gave the full-length DNA sequence for PRO1317 (designated herein as DNA71166-1685 [FIG. 19, SEQ ID NO:19]; and the derived protein sequence for PRO1317.

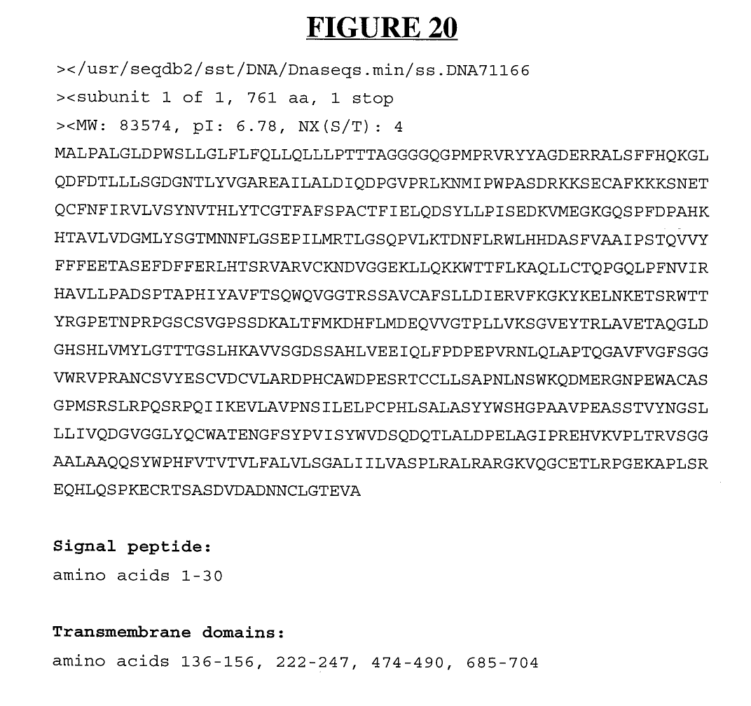

The entire coding sequence of PRO1317 is shown in FIG. 19 (SEQ ID NO:19). Clone DNA71166-1685 contains a single open reading frame with an apparent translational initiation site at nucleotide positions 105-107 and an apparent stop codon at nucleotide positions 2388-2390. The predicted polypeptide precursor is 761 amino acids long and has an estimated molecular weight of about 83,574 daltons and a pI of about 6.78 (FIG. 20; SEQ ID NO:20).

An analysis of the Dayhoff database (version 35.45 SwissProt 35), using a WU-BLAST2 sequence alignment analysis of the full-length sequence shown in FIG. 20 (SEQ ID NO:20), revealed significant homology between the PRO1317 amino acid sequence and Dayhoff sequence no. 148745. Homology was also revealed between the PRO1317 amino acid sequence the following Dayhoff sequences: I48746, GEN13418, P_W58540, P_{—}217657, MUSC1_{—}1, P_{—}471380, U73167_{—}5, HSU33920_{—}1, and GG828240_{—}1.

Clone DNA71166-1685 was deposited with the ATCC on Oct. 20, 1998, and is assigned ATCC deposit no. 203355.

### Example 13

### Isolation of cDNA Clones Encoding Human PRO4334 Polypeptides [UNQ1889]

Use of the signal sequence algorithm described in Example 3 above allowed identification of an EST cluster sequence from the Incyte database. This EST cluster sequence was then compared to a variety of expressed sequence tag (EST) databases which included public EST databases (e.g., GenBank) and a proprietary EST DNA database (LIFESEQ®, Incyte Pharmaceuticals, Palo Alto, Calif.) to identify existing homologies. The homology search was performed using the computer program BLAST or BLAST2 (Altshul et al., Methods in Enzymology 266:460-480 (1996)). Those comparisons resulting in a BLAST score of 70 (or in some cases 90) or greater that did not encode known proteins were clustered and assembled into a consensus DNA sequence with the program “phrap” (Phil Green, University of Washington, Seattle, Wash.). The consensus sequence obtained therefrom is herein designated DNA56421.

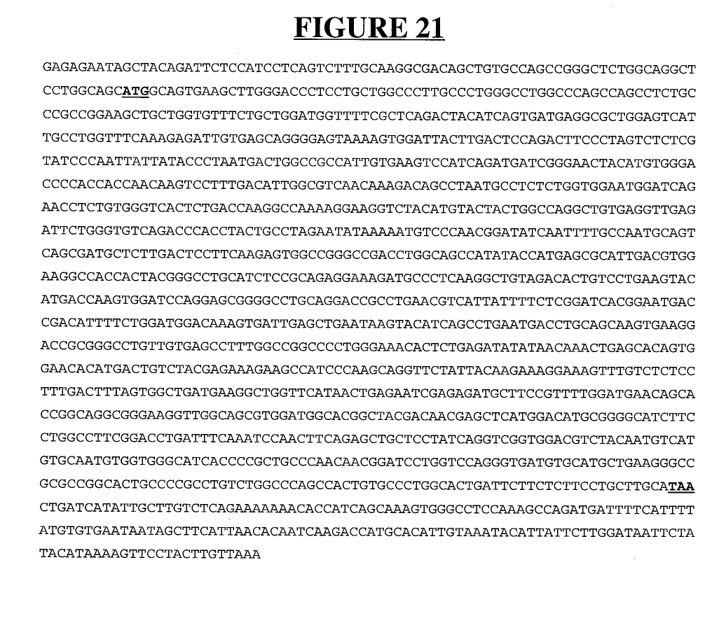

In light of an observed sequence homology between the DNA56421 sequence and an EST sequence contained within the Incyte EST clone no. 3347532, the Incyte clone was purchased and the cDNA insert was obtained and sequenced. The sequence of this cDNA insert is shown in FIG. 21 and is herein designated as DNA59608-2577.

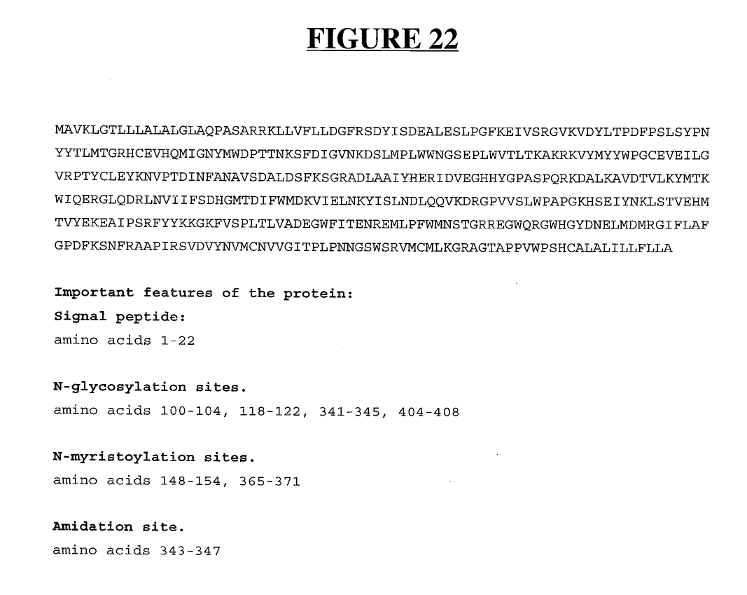

The full length clone shown in FIG. 21 contained a single open reading frame with an apparent translational initiation site at nucleotide positions 83-85 and ending at the stop codon found at nucleotide positions 1404-1406 (FIG. 21; SEQ ID NO:21). The predicted polypeptide precursor (FIG. 22, SEQ ID NO:22) is 440 amino acids long. PRO4334 has a calculated molecular weight of approximately 50,211 daltons and an estimated pI of approximately 8.29.

An analysis of the Dayhoff database (version 35.45 SwissProt 35), using a WU-BLAST2 sequence alignment analysis of the full-length sequence shown in FIG. 22 (SEQ ID NO:22), revealed homology between the PRO4334 amino acid sequence and the following Dayhoff sequences incorporated herein: AB020686_{—}1, PC1_HUMAN, P_R79148, PC1_MOUSE, RNU78788_{—}1, RATPDIB_{—}1, P_W75859, AC005587_{—}1, P_R86595 and PPD1_BOVIN.

Clone DNA59608-2577 was deposited with the ATCC on Mar. 23, 1999 and is assigned ATCC deposit no. 203870.

### Example 14

### Isolation of cDNA Clones Encoding Human PRO4395 Polypeptides [UNQ921]

A proprietary EST DNA database (LIFESEQ™, Incyte Pharmaceuticals, Palo Alto, Calif.) was searched and an EST, was identified, which showed homology with the fibrillin protein.

Based on the DNA38228 consensus sequence oligonucleotides were synthesized: 1) to identify by PCR a cDNA library that contained the sequence of interest, and 2) for use as probes to isolate a clone of the full-length coding sequence for PRO4395. Forward and reverse PCR primers generally range from 20 to 30 nucleotides and are often designed to give a PCR product of about 100-1000 bp in length. The probe sequences are typically 40-55 bp in length. In some cases, additional oligonucleotides are synthesized when the consensus sequence is greater than about 1-1.5 kbp. In order to screen several libraries for a full-length clone, DNA from the libraries was screened by PCR amplification, as per Ausubel et al., Current Protocols in Molecular Biology, supra, with the PCR primer pair. A positive library was then used to isolate clones encoding the gene of interest using the probe oligonucleotide and one of the primer pairs.

PCR primers (forward and reverse) were synthesized:

forward PCR primer

(SEQ ID NO:56)

5′CCCGTGTCTGAAGTCTTCAGGCG3′
and
reverse PCR primer

(SEQ ID NO:57)

5′TCCAGACAGTATGGCTTCTCCCGC3′.

Additionally, a synthetic oligonucleotide hybridization probe was constructed from the consensus DNA38228 sequence which had the following nucleotide sequence:

hybridization probe

(SEQ ID NO:58)

5′ATTACGACGTTTGTGCCGAGGCTCCCTGTGAACAGCAGTGCACGG3′.

In order to screen several libraries for a source of a full-length clone, DNA from the libraries was screened by PCR amplification with the PCR primer pair identified above. A positive library was then used to isolate clones encoding the PRO4395 gene using the probe oligonucleotide and one of the PCR primers.

RNA for construction of the cDNA libraries was isolated from human fetal kidney tissue. The cDNA libraries used to isolate the cDNA clones were constructed by standard methods using commercially available reagents such as those from Invitrogen, San Diego, Calif. The cDNA was primed with oligo dT containing a NotI site, linked with blunt to SalI hemikinased adaptors, cleaved with NotI, sized appropriately by gel electrophoresis, and cloned in a defined orientation into a suitable cloning vector (such as pRKB or pRKD; pRK5B is a precursor of pRK5D that does not contain the SfiI site; see, Holmes et al., Science, 253:1278-1280 (1991)) in the unique XhoI and NotI sites.

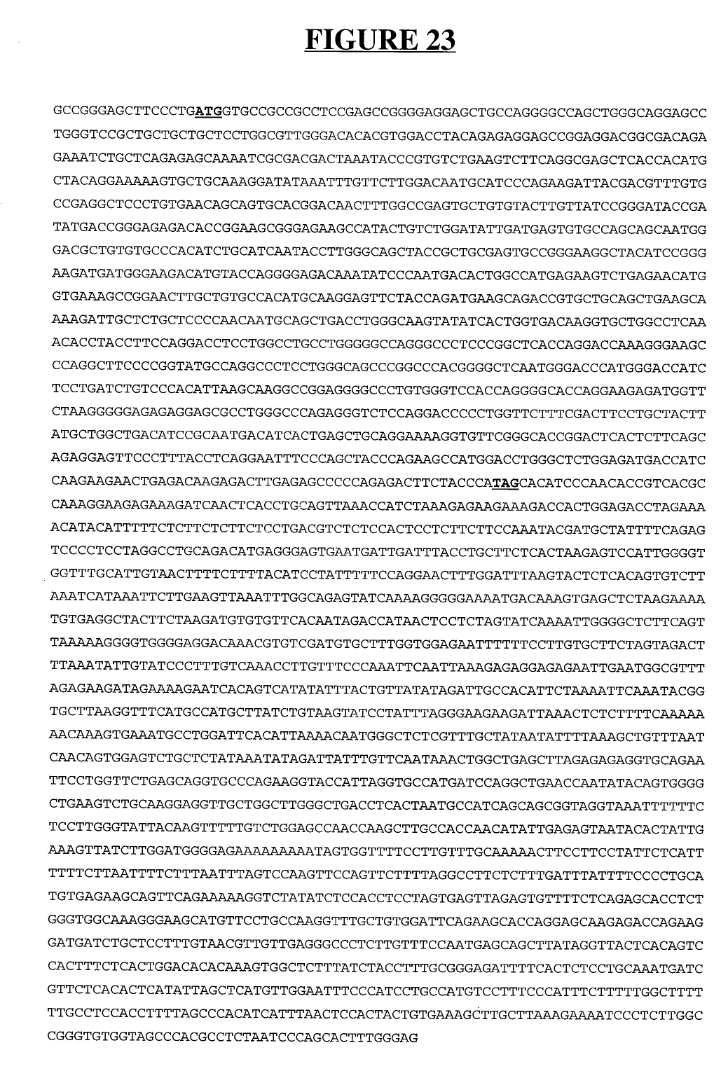

DNA sequencing of the clones isolated as described above gave the full-length DNA sequence for PRO4395 (designated herein as DNA80840-2605 [FIG. 23, SEQ ID NO:23]; and the derived protein sequence for PRO4395.

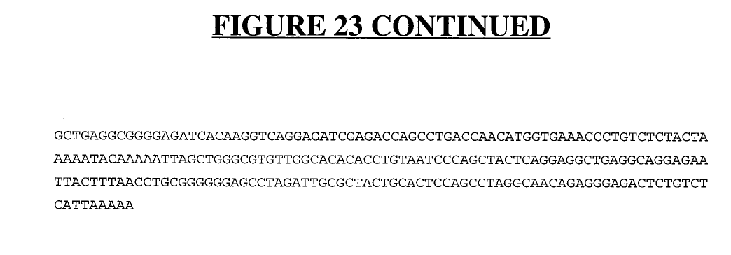

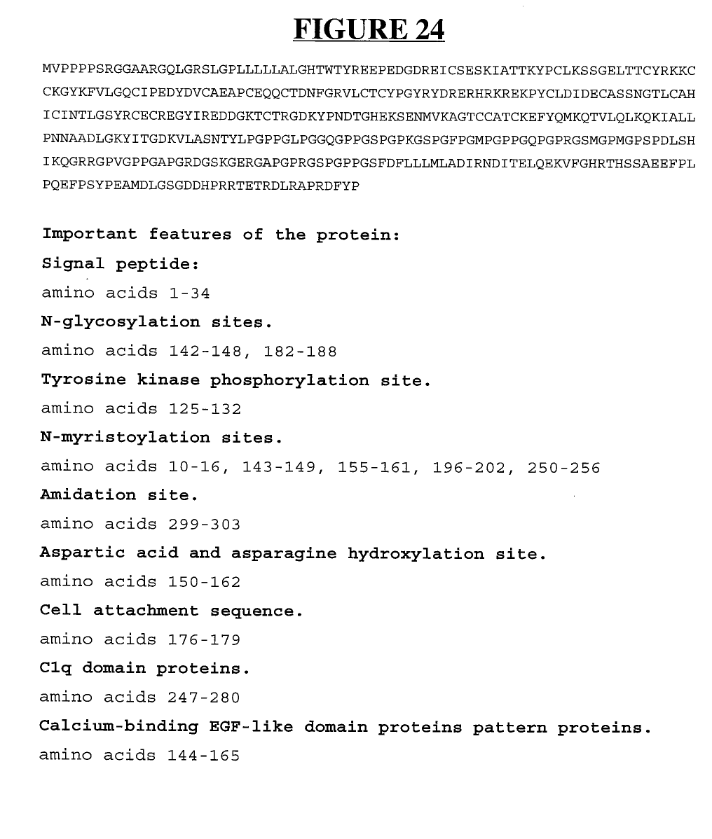

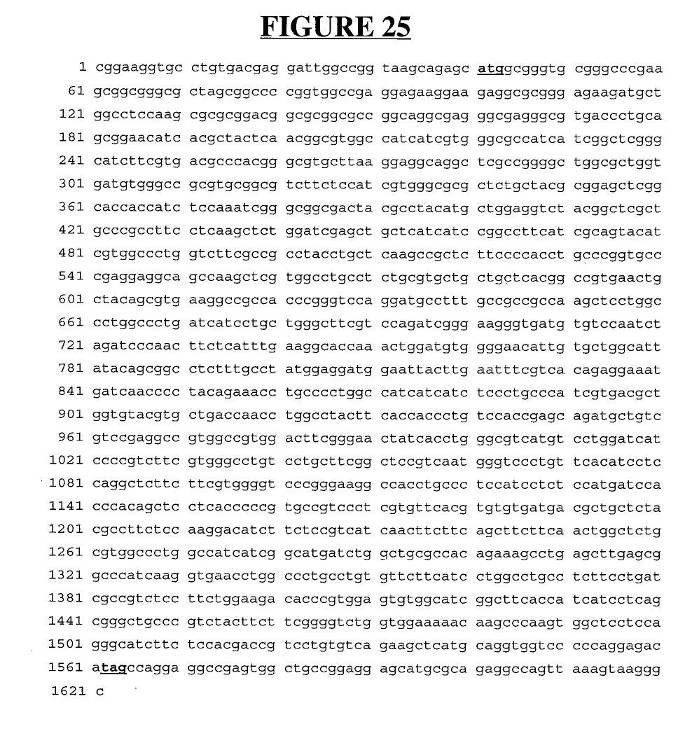

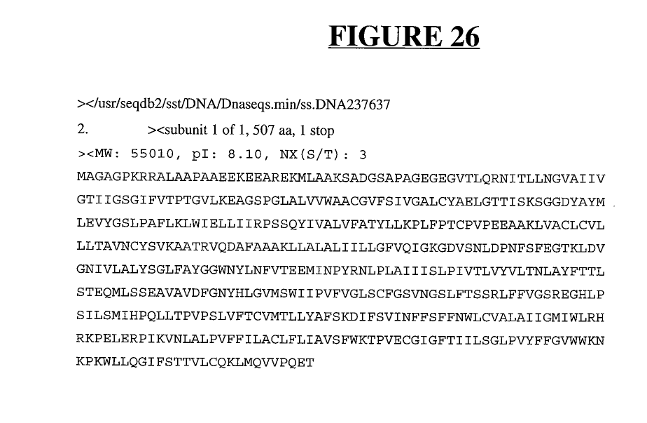

The entire coding sequence of PRO4395 is shown in FIG. 23 (SEQ ID NO:23). Clone DNA80840-2605 contains a single open reading frame with an apparent translational initiation site at nucleotide positions 17-19, and an apparent stop codon at nucleotide positions 1235-1237 of FIG. 23. The predicted polypeptide precursor is 406 amino acids long (FIG. 24; SEQ ID NO:24). Clone DNA80840-2605 (UNQ1921), designated as DNA80840-2605 has been deposited with ATCC on Apr. 20, 1999 and is assigned ATCC deposit no. 203949. The full-length PRO4395 protein shown in FIG. 24 has an estimated molecular weight of about 44103 daltons and a pI of about 7.93.

An analysis of the Dayhoff database (version 35.45 SwissProt 35), using a WU-BLAST2 sequence alignment analysis of the full-length sequence shown in FIG. 24 (SEQ ID NO:24), revealed homology between the PRO4395 amino acid sequence and the following Dayhoff sequences (sequences and related text incorporated herein): P_W07539, P_W57645, P_W57646, P_R95115, _R93250, P_R37740, _W57651, P_R37741, P_W01418, and P_R93254.

### Example 15

### Isolation of cDNA Clones Encoding Human PRO9799 Polypeptides [UNQ3018]

1. Preparation of oligo dT primed cDNA library

mRNA was isolated from human testis tissue using reagents and protocols from Invitrogen, San Diego, Calif. (Fast Track 2). This RNA was used to generate an oligo dT primed cDNA library in the vector pRK5D using reagents and protocols from Life Technologies, Gaithersburg, Md. (Super Script Plasmid System). In this procedure, the double stranded cDNA was sized to greater than 1000 bp and the SalI/NotI linkered cDNA was cloned into XhoI/NotI cleaved vector. pRK5D is a cloning vector that has an SP6 transcription initiation site followed by an SfiI restriction enzyme site preceding the XhoI/NotI cDNA cloning sites.

2. Preparation of Random Primed cDNA Library

A secondary cDNA library was generated in order to preferentially represent the 5′ ends of the primary cDNA clones. SP6 RNA was generated from the primary library (described above), and this RNA was used to generate a random primed cDNA library in the vector pSST-AMY.0 using reagents and protocols from Life Technologies (Super Script Plasmid System, referenced above). In this procedure the double stranded cDNA was sized to 500-1000 bp, linkered with blunt to NotI adaptors, cleaved with SfiI, and cloned into SfiI/NotI cleaved vector. pSST-AMY.0 is a cloning vector that has a yeast alcohol dehydrogenase promoter preceding the cDNA cloning sites and the mouse amylase sequence (the mature sequence without the secretion signal) followed by the yeast alcohol dehydrogenase terminator, after the cloning sites. Thus, cDNAs cloned into this vector that are fused in frame with the amylase sequence will lead to the secretion of amylase from appropriately transfected yeast colonies.

3. Transformation and Detection

DNA from the library described in paragraph 2 above was chilled on ice to which was added electrocompetent DH10B bacteria (Life Technologies, 20 ml). The bacteria and vector mixture was then electroporated as recommended by the manufacturer. Subsequently, SOC media (Life Technologies, 1 ml) was added and the mixture was incubated at 37° C. for 30 minutes. The transformants were then plated onto 20 standard 150 mm LB plates containing ampicillin and incubated for 16 hours (37° C.). Positive colonies were scraped off the plates and the DNA was isolated from the bacterial pellet using standard protocols, e.g. CsCl-gradient. The purified DNA was then carried on to the yeast protocols below.

The yeast methods were divided into three categories: (1) Transformation of yeast with the plasmid/cDNA combined vector; (2) Detection and isolation of yeast clones secreting amylase; and (3) PCR amplification of the insert directly from the yeast colony and purification of the DNA for sequencing and further analysis.

The yeast strain used was HD56-5A (ATCC-90785). This strain has the following genotype: MAT alpha, ura3-52, leu2-3, leu2-112, his3-11, his3-15, MAL⁺, SUC⁺, GAL⁺. Preferably, yeast mutants can be employed that have deficient post-translational pathways. Such mutants may have translocation deficient alleles in sec71, sec72, sec62, with truncated sec71 being most preferred. Alternatively, antagonists (including antisense nucleotides and/or ligands) which interfere with the normal operation of these genes, other proteins implicated in this post translation pathway (e.g., SEC61p, SEC72p, SEC62p, SEC63p, TDJ1p or SSA1p-4-p) or the complex formation of these proteins may also be preferably employed in combination with the amylase-expressing yeast.

Transformation was performed based on the protocol outlined by Gietz et al., Nucl. Acid. Res., 20:1425 (1992). Transformed cells were then inoculated from agar into YEPD complex media broth (100 ml) and grown overnight at 30° C. The YEPD broth was prepared as described in Kaiser et al., Methods in Yeast Genetics, Cold Spring Harbor Press, Cold Spring Harbor, N.Y., p. 207 (1994). The overnight culture was then diluted to about 2×10⁶ cells/ml (approx. OD₆₀₀=0.1) into fresh YEPD broth (500 ml) and regrown to 1×10⁷ cells/ml (approx. OD₆₀₀=0.4-0.5).

The cells were then harvested and prepared for transformation by transfer into GS3 rotor bottles in a Sorval GS3 rotor at 5,000 rpm for 5 minutes, the supernatant discarded, and then resuspended into sterile water, and centrifuged again in 50 ml falcon tubes at 3,500 rpm in a Beckman GS-6KR centrifuge. The supernatant was discarded and the cells were subsequently washed with LiAc/TE (10 ml, 10 mM Tris-HCl, 1 mM EDTA pH 7.5, 100 mM Li₂OOCCH₃), and resuspended into LiAc/TE (2.5 ml).

Transformation took place by mixing the prepared cells (1001) with freshly denatured single stranded salmon testes DNA (Lofstrand Labs, Gaithersburg, Md.) and transforming DNA (1 μg, vol. <10 μl) in microfuge tubes. The mixture was mixed briefly by vortexing, then 40% PEG/TE (600 μl, 40% polyethylene glycol-4000, 10 mM Tris-HCl, 1 mM EDTA, 100 mM Li₂OOCCH₃, pH 7.5) was added. This mixture was gently mixed and incubated at 30° C. while agitating for 30 minutes. The cells were then heat shocked at 42° C. for 15 minutes, and the reaction vessel centrifuged in a microfuge at 12,000 rpm for 5-10 seconds, decanted and resuspended into TE (500 μl, 10 mM Tris-HCl, 1 mM EDTA pH 7.5) followed by recentrifugation. The cells were then diluted into TE (1 ml) and aliquots (200 μl) were spread onto the selective media previously prepared in 150 mm growth plates (VWR).

Alternatively, instead of multiple small reactions, the transformation was performed using a single, large scale reaction, wherein reagent amounts were scaled up accordingly.

The selective media used was a synthetic complete dextrose agar lacking uracil (SCD-Ura) prepared as described in Kaiser et al., Methods in Yeast Genetics, Cold Spring Harbor Press, Cold Spring Harbor, N.Y., p. 208-210 (1994). Transformants were grown at 30° C. for 2-3 days.

The detection of colonies secreting amylase was performed by including red starch in the selective growth media. Starch was coupled to the red dye (Reactive Red-120, Sigma) as per the procedure described by Biely et al., Anal. Biochem., 172:176-179 (1988). The coupled starch was incorporated into the SCD-Ura agar plates at a final concentration of 0.15% (w/v), and was buffered with potassium phosphate to a pH of 7.0 (50-100 mM final concentration).

The positive colonies were picked and streaked across fresh selective media (onto 150 mm plates) in order to obtain well isolated and identifiable single colonies. Well isolated single colonies positive for amylase secretion were detected by direct incorporation of red starch into buffered SCD-Ura agar. Positive colonies were determined by their ability to break down starch resulting in a clear halo around the positive colony visualized directly.

4. Isolation of DNA by PCR Amplification

When a positive colony was isolated, a portion of it was picked by a toothpick and diluted into sterile water (30 μl) in a 96 well plate. At this time, the positive colonies were either frozen and stored for subsequent analysis or immediately amplified. An aliquot of cells (5 μl) was used as a template for the PCR reaction in a 25 μl volume containing: 0.5 μl Klentaq (Clontech, Palo Alto, Calif.); 4.0 μl 10 mM dNTP's (Perkin Elmer-Cetus); 2.5 μl Klentaq buffer (Clontech); 0.25 μl forward oligo 1; 0.25 μl reverse oligo 2; 12.5 μl distilled water. The sequence of the forward oligonucleotide 1 was:

(SEQ ID NO:41)

5′-TGTAAAACGACGGCCAGTTAAATAGACCTGCAATTATTAATCT-3′

The sequence of reverse oligonucleotide 2 was:

(SEQ ID NO:42)

5′-CAGGAAACAGCTATGACCACCTGCACACCTGCAAATCCATT-3′

PCR was then performed as follows:

| | | | |
|---|---|---|---|
| a. | | Denature | 92° C., 5 minutes |
| b. | 3 cycles of: | Denature | 92° C., 30 seconds |
| | | Anneal | 59° C., 30 seconds |
| | | Extend | 72° C., 60 seconds |
| c. | 3 cycles of: | Denature | 92° C., 30 seconds |
| | | Anneal | 57° C., 30 seconds |
| | | Extend | 72° C., 60 seconds |
| d. | 25 cycles of: | Denature | 92° C., 30 seconds |
| | | Anneal | 55° C., 30 seconds |
| | | Extend | 72° C., 60 seconds |
| e. | | Hold | 4° C. |

The underlined regions of the oligonucleotides disclosed above annealed to the ADH promoter region and the amylase region, respectively, and amplified a 307 bp region from vector pSST-AMY.0 when no insert was present. Typically, the first 18 nucleotides of the 5′ end of these oligonucleotides contained annealing sites for the sequencing primers. Thus, the total product of the PCR reaction from any empty vector was 343 bp. However, signal sequence-fused cDNA resulted in considerably longer nucleotide sequences.

Following the PCR, an aliquot of the reaction (5 μl) was examined by agarose gel electrophoresis in a 1% agarose gel using Tris-Borate-EDTA (TBE) buffering system as described by Sambrook et al., supra. Clones resulting in a single strong PCR product larger than 400 bp were further analyzed by DNA sequencing after purification with a 96 Qiaquick PCR clean-up column (Qiagen Inc., Chatsworth, Calif.).

5. Identification of Full-length Clone

A cDNA sequence isolated in the above screen is herein designated DNA82953. An expressed sequence tag (EST) DNA database (Incyte Pharmaceuticals, Palo Alto, Calif.) was searched and an EST was identified which showed homology to DNA82953.

EST clone no. 1933186 was then purchased from Incyte Pharmaceuticals, Palo Alto, Calif. and the cDNA insert of that clone was obtained and sequenced in entirety.

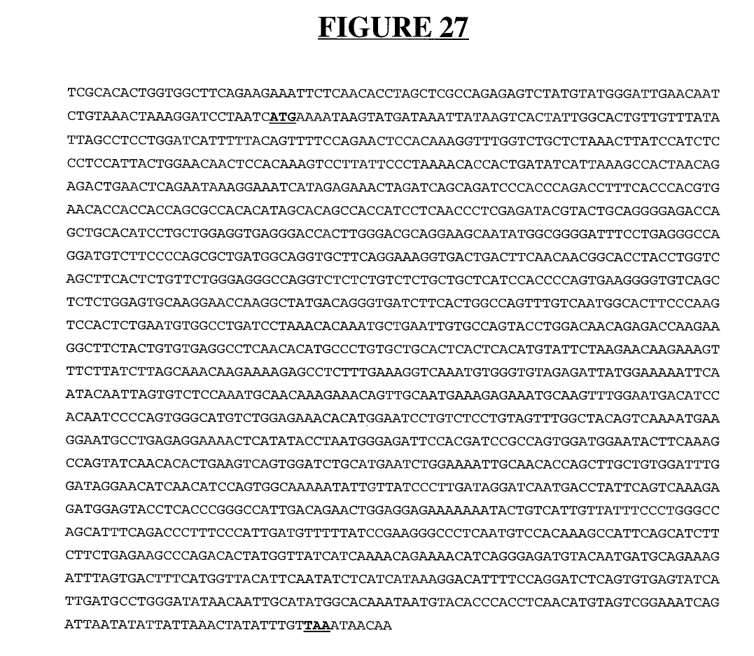

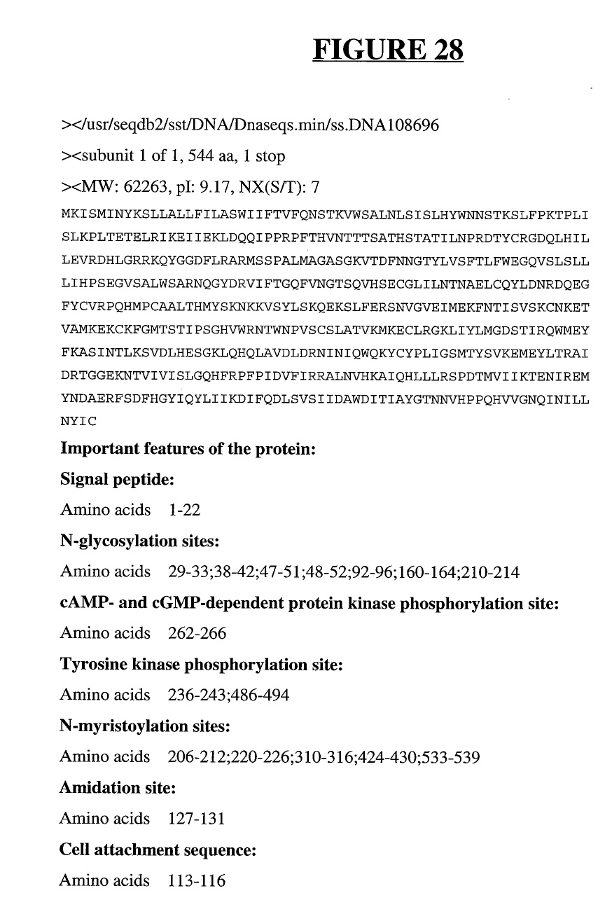

The entire nucleotide sequence of the clone, designated herein as DNA108696-2966, is shown in FIG. 27 (SEQ ID NO:27). The DNA108696-2966 clone contains a single open reading frame with an apparent translational initiation site at nucleotide positions 98-100 and a stop signal at nucleotide positions 1730-1732 (FIG. 27, SEQ ID NO:27). The predicted polypeptide precursor is 544 amino acids long, has a calculated molecular weight of approximately 62,263 daltons and an estimated pI of approximately 9.17. Analysis of the full-length PRO9799 sequence shown in FIG. 28 (SEQ ID NO:28) evidences the presence of a variety of important polypeptide domains as shown in FIG. 28, wherein the locations given for those important polypeptide domains are approximate as described above. Clone DNA108696-2966 has been deposited with ATCC on Aug. 1, 2000 and is assigned ATCC deposit no. PTA-2315.

An analysis of the Dayhoff database (version 35.45 SwissProt 35), using the ALIGN-2 sequence alignment analysis of the full-length sequence shown in FIG. 28 (SEQ ID NO:28), evidenced sequence identity between the PRO9799 amino acid sequence and the following Dayhoff sequences: BB61_RABIT and AK000134_{—}1.

### Example 16

### Isolation of cDNA Clones Encoding Human PRO21175 Polypeptides [UNQ03096]

An expressed sequence tag (EST) DNA database from Merck/Washington University was searched and an EST was identified which showed homology to Interleukin-17.

A pool of 50 different human cDNA libraries from various tissues was used in cloning. The cDNA libraries used to isolate the cDNA clones encoding human PRO21175 were constructed by standard methods using commercially available reagents such as those from Invitrogen, San Diego, Calif. The cDNA was primed with oligo dT containing a NotI site, linked with blunt to SalI hemikinased adaptors, cleaved with NotI, sized appropriately by gel electrophoresis, and cloned in a defined orientation into a suitable cloning vector (such as pRKB or pRKD; pRK5B is a precursor of pRK5D that does not contain the SfiI site; see, Holmes et al., Science, 253:1278-1280 (1991)) in the unique XhoI and NotI.

Oligonucleotides probes based upon the above described EST sequence were then synthesized: 1) to identify by PCR a cDNA library that contained the sequence of interest, and 2) for use as probes to isolate a clone of the full-length coding sequence for PRO21175. Forward and reverse PCR primers generally range from 20 to 30 nucleotides and are often designed to give a PCR product of about 100-1000 bp in length. The probe sequences are typically 40-55 bp in length. In order to screen several libraries for a full-length clone, DNA from the libraries was screened by PCR amplification, as per Ausubel et al., Current Protocols in Molecular Biology, supra, with the PCR primer pair. A positive library was then used to isolate clones encoding the gene of interest using the probe oligonucleotide and one of the primer pairs.

The oligonucleotide probes employed were as follows:

forward PCR primer

(SEQ ID NO:59)

5′-GCTCAGTGCCTTCCACCACACGC-3′
reverse PCR primer

(SEQ ID NO:60)

5′-CTGCGTCCTTCTCCGGCTCGG-3′
hybridization probe

(SEQ ID NO:61)

5′CGTTCCGTCTACACCGAGGCCTACGTCACCATCCCCGTGGGCTGC-3′

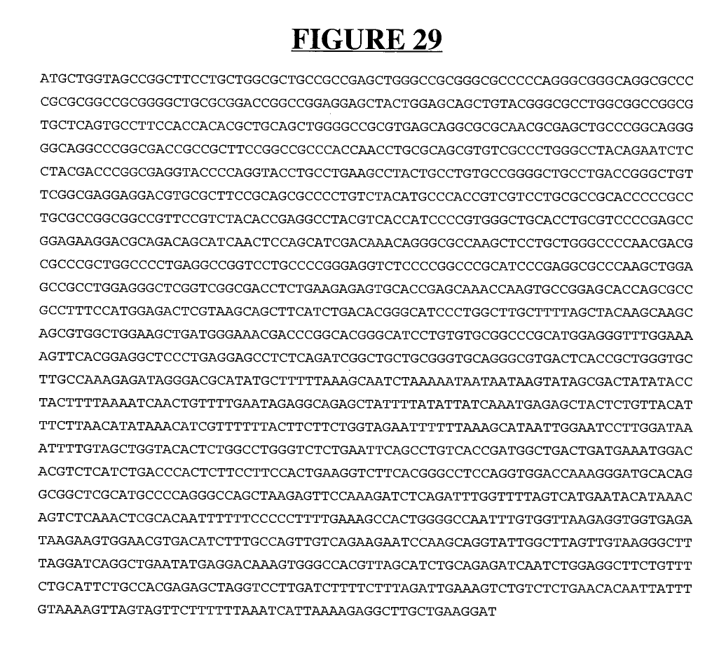

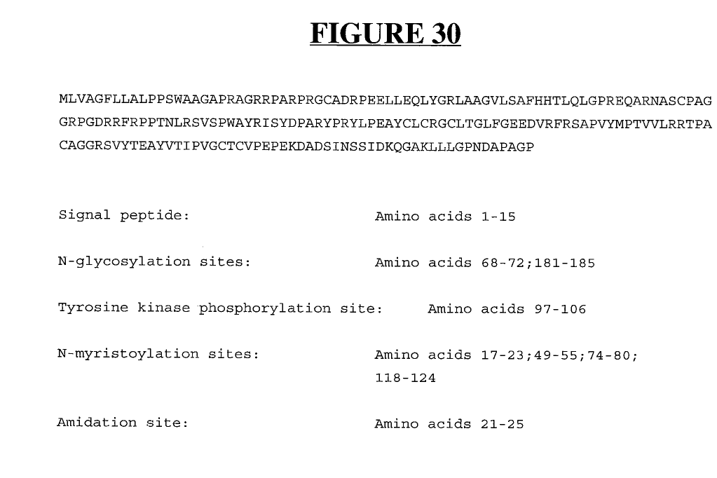

A full length clone was identified that contained a single open reading frame with an apparent translational initiation site at nucleotide positions 1-3 and a stop signal at nucleotide positions 607-609 (FIG. 29, SEQ ID NO:29), herein identified as DNA173894-2947. The predicted polypeptide precursor is 202 amino acids long, has a calculated molecular weight of approximately 21,879 daltons and an estimated pI of approximately 9.3. Analysis of the full-length PRO21175 sequence shown in FIG. 30 (SEQ ID NO:30) evidences the presence of a variety of important polypeptide domains as shown in FIG. 30, wherein the locations given for those important polypeptide domains are approximate as described above. Chromosome mapping evidences that the PRO21175-encoding nucleic acid maps to 13q11 in humans. Clone DNA173894-2947 has been deposited with ATCC on Jun. 20, 2000 and is assigned ATCC deposit no. PTA-2108.

Analysis of the amino acid sequence of the isolated full-length PRO21175 suggests that it possesses similarity with IL-17, thereby indicating that PRO21175 may be a novel cytokine and is herein designated IL-17D. Specifically, an analysis of the protein database (version 35.45 SwissProt 35), using the ALIGN-2 sequence alignment analysis of the full-length sequence shown in FIG. 30 (SEQ ID NO:30), evidenced sequence identity between the PRO21175 amino acid sequence and the following sequence: AF152099_{—}1.

### Example 17

### Isolation of cDNA Clones Encoding Human PRO19837 Polypeptides [UNQ5931]

The extracellular domain (ECD) sequences (including the secretion signal sequence, if any) from about 950 known secreted proteins from the Swiss-Prot public database were used to search EST databases. The EST databases included a proprietary EST database (LIFESEQ®, Incyte Pharmaceuticals, Palo Alto, Calif.). The search was performed using the computer program BLAST or BLAST2 [Altschul et al., Methods in Enzymology, 266:460-480 (1996)] as a comparison of the ECD protein sequences to a 6 frame translation of the EST sequences. Those comparisons resulting in a BLAST score of 70 (or in some cases, 90) or greater that did not encode known proteins were clustered and assembled into consensus DNA sequences with the program “phrap” (Phil Green, University of Washington, Seattle, Wash.).

A consensus DNA sequence was assembled relative to other EST sequences using phrap as described above. This consensus sequence is herein designated DNA131736. In some cases, the DNA131736 consensus sequence derives from an intermediate consensus DNA sequence which was extended using repeated cycles of BLAST and phrap to extend that intermediate consensus sequence as far as possible using the sources of EST sequences discussed above.

Based on the DNA131736 consensus sequence, flip cloning was performed. Oligonucleotides were synthesized: 1) to identify by PCR a cDNA library that contained the sequence of interest, and 2) for use as probes to isolate a clone of the full-length coding sequence for PRO19837. Forward and reverse PCR primers generally range from 20 to 30 nucleotides and are often designed to give a PCR product of about 100-1000 bp in length.

The probe sequences are typically 40-55 bp in length. In some cases, additional oligonucleotides are synthesized when the consensus sequence is greater than about 1-1.5 kbp. In order to screen several libraries for a full-length clone, DNA from the libraries was screened by Flip PCR amplification, as per Schanke et al., BioTechniques, 16:414-416 (1994), with the PCR primer pair. A positive library was then used to isolate clones encoding the gene of interest using the probe oligonucleotide and one of the primer pairs.

PCR primers (forward and reverse) were synthesized:

| | |
|---|---|
| forward PCR primer | |
| 5′-CCACCTTCCTCCCCTTGTGC-3′ | (SEQ ID NO:62) |
| reverse PCR primer | |
| 5′-CAGGTGTAGCAGCCGGCATC-3′ | (SEQ ID NO:63) |

Additionally, a synthetic oligonucleotide hybridization probe was constructed from the consensus DNA131736 sequence which had the following nucleotide sequence

hybridization probe

(SEQ ID NO:64)

5′-CAGTCATCGTGGTGCCCCCCAAGAACAGCACAGTCAATGC-3′

RNA for construction of the cDNA libraries was isolated from human tissue. The cDNA libraries used to isolate the cDNA clones were constructed by standard methods using commercially available reagents such as those from Invitrogen, San Diego, Calif. The cDNA was primed with oligo dT containing a NotI site, linked with blunt to SalI hemikinased adaptors, cleaved with NotI, sized appropriately by gel electrophoresis, and cloned in a defined orientation into a suitable cloning vector (such as pRKB or pRKD; pRK5B is a precursor of pRK5D that does not contain the SfiI site; see, Holmes et al., Science, 253:1278-1280 (1991)) in the unique XhoI and NotI sites.

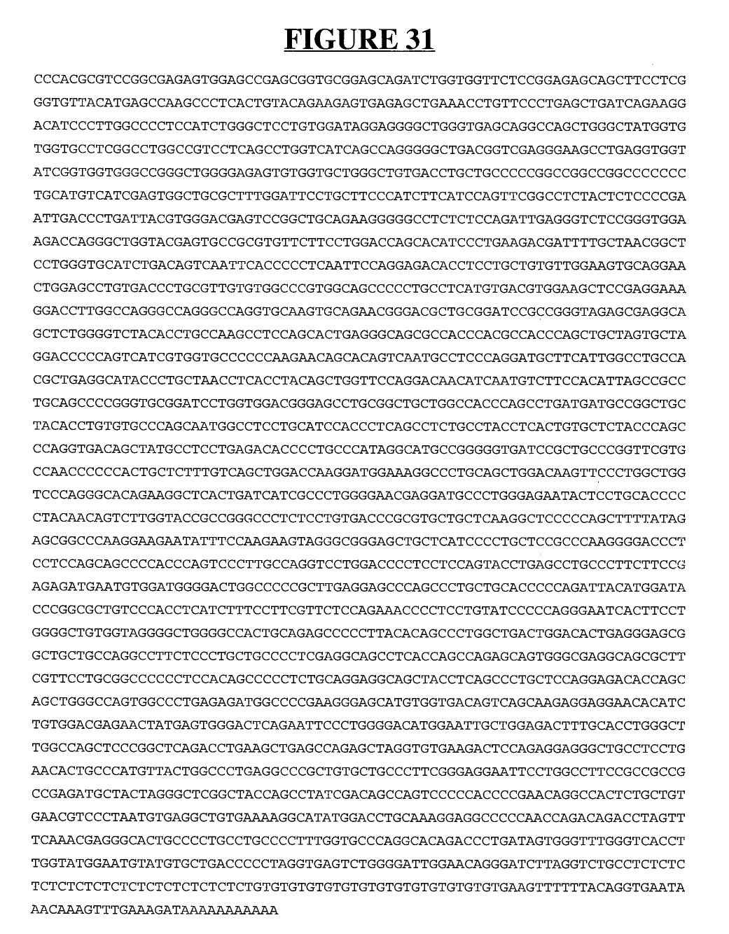

DNA sequencing of the clones isolated as described above gave the full-length DNA sequence for a full-length PRO19837 polypeptide (designated herein as DNA148009-2889 [FIG. 31, SEQ ID NO: 31]) and the derived protein sequence for that PRO19837 polypeptide.

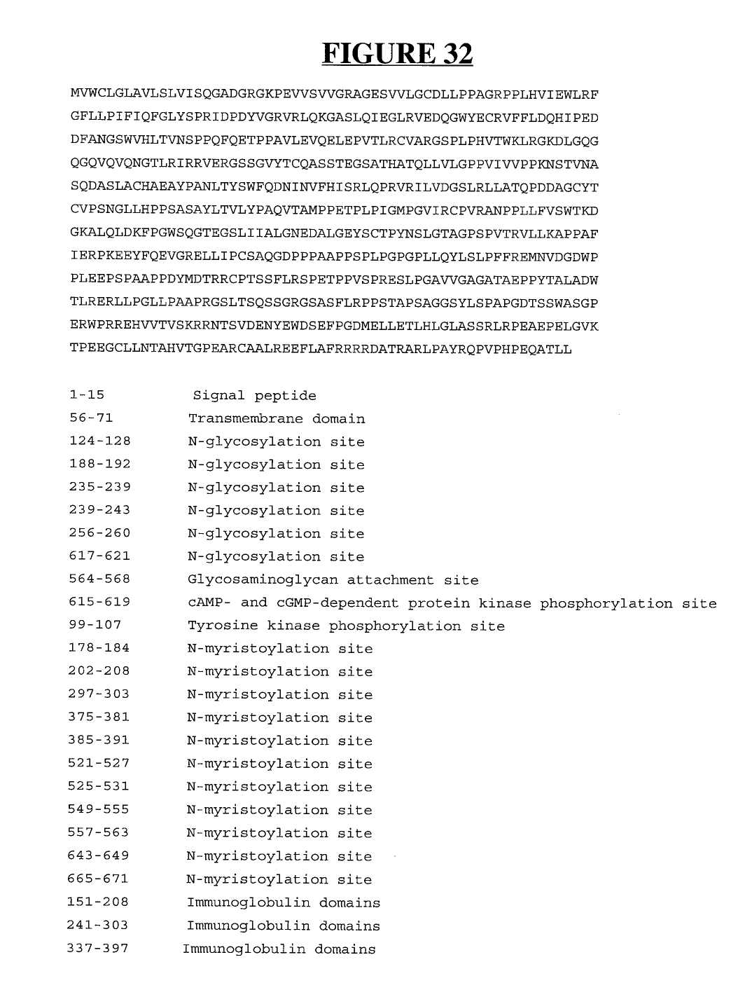

The full length clone identified above contained a single open reading frame with an apparent translational initiation site at nucleotide positions 217-219 and a stop signal at nucleotide positions 2368-2370 (FIG. 31, SEQ ID NO: 31). The predicted polypeptide precursor is 717 amino acids long, has a calculated molecular weight of approximately 77,750 daltons and an estimated pI of approximately 5.92. Analysis of the full-length PRO19837 sequence shown in FIG. 32 (SEQ ID NO: 32) evidences the presence of a variety of important polypeptide domains as shown in FIG. 32, wherein the locations given for those important polypeptide domains are approximate as described above. Clone DNA148009-2889 has been deposited with ATCC on May 9, 2000 and is assigned ATCC Deposit No. PTA-1839.

An analysis of the Dayhoff database (version 35.45 SwissProt 35), using the ALIGN-2 sequence alignment analysis of the full-length sequence shown in FIG. 32 (SEQ ID NO: 32), evidenced sequence identity between the PRO19837 amino acid sequence and the following Dayhoff sequences: AF181644_{—}1, AF200348_{—}1, P_W81030, AB032602_{—}1, AF217525_{—}1, P_Y08404, AF040990_{—}1, P_W82937, AB013802_{—}1, and OPCM_HUMAN.

### Example 18

### Isolation of cDNA Clones Encoding Human PRO21331 Polypeptides [UNQ6427]

The extracellular domain (ECD) sequences (including the secretion signal sequence, if any) from about 950 known secreted proteins from the Swiss-Prot public database were used to search EST databases. The EST databases included (1) public EST databases (e.g., GenBank), (2) a proprietary EST database (LIFESEQ®, Incyte Pharmaceuticals, Palo Alto, Calif.), and (3) a proprietary EST database from Genentech. The search was performed using the computer program BLAST or BLAST2 [Altschul et al., Methods in Enzymology, 266:460-480 (1996)] as a comparison of the ECD protein sequences to a 6 frame translation of the EST sequences. Those comparisons resulting in a BLAST score of 70 (or in some cases, 90) or greater that did not encode known proteins were clustered and assembled into consensus DNA sequences with the program “phrap” (Phil Green, University of Washington, Seattle, Wash.).

A consensus DNA sequence was assembled relative to other EST sequences using phrap as described above. This consensus sequence is herein designated DNA139100. In some cases, the consensus sequence derives from an intermediate consensus DNA sequence which was extended using repeated cycles of BLAST and phrap to extend that intermediate consensus sequence as far as possible using the sources of EST sequences discussed above.

Based on the DNA139100 consensus sequence, oligonucleotides were synthesized: 1) to identify by PCR a cDNA library that contained the sequence of interest, and 2) for use as probes to isolate a clone of the full-length coding sequence for PRO21331. Forward and reverse PCR primers generally range from 20 to 30 nucleotides and are often designed to give a PCR product of about 100-1000 bp in length. The probe sequences are typically 40-55 bp in length. In some cases, additional oligonucleotides are synthesized when the consensus sequence is greater than about 1-1.5 kbp. In order to screen several libraries for a full-length clone, DNA from the libraries was screened by PCR amplification, as per Ausubel et al., Current Protocols in Molecular Biology, supra, with the PCR primer pair. A positive library was then used to isolate clones encoding the gene of interest using the probe oligonucleotide and one of the primer pairs.

PCR primers (forward and reverse) were synthesized:

| | |
|---|---|
| forward PCR primer | |
| 5′-GCATCTGGGAAATTGGAGCTGAC-3′ | (SEQ ID NO:65) |
| reverse PCR primer | |
| 5′-GCACACATCCCATAGGGACAGC-3′ | (SEQ ID NO:66) |

Additionally, a synthetic oligonucleotide hybridization probe was constructed from the consensus DNA139100 sequence which had the following nucleotide sequence

hybridization probe

(SEQ ID NO:67)

5′-GCTGAGCTCCCTGCAAGCCCTGGATCTTAGCTGGAACGC-3′

RNA for construction of the cDNA libraries was isolated from a mixture of human tissues. The cDNA libraries used to isolate the cDNA clones were constructed by standard methods using commercially available reagents such as those from Invitrogen, San Diego, Calif. The cDNA was primed with oligo dT containing a NotI site, linked with blunt to SalI hemikinased adaptors, cleaved with NotI, sized appropriately by gel electrophoresis, and cloned in a defined orientation into a suitable cloning vector (such as pRK5B or pRK5D; pRK5B is a precursor of pRK5D that does not contain the SfiI site; see, Holmes et al., Science, 253:1278-1280 (1991)) in the unique XhoI and NotI sites.

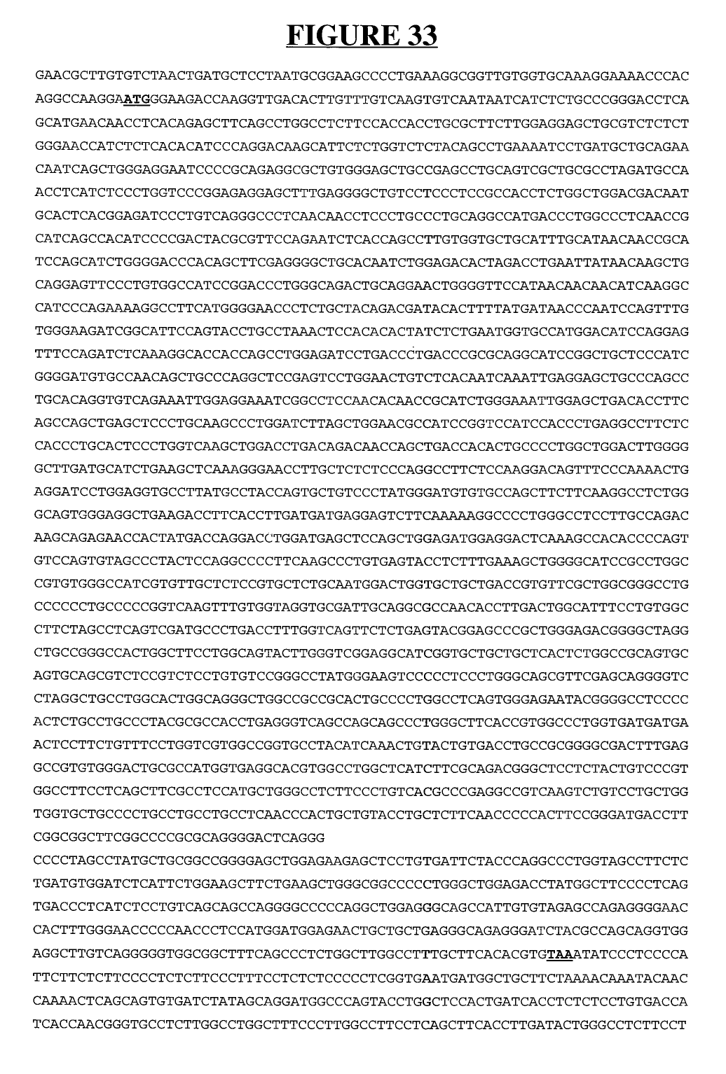

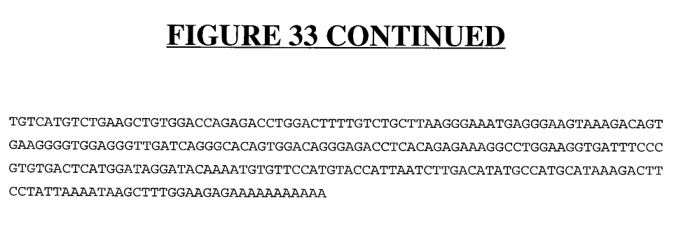

DNA sequencing of the clones isolated as described above gave the full-length DNA sequence for a full-length PRO21331 polypeptide (designated herein as DNA175959-2948 [FIG. 33, SEQ ID NO: 33]) and the derived protein sequence for that PRO21331 polypeptide.

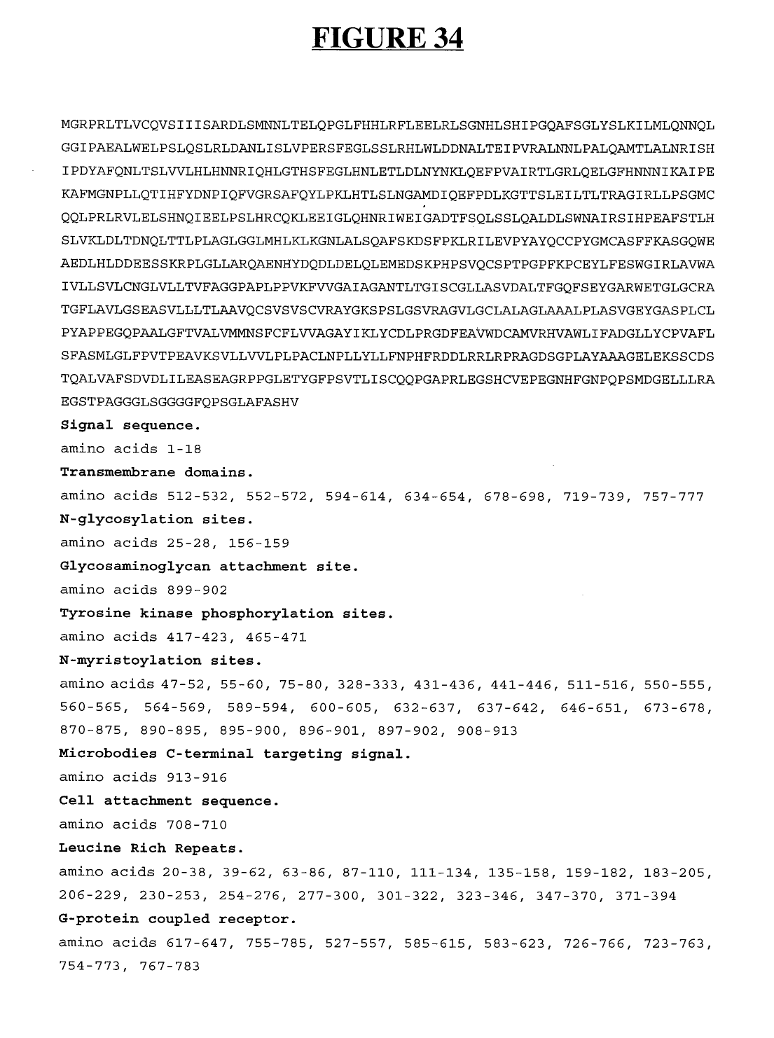

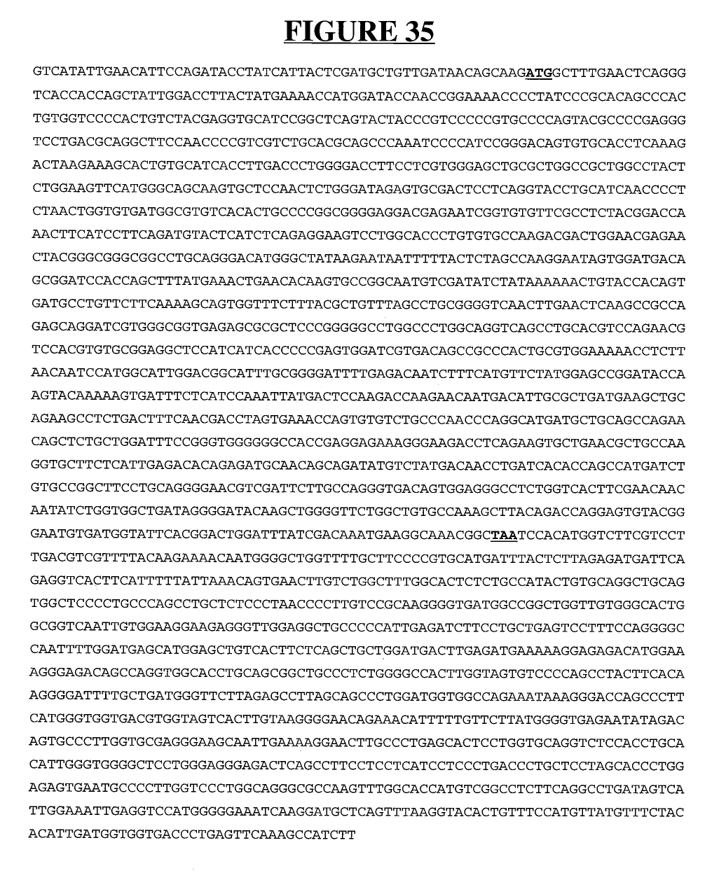

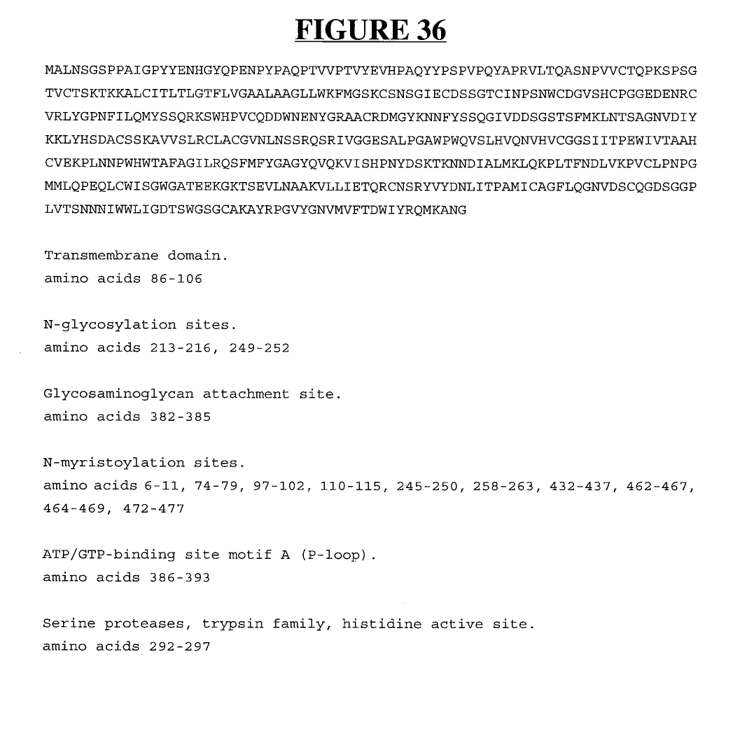

The full length clone identified above contained a single open reading frame with an apparent translational initiation site at nucleotide positions 85-87 and a stop signal at nucleotide positions 2830-2832 (FIG. 33, SEQ ID NO:33). The predicted polypeptide precursor is 915 amino acids long, has a calculated molecular weight of approximately 99,267 daltons and an estimated pI of approximately 5.93. Analysis of the full-length PRO21331 sequence shown in FIG. 34 (SEQ ID NO:34) evidences the presence of a variety of important polypeptide domains as shown in FIG. 34, wherein the locations given for those important polypeptide domains are approximate as described above. Clone DNA175959-2948 has been deposited with ATCC on Jul. 18, 2000 and is assigned ATCC deposit no. PTA-2248.

An analysis of the Dayhoff database (version 35.45 SwissProt 35), using the ALIGN-2 sequence alignment analysis of the full-length sequence shown in FIG. 34 (SEQ ID NO:34), evidenced sequence identity between the PRO21331 amino acid sequence and the following Dayhoff sequences: P_Y53575, P_Y53574, P_W93889, AF061444_{—}1, AF110818_{—}1, P_Y42168, AF257182_{—}1, P_W82318, P_Y53571, FSHR_HUMAN.

### Example 19

### Isolation of cDNA Clones Encoding Human PRO697 Polypeptides [UNQ361]

The extracellular domain (ECD) sequences (including the secretion signal, if any) of from about 950 known secreted proteins from the Swiss-Prot public protein database were used to search expressed sequence tag (EST) databases. The EST databases included public EST databases (e.g., GenBank, Merck/Wash. U) and a proprietary EST DNA database (LIFESEQ®, Incyte Pharmaceuticals, Palo Alto, Calif.). The search was performed using the computer program BLAST or BLAST2 (Altshul et al., Methods in Enzymology 266:460-480 (1996)) as a comparison of the ECD protein sequences to a 6 frame translation of the EST sequence. Those comparisons resulting in a BLAST score of 70 (or in some cases 90) or greater that did not encode known proteins were clustered and assembled into consensus DNA sequences with the program “phrap” (Phil Green, University of Washington, Seattle, Wash.)

A consensus DNA sequence was assembled relative to other EST sequences using phrap including Incyte clone 1910755. The consensus DNA sequence was extended using repeated cycles of BLAST and phrap to extend the consensus sequence as far as possible using the sources of EST sequences discussed above (also sometimes referred to as DNA43052). Based on this consensus sequence, oligonucleotides were synthesized: 1) to identify by PCR a cDNA library that contained the sequence of interest, and 2) for use as probes to isolate a clone of the full-length coding sequence for PRO697. Forward and reverse PCR primers generally range from 20 to 30 nucleotides and are often designed to give a PCR product of about 100-1000 bp in length. The probe sequences are typically 40-55 bp in length. In some cases, additional oligonucleotides are synthesized when the consensus sequence is greater than about 1-1.5 kbp. In order to screen several libraries for a full-length clone, DNA from the libraries was screened by PCR amplification, as per Ausubel et al., Current Protocols in Molecular Biology, with the PCR primer pair. A positive library was then used to isolate clones encoding the gene of interest using the probe oligonucleotide and one of the primer pairs.

A pair of PCR primers (forward and reverse) were synthesized:

| | |
|---|---|
| forward PCR primer | |
| 5′-CCTGGCTCGCTGCTGCTGCTC-3; | (SEQ ID NO:68) |
| and | |
| reverse PCR primer | |
| 5′-CCTCACAGGTGCACTGCAAGCTGTC-3′. | (SEQ ID NO:69) |

Additionally, a synthetic oligonucleotide hybridization probe was constructed from the consensus sequence which had the following nucleotide sequence:

hybridization probe

(SEQ ID NO:70)

5′-CTCTTCCTCTTTGGCCAGCCCGACTTCTCCTACAAGCGCAGAATTG
C-3′.

In order to screen several libraries for a source of a full-length clone, DNA from the libraries was screened by PCR amplification with the PCR primer pair identified above. A positive library was then used to isolate clones encoding the PRO697 gene using the probe oligonucleotide and one of the PCR primers.

RNA for construction of the cDNA libraries was isolated from human fetal kidney tissue (LIB227). The cDNA libraries used to isolate the cDNA clones were constructed by standard methods using commercially available reagents such as those from Invitrogen, San Diego, Calif. The cDNA was primed with oligo dT containing a NotI site, linked with blunt to SalI hemikinased adaptors, cleaved with NotI, sized appropriately by gel electrophoresis, and cloned in a defined orientation into a suitable cloning vector (such as pRKB or pRKD; pRK5B is a precursor of pRK5D that does not contain the SfiI site; see, Holmes et al., Science, 253:1278-1280 (1991)) in the unique XhoI and NotI sites.

DNA sequencing of the clones isolated as described above gave the full-length DNA sequence for PRO697 [herein designated as UNQ361 (DNA50920-1325)] (SEQ ID NO:37) and the derived protein sequence for PRO697.

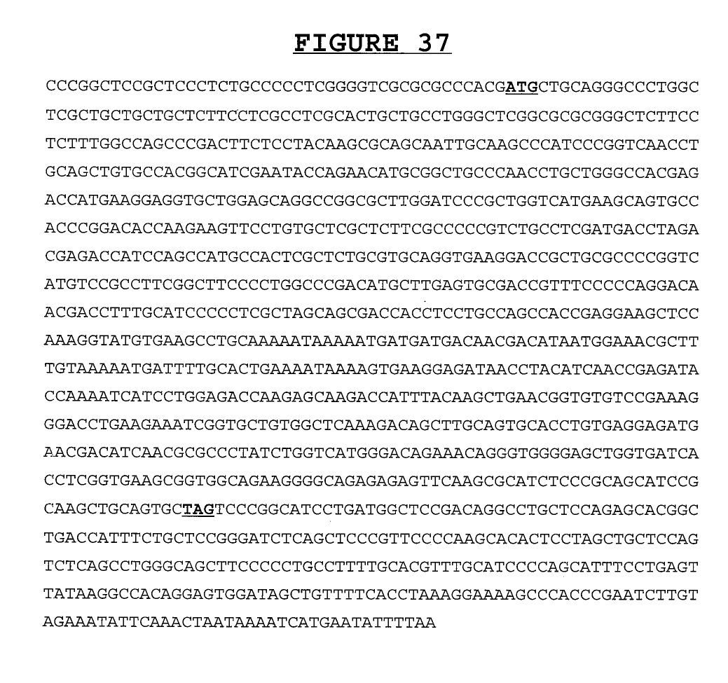

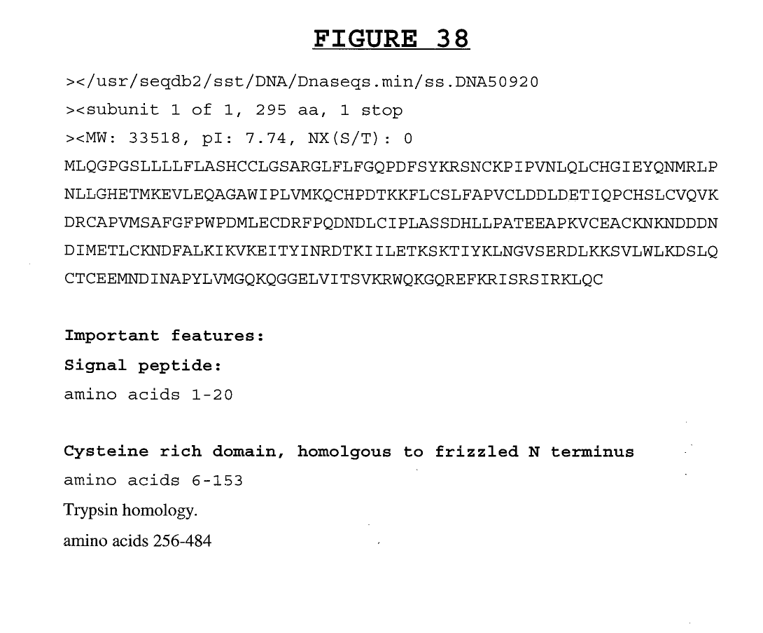

The entire nucleotide sequence of UNQ361 (DNA50920-1325) is shown in FIG. 37 (SEQ ID NO:37). Clone UNQ361 (DNA50920-1325) contains a single open reading frame with an apparent translational initiation site at nucleotide positions 44-46 and ending at the stop codon at nucleotide positions 929-931 (FIG. 37). The predicted polypeptide precursor is 295 amino acids long (FIG. 38; SEQ ID NO:38). The full-length PRO697 protein shown in FIG. 38 has an estimated molecular weight of about 33518 D and a pI of about 7.74. Clone UNQ361 (DNA50920-1325) was deposited with the ATCC on 26 Mar. 1998 (ATCC accession number 209700). It is understood that the deposited clone contains the actual sequence, and that the sequences provided herein are representative based on current sequencing techniques.

Analysis of the amino acid sequence of the full-length PRO697 polypeptide suggests that portions of it possess significant sequence identity with sFRPs, thereby indicating that PRO697 may be a novel sFRP family member.

Still analyzing the amino acid sequence of PRO697, the signal peptides is at about amino acids 1-20 of SEQ ID NO:38. The cystein rich domain, having identity with the frizzled N-terminus, is at about amino acids 6-153 of SEQ ID NO:38. The corresponding nucleotides can routinely be determined from the sequences provided herein.

### Example 20

### Isolation of cDNA Clones Encoding Human PRO1480 Polypeptides [UNQ749]

The extracellular domain (ECD) sequences (including the secretion signal sequence, if any) from about 950 known secreted proteins from the Swiss-Prot public database were used to search EST databases. The EST databases included public EST databases (e.g., GenBank), and a proprietary EST database (LIFESEQ®, Incyte Pharmaceuticals, Palo Alto, Calif.). The search was performed using the computer program BLAST or BLAST2 [Altschul et al., Methods in Enzymology, 266:460-480 (1996)] as a comparison of the ECD protein sequences to a 6 frame translation of the EST sequences. Using these methods, Incyte EST Nos. 550415 and 1628847 were identified as sequences of interest having BLAST scores of 70 or greater that did not encode known proteins. These sequences were clustered and assembled into a consensus DNA sequence with the program “phrap” (Phil Green, University of Washington, Seattle, Wash.). This consensus sequence is designated herein as “DNA1395”.

In addition, the “DNA1395” consensus sequence was extended using repeated cycles of BLAST and phrap to extend the consensus sequence as far as possible using the sources of EST sequences discussed above. The extended consensus sequence is designated herein as “DNA40642”.

Based on the DNA40642 consensus sequence, oligonucleotides were synthesized: 1) to identify by PCR a cDNA library that contained the sequence of interest, and 2) for use as probes to isolate a clone of the full-length coding sequence for PRO1480. Forward and reverse PCR primers generally range from 20 to 30 nucleotides and are often designed to give a PCR product of about 100-1000 bp in length. The probe sequences are typically 40-55 bp in length. In some cases, additional oligonucleotides are synthesized when the consensus sequence is greater than about 1-1.5 kbp. In order to screen several libraries for a full-length clone, DNA from the libraries was screened by PCR amplification, as per Ausubel et al., Current Protocols in Molecular Biology, supra, with the PCR primer pair. A positive library was then used to isolate clones encoding the gene of interest using the probe oligonucleotide and one of the primer pairs.

PCR primers (forward and reverse) were synthesized:

| | |
|---|---|
| forward PCR primer: | |
| AGCCCGTGCAGAATCTGCTCCTGG | (40642.f1; SEQ ID NO:71) |

reverse PCR primers:

| | |
|---|---|
| TGAAGCCAGGGCAGCGTCCTCTGG; | (40642.r1; SEQ ID NO:72) |
| GTACAGGCTGCAGTTGGC | (40642.r2; SEQ ID NO:73) |

Additionally, synthetic oligonucleotide hybridization probes were constructed from the consensus DNA40642 sequence which had the following nucleotide sequence:

hybridization probes:

(40642.p1; SEQ ID NO:74)

AGAAGCCATGTGAGCAAGTCCAGTTCCAGCCCAACACAGTG;

(40642.p2; SEQ ID NO:75)

GAGCTGCAGATCTTCTCATCGGGACAGCCCGTGCAGAATCTGCTC.

In order to screen several libraries for a source of a full-length clone, DNA from the libraries was screened by PCR amplification with the PCR primer pair identified above. A positive library was then used to isolate clones encoding the PRO1480 gene using the probe oligonucleotide and one of the PCR primers.

RNA for construction of the cDNA libraries was isolated from human fetal kidney tissue. The cDNA libraries used to isolated the cDNA clones were constructed by standard methods using commercially available reagents such as those from Invitrogen, San Diego, Calif. The cDNA was primed with oligo dT containing a NotI site, linked with blunt to SalI hemikinased adaptors, cleaved with NotI, sized appropriately by gel electrophoresis, and cloned in a defined orientation into a suitable cloning vector (such as pRKB or pRKD; pRK5B is a precursor of pRK5D that does not contain the SfiI site; see, Holmes et al., Science, 253:1278-1280 (1991)) in the unique XhoI and NotI sites.

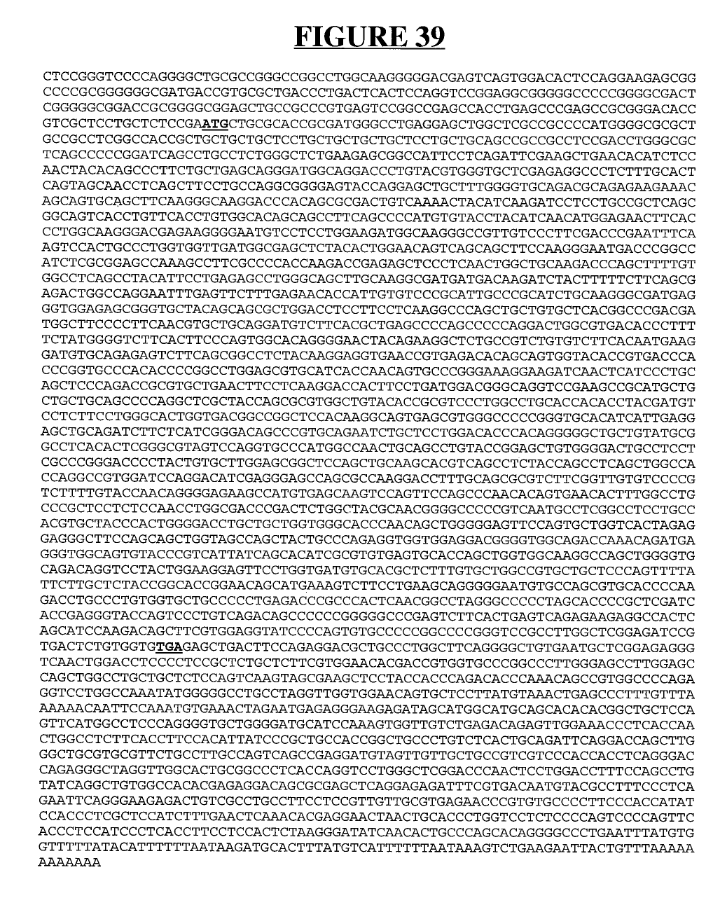

DNA sequencing of the clones isolated as described above gave the full-length DNA sequence for PRO1480, designated herein as DNA67962-1649 [FIG. 39, SEQ ID NO:39]; and the derived protein sequence for PRO1480.

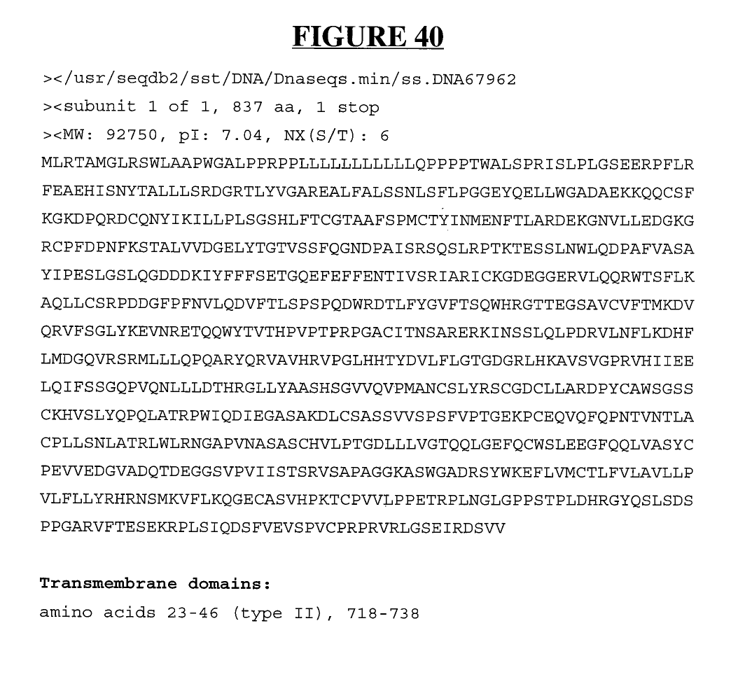

The entire coding sequence of PRO1480 is shown in FIG. 39 (SEQ ID NO:39). Clone DNA67962-1649 contains a single open reading frame with an apparent translational initiation site at nucleotide positions 241-243, and an apparent stop codon at nucleotide positions 2752-2754. The predicted polypeptide precursor is 837 amino acids long. The full-length PRO1480 protein shown in FIG. 40 (SEQ ID NO:40) has an estimated molecular weight of about 92,750 daltons and a pI of about 7.04. Additional features include: transmembrane domains at about amino acids 23-46 (type II) and 718-738; potential N-glycosylation sites at about amino acids 69-72, 96-99, 165-168, 410-413, 525-528, and 630-633; and a leucine zipper pattern at about amino acids 12-33.

An analysis of the Dayhoff database (version 35.45 SwissProt 35), using a WU-BLAST2 sequence alignment analysis of the full-length sequence shown in FIG. 40 (SEQ ID NO:40), revealed significant homology between the PRO1480 amino acid sequence and Dayhoff sequence 148746. Homology was also shown between the PRO1480 amino acid sequence and the following Dayhoff sequences: S66498; P_W17658; MMU69535_{—}1; HSU60800_{—}1; I48745; A49069; I48747; GGU28240_{—}1; and AF022946_{—}1.

Clone DNA67962-1649 (UNQ749) has been deposited with ATCC on Sep. 29, 1998 and is assigned ATCC deposit no. 203291.

### Example 21

### Generation and Analysis of Mice Comprising PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 Gene Disruptions

To investigate the role of PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides, disruptions in PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 genes were produced by homologous recombination. Specifically, transgenic mice comprising disruptions in PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 genes (i.e., knockout mice) were created by either gene targeting or gene trapping. Mutations were confirmed by southern blot analysis to confirm correct targeting on both the 5′ and 3′ ends. Gene-specific genotyping was also performed by genomic PCR to confirm the loss of the endogenous native transcript as demonstrated by RT-PCR using primers that anneal to exons flanking the site of insertion. Targeting vectors were electroporated into 129 strain ES cells and targeted clones were identified. Targeted clones were microinjected into host blastocysts to produce chimeras. Chimeras were bred with C57 animals to produce F1 heterozygotes. Heterozygotes were intercrossed to produce F2 wildtype, heterozygote and homozygote cohorts which were used for phenotypic analysis. Rarely, if not enough F1 heterozygotes were produced, the F1 hets were bred to wildtype C57 mice to produce sufficient heterozygotes to breed for cohorts to be analyzed for a phenotype. All phenotypic analysis was performed from 12-16 weeks after birth.

### Overall Summary of Phenotypic Results

### A. Generation and Analysis of Mice Comprising DNA35880-1160 (UNQ223) Gene Disruptions

In these knockout experiments, the gene encoding PRO256 polypeptides (designated as DNA35880-1160 (UNQ223) was disrupted. The gene specific information for these studies is as follows: the mutated mouse gene corresponds to nucleotide reference: NM_{—}016907. ACCESSION:NM_{—}016907 NID:8394350 or Mus musculus Mus musculus serine protease inhibitor, Kunitz type 1 (Spint1); protein reference: Q99J04. ACCESSION:Q99J04 NID: or Mus musculus (Mouse). SERINE PROTEASE INHIBITOR, KUNITZ TYPE 1. MOUSESPTRNRDB; the human gene sequence reference: NM_{—}003710. ACCESSION:NM_{—}003710 NID:450-4328 or Homo sapiens Homo sapiens serine protease inhibitor, Kunitz type 1 (SPINT1); the human protein sequence corresponds to reference: O43278. ACCESSION:O43278 NID: or Homo sapiens (Human). KUNITZ-TYPE PROTEASE INHIBITOR 1 PRECURSOR (HEPATOCYTE GROWTH FACTOR ACTIVATOR INHIBITOR TYPE 1) (HAI-1). HUMANSPTRNRDB.

The mouse gene of interest is Spint1 (serine protease inhibitor, Kunitz type 1), ortholog of human SPINT1. Aliases include HAI-1, HAI, HAI1, Kunitz-type protease inhibitor 1, and hepatocyte growth factor activator inhibitor 1.

SPINT1 is an integral membrane protein expressed on epithelial cells and white matter astrocytes that inhibits hepatocyte growth factor activator (HGFA) and matriptase, serine proteases that convert pro-hepatocyte growth factor (HGF) to biologically active HGF. SPINT1 is found not only in membranes from epithelia but also in blood and milk, indicating that the protein can be proteolytically cleaved and released into extracellular fluid. SPINT1 is likely to regulate tissue regeneration and tumorigenesis involving HGF signaling. SPINT1 appears to be upregulated in carcinomas from tissues such as ovaries and mammary gland (Kirchhofer et al., J Biol. Chem. 278(38):36341-9 (2003); Oberst et al., Am J. Pathol. 158(4):1301-11 (2001); Itoh et al., Am J Physiol Gastrointest Liver Physiol. 278(4):G635-43 (2000); Yamada et al., Exp Neurol. 153(1):60-4 (1998)).

Targeted or gene trap mutations were generated in strain 129SvEv^{Brd}-derived embryonic stem (ES) cells. The chimeric mice were bred to C57BL/6J albino mice to generate F1 heterozygous animals. These progeny were intercrossed to generate F2 wild type, heterozygous, and homozygous mutant progeny. On rare occasions, for example when very few F1 mice were obtained from the chimera, F1 heterozygous mice were crossed to 129SvEv^{Brd}/C57 hybrid mice to yield additional heterozygous animals for the intercross to generate the F2 mice.

| | wt | het | hom | Total |
|---|---|---|---|---|
| Observed | 19 | 35 | 0 | 54 |
| Expected | 13.5 | 27 | 13.5 | 54 |

Chi-Sq. = 18.11
Significance = 0.00012
(hom/n) = 0.00
Avg. Litter Size = 5

Retroviral insertion occurred in the intron between coding exons 1 and 2 (NCBI accession NM_{—}016907.2)

Wild-type expression of the target gene was detected in embryonic stem (ES) cells and, among the 13 adult tissue samples tested by RT-PCR, in thymus, spleen, lung, kidney, testis, and small intestine and colon. Due to lethality, transcript expression analysis was not performed. UNQ223 shows a dynamic expression pattern during development. It is expressed in several patterning centers including the tail bud and the AER of the limbs (this is the fast growing edge of the limb bud). It is also expressed in the yolk sac and the intersomitic blood vessels (probably in blood cells).

1. Phenotypic Analysis (for Disrupted Gene: DNA35880-1160 (UNQ223)

(a) Overall Phenotypic Summary:

Mutation of the gene encoding the ortholog of human serine protease inhibitor, Kunitz type 1 (SPINT1) resulted in lethality of (−/−) mutants. No notable phenotype was observed for (+/−) mice. Thus, DNA35880-1160 or its encoding polypeptide PRO256 must be essential for embryonic development. In particular, embryonic lethality appears to be due to a possible placental defect.

UNQ223 shows a dynamic expression pattern during development. It is expressed in several patterning centers including the tail bud and the AER of the limbs. It is also expressed in the yolk sac and the intersomitic blood vessels (probably in blood cells). Specifically, UNQ223 expression in mouse embryos shows staining in small vessels (circulating blood cells); yolk sac; AER of the limb (fast growing edge of the limb bud); bronchial arches; and optic vesicle (E10.5 and E11.5).

Discussion Related to Embryonic Developmental Abnormality of Lethality:

Embryonic lethality in knockout mice usually results from various serious developmental problems including but not limited to neurodegenerative diseases, angiogenic disorders, inflammatory diseases, or where the gene/protein has an important role in basic cell signaling processes in many cell types. In addition, embryonic lethals are useful as potential cancer models. Likewise, the corresponding heterozygous (+/−) mutant animals are particularly useful when they exhibit a phenotype and/or a pathology report which reveals highly informative clues as to the function of the knocked-out gene. For instance, EPO knockout animals were embryonic lethals, but the pathology reports on the embryos showed a profound lack of RBCs.

UNQ223 is a membrane-associated serine protease inhibitor called Hepatocyte growth factor (HGF) activator inhibitor type 1 (HAI-1). UNQ223 was identified by Genentech Scientist Daniel Kirchhofer. It is hypothesized to modulate the activity of the HGF/c-Met receptor system, which plays a well-documented role in tissue regeneration, morphogenesis and tumorigenesis. Analysis of the UNQ223 knockout mice show that HAI-1 plays a critical role in placenta development. Further characterization will determine whether UNQ223 is required for the differentiation and proliferation of placental trophoblast cells or whether it plays a role in the invasion of maternal decidual tissue by the embryonic cells.

Using a combination of RNA whole mount in situ and gal staining a careful examination of UNQ223 expression during early embryo development has been performed. This analysis demonstrates that at gastrulation stages, UNQ223 expression is restricted to 2 specific domains; the definitive endoderm in the mid-line of the embryo and in the extra-embryonic chorion. The chorionic tissue is destined to become the labyrinthine layer of the placenta. This is the region that acts as the interface between embryonic and maternal blood supplies. At later stages, UNQ223 is expressed in the labyrinth layer. At mid-gestation, UNQ223 is also expressed in multiple domains that represent signaling centers that orchestrate embryonic patterning events. For example, the apical ectodermal ridge (AER) which is involved in patterning the developing limb bud. UNQ223 also appears to be expressed in the circulating blood.

UNQ223 knockout mice have been shown to die between 9.5-11.5d of development. They are growth retarded and display developmental abnormalities consistent with abnormal placental development. Preliminary data examining the expression of a number of marker genes during placental development reveals that the labyrinthine layer of the placenta is greatly reduced in size in the UNQ223 homozygotes. Formation of labyrinthine layer of the placenta requires a carefully orchestrated invasion of embryonic trophoblasts into maternal decidual tissue. A similar role for HAI-1 may be important in regulating tumor invasiveness.

B. Generation and Analysis of Mice Comprising DNA212937 (UNQ281) Gene Disruptions

In these knockout experiments, the gene encoding PRO34421 polypeptides (designated as DNA212937 (UNQ281) was disrupted. The gene specific information for these studies is as follows: the mutated mouse gene corresponds to nucleotide reference: NM_{—}019397 or Mus musculus EGF-like-domain, multiple 6 (Egfl6); protein reference: NP-062270 or EGF-like-domain, multiple 6 [Mus musculus]; the human gene sequence reference: NM_{—}015507 or Homo sapiens EGF-like-domain, multiple 6 (EGFL6); the human protein sequence corresponds to reference: NP_{—}056322. epidermal growth factor-like protein 6 precursor; MAM domain- and EGF domain-containing protein precursor; EGF repeat-containing protein 6 precursor; EGF repeat-containing protein 6 [Homo sapiens].

The disrupted mouse gene is Egfl6 (EGF-like-domain, multiple 6), which is the ortholog of human EGFL6. Synonyms and aliases include W80, MAEG, DKFZp564P2063, EGF repeat-containing protein 6, EGF repeat-containing protein 6 precursor, and MAM domain- and EGF domain-containing protein precursor.

EGFL6 is a secreted protein encoded on the human and mouse X chromosomes that has been proposed as a factor involved in developmental disorders (Buchner, Orfanelli et al., Genomics 65(1):16-23 (2000)). Transcripts for the protein are found in placenta as well as brain, fetal, and lung tumors. Additionally, EGFL6 is expressed in the dermatone and dermatone derivatives. In mouse embryos, UNQ281 is expressed in the ventral somites (sclerotome (which are the precursors for vertebrae and ribs). In limbs, expression occurs in bones prior to ossification.

This project is X-linked.

### Summary of X-linked Gene Distribution by Sex and Genotype

(Only the agouti pups from the male chimeras are included.)

### Summary of X-linked Gene Distributions for Sex by Genotype

| | Agouti F1 (M | | | F1a | | |
|---|---|---|---|---|---|---|
| Progeny | chimera × wt) | | Progeny | (F het × wt) | | |
| Sex | wt | het | Sex | wt | het | hemi |
| M | 3 | 0 | M | 12 | n/a | 24 |
| F | 0 | 13 | F | 15 | 16 | n/a |

Targeted or gene trap mutations are generated in strain 129SvEvBrd-derived embryonic stem (ES) cells. The chimeric mice are bred to C57BL/6J albino mice to generate F1 female heterozygous animals. These progeny are crossed to hybrid 129SvEV^{Brd}/C57 F1 mice, derived from crossing 129SvEv^{Brd} mice to C57BL/6J mice, to generate F1A wild-type, female heterozygous, and male hemizygous mice. Level I phenotypic analysis is performed on mice from this generation.

| | wt | het | hom | Total |
|---|---|---|---|---|
| Observed | 17 | 14 | 28 | 59 |
| Expected | 14.75 | 29.5 | 14.75 | 59 |

Chi-Sq. = 20.39
Significance = 0.00004
(hom/n) = 0.47
Avg. Litter Size = 7

Mutation Type: Homologous Recombination (standard). Coding exon 1 was targeted (NM-019397).

Wild-type expression of the target gene was detected in embryonic stem (ES) cells and, among the 19 adult tissue samples tested by RT-PCR, in brain, spinal cord, spleen, lung, and kidney. Disruption of the target gene was confirmed by Southern hybridization analysis.

1. Phenotypic Analysis (for Disrupted Gene: DNA212937 (UNQ281)

(a) Overall Phenotypic Summary:

This mutation is in an X-linked gene. Both male and female wild-type mice were analyzed, whereas only male hemizygous mutant and female heterozygous mice were analyzed. Mutation of the gene encoding the ortholog of human EGF-like-domain, multiple 6 (EGFL6) resulted in decreased IgG1 and IgG2a responses to ovalbumin challenge in male (0/−) mice. The male (0/−) mice also exhibited increased serum insulin levels. In addition, the male (0/−) mice exhibited an increased mean body weight and fat when compared with their gender-matched littermates and the historical means. The knockout mice also exhibited an increased total tissue mass (TTM), lean body mass (LBM) and bone mineral related measurements. Gene disruption was confirmed by Southern blot.

(b) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders.

In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following tests were performed:

Ovalbumin Challenge

Procedure: This assay was carried out on 7 wild types and 8 homozygotes. Chicken ovalbumin (OVA) is a T-cell dependent antigen, which is commonly used as a model protein for studying antigen-specific immune responses in mice. OVA is non-toxic and inert and therefore will not cause harm to the animals even if no immune response is induced. The murine immune response to OVA has been well characterized, to the extent that the immunodominant peptides for eliciting T cell responses have been identified. Anti-OVA antibodies are detectable 8 to 10 days after immunization using enzyme-linked immunosorbent assay (ELIZA), and determination of different isotypes of antibodies gives further information on the complex processes that may lead to a deficient response in genetically engineered mice.

As noted above, this protocol assesses the ability of mice to raise an antigen-specific immune response. Animals were injected IP with 50 mg of chicken ovalbumin emulsified in Complete Feund's Adjuvant and 14 days later the serum titer of anti-ovalbumin antibodies (IgM, IgG1 and IgG2 subclasses) was measured. The amount of OVA-specific antibody in the serum sample is proportional to the Optical Density (OD) value generated by an instrument that scans a 96-well sample plate. Data was collected for a set of serial dilutions of each serum sample.

Results of this challenge: The male (0/−) mice exhibited a decreased mean serum IgG1 and IgG2a response to the ovalbumin challenge when compared with their (+/+) littermates. Thus, these knockout mice exhibited a decreased ability to elicit an OVA-specific antibody response to the T-cell dependent OVA antigen.

In summary, ovalbumin challenge studies indicate that knockout mice deficient in the gene encoding PRO34421 polypeptides exhibit immunological abnormalities when compared with their wild-type littermates.

In one instance, the mutant mice exhibited a decreased ability to elicit an immunological response when challenged with the T-cell dependent OVA antigen. This suggests that PRO34421 polypeptides or their agonists would be important agents which could stimulate the immune system (such as T cell proliferation) and would find utility in the cases wherein this effect would be beneficial to the individual such as in the case of leukemia, and other types of cancer, and in immunocompromised patients, such as AIDS sufferers. Accordingly, inhibitors (antagonists) of PRO34421 polypeptides would be useful in inhibiting the immune response and would be useful candidates for suppressing harmful immune responses, e.g. in the case of graft rejection or graft-versus-host diseases.

(c) Phenotypic Analysis: Metabolism-Blood Chemistry

In the area of metabolism, targets may be identified for the treatment of diabetes. Blood chemistry phenotypic analysis includes measuring serum insulin levels as an indicator of changes in glucose metabolism. Abnormal glucose metabolism can be related to the following disorders or conditions: Diabetes Type 1 and Type 2, Syndrome X, various cardiovascular diseases and/or obesity.

Insulin Data:

Test Description: Lexicon Genetics uses the Cobra II Series Auto-Gamma Counting System in its clinical settings for running quantitative Insulin assays on mice.

Results:

Blood chemistry analysis of serum insulin levels resulted in the male hemizygous (0/−) mutant mice exhibiting increased median serum insulin levels when compared with their gender-matched (0/+) littermates and the historical mean. Thus, knockout mice exhibited a phenotypic pattern of elevated insulin levels which can be related to an abnormal glucose metabolism. In addition, the male (0/−) mutant mice showed an increase in total body fat compared with their gender matched (0/+) littermates. This coupled with the observation of increased mean body weight in male (0/−) mutant mice suggests an obesity phenotype.

(d) Bone Metabolism: Radiology Phenotypic Analysis

In the area of bone metabolism, targets were identified herein for the treatment of arthritis, osteoporosis, osteopenia and osteopetrosis as well as identifying targets that promote bone healing. Tests included:

DEXA for measurement of bone mineral density on femur and vertebra

MicroCT for very high resolution and very high sensitivity measurements of bone mineral density for both trabecular and cortical bone.

Dexa Analysis—Test Description:

Procedure: A cohort of 4 wild type, 4 heterozygotes and 8 hemizygotes were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

DEXA Results: The male (0/−) mice exhibited an increased mean bone mineral content (BMC), bone mineral density (BMD) in total body, femur, and vertebrae when compared with their gender-matched (+/+) littermates and the historical means. In addition, the male mutant (0/−) mice demonstrated an increased total tissue mass (TTM), lean body mass (LBM) and fat percentages. These results are consistent with abnormal bone metabolism. These results indicate that the knockout mutant phenotype would be associated with such bone abnormalities as osteopetrosis. Osteopetrosis is a condition characterized by abnormal thickening and hardening of bone and abnormal fragility of the bones. As such, PRO34421 polypeptides or agonists thereof would be beneficial for the treatment of osteopetrosis. A phenotype associated with an increased bone mineral content, and total body and femoral bone mineral density suggests that agents which mimic these effects (e.g. antagonists of PRO34421 polypeptides) would be useful in bone healing.

C. Generation and Analysis of Mice Comprising DNA41379-1236 (UNQ295) Gene Disruptions

In these knockout experiments, the gene encoding PRO334 polypeptides (designated as DNA41379-1236 (UNQ295) was disrupted. The gene specific information for these studies is as follows: the mutated mouse gene corresponds to nucleotide reference:NM_{—}033525 or Mus musculus nephronectin (Npnt); protein reference: Q923T5. ACCESSION:Q923T5 NID: or Mus musculus (Mouse). NEPHRONECTIN. MOUSESPTRNRDB; the human gene sequence reference:NM_{—}198278 or Homo sapiens hypothetical protein LOC255743 (LOC255743); the human protein sequence corresponds to reference: IPI00375223. ACCESSION:IPI00375223 NID: or Homo sapiens (Human). HYPOTHETICAL PROTEIN LOC255743. IPI_human.

The disrupted locus is nephronectin (Npnt), which is the ortholog of human hypothetical protein LOC255743. Aliases include Nctn, POEM, AA682063, and I110009H02Rik.

Npnt is an extracellular matrix protein that functions as a ligand for integrin alpha8beta1. The protein contains a signal peptide, five epidermal growth factor-like repeats, a mucin region with an RGD sequence, and a C-terminal “meprin A5 receptor protein tyrosine phosphatase mu” (MAM) domain, all of which are found in secreted proteins or extracellular domains of membrane proteins. Npnt is strongly expressed in the developing kidney tubules, parathyroid and thyroid glands, bone, tooth germ, and endocrine organs of the brain (Morimura et al., J Biol. Chem. 276(45):42172-81 (2001)). Npnt is synthesized by ureteric epithelial cells and forms a complex with integrin alpha8beta1 in embryonic kidney, suggesting a role in kidney development (Brandenberger et al., J. Cell Biol. 154(2):447-58 (2001); Miner, J. H., J. Cell Biol. 154(2):257-9 (2001)).

Targeted or gene trap mutations were generated in strain 129SvEv^{Brd}-derived embryonic stem (ES) cells. The chimeric mice were bred to C57BL/6J albino mice to generate F1 heterozygous animals. These progeny were intercrossed to generate F2 wild type, heterozygous, and homozygous mutant progeny. On rare occasions, for example when very few F1 mice were obtained from the chimera, F1 heterozygous mice were crossed to 129SvEv^{Brd}/C57 hybrid mice to yield additional heterozygous animals for the intercross to generate the F2 mice. Level I phenotypic analysis was performed on mice from this generation as described below.

| | wt | het | hom | Total |
|---|---|---|---|---|
| Observed | 17 | 36 | 3 | 56 |
| Expected | 14 | 28 | 14 | 56 |

Chi-Sq. = 11.57
Significance = 0.00307
(hom/n) = 0.05
Avg. Litter Size = 7

Mutation Type: Homologous Recombination (standard). Coding exons 1 and 2 were targeted (NCBI accession AY035899).

Wild-type expression of the target gene was detected in embryonic stem (ES) cells and in all 13 adult tissue samples tested by RT-PCR, except tail. Disruption of the target gene was confirmed by Southern hybridization analysis.

UNQ295/nephronectin is expressed in the developing kidney and is hypothesized to be the ligand for alpha8beta1 integrin which is essential for normal kidney development. Only a proportion of alpha8beta 1 integrin knock-out mutant mice are viable and these have only one kidney.

1. Phenotypic Analysis (for Disrupted Gene: DNA41379-1236 (UNQ295)

(a) Overall Phenotypic Summary

Mutation of the gene encoding nephronectin (Npnt) resulted in greatly reduced viability of (−/−) mutants. The single male (−/−) mouse available for testing showed signs of growth retardation. This mutant mouse exhibited decreased mean body weight and total tissue mass as well as decreased bone mineral density and decreased vertebral trabecular bone volume and thickness, and femoral midshaft cortical thickness when compared with its gender-matched (+/+) and (+/−) littermates and the historical mean. Genetic data indicated that this mutation resulted in greatly reduced viability of the homozygous mutants. There was a significant reduction in the number of homozygotes surviving. Only three homozygous mutant mice were available for analysis (out of the predicted or expected 14 homozygotes). Heterozygous mice showed an improved glucose tolerance but there was only one heterozygous strain mouse available for testing. Gene disruption was confirmed by Southern blot.

Discussion Related to Embryonic Developmental Abnormality of Lethality:

Embryonic lethality in knockout mice usually results from various serious developmental problems including but not limited to neuro-degenerative diseases, angiogenic disorders, inflammatory diseases, or where the gene/protein has an important role in basic cell signaling processes in many cell types. In addition, embryonic lethals are useful as potential cancer models. Likewise, the corresponding heterozygous (+/−) mutant animals are particularly useful when they exhibit a phenotype and/or a pathology report which reveals highly informative clues as to the function of the knocked-out gene. For instance, EPO knockout animals were embryonic lethals, but the pathology reports on the embryos showed a profound lack of RBCs.

UNQ295 (nephronectin) is an extracellular matrix protein with 5 EGF-like repeats, a MAM domain and an RGD sequence. Published data indicates that UNQ295 is a ligand for the 8 1 integrin. Further analysis of the UNQ295 knockout mice demonstrate that nephronectin is required for kidney development and may be important for migration and differentiation of neural crest derived lineages.

By analyzing the gal reporter in the UNQ295 knockout mice, the dynamic expression pattern of nephronectin during embryonic development can be documented. Nephronectin is expressed in the developing kidney in the ureteric bud epithelium adjacent to the metenephric mesenchyme. This mesenchymal tissue is known to express 8 1 integrin. Preliminary data from heterozygous intercrosses dissected around birth show that UNQ295 is required for a normal kidney to develop. Two out of 5 homozygotes exhibited complete kidney agenesis, 1 had small kidneys and 2 had no obvious kidney abnormalities. These observations indicate that UNQ295 plays a critical role in the epithelial-mesenchymal interactions that occur during kidney morphogenesis. UNQ295 may play a similar role during tumor progression particularly renal carcinomas.

In rostral regions of the embryo, UNQ295 is expressed in cephalic neural crest cells. This population of cells goes on to form numerous structures in the head including the meninges surrounding the brain. This expression pattern is particularly interesting because microarray analysis data shows a significant and very large elevation in expression of UNQ295 in a neural crest derived brain tumor called a meningioma.

UNQ295 is expressed at high levels in the developing sclerotome. This tissue gives rise to the vertebrae and the ribs. Although UNQ295 knockouts show no obvious defects in either vertebrae or ribs, this may be due to the fact that another highly related molecule called Egfl6 (UNQ281) is expressed in the same sclerotome cells and may compensate for loss of UNQ295 activity. UNQ281 has also been knocked out in the Lexicon screen and homozygous mice are viable. Interestingly the single UNQ295 homozygous mouse that survived to the first pass phenotypic stage in current screening, displayed defects in bone thickness and density indicating that this molecule may play a role in bone disease.

(b) Bone Metabolism: Radiology Phenotypic Analysis

Procedure:

Radiologic phenotypic analysis was performed. In the area of bone metabolism, targets can be identified for the treatment of arthritis, osteoporosis, osteopenia and osteopetrosis as well as identifying targets that promote bone healing. Tests include DEXA and microCT for very high resolution and very high sensitivity measurements of bone mineral density for both trabecular and cortical bone.

DEXA Analysis—Test Description:

Procedure: A cohort of 4 wild type, 8 heterozygotes and one homozygote were tested in this assay. Dual Energy X-ray Absorptiometry (DEXA) has been used successfully to identify changes in total tissue mass (TTM).

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI, i.e., whole body, vertebrae, and both femurs).

DEXA Results:

As summarized above, the single male (−/−) mice available for testing exhibited decreased mean body weight, decreased mean total tissue mass and bone mineral density when compared with their gender-matched (+/) littermates and the historical means. The two (2) female (−/−) mice exhibited decreased mean percent total body fat when compared with their (+/+) and (+/−) littermates and the historical means.

Bone microCT Analysis:

Procedure: MicroCT was also used to get very sensitive measurements of BMD. The μCT40 scans dissected bones and provides detailed information on bone mass and architecture. One vertebra and 1 femur were taken from a cohort of 4 wild type and 3 homozygous mice. Measurements were taken of lumbar 5 veterbra traebecular bone volume, traebecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

Micro-CT Analysis Results:

The single male (−/−) mutant available for testing exhibited a notably decreased vertebral trabecular bone volume and thickness, and femoral midshaft cross-sectional cortical thickness when compared with gender-matched (+/+) littermates and the historical means.

These results demonstrate that knockout mutant mice exhibit abnormal bone metabolism with significant bone loss similar to osteoporosis characterized by decrease in bone mass with decreased density and possibly fragility leading to bone fractures. Thus, it appears that PRO334 or its encoding gene plays a vital role in embryonic development and viability. PRO334 polypeptides would be especially important for maintaining bone homeostasis and would be useful for bone healing or for the treatment of arthritis or osteoporosis, whereas PRO334 antagonists would lead to abnormal or pathological bone disorders including inflammatory diseases associated with abnormal bone metabolism including but not limited to arthritis, osteoporosis and osteopenia. In addition to these studies, (−/−) mutant mice showed signs of growth retardation. Such growth disorders are associated with the phenotype or physiological condition associated with tissue wasting diseases such as diabetes or cachexia. Thus, PRO334 polypeptides or agonists thereof would be useful for treating diabetes or cachexia.

D. Generation and Analysis of Mice Comprising DNA54228-1366-1 (UNQ408) Gene Disruptions

In these knockout experiments, the gene encoding PRO770 polypeptides (designated as DNA54228-1366-1 (UNQ408) was disrupted. The gene specific information for these studies is as follows: the mutated mouse gene corresponds to nucleotide reference: NM_{—}023881 or Mus musculus resistin like beta (Retnlb); protein reference: NP_{—}076370 or resistin like beta; found in inflammatory zone 2 [Mus musculus]; the human gene sequence reference: NM-032579 or Homo sapiens resistin like beta (RETNLB); the human protein sequence corresponds to reference: NP_{—}115968 or colon and small intestine-specific cysteine-rich protein precursor; cysteine-rich secreted A12-alpha-like protein 1; found in inflammatory zone 1 [Homo sapiens].

The disrupted mouse gene is Retnlb (resistin like beta), ortholog of human RETNLB (resistin like beta). Aliases include FIZZ1, Fizz2, Relmb, RELMbeta, 9030012B21Rik, HXCP2, found in inflammatory zone 1, found in inflammatory zone 2, cysteine-rich secreted A12-alpha-like protein 1, colon and small intestine-specific cysteine-rich protein precursor, and putative colon carcinoma-related protein precursor (CCRG).

RETNLB is a cysteine-rich protein that shows increased expression during allergic pulmonary inflammation in hypertrophic, hyperplastic bronchial epithelium. Additionally, during such inflammation RETNLB appears in type II alveolar pneumocytes (Holcomb et al., EMBO J. 19(15):4046-55 (2000)). RETNLB is secreted by goblet cells of the intestine in response to bacterial colonization (He et al., Gastroenterology 125(5): 1388-97 (2003)). Unlike resistin (RETN) and RETNLA, RETNLB is apparently not expressed in adipose tissues nor associated with insulin resistance (Beltowski, J., Med Sci Monit. 9(2):RA55-61 (2003)); it is reported to be expressed only in the gastrointestinal tract (Steppan et al., Proc Natl Acad Sci USA. 98(2):502-6 (2001)). RETNLB (CCRG) stimulates the proliferation of colon cancer cells (De Young, In Vivo. 16(4):239-48 (2002)).

Targeted or gene trap mutations were generated in strain 129SvEv^{Brd}-derived embryonic stem (ES) cells. The chimeric mice were bred to C57BL/6J albino mice to generate F1 heterozygous animals. These progeny were intercrossed to generate F2 wild type, heterozygous, and homozygous mutant progeny. On rare occasions, for example when very few F1 mice were obtained from the chimera, F1 heterozygous mice were crossed to 129SvEv^{Brd}/C57 hybrid mice to yield additional heterozygous animals for the intercross to generate the F2 mice. Level I phenotypic analysis was performed on mice from this generation as described below.

| | wt | het | hom | Total |
|---|---|---|---|---|
| Observed | 13 | 42 | 22 | 77 |
| Expected | 19.25 | 38.5 | 19.25 | 77 |

Chi-Sq. = 2.74
Significance = 0.25407
(hom/n) = 0.29
Avg. Litter Size = 8

Mutation Type: Homologous Recombination (standard). Coding exons 1 through 3 were targeted (NCBI accession NM_{—}023881.1).

Wild-type expression of the target gene was detected in thymus and small intestine and colon among the 13 adult tissue samples tested by RT-PCR. Disruption of the target gene was confirmed by Southern hybridization analysis.

1. Phenotypic Analysis (for Disrupted Gene: DNA54228-1366-1 (UNQ408)

(a) Overall Phenotypic Summary

Mutation of the gene encoding the ortholog of human resistin like beta (RETNLB) resulted in a decreased anxiety-related response in mutant (−/−) mice. In addition the (−/−) mice showed an increased bone mineral content and density and increased midshaft femur total area but a decrease in trabecular bone volume and connectivity density compared with their gender-matched littermates. Gene disruption was confirmed by Southern blot.

### (b) Phenotypic Analysis: CNS/Neurology

In the area of neurology, analysis focused herein on identifying in vivo validated targets for the treatment of neurological and psychiatric disorders including depression, generalized anxiety disorders, attention deficit hyperactivity disorder, obsessive compulsive disorder, schizophrenia, cognitive disorders, hyperalgesia and sensory disorders. Neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, specific phobia, substance-induced anxiety disorder, acute alcohol withdrawal, obsessive compulsive disorder, agoraphobia, bipolar disorder I or II, bipolar disorder not otherwise specified, cyclothymic disorder, depressive disorder, major depressive disorder, mood disorder, substance-induced mood disorder. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

Procedure:

Behavioral screens were performed on a cohort of 4 wild type, 4 heterozygous and 8 homozygous mutant mice. All behavioral tests were done between 12 and 16 weeks of age unless reduced viability necessitates earlier testing. These tests included open field to measure anxiety, activity levels and exploration.

Open Field Test:

Several targets of known drugs have exhibited phenotypes in the open field test. These include knockouts of the seratonin transporter, the dopamine transporter (Giros et al., Nature. 1996 Feb. 15; 379(6566):606-12), and the GABA receptor (Homanics et al., Proc Natl Acad Sci USA. 1997 Apr. 15; 94(8):4143-8). An automated open-field assay was customized to address changes related to affective state and exploratory patterns related to learning. First, the field (40×40 cm) was selected to be relatively large for a mouse, thus designed to pick up changes in locomotor activity associated with exploration. In addition, there were 4 holes in the floor to allow for nose-poking, an activity specifically related to exploration. Several factors were also designed to heighten the affective state associated with this test. The open-field test is the first experimental procedure in which the mice are tested, and the measurements that were taken were the subjects' first experience with the chamber. In addition, the open-field was brightly lit. All these factors will heighten the natural anxiety associated with novel and open spaces. The pattern and extent of exploratory activity, and especially the center-to-total distance traveled ratio, may then be able to discern changes related to susceptibility to anxiety or depression. A large arena (40 cm×40 cm, VersaMax animal activity monitoring system from AccuScan Instruments) with infrared beams at three different levels was used to record rearing, hole poke, and locomotor activity. The animal was placed in the center and its activity was measured for 20 minutes. Data from this test was analyzed in five, 4-minute intervals. The total distance traveled (cm), vertical movement number (rearing), number of hole pokes, and the center to total distance ratio were recorded.

The propensity for mice to exhibit normal habituation responses to a novel environment is assessed by determining the overall change in their horizontal locomotor activity across the 5 time intervals. This calculated slope of the change in activity over time is determined using normalized, rather than absolute, total distance traveled. The slope is determined from the regression line through the normalized activity at each of the 5 time intervals. Normal habituation is represented by a negative slope value.

Results: A notable difference was observed during open field activity testing. The (−/−) mice exhibited an increased median sum time in the center area when compared with their gender-matched (+/+) littermates, which is indicative of a decreased anxiety-like response in the mutants. [The (−/−) mice exhibited a lower delta T values suggesting decreased anxiety-related effects] Thus, knockout mice demonstrated a phenotype consistent with depressive disorders, schizophrenia and/or bipolar disorders. Thus, PRO770 polypeptides and agonists thereof would be useful for the treatment or amelioration of the symptoms associated with depressive disorders.

(c) Bone Metabolism: Radiology Phenotypic Analysis

In the area of bone metabolism, targets were identified herein for the treatment of arthritis, osteoporosis, osteopenia and osteopetrosis as well as identifying targets that promote bone healing. Tests included:

DEXA for measurement of bone mineral density on femur and vertebra

MicroCT for very high resolution and very high sensitivity measurements of bone mineral density for both trabecular and cortical bone.

Dexa Analysis—Test Description:

Procedure: A cohort of 4 wild type, 4 heterozygotes and 8 homozygotes were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Bone microCT Analysis:

Procedure: MicroCT was also used to get very sensitive measurements of BMD. One vertebra and 1 femur were taken from a cohort of 4 wild type and 8 homozygous mice. Measurements were taken of lumbar 5 veterbra traebecular bone volume, traebecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

DEXA and microCT Results:

The female (−/−) mice exhibited increased mean bone mineral content (BMC), and BMC/LBM and bone mineral density (BMD) when compared with their gender-matched (+/+) littermates. In addition, micro CT results showed the (−/−) mice to have an increased midshaft femur total area, but a decrease in trabecular bone volume and connectivity density. These results indicate that the knockout mutant phenotype is associated with such bone abnormalities as osteopetrosis. Osteopetrosis is a condition characterized by abnormal thickening and hardening of bone and abnormal fragility of the bones. As such, PRO770 polypeptides or agonists thereof would be beneficial for the treatment of osteopetrosis. A phenotype associated with an increased bone mineral content, and total body and femoral bone mineral density suggests that agents which mimic these effects (e.g. antagonists of PRO770 polypeptides) would be useful in bone healing.

E. Generation and Analysis of Mice Comprising DNA53977-1371 (UNQ484) Gene Disruptions

In these knockout experiments, the gene encoding PRO983 polypeptides (designated as DNA53977-1371 (UNQ484) was disrupted. The gene specific information for these studies is as follows: the mutated mouse gene corresponds to nucleotide reference: NM_{—}019806 or ACCESSION:NM_{—}019806 NID: gi 9790282 ref NM_{—}019806.1 or Mus musculus vesicle-associated membrane protein, associated protein B and C (Vapb); protein reference: Q9QY76 or ACCESSION:Q9QY76 NID:or Mus musculus (Mouse). VAMP-ASSOCIATED PROTEIN 33B. MOUSESPTRNRDB; the human gene sequence reference: NM_{—}004738 or ACCESSION:NM-004738 NID: orgi 4759301 ref NM_{—}004738.1 Homo sapiens VAMP (vesicle-associated membrane protein)-associated protein B and C (VAPB); the human protein sequence corresponds to reference: O95292 or ACCESSION:095292 NID: or Homo sapiens (Human). VAMP-ASSOCIATED PROTEIN B (DJ1018E9.1) (VAMP (VESICLE-ASSOCIATED MEMBRANE PROTEIN)-ASSOCIATED PROTEIN B AND C). HUMANSPTRNRDB.

The mouse gene of interest is Vapb (vesicle-associated membrane protein, associated protein B and C), ortholog of human VAPB (VAMP [vesicle-associated membrane protein]-associated protein B and C). Aliases include VAP33b, D2Abb2e, Vamp33b, VAMP-associated protein 33b, VAPC, VAP-B, VAP-C, VAMP-associated protein B, VAMP-associated protein C, and VAMP-associated 33 kDa protein.

VAPB is aubiquitous type. IV membrane protein found in the endoplasmic reticulum (Skehel et al., 2000). The protein contains a conserved N-terminal domain, a coiled-coil domain, and a C-terminal transmembrane domain. VAPC, an alternative product of the same gene, consists of the 70 conserved N-terminal residues but lacks the coiled-coil domain and transmembrane segment. VAPB associates with v-SNARE (soluble N-ethylmaleimide sensitive factor attachment protein receptors) proteins VAMP1 (vesicle-associated membrane protein-1) and VAMP2 (Nishimura et al., Biochem Biophys Res Commun 254(1):21-6 (1999)). VAPB is likely to be involved in vesicle trafficking or the control of neurotransmitter release (Skehel et al., Proc Natl Acad Sci USA 97(3):1101-6 (2000); Foster et al., Traffic 1(6):512-21 (2000)). In Drosophila, DVAP-33A, a homolog of human VAPB, plays a role in controlling the number of synaptic boutons at neuromuscular junctions (Pennetta et al., Neuron 35(2):291-306 (2002)).

Targeted or gene trap mutations were generated in strain 129SvEv^{Brd}-derived embryonic stem (ES) cells. The chimeric mice were bred to C57BL/6J albino mice to generate F1 heterozygous animals. These progeny were intercrossed to generate F2 wild type, heterozygous, and homozygous mutant progeny. On rare occasions, for example when very few F1 mice were obtained from the chimera, F1 heterozygous mice were crossed to 129SvEv^{Brd}/C57 hybrid mice to yield additional heterozygous animals for the intercross to generate the F2 mice. Level I phenotypic analysis was performed on mice from this generation as described below.

| | wt | het | hom | Total |
|---|---|---|---|---|
| Observed | 11 | 34 | 23 | 68 |
| Expected | 17 | 34 | 17 | 68 |

Chi-Sq. = 4.24
Significance = 0.12031
(hom/n) = 0.34
Avg. Litter Size = 8

Mutation Type: Retroviral Insertion (OST). Retroviral insertion occurred in the intron between coding exons 1 and 2 (NCBI accession NM_{—}019806.3).

Wild-type expression of the target gene was detected in embryonic stem (ES) cells and in all 13 adult tissue samples tested by RT-PCR. RT-PCR analysis revealed that the transcript was absent in the (−/−) mouse analyzed (F-73).

1. Phenotypic Analysis (for Disrupted Gene: DNA53977-1371 (UNQ484)

(a) Overall Phenotypic Summary

Mutation of the gene encoding the ortholog of human VAMP (vesicle-associated membrane protein)-associated protein B and C (VAPB) resulted in enhanced motor coordination in (−/−) mice. In addition, the male (−/−) mice exhibited decreased total tissue mass and fat while female (−/−) mice showed an increased total tissue mass and fat. Transcript was absent by RT-PCR.

### (b) Phenotypic Analysis: CNS/Neurology

In the area of neurology, analysis focused herein on identifying in vivo validated targets for the treatment of neurological and psychiatric disorders including depression, generalized anxiety disorders, attention deficit hyperactivity disorder, obsessive compulsive disorder, schizophrenia, cognitive disorders, hyperalgesia and sensory disorders. Neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, specific phobia, substance-induced anxiety disorder, acute alcohol withdrawal, obsessive compulsive disorder, agoraphobia, bipolar disorder I or II, bipolar disorder not otherwise specified, cyclothymic disorder, depressive disorder, major depressive disorder, mood disorder, substance-induced mood disorder. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

Procedure:

Behavioral screens were performed on a cohort of 4 wild type, 4 heterozygous and 8 homozygous mutant mice. All behavioral tests were done between 12 and 16 weeks of age unless reduced viability necessitates earlier testing. These tests included open field to measure anxiety, activity levels and exploration.

Inverted Screen Test Data:

The Inverted Screen is used to measure motor strength/coordination. Untrained mice were placed individually on top of a square (7.5 cm×7.5 cm) wire screen which was mounted horizontally on a metal rod. The rod was then rotated 180 degrees so that the mice were on the bottom of the screens. The following behavioral responses were recorded over a 1 min testing session: fell off, did not climb, and climbed up.

| Genotype | Ratio Fell Down | % | Ratio Climbed up | % |
|---|---|---|---|---|
| +/+ (n = 4) | 0/4 | 0 | 2/4 | 50 |
| +/− (n = 4) | 0/4 | 0 | 3/4 | 75 |
| −/− (n = 8) | 0/8 | 0 | 8/8 | *100 |

*coding indicates a notable difference.

A motor strength deficit is apparent when there is a 50% point difference between (−/−) or (+/−) mice and (+/+) mice for the fell down response. 0/8 or 1/8 (−/−) or (+/−) mice not climbing indicates impaired motor coordination. 7/8 or 8/8(−/−) or (+/−) mice climbing up indicates enhanced motor coordination.

Results:

The Inverted Screen Test is designed to measure basic Sensory & motor observations: All 8 (−/−) mutant mice (100%) climbed up the inverted screen, whereas only 2/4 (+/+) mice (M−121 and F-116) climbed up, suggesting enhanced motor coordination in the mutants. Thus changes in the neuromuscular junction could be of interest.

These observations suggest that the homozygotes (−/−) exhibit an enhanced motor coordination suggestive of enhanced neuromuscular abilities or a positive neurological phenotype. Thus, antagonists to PRO983 polypeptides or its encoding gene would be useful in treating or ameliorating impaired neuromuscular conditions.

PRO983 polypeptides or agonists thereof would be expected to mimic or be associated with such neuromuscular disorders or diseases.

F. Generation and Analysis of Mice Comprising DNA57129-1413 (UNQ493) Gene Disruptions

In these knockout experiments, the gene encoding PRO1009 polypeptides (designated as DNA57129-1413 (UNQ493) was disrupted. The gene specific information for these studies is as follows: the mutated mouse gene corresponds to nucleotide reference: NM-153807 or Mus musculus cDNA sequence BC018371 (BC018371); protein reference: □8VCW8 or ACCESSION:Q8VCW8 NID: or Mus musculus (Mouse). SIMILAR TO HYPOTHETICAL PROTEIN FLJ20920. MOUSESPTRNRDB; the human gene sequence reference: NM_{—}025149 or ACCESSION:NM_{—}025149 NID:13376740 Homo sapiens Homo sapiens hypothetical protein FLJ20920 (FLJ20920); the human protein sequence corresponds to reference:Q9H7G2. Hypothetical protein FLJ20920.

The disrupted mouse gene is a hypothetical protein (interim name, MGC25878), which is orthologous to human hypothetical protein FLJ20920. Aliases include “cDNA sequence BC018371” and “clone DNA57129 AVYV493”.

FLJ20920 is likely to be a mitochondrial enzyme; it contains an N-terminal mitochondrial transit peptide and an AMP-binding enzyme domain (Pfam PF00501). Other proteins sharing the motif include firefly luciferase, long-chain fatty acid-CoA ligase, acetyl-CoA ligase, and acetyl-CoA. KOG (clusters of orthologous groups for eukaryotic complete genomes) analysis suggests that FLJ20920 functions as a long-chain fatty acid acyl-CoA ligase (KOG1177).

Targeted or gene trap mutations were generated in strain 129SvEv^{Brd}-derived embryonic stem (ES) cells. The chimeric mice were bred to C57BL/6J albino mice to generate F1 heterozygous animals. These progeny were intercrossed to generate F2 wild type, heterozygous, and homozygous mutant progeny. On rare occasions, for example when very few F1 mice were obtained from the chimera, F1 heterozygous mice were crossed to 129SvEv^{Brd}/C57 hybrid mice to yield additional heterozygous animals for the intercross to generate the F2 mice. Level I phenotypic analysis was performed on mice from this generation

| | wt | het | hom | Total |
|---|---|---|---|---|
| Observed | 19 | 37 | 16 | 72 |
| Expected | 18 | 36 | 18 | 72 |

Chi-Sq. = 0.31
Significance = 0.85832
(hom/n) = 0.22
Avg. Litter Size = 7

Mutation Information: Mutation Type: Retroviral Insertion (OST). Retroviral insertion occurred in the intron between coding exons 1 and 2 (NCBI accession number NM_{—}153807.1).

Wild-type expression of the target gene was detected in embryonic stem (ES) cells and in all 13 adult tissue samples tested by RT-PCR. RT-PCR analysis revealed that the transcript was absent in the (−/−) mouse analyzed (M-114).

1. Phenotypic Analysis (for Disrupted Gene: DNA57129-1413 (UNQ493)

(a) Overall Phenotypic Summary:

Mutation of the gene encoding the ortholog of a human hypothetical mitochondrial enzyme (FLJ20920) resulted in the (−/−) mutant mice showing reduced levels of RBCs, platelets, hemoglobin and hematocrit In addition, the mutant (−/−) mice exhibited exophthalumus. Transcript was absent by RT-PCR.

(b) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been-extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders.

In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following tests were performed:

Hematology:

Test Description Blood tests are carried out by Abbott's Cell-Dyn 3500R, an automated hematology analyzer. Some of its features include a five-part WBC differential. ‘Patient’ reports can cover over 22 parameters in all.

Hematology observations indicated that the (−/−) mice showed reduced levels of RBCs, platelets, hemoglobin and the hematocrit compared with their gender-matched littermates. These results showed that the knockout mice had an anemia-like phenotype. Thus antagonists to PRO1009 would mimic this negative phenotype, whereas PRO1009 or agonists thereof would be important in maintaining a normal hematocrit and oxygen-carrying capacity of the red blood cells.

G. Generation and Analysis of Mice Comprising DNA59606-1471 (UNQ550) Gene Disruptions

In these knockout experiments, the gene encoding PRO1107 polypeptides (designated as DNA59606-1471 (UNQ550) was disrupted. The gene specific information for these studies is as follows: the mutated mouse gene corresponds to nucleotide reference: NM_{—}032003 or Mus musculus ectonucleotide pyrophosphatase/phosphodiesterase 5 (Enpp5); protein reference: NP_{—}114392 or ectonucleotide pyrophosphatase/phosphodiesterase 5 [Mus musculus]; the human gene sequence reference: NM_{—}021572 or Homo sapiens ectonucleotide pyrophosphatase/phosphodiesterase 5 (putative function) (ENPP5); the human protein sequence corresponds to reference: NP_{—}067547 or ectonucleotide pyrophosphatase/phosphodiesterase 5 (putative function) [Homo sapiens].

The disrupted mouse gene is Enpp5 (ectonucleotide pyrophosphatase/phosphodiesterase 5), which is orthologous to human ENPP5.

ENPP5 contains a type I phosphodiesterase/nucleotide pyrophosphatase motif (Pfam PF01663). Such phosphatases include human plasma-cell membrane glycoprotein PC-1, alkaline phosphodiesterase I, and nucleotide pyrophosphatases. These enzymes catalyze the cleavage of phosphodiester and phosphosulfate bonds in NAD, deoxynucleotides, and nucleotide sugars, thereby generating various nucleoside 5′-monophosphates. A comparison of structural features of nucleotide pyrophosphatases/phosphodiesterases (including ENPP5) indicated they all share similar catalytic functions (Gijsbers et al., J Biol Chem 276(2):1361-8 (2001)).

Targeted or gene trap mutations were generated in strain 129SvEv^{Brd}-derived embryonic stem (ES) cells. The chimeric mice were bred to C57BL/6J albino mice to generate F1 heterozygous animals. These progeny were intercrossed to generate F2 wild type, heterozygous, and homozygous mutant progeny. On rare occasions, for example when very few F1 mice were obtained from the chimera, F1 heterozygous mice were crossed to 129SvEv^{Brd}/C57 hybrid mice to yield additional heterozygous animals for the intercross to generate the F2 mice. Level I phenotypic analysis was performed on mice from this generation as shown below.

| | wt | het | hom | Total |
|---|---|---|---|---|
| Observed | 12 | 33 | 20 | 65 |
| Expected | 16.25 | 32.5 | 16.25 | 65 |

Chi-Sq. = 1.98
Significance = 0.37072
(hom/n) = 0.31
Avg. Litter Size = 6

Mutation Information: Mutation Type: Homologous Recombination (standard). Coding exon 1 was targeted (NCBI accession NM_{—}032003.1).

Wild-type expression of the target gene was detected in embryonic stem (ES) cells and, among the 13 adult tissue samples tested by RT-PCR, in kidney, prostate, testes, liver; skeletal muscle; bone; stomach, small intestine, and colon; heart; and adipose. Disruption of the target gene was confirmed by Southern hybridization analysis.

1. Phenotypic Analysis (for Disrupted Gene: DNA59606-1471 (UNQ550)

(a) Overall Phenotypic Summary:

Mutation of the gene encoding the ortholog of human ectonucleotide pyrophosphatase/phosphodiesterase 5 (ENPP5) resulted in decreased mean serum insulin levels in (−/−) mice. In addition the homozygous (−/−) mice exhibited growth retardation (decreased mean body weight and length) and notable bone metabolism disorders with decreased bone mineral density measurements. CAT-Scan results showed that the (−/−) mice had cardiomegaly and impaired renal function. Mature cataracts had also formed in both the right and left eyes of several of the mutant homozygous mice. Gene disruption was confirmed by Southern blot.

(b) Phenotypic Analysis: Metabolism-Blood Chemistry

In the area of metabolism, targets may be identified for the treatment of diabetes. Blood chemistry phenotypic analysis includes measuring serum insulin levels as an indicator of changes in glucose metabolism. Abnormal glucose metabolism can be related to the following disorders or conditions: Diabetes Type 1 and Type 2, Syndrome X, various cardiovascular diseases and/or obesity.

Insulin Data:

Test Description: Lexicon Genetics uses the Cobra II Series Auto-Gamma Counting System in its clinical settings for running quantitative Insulin assays on mice.

Results: The male and female (−/−) mice exhibited decreased mean serum insulin levels when compared with their gender-matched (+/+) littermates and the historical mean. (Analyzed wt/het/hom: 4/6/9). In addition, (−/−) mice were observed to exhibit decreased mean body weight and body length compared to the gender-matched littermates and the historical mean suggestive of growth retardation or possibly as a result of the tissue-wasting disease associated with diabetes or cachexia. Thus, mutants deficient in the gene encoding PRO1107 polypeptides exhibited a negative phenotype associated with diabetes. PRO1107 polypeptides and agonists thereof would therefore be expected to play an important role in maintaining normal glucose metabolism and would be useful in the treatment of diabetes or tissue wasting disease such as cachexia.

(c) Bone Metabolism: Radiology Phenotypic Analysis

Procedure:

As noted above, the mutant (−/−) mice showed signs of growth retardation (decreased mean body weight and mean body length when compared with gender-matched (+/+) littermates and the historical mean). For this reason radiologic phenotypic analysis was performed. In the area of bone metabolism, targets can be identified for the treatment of arthritis, osteoporosis, osteopenia and osteopetrosis as well as identifying targets that promote bone healing. Tests include microCT for very high resolution and very high sensitivity measurements of bone mineral density for both trabecular and cortical bone.

DEXA Analysis—Test Description:

Procedure: A cohort of 4 wild type, 4 heterozygotes and 8 homozygotes were tested in this assay. Dual Energy X-ray Absorptiometry (DEXA) has been used successfully to identify changes in total tissue mass (TTM).

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI, i.e., whole body, vertebrae, and both femurs).

DEXA Results:

The (−/−) mice exhibited notably decreased total tissue mass, lean body mass, total fat mass, bone mineral content, and bone mineral density in total body and femur when compared with their gender-matched (+/+) littermates and the historical means.

Bone microCT Analysis:

Procedure: MicroCT was also used to get very sensitive measurements of BMD. The μCT40 scans dissected bones and provides detailed information on bone mass and architecture. One vertebra and 1 femur were taken from a cohort of 4 wild type and 3 homozygous mice. Measurements were taken of lumbar 5 veterbra traebecular bone volume, traebecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

Micro-CT Analysis Results:

The (−/−) mice exhibited decreased vertebral trabecular bone measurements and femoral mid-shaft cross-sectional measurements when compared with its gender-matched (+/+) littermates and the historical means.

CAT-Scan Protocol:

Mice were injected with a CT contrast agent, Omnipaque 300 (Nycomed Amershan, 300 mg of iodine per ml, 0.25 ml per animal, or 2.50-3.75 g iodine/kg of body weight) intraperitoneally. After resting in the cage for ˜10 minutes, the mouse was then sedated by intraperitoneal injection of Avertin (1.25% 2,2,2,-tribromoethanol, 20 ml/kg body weight). A CAT-scan was performed using a MicroCAT scanner (ImTek, Inc.) with the anesthetized animal lying prone on the test bed. Three dimensional images were reconstructed by the Feldkamp algorithm in a cluster of workstations using an ImTek 3D RECON software.

CAT-Scan Results:

The (−/−) mice analyzed exhibited cardiomegaly and impaired renal function. CAT scan of the thorax showed an enlarged heart about twice its normal size. Very little urine in the urinary bladder suggested delayed or deranged excretion.

These results demonstrate that knockout mutant mice exhibit abnormal bone metabolism with significant bone loss similar to osteoporosis characterized by decrease in bone mass with decreased density and possibly fragility leading to bone fractures. In addition, the CAT-Scan results indicated that the (−/−) mutant mice had developed cardiomegaly which can be associated with cardiovascular diseases. The CAT scan of the thorax showed an enlarged heart about twice the normal size. Very little urine was found in the urinary bladder suggesting delayed or deranged excretion. Renal impairment was also noted. Thus, PRO1107 polypeptides would be especially important both for maintaining normal renal function, normal glucose metabolism as well as maintaining bone homeostasis. Antagonists or inhibitors of PRO1107 polypeptides or its encoding DNA, on the other hand, would lead to abnormal or pathological bone disorders similar to osteoporosis. As noted above, (−/−) mutant mice showed signs of growth retardation. Such growth disorders may be associated with the phenotype or physiological condition associated with tissue wasting diseases such as diabetes or cachexia.

(d) Cardiovascular Phenotypic Analysis:

In the area of cardiovascular biology, phenotypic testing was performed to identify potential targets for the treatment of cardiovascular, endothelial or angiogenic disorders. One such phenotypic test included optic fundus photography and angiography to determine the retinal arteriovenous ratio (A/V ratio) in order to flag various eye abnormalities. An abnormal A/V ratio signals such systemic diseases or disorders that may be related to the vascular disease of hypertension (and any disease that causes hypertension, e.g. atherosclerosis), diabetes or other ocular diseases corresponding to opthalmological disorders. Such eye abnormalities may include but are not limited to the following: retinal abnormality is retinal dysplasia, various retinopathies, restenosis, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstom's syndrome, Cockayne's syndrome, dysplasia spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie syndrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis.

One such phenotypic test included optic fundus photography and angiography to determine the retinal arteriovenous ratio (A/V ratio) in order to flag various eye abnormalities. An abnormal A/V ratio signals such systemic diseases or disorders that may be related to the vascular disease of hypertension (and any disease that causes hypertension, e.g. atherosclerosis), diabetes or other ocular diseases corresponding to opthalmological disorders. Such eye abnormalities may include but are not limited to the following: retinal abnormalities are retinal dysplasia, various retinopathies, restenosis, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstrom's syndrome, Cockayne's syndrome, dysplasia spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis.

Procedure: A cohort of 4 wild type, 5 heterozygotes and 9 homozygotes were tested in this assay. Optic fundus photography was performed on conscious animals using a Kowa Genesis small animal fundus camera modified according to Hawes and coauthors (Hawes et al., 1999 Molecular Vision 1999; 5:22). Intra-peritoneal injection of fluorescein permitted the acquisition of direct light fundus images and fluorescent angiograms for each examination. In addition to direct opthalmological changes, this test can detect retinal changes associated with systemic diseases such as diabetes and atherosclerosis or other retinal abnormalities. Pictures were provided of the optic fundus under normal light. The angiographic pictures allowed examination of the arteries and veins of the eye. In addition an artery to vein (A/V) ratio was determined for the eye.

Ophthalmology analysis was performed on generated F2 wild type, heterozygous, and homozygous mutant progeny using the protocol described above. Specifically, the A/V ratio was measured and calculated according to the fundus images with Kowa COMIT+software. This test takes color photographs through a dilated pupil: the images help in detecting and classifying many diseases. The artery to vein ratio (A/V) is the ratio of the artery diameter to the vein diameter (measured before the bifurcation of the vessels). Many diseases will influence the ratio, i.e., diabetes, cardiovascular disorders, papilledema, optic atrophy or other eye abnormalities such as retinal degeneration (known as retinitis pigmentosa) or retinal dysplasia, vision problems or blindness. Thus, phenotypic observations which result in an increased artery-to-vein ratio in homozygous (−/−) and heterozygous (+/−) mutant progeny compared to wildtype (+/+) littermates would be indicative of such pathological conditions.

Results: In this study, optic fundus photography showed that several of the (−/−) mice exhibited signs of retinal abnormalities. The (−/−) mice exhibited mature cataracts in both the right and left eyes when compared with their (+/+) littermates. A slight increase in artery to ratio was also seen. In summary, by knocking out the gene identified as DNA59606-1471 (UNQ550) which encodes PRO1107 polypeptides, homozygous mutant progeny exhibit phenotypes which are associated with cataract formation and/or other opthalmological disorders. Such detected ophthalmology changes are most commonly associated with cardiovascular systemic diseases. In particular, cataract formation may be indicative of a cardiovascular complication related to disturbances in the blood coagulation cascade. Cataracts are also associated with such systemic diseases as: Human Down's syndrome, Hallerman-Streiff syndrome, Lowe syndrome, galactosemia, Marfan syndrome, Trismoy 13-15 condition, Alport syndrome, myotonic dystrophy, Fabry disease, hypothroidisms, Conradi syndrome. Thus, antagonists of PRO1107 encoding genes would lead to similar pathological changes, whereas agonists would be useful as therapeutic agents in the prevention of cataract formation and/or the underlying cardiovascular disease or opthalmological disorders.

H. Generation and Analysis of Mice Comprising DNA60625-1507 (UNQ588) Gene Disruptions

In these knockout experiments, the gene encoding PRO1158 polypeptides (designated as DNA60625-1507 (UNQ588) was disrupted. The gene specific information for these studies is as follows: the mutated mouse gene corresponds to nucleotide reference: BC024462 or ACCESSION:BC024462 NID: 19354470 or Mus musculus Mus musculus, similar to Unknown (protein for IMAGE:3610257), clone MGC:37326 IMAGE:4975562; protein reference: □8QZT4 or ACCESSION:Q8QZT4 NID: or Mus musculus (Mouse). Similar to unknown (Protein for IMAGE:3610257). MOUSESPTRNRDB; the human gene sequence reference: NM_{—}174881 or ACCESSION: or NM_{—}174881 NID: gi 28144892 ref NM_{—}174881.1 Homo sapiens crumbs homolog 3 (Drosophila) (CRB3), transcript variant 3; the human protein sequence corresponds to reference: Q8WVA0. ACCESSION:Q8WVA0 NID: Homo sapiens (Human). Hypothetical protein. HUMANSPTRNRDB.

The disrupted mouse gene is Crb3 (crumbs homolog 3 [Drosophila]), ortholog of human CRB3. Aliases include crumbs 3 isoform a and crumbs 3 isoform b.

CRB3, a transmembrane protein located at tight junctions where apical and basolateral membranes meet, is an apical polarity determinant in epithelial cells. CRB3 consists of a short extracellular domain and a phylogenetically conserved intracellular domain, which enables complex formation with cytoplasmic scaffold proteins Pals1 (protein associated with Lin-7) and PATJ (Pals 1-associated tight junction protein). CRB3 is likely to play a role in biogenesis of tight junctions and establishment of epithelial cell polarity (Makarova et al., Gene 302(1-2):21-9 (2003)).

Targeted or gene trap mutations were generated in strain 129SvEv^{Brd}-derived embryonic stem (ES) cells. The chimeric mice were bred to C57BL/6J albino mice to generate F1 heterozygous animals. These progeny were intercrossed to generate F2 wild type, heterozygous, and homozygous mutant progeny. On rare occasions, for example when very few F1 mice were obtained from the chimera, F1 heterozygous mice were crossed to 129SvEv^{Brd}/C57 hybrid mice to yield additional heterozygous animals for the intercross to generate the F2 mice. Level I phenotypic analysis was performed on mice from this generation as shown below.

| | wt | het | hom | Total |
|---|---|---|---|---|
| Observed | 24 | 36 | 11 | 71 |
| Expected | 17.75 | 35.5 | 17.75 | 71 |

Chi-Sq. = 4.77
Significance = 0.09188
(hom/n) = 0.15
Avg. Litter Size = 7
Note:
Homozygous lethal. Of the 30 (−/−) mutants identified, 11 newborn pups were dead at the time of genotyping. The remaining dead (−/−) mutants were collected as embryos. Thus, DNA60625-1507 or its encoding polypeptide PRO1158 must be essential for embryonic development.

Mutation Information: Mutation Type: Homologous Recombination (standard). Exons 1 through 4 were targeted (NCBI accession BC024462.1).

Wild-type expression of the target gene was detected in embryonic stem (ES) cells and in all 13 adult tissue samples tested by RT-PCR. Disruption of the target gene was confirmed by Southern hybridization analysis.

1. Phenotypic Analysis (for Disrupted Gene: DNA60625-1507 (UNQ588)

(a) Overall Phenotypic Summary

Mutation of the gene encoding the ortholog of human homolog 3 of Drosophila crumbs (CRB3) resulted in lethality of (−/−) mutants. About half of the knockouts die in utero and half at birth. Nineteen dead mutants were collected as embryos. Male and female heterozygous (+/−) mice showed a decrease in total tissue mass and total body fat levels. Heterozygous (+/−) mice showed an enhanced circadian rhythm-trend towards increased dark to light activity. Gene disruption was confirmed by Southern blot.

Discussion Related to Embryonic Developmental Abnormality of Lethality:

Embryonic lethality in knockout mice usually results from various serious developmental problems including but not limited to neurodegenerative diseases, angiogenic disorders, inflammatory diseases, or where the gene/protein has an important role in basic cell signaling processes in many cell types. In addition, embryonic lethals are useful as potential cancer models. Likewise, the corresponding heterozygous (+/−) mutant animals are particularly useful when they exhibit a phenotype and/or a pathology report which reveals highly informative clues as to the function of the knocked-out gene. For instance, EPO knockout animals were embryonic lethals, but the pathology reports on the embryos showed a profound lack of RBCs.

Crumbs homolog 3 (UNQ588) is a transmembrane protein expressed at tight junctions. Analysis of the UNQ588 knockout mice allows investigation of potential roles for UNQ588 in biogenesis of tight junctions and establishment of epithelial cell polarity. Some cancers are thought to occur in epithelial tissues as a result of defective tight junctions that become chronically leaky to growth factors. UNQ 588 could play a role in this process especially since microarray data has shown that UNQ588 is significantly upregulated in numerous ovarian adenocarcinomas.

Analysis of the UNQ588 knockout mice reveal that homozygotes die within the first hour after birth. All organs look grossly normal except for the lungs which fail to inflate. Thus it appears that UNQ588 is required for lung maturation. As such the UNQ588 knockout mouse could provide a model for infant Respiratory Distress Disorder (RDS).

(b) Phenotypic Analysis: CNS/Neurology

In the area of neurology, analysis focused herein on identifying in vivo validated targets for the treatment of neurological and psychiatric disorders including depression, generalized anxiety disorders, attention deficit hyperactivity disorder, obsessive compulsive disorder, schizophrenia, cognitive disorders, hyperalgesia and sensory disorders. Neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, specific phobia, substance-induced anxiety disorder, acute alcohol withdrawal, obsessive compulsive disorder, agoraphobia, bipolar disorder I or II, bipolar disorder not otherwise specified, cyclothymic disorder, depressive disorder, major depressive disorder, mood disorder, substance-induced mood disorder. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

Procedure:

Behavioral screens were performed on a cohort of 4 wild type and 4 heterozygous mutant mice. All behavioral tests were done between 12 and 16 weeks of age unless reduced viability necessitates earlier testing. These tests included open field to measure anxiety, activity levels and exploration.

Circadian Rhythms:

Test Description:

Wild type and heterozygous mice were individually housed at 4 pm on the first day of testing in 48.2 cm×26.5 cm home cages and administered food and water ad libitum. Animals were exposed to a 12-hour light/dark cycle with lights turning on at 7 am and turning off at 7 pm. The system software records the number of beam interruptions caused by the animal's movements, with beam breaks automatically divided into ambulations. Activity was recorded in 60, one-hour intervals during the three-day test. Data generated were displayed by median activity levels recorded for each hour (circadian rhythm) and median total activity during each light/dark cycle (locomotor activity) over the three-day testing period.

Results:

Notable differences were observed during home-cage activity testing. The (+/−) mice exhibited increased ambulatory counts during the day 2 light period when compared with their (+/+) littermates. In addition, the (−/−) mice exhibited increased light-to-dark and light-to-total activity ratios when compared with their (+/+) littermates, suggesting an abnormal circadian rhythm response in the mutants.

These results indicate that the heterozygous mutant mice exhibit abnormal circadian rhythms which are usually associated with sleep disorders and/or anxiety like behavior. Thus, antagonists of PRO1158 polypeptides or its encoded gene would be expected to exhibit similar abnormal behavior. On the other hand, PRO1158 polypeptides or agonists thereof, would be useful in the treatment of such neurological disorders including sleep disorders or other anxiety-like symptoms.

I. Generation and Analysis of Mice Comprising DNA60775-1532 (UNQ633) Gene Disruptions

In these knockout experiments, the gene encoding PRO1250 polypeptides (designated as DNA60775-1532 (UNQ633) was disrupted. The gene specific information for these studies is as follows: the mutated mouse gene corresponds to nucleotide reference: AK006541 or Mus musculus adult male testis cDNA, RIKEN full-length enriched library, clone: 1700030F05 product:fatty acid Coenzyme A ligase, long chain 5, full insert sequence; protein reference: AAH31544—similar to fatty acid Coenzyme A ligase, long chain 5 [Mus musculus]; the human gene sequence reference: NM_{—}016234 or Homo sapiens fatty-acid-Coenzyme A ligase, long-chain 5 (FACL5); the human protein sequence corresponds to reference: □9ULC5 or Long-chain-fatty-acid—CoA ligase 5 (Long-chain acyl-CoA synthetase 5) (LACS 5).

The mutated mouse gene is fatty acid coenzyme A ligase, long chain 5 (Fac15), ortholog (FACL5). Aliases include 1700030F05Rik, ACS2, ACS5, long-chain acyl-CoA synthetase 5, fatty acid coenzyme A ligase 5, and long-chain fatty acid coenzyme A ligase 5.

Coenzyme A ligases are encoded by multiple genes. In general, all isozymes have multiple substrates consisting of various chain lengths. The enzyme catalyzes the formation of acyl-CoA from fatty acid (utilizing ATP and CoA). Coenzyme A ligases cooperate in some fashion with fatty acid transport proteins to import fatty acids across cell membranes (Martin et al., J Biol Chem 272(45):28210-7 (1997)).

ACS5 is expressed in intestinal epithelial cells and proliferating preadipocytes (Oikawa et al., J Biochem (Tokyo) 124(3):679-85 (1998)). FACL5 has been implicated as a factor in glioma cell growth and malignant gliomas (Yamashita et al., Oncogene 19(51):5919-25 (2000)). Rat Fac15 can be inhibited by iriacsin C and thiazolidinediones (Kim et al., J Biol Chem 276(27):24667-73 (2001)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 21 | 41 | 22 | 84 |
| Expected | 21 | 42 | 21 | 84 |

Chi-Sq. = 0.07
Significance = 0.96492
(hom/n) = 0.26
Avg. Litter Size = 8

Mutation Information: Mutation Type: Homologous Recombination (standard). Coding exons 15 through 17 were targeted.

Wild-type expression of the target gene was detected in embryonic stem (ES) cells and in all 19 adult tissue samples tested by RT-PCR. Disruption of the target gene was confirmed by Southern hybridization analysis.

UNQ633 in mouse embryos showed a strong expression signal in fetal livers (E11.5 to E12.5). UNQ633 expression in mouse embryos also showed a weak signal in a subset of small blood vessels (E10.5 mid trunk).

1. Phenotypic Analysis (for Disrupted Gene: DNA60775-1532 (UNQ633)

(a) Overall Phenotypic Summary

Mutation of the gene encoding the ortholog of human fatty-acid-coenzyme A ligase, long-chain 5 (FACL5) resulted in decreased bone mineral content and density measurements and a decreased platelet count in (−/−) mice. In addition, the (−/−) mice exhibited a decreased mean body weight and mean body length when compared with their gender-matched (+/+) littermates and the historical means. Gene disruption was confirmed by Southern blot.

(b) Bone Metabolism: Radiology Phenotypic Analysis

Procedure:

As noted above, the mutant (−/−) mice showed signs of growth retardation (decreased mean body weight and mean body length when compared with gender-matched (+/+) littermates and the historical mean). (Analyzed wt/het/hom: 14/34/17). For this reason radiologic phenotypic analysis was performed. In the area of bone metabolism, targets can be identified for the treatment of arthritis, osteoporosis, osteopenia and osteopetrosis as well as identifying targets that promote bone healing. Tests include microCT for very high resolution and very high sensitivity measurements of bone mineral density for both trabecular and cortical bone.

DEXA Analysis—Test Description:

Procedure: A cohort of 4 wild type, 4 heterozygotes and 8 homozygotes were tested in this assay. Dual Energy X-ray Absorptiometry (DEXA) has been used successfully to identify changes in total tissue mass (TTM).

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI, i.e., whole body, vertebrae, and both femurs).

DEXA Results:

The male (−/−) mice exhibited a decreased mean bone mineral content, bone mineral content index (BMC/LMB), and total body and femoral bone mineral density when compared with their gender-matched (+/+) littermates and the historical means. The female (−/−) mice exhibited decreased mean volumetric, total body, and femoral bone mineral density.

Bone microCT Analysis:

Procedure: MicroCT was also used to get very sensitive measurements of BMD. The μCT40 scans dissected bones and provides detailed information on bone mass and architecture. One vertebra and 1 femur were taken from a cohort of 4 wild type and 3 homozygous mice. Measurements were taken of lumbar 5 veterbra traebecular bone volume, traebecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

Micro-CT Analysis Results:

The male (−/−) mice exhibited decreased mean vertebral trabecular thickness and femoral midshaft cortical thickness and cross-sectional area when compared with their gender-matched (+/+) littermates and the historical means. These results demonstrate that knockout mutant mice exhibit abnormal bone metabolism with significant bone loss similar to osteoporosis characterized by decrease in bone mass with decreased density and possibly fragility leading to bone fractures. Thus, PRO1250 polypeptides would be especially important for maintaining bone homeostasis and would be useful for bone healing or for the treatment of arthritis or osteoporosis, whereas antagonists or inhibitors of PRO1250 polypeptides or its encoding DNA would lead to abnormal or pathological bone disorders similar to osteoporosis. In addition to these studies, (−/−) mutant mice showed signs of growth retardation. Such growth disorders are associated with the phenotype or physiological condition associated with tissue wasting diseases such as diabetes or cachexia.

(c) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc.

In the area of immunology, targets were identified herein for the treatment of inflammation and inflammatory disorders. Immune related diseases could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following test was performed:

Hematology Analysis:

Test Description: Blood tests are carried out by Abbott's Cell-Dyn 3500R, an automated hematology analyzer. Some of its features include a five-part WBC differential. ‘Patient’ reports can cover over 22 parameters in all.

Results:

The (−/−) mice exhibited a notably decreased mean platelet count when compared with their (+/+) littermates and the historical mean. These results indicate that PRO1250 polypeptides or its encoding DNA are important for normal blood clotting. Thus, mutant mice deficient in the DNA60775-1532 gave rise to a negative phenotype resulting in coagulation disorders. PRO1250 polypeptides or agonists thereof would be useful in treating disorders related to abnormal blood coagulation such as hemophilia.

J. Generation and Analysis of Mice Comprising DNA71166-1685 (UNQ783) Gene Disruptions

In these knockout experiments, the gene encoding PRO1317 polypeptides (designated as DNA71166-1685 (UNQ783) was disrupted. The gene specific information for these studies is as follows: the mutated mouse gene corresponds to nucleotide reference: NM_{—}013658 or Mus musculus sema domain, immunoglobulin domain (Ig), transmembrane domain (TM) and short cytoplasmic domain, (semaphorin) 4A (Sema4a); protein reference: Q62178 or ACCESSION:Q62178 NID: Mus musculus (Mouse). SEMAPHORIN 4A PRECURSOR (SEMAPHORIN B) (SEMA B). MOUSESPTRNRDB; the human gene sequence reference: NM_{—}022367 or Homo sapiens hypothetical protein FLJ12287 similar to semaphorins (FLJ12287); the human protein sequence corresponds to reference: Q9H3S1 or ACCESSION:Q9H3S1 NID: Homo sapiens (Human). Semaphorin 4A precursor (Semaphorin B) (Sema B). sp_tr_nrdb.

The mouse gene of interest is Sema4a (sema domain, immunoglobulin domain (Ig), transmembrane domain (TM) and short cytoplasmic domain, (semaphorin) 4A), which is orthologous with a human gene represented by Homo sapiens hypothetical protein FLJ12287 similar to semaphorins (FLJ12287), mRNA (NCBI accession NM_{—}022367). Aliases include SemB and Semab.

Sema4a is a type I plasma membrane protein that contains an N-terminal signal peptide sequence, a semaphorin domain, a plexin, semaphorin, and integrin (PSI) domain, and a C-terminal transmembrane segment (Puschel et al., Neuron 14(5):941-8 (1995)). Sema4a may be a ligand, interacting with receptors, such as plexin and neuropilin, on neurons (Chen et al., Nat Neurosci 1(6):436-9 (1998)) and with receptor TIM2 on dendritic cells of the immune system (Kikutani and Kumanogoh, Nat Rev Immunol 3(2): 159-67 (2003)). Sema4a is differentially expressed in the primary olfactory pathway during neuron regeneration and development and is likely to act as a chemorepellent, guiding axon projection of olfactory receptor neurons (Williams-Hogarth et al., J Come Neurol 423(4):565-78 (2000)). Sema4a is also expressed on dendritic cells and is likely to participate in T-cell activation by binding with TIM2, a receptor expressed on the surface of activated T-cells (Kikutani and Kumanogoh, Kikutani and Kumanogob, Nat Rev Immunol 3(2): 159-67 (2003).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 13 | 34 | 22 | 69 |
| Expected | 17.25 | 34.5 | 17.25 | 69 |

Chi-Sq. = 2.36
Significance = 0.30692
(hom/n) = 0.32
Avg. Litter Size = 7

Mutation Information Mutation Type: Retroviral Insertion (OST). Retroviral insertion occurred in the intron between coding exons 10 and 11 (NCBI accession NM_{—}013658.2).

Wild-type expression of the target gene was detected in embryonic stem (ES) cells and in all 13 adult tissue samples tested by RT-PCR, except testis. RT-PCR analysis revealed that the transcript was absent in the (−/−) mouse analyzed (M-75).

1. Phenotypic Analysis (for Disrupted Gene: DNA (UNQ783)

(a) Overall Phenotypic Summary

Mutation of the gene encoding the ortholog of a human hypothetical semaphorin (FLJ12287) resulted in severe retinal degeneration in and notably attenuated retinal vessels and a decreased mean retinal artery-to-vein ratio. The (−/−) mice also exhibited an increased skin fibroblast proliferation rate indicative of solid tumor formation. Serum immunoglobulin measurements showed that mutant (−/−) mice had notably increased mean serum IgG 1, IgG3, IgA, IgG2a and IgG2b levels when compared with their (+/+) littermates. The KO mice also showed a decreased insulin and glucose tolerance. Transcript was absent by RT-PCR.

(b) Cardiovascular Phenotypic Analysis:

In the area of cardiovascular biology, phenotypic testing was performed to identify potential targets for the treatment of cardiovascular, endothelial or angiogenic disorders. One such phenotypic test included optic fundus photography and angiography to determine the retinal arteriovenous ratio (A/V ratio) in order to flag various eye abnormalities. An abnormal A/V ratio signals such systemic diseases or disorders that may be related to the vascular disease of hypertension (and any disease that causes hypertension, e.g. atherosclerosis), diabetes or other ocular diseases corresponding to opthalmological disorders. Such eye abnormalities may include but are not limited to the following: retinal abnormality is retinal dysplasia, various retinopathies, restenosis, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstom's syndrome, Cockayne's syndrome, dysplasia spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie syndrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis.

One such phenotypic test included optic fundus photography and angiography to determine the retinal arteriovenous ratio (A/V ratio) in order to flag various eye abnormalities. An abnormal A/V ratio signals such systemic diseases or disorders that may be related to the vascular disease of hypertension (and any disease that causes hypertension, e.g. atherosclerosis), diabetes or other ocular diseases corresponding to opthalmological disorders. Such eye abnormalities may include but are not limited to the following: retinal abnormalities are retinal dysplasia, various retinopathies, restenosis, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstrom's syndrome, Cockayne's syndrome, dysplasia spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis.

Procedure: A cohort of 4 wild type, 4 heterozygotes and 8 homozygotes were tested in this assay. Optic fundus photography was performed on conscious animals using a Kowa Genesis small animal fundus camera modified according to Hawes and coauthors (Hawes et al., 1999 Molecular Vision 1999; 5:22). Intra-peritoneal injection of fluorescein permitted the acquisition of direct light fundus images and fluorescent angiograms for each examination. In addition to direct opthalmological changes, this test can detect retinal changes associated with systemic diseases such as diabetes and atherosclerosis or other retinal abnormalities. Pictures were provided of the optic fundus under normal light. The angiographic pictures allowed examination of the arteries and veins of the eye. In addition an artery to vein (A/V) ratio was determined for the eye.

Ophthalmology analysis was performed on generated F2 wild type, heterozygous, and homozygous mutant progeny using the protocol described above. Specifically, the A/V ratio was measured and calculated according to the fundus images with Kowa COMIT+software. This test takes color photographs through a dilated pupil: the images help in detecting and classifying many diseases. The artery to vein ratio (A/V) is the ratio of the artery diameter to the vein diameter (measured before the bifurcation of the vessels). Many diseases will influence the ratio, i.e., diabetes, cardiovascular disorders, papilledema, optic atrophy or other eye abnormalities such as retinal degeneration (known as retinitis pigmentosa) or retinal dysplasia, vision problems or blindness. Thus, phenotypic observations which result in an increased artery-to-vein ratio in homozygous (−/−) and heterozygous (+/−) mutant progeny compared to wildtype (+/+) littermates would be indicative of such pathological conditions.

Results: In this study, optic fundus photography showed that (−/−) mice exhibited signs of severe retinal degeneration, namely notably attenuated retinal vessels and a decreased mean artery-to-vein (A/V) ratio when compared with their (+/+) littermates. Angiograms demonstrated that the mutant (−/−) mice showed attenuated retinal vessels with microaneurysms. Likewise, microscopic observations showed both bilateral retinal degeneration and notably decreased lens size in the mutant (−/−) mice. In summary, by knocking out the gene identified as DNA71166-1685 encoding PRO1317 polypeptides, homozygous mutant progeny exhibit phenotypes which are associated with retinal degeneration. Such detected retinal changes are most commonly associated with cardiovascular systemic diseases or disorders that may be related to the vascular disease of hypertension (and any disease that causes hypertension, e.g. atherosclerosis), diabetes or other ocular diseases corresponding to opthalmological disorders such as retinal degeneration. Thus, antagonists of PRO1317 encoding genes would lead to similar pathological retinal changes, whereas agonists would be useful as therapeutic agents in the treatment of hypertension, atherosclerosis or other opthalmological disorders including retinal degeneration and diseases associated with this condition (as indicated above).

(c) Oncology Phenotypic Analysis

In the area of oncology, targets were identified herein for the treatment of solid tumors, lymphomas and leukemia.

Adult Skin Cell Proliferation:

Procedure: Skin cells were isolated from 16 week old animals (2 wild type and 4 homozygotes). These were developed into primary fibroblast cultures and the fibroblast proliferation rates were measured in a strictly controlled protocol. The ability of this assay to detect hyper-proliferative and hypo-proliferative phenotypes has been demonstrated with p53 and Ku80. Proliferation was measured using Brdu incorporation:

Specifically, in these studies the skin fibroblast proliferation assay was used. An increase in the number of cells in a standardized culture was used as a measure of relative proliferative capacity. Primary fibroblasts were established from skin biopsies taken from wild type and mutant mice. Duplicate or triplicate cultures of 0.05 million cells were plated and allowed to grow for six days. At the end of the culture period, the number of cells present in the culture was determined using a electronic particle counter.

Results: The female (−/−) mice exhibited an increased skin fibroblast proliferation rate when compared with their gender-matched (+/+) littermates and the historical mean. [Analyzed wt/het/hom: 2/0/4] Thus, homozygous mutant mice demonstrated a hyper-proliferative phenotype. As suggested by these observations, PRO1317 polypeptides or agonists thereof have a tumor suppressive phenotype and would be useful in decreasing abnormal cell proliferation.

(d) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc.

In the area of immunology, targets were identified herein for the treatment of inflammation and inflammatory disorders. Immune related diseases could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

Serum Immunoglobulin Isotyping Assay:

The Serum Immunoglobulin Isotyping Assay was performed using a Cytometric Bead Array (CBA) kit.

This assay was used to rapidly identify the heavy and light chain isotypes of a mouse monoclonal antibody in a single sample. The values expressed are “relative fluorescence units” and are based on the detection of kappa light chains. Any value <6 is not significant.

Results:

Mutant (−/−) mice exhibited a notable elevation of the mean serum IgG1, IgG3, IgA, IgG2a and IgG2b levels compared to their gender-matched (+/+) littermates. IgG immunoglobulins have neutralization effects on toxins and to a lesser extent are important for activation of the complement system. The observed phenotype suggests that the PRO1317 polypeptide is a negative regulator of inflammatory responses. These immunological abnormalities suggest that inhibitors (antagonists) of PRO1317 polypeptides would be important agents which could stimulate the immune system (such as T cell proliferation) and would find utility in the cases wherein this effect would be beneficial to the individual such as in the case of leukemia, and other types of cancer, and in immunocompromised patients, such as AIDS sufferers. Accordingly, PRO1317 polypeptides or agonists thereof would be useful for inhibiting the immune response and would be useful candidates for suppressing harmful immune responses, e.g. in the case of graft rejection or graft-versus-host diseases.

(e) Phenotypic Analysis: Metabolism-Blood Chemistry—Glucose Tolerance Test

In the area of metabolism, targets may be identified for the treatment of diabetes. Blood chemistry phenotypic analysis includes measuring insulin levels and glucose tolerance tests to measure insulin sensitivity and changes in glucose metabolism. Abnormal glucose tolerance test results may indicate but may not be limited to the following disorders or conditions: Diabetes Type 1 and Type 2, Syndrome X, various cardiovascular diseases and/or obesity.

Procedure: A cohort of 2 wild type and 4 homozygote mice were used in this assay. The glucose tolerance test is the standard for defining impaired glucose homeostasis in mammals. Glucose tolerance tests were performed using a Lifescan glucometer. Animals were injected IP at 2 g/kg with D-glucose delivered as a 20% solution and blood glucose levels were measured at 0, 30, 60 and 90 minutes after injection.

Results: These studies indicated that (−/−) mice exhibit decreased insulin levels and decreased glucose tolerance in the presence of normal fasting glucose at all 3 intervals tested when compared with their gender-matched (+/+) littermates and the historical means. Thus, knockout mice exhibited the phenotypic pattern of an impaired glucose homeostasis, PRO1317 or its encoding gene would be useful in the treatment of impaired glucose homeostasis and/or diabetes.

K. Generation and Analysis of Mice Comprising DNA59608-2577 (UNQ1889) Gene Disruptions

In these knockout experiments, the gene encoding PRO4334 polypeptides (designated as DNA59608-2577 (UNQ 1889) was disrupted. The gene specific information for these studies is as follows: the mutated mouse gene corresponds to nucleotide reference: AK046797 or Mus musculus 10 days neonate medulla oblongata cDNA, RIKEN full-length enriched library, clone:B830010K19 product:hypothetical Type I phosphodiesterase/nucleotide pyrophosphatase containing protein, full insert sequence; protein reference: Q8BGN3 or ACCESSION:Q8BGN3 NID: Mus musculus (Mouse). Hypothetical type I phosphodiesterase/nucleotide pyrophosphatase containing protein. sp_tr_nrdb; the human gene sequence reference: AK057370 or ACCESSION:AK057370 NID:16553044 Homo sapiens Homo sapiens cDNA FLJ32808 fis, clone TESTI2002707, weakly similar to PLASMA-CELL MEMBRANE GLYCOPROTEIN PC-1 [INCLUDES: ALKALINE PHOSPHODIESTERASE I (EC 3.1.4.1); NUCLEOTIDE PYROPHOSPHATASE (EC 3.6.1.9) (NPPASE)]; the human protein sequence corresponds to reference: □96M57. ACCESSION:Q96M57 NID: Homo sapiens (Human). Hypothetical protein FLJ32808 (Similar to ectonucleotide pyrophosphatase/phosphodiesterase 5). HUMANSPTRNRDB.

The mouse gene of interest is represented by a cDNA defined as “hypothetical type I phosphodiesterase/nucleotide pyrophosphatase-containing protein, full insert sequence” (AK046797), which is the ortholog of human ectonucleotide pyrophosphatase/phosphodiesterase 6 (ENPP6). Aliases include RIKEN cDNA B830047L21 gene.

ENPP6 is a hypothetical enzyme that catalyzes the cleavage of phosphodiester and phosphosulfate bonds in NAD, deoxynucleotides, and nucleotide sugars. ENPP6 contains a signal peptide, a type I phosphodiesterase/nucleotide pyrophosphatase domain, and a potential C-terminal GPI anchor, suggesting that the enzyme is located on the extracellular surface of the plasma membrane or secreted. The biological role of this protein is not known.

Targeted or gene trap mutations are generated in strain 129SvEvBrd-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEvBrd/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 14 | 39 | 20 | 73 |
| Expected | 18.25 | 36.5 | 18.25 | 73 |

Chi-Sq. = 1.33
Significance = 0.51459
(hom/n) = 0.27
Avg. Litter Size = 7

Mutation Information: Mutation Type: Homologous Recombination (standard). Coding exon 1 was targeted (NCBI accession NM 177304.1).

Wild-type expression of the target gene was detected in brain, spinal cord, eye, kidney, liver, and heart among the 13 adult tissue samples tested by RT-PCR. Disruption of the target gene was confirmed by Southern hybridization analysis.

1. Phenotypic Analysis (for Disrupted Gene: DNA59608-2577 (UNQ1889)

(a) Overall Phenotypic Summary

Mutation of the gene encoding the ortholog of human ectonucleotide pyrophosphatase/phosphodiesterase 6 (ENPP6) resulted in male (−/−) mice exhibiting an increased mean percent body fat suggestive of obesity. Female (−/−) mice exhibited an elevated bone mineral density. Gene disruption was confirmed by Southern blot.

(b) Phenotype Analysis: Body Mass:

Dexa Analysis—Test Description:

Procedure: A cohort of 4 wild type, 4 heterozygous and 10 homozygous mice were tested in this assay. Dual Energy X-ray Absorptiometry (DEXA) has been used successfully to identify changes in total tissue mass (TTM).

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI, i.e., whole body, vertebrae, and both femurs).

Results:

The (−/−) mutant mice analyzed by DEXA exhibited a increased mean percent body fat when compared with their (+/+) littermates, suggestive of obesity in these mutants. Thus, PRO4334 or agonists thereof would be useful in the treatment of metabolic disorders such as obesity. In addition, female (−/−) mice exhibited an increased bone mineral density. These results indicate that the knockout mutant phenotype would be associated with such bone abnormalities as osteopetrosis. Osteopetrosis is a condition characterized by abnormal thickening and hardening of the bone leading to abnormal fragility. As such, PRO4334 polypeptides or agonists thereof may be beneficial for the treatment of osteopetrosis. A phenotype associated with an increased bone mineral density suggests that agents which mimic this negative phenotype (e.g. antagonists of PRO4334) would play a role in bone healing.

L. Generation and Analysis of Mice Comprising DNA80840-2605 (UNQ1921) Gene Disruptions

In these knockout experiments, the gene encoding PRO4395 polypeptides (designated as DNA80840-2605 (UNQ1921) was disrupted. The gene specific information for these studies is as follows: the mutated mouse gene corresponds to nucleotide reference: NM_{—}178793 or Mus musculus RIKEN cDNA 9430093N24 gene (9430093N24Rik); protein reference: □8BFW1 or ACCESSION:Q8BFW1 NID: Mus musculus (Mouse). Weakly similar to oncofetal-laminin binding collagen. sp_tr_nrdb; the human gene sequence reference: NM_{—}133459 or Homo sapiens KIAA1983 protein (FLJ30681); the human protein sequence corresponds to reference: □8TF19 or ACCESSION:Q8TF19 NID: Homo sapiens (Human). Hypothetical protein KIAA1983 (Fragment). sp_tr_nrdb.

The disrupted gene is represented by NCBI accession NM_{—}178793, which is the ortholog of human KIAA1983 protein. Aliases included FLJ30681 and 9430093N24Rik.

KIAA1983 is a hypothetical secreted protein, containing a signal peptide, an EGF-like domain (SMART accession SM00001), a calcium-binding EGF-like domain (SMART accession SM00179), and a collagen triple helix repeat (InterPro accession IPR008160). The protein shares characteristics with the EFG/Laminin superfamily of proteins as defined by SCOP software (Murzin et al., J Mol Biol 247(4):536-40 (1995)). Various annotations describe the hypothetical protein as “weakly similar to oncofetal-laminin binding collagen” (e.g., SwissProt Q8BJC3).

Targeted or gene trap mutations were generated in strain 129SvEv^{Brd}-derived embryonic stem (ES) cells. The chimeric mice were bred to C57BL/6J albino mice to generate F1 heterozygous animals. These progeny were intercrossed to generate F2 wild type, heterozygous, and homozygous mutant progeny. On rare occasions, for example when very few F1 mice were obtained from the chimera, F1 heterozygous mice were crossed to 129SvEv^{Brd}/C57 hybrid mice to yield additional heterozygous animals for the intercross to generate the F2 mice. Disruption of the target gene was confirmed by Southern hybridization analysis. Level I phenotypic analysis was performed on mice from this generation.

| | wt | het | hom | Total |
|---|---|---|---|---|
| Observed | 20 | 42 | 0 | 62 |
| Expected | 15.5 | 31 | 15.5 | 62 |

Chi-Sq. = 20.71
Significance = 0.00003
(hom/n) = 0.00
Avg. Litter Size = 7

Summary: Homozygous lethal
Thus DNA80840-2605 or its encoded polypeptide PRO4395 must be essential for embryonic development.

Mutation Information: Mutation Type: Homologous Recombination (standard). Coding exon 1 was targeted (NCBI accession AK028377.1).

Wild-type expression of the target gene was detected in all 44 tissue samples tested by RT-PCR, except pancreas, stomach, uterus, bladder, gall bladder, spinal cord, trachea, aorta, and eye. Disruption of the target gene was confirmed by Southern hybridization analysis.

1. Phenotypic Analysis (for Disrupted Gene: DNA80840-2605 (UNQ1921)

(a) Overall Phenotypic Summary

Mutation of the gene encoding the ortholog of human KIAA1983 protein (FLJ30681) resulted in lethality of (−/−) mutants. A growth retardation and bone abnormalities were observed for (+/−) mice in that the heterozygous mice exhibited a decreased total tissue mass, decreased total body fat and decreased bone mineral density compared to their gender-matched (+/+) littermates. In addition, seven out of eight heterozygous mice had exophthalmus. Gene disruption was confirmed by Southern blot.

Discussion Related to Embryonic Developmental Abnormality of Lethality:

Embryonic lethality in knockout mice usually results from various serious developmental problems including but not limited to neurodegenerative diseases, angiogenic disorders, inflammatory diseases, or where the gene/protein has an important role in basic cell signaling processes in many cell types. In addition, embryonic lethals are useful as potential cancer models. Likewise, the corresponding heterozygous (+/−) mutant animals are particularly useful when they exhibit a phenotype and/or a pathology report which reveals highly informative clues as to the function of the knocked-out gene. For instance, EPO knockout animals were embryonic lethals, but the pathology reports on the embryos showed a profound lack of RBCs.

UNQ1921 is a novel SPDI gene containing an EGF-like domain and a collagen triple helix domain. It is a hypothetical secreted protein similar to laminin superfamily members which may be involved in cell adhesion and/or migration. Analysis of the UNQ1921 knockout mice indicates that UNQ1921 plays a role in liver development and hematopoiesis.

Gal expression analysis demonstrates that UNQ1921 is first expressed at 9.5d in the septum transversum. This structure is important for liver development and its derivatives go on to form the diaphragm and the pericardium surrounding the heart. A couple of days later UNQ1921 is expressed in highly restricted domains in or near migratory cells. It is expressed in the cephalic neural crest and around subpopulations of muscle precursors. UNQ1921 knockout mice die around birth. They are pale and exhibit severe interstitial edema. The edema is evident as early as 14.5d and is normally attributed to defects in one or more of 4 systems; liver, hematopoiesis, lymphatic or cardio-vascular. The livers are greatly reduced in size in UNQ1921 mutants. Defective liver function could result in reduced levels of albumin in the blood stream that would lead to a decrease in osmotic pressure which could account for the interstitial edema seen in the UNQ1921 mutants.

Regions of the UNQ1921 mutant livers are white in color whereas the control livers at this stage are completely red. Sections through mutant livers reveal that these white regions have reduced numbers of hematopoietic cells. Higher magnification of sections shows a correlation between the health of the developing hepatocytes and the numbers of viable hematopoietic cells. There is evidence to suggest that colonization of the embryonic liver by hematopoietic stem cells (HSCs) at 12.5d occurs by active migration of HSCs from the AGM (aorta, mesenephros, gonad) and the yolk sac. 1 integrin plays a vital role in this migration. LNQ1921 contains an RGD motif that is capable of binding integrins. These observations support a similar role of UNQ1921 for mediating migration of HSCs to the liver.

UNQ1921 is also expressed in the pericardium. The pericardium at 17.5d is normally closely associated with the underlying myocardium. In the UNQ1921 mutant hearts the pericardium is seen to be only loosely associated with the myocardium. It is possible that reduced cardio-vascular function resulting from an abnormal pericardium could affect blood pressure and result in interstitial edema.

UNQ1921 appears to be important in liver function and hematopoiesis. It is possible that UNQ1921 is expressed in stellar cells of adult livers. These cells are thought to be derived from the septum transversum where UNQ1921 is expressed. They are the cells that are responsible for eliciting a regenerative response to liver damage. These cells are also over-active in fibrotic and cirrhotic livers. Thus, UNQ1921 could play a role in these types of liver conditions.

(b) Bone Metabolism: Radiology Phenotypic Analysis

In the area of bone metabolism, targets were identified herein for the treatment of arthritis, osteoporosis, osteopenia and osteopetrosis as well as identifying targets that promote bone healing.

Dexa Analysis—Test Description:

Procedure: A cohort of 4 wild type, and 4 heterozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI, i.e., whole body, vertebrae, and both femurs).

DEXA Results:

As summarized above, The male (+/−) mice exhibited decreased mean total tissue mass and total body fat when compared with their gender-matched (+/+) littermates and the historical means suggestive of growth retardation in these mutants. In addition, the (+/) mice exhibited a decreased bone mineral density. This in conjunction with the observations of a decreased mean total tissue mass and total body fat suggests a tissue wasting condition such as cachexia in these heterozygous mice. Thus, PRO4395 polypeptides or agonists thereof appear to be essential for growth and development.

M. Generation and Analysis of Mice Comprising DNA237637 (UNQ2239) Gene Disruptions

In these knockout experiments, the gene encoding PRO49192 polypeptides (designated as DNA237637 (UNQ2239) was disrupted. The gene specific information for these studies is as follows: the mutated mouse gene corresponds to nucleotide reference: NM_{—}011404 or Mus musculus solute carrier family 7 (cationic amino acid transporter, y system), member 5 (Slc7a5); protein reference: BAA90556 or L-type amino acid transporter 1 [Mus musculus]; the human gene sequence reference: NM_{—}003486 or Homo sapiens solute carrier family 7 (cationic amino acid transporter, y system), member 5 (SLC7A5); the human protein sequence corresponds to reference: NP_{—}003477 or solute carrier family 7 (cationic amino acid transporter, y system), member 5; Membrane protein E16; Solute carrier family 7, member 5; 4F2 light chain [Homo sapiens].

The disrupted mouse gene is Slc7a5 (solute carrier family 7 [cationic amino acid transporter, y system], member 5), ortholog of human SLC7A5. Aliases include TA1, D0H16S474E, E16, CD98, CD98 light chain, LAT1, MPE16, D16S469E, 4F2 light chain, and membrane protein E16.

SLC7A5 was first identified as an expressed sequence in activated lymphocytes (Gaugitsch et al., J Biol Chem 267(16):11267-73 (1992)). SLC7A5 forms heterodimers with the human cell-surface glycoprotein 4F2 heavy chain (SLC3A2); the heterodimeric complex then facilitates L-type amino-acid transport (Mastroberardino et al., Nature 395(6699):288-91 (1998)).

SLC7 family members are generally considered to act as heterodimeric amino acid transporters. Lack of the heteromeric partner of SLC7A5 (i.e., Slc3a2) results in embryonic lethality in mice (Tsumura et al., Biochem Biophys Res Commun 308(4):847-51. (2003).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 19 | 37 | 0 | 56 |
| Expected | 14 | 28 | 14 | 56 |

Chi-Sq. = 18.68
Significance = 0.00009
(hom/n) = 0.00
Avg. Litter Size = 6

### Summary: Lethality

Mutation Information: Mutation Type: Homologous Recombination (standard). Coding exon 1 was targeted (NCBI accession NM_{—}011404.2).

Wild-type expression of the target gene was detected in embryonic stem (ES) cells and in all 18 adult tissue samples tested by RT-PCR, except eye; LPS liver; skeletal muscle; bone; stomach, small intestine, and colon; adipose; skin fibroblast; and prostate. Disruption of the target gene was confirmed by Southern hybridization analysis.

1. Phenotypic Analysis (for Disrupted Gene: DNA237637 (UNQ2239)

(a) Overall Phenotypic Summary

Mutation of the gene encoding the ortholog of human solute carrier family 7 [cationic amino acid transporter, y+system], member 5 (SLC7A5) resulted in lethality of the (−/−) mutants. The heterozygous (+/−) mice showed an increased total tissue mass, lean body mass, total body fat, bone mineral density and bone mineral content. Among the three (+/−) mice examined, one mouse had calculus in the left kidney. Gene disruption was confirmed by Southern blot.

Discussion Related to Embryonic Developmental Abnormality of Lethality:

Embryonic lethality in knockout mice usually results from various serious developmental problems including but not limited to neurodegenerative diseases, angiogenic disorders, inflammatory diseases, or where the gene/protein has an important role in basic cell signaling processes in many cell types. In addition, embryonic lethals are useful as potential cancer models. Likewise, the corresponding heterozygous (+/−) mutant animals are particularly useful when they exhibit a phenotype and/or a pathology report which reveals highly informative clues as to the function of the knocked-out gene. For instance, EPO knockout animals were embryonic lethals, but the pathology reports on the embryos showed a profound lack of RBCs.

UNQ2239 is an amino acid transporter called E16 that was previously identified by applicants as a novel tumor antigen. It exhibits very high levels of expression in many cancer cell lines, especially lung and breast. Analysis of the UNQ2239 knockout mice has allowed applicants to visualize for the first time UNQ2239 expression in vasculature. We have observed that UNQ2239 is required very early during mouse development.

Using a combination of RNA whole mount in situ and gal staining UNQ2239 has been shown to be expressed specifically in the developing micro-vasculature. Expression in 12.5d placentas occurs exclusively in the labyrinth layer which contains the fetal vasculature component of the placenta.

Genotyping analysis of embryos from UNQ2239 heterozygous intercrosses indicates that UNQ2239 is required for embryonic development at a stage prior to gastrulation (7.5d). It is known that amino acids are essential for the process of blastulation to occur at 3.5d of embryonic development.

(b) Bone Metabolism: Radiology Phenotypic Analysis

In the area of bone metabolism, targets were identified herein for the treatment of arthritis, osteoporosis, osteopenia and osteopetrosis as well as identifying targets that promote bone healing. Tests included:

DEXA for measurement of bone mineral density on femur and vertebra

MicroCT for very high resolution and very high sensitivity measurements of bone mineral density for both trabecular and cortical bone.

Dexa Analysis—Test Description:

Procedure: A cohort of 4 wild type and 4 heterozygotes were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

DEXA Results:

The (+/−) heterozygous mice exhibited an increase in total tissue mass, lean body mass, total body fat, bone mineral density and bone mineral content compared with their gender-matched (+/+) littermates.

Bone microCT Analysis:

Procedure: MicroCT was also used to get very sensitive measurements of BMD. One vertebra and 1 femur were taken from a cohort of 4 wild type and 4 heterozygous mice. Measurements were taken of lumbar 5 veterbra traebecular bone volume, traebecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

MicroCT Results:

The (+/−) heterozygous mice showed increased bone measurements. These results coupled with the DEXA results cited above demonstrate that heterozygous mice exhibit a phenotype that is associated with obesity as well as such bone abnormalities as osteopetrosis. Osteopetrosis is a condition characterized by abnormal thickening and hardening of the bone leading to abnormal fragility. As such, PRO49192 polypeptides or agonists thereof are essential for normal bone development and would be beneficial for the treatment of osteopetrosis. A phenotype associated with an increased bone mineral density suggests that agents which mimic this negative phenotype (e.g. antagonists of PRO49192) would play a role in bone healing.

N. Generation and Analysis of Mice Comprising DNA108696-2966 (UNQ3018) Gene Disruptions

In these knockout experiments, the gene encoding PRO9799 polypeptides (designated as DNA108696-2966 (UNQ3018) was disrupted. The gene specific information for these studies is as follows: the mutated mouse gene corresponds to nucleotide reference: NM_{—}030069 or Mus musculus RIKEN cDNA 4432416J03 gene (4432416J03Rik); protein reference: NP_{—}084345 or RIKEN cDNA4432416J03 [Mus musculus]; the human gene sequence reference: NM_{—}152315 or Homo sapiens hypothetical protein MGC34290 (MGC34290); the human protein sequence corresponds to reference: NP_{—}689528 or hypothetical protein MGC34290 [Homo sapiens].

The disrupted mouse gene is represented by a NCBI accession NM_{—}030069, ortholog of human hypothetical protein MGC34290.

By bioinformatic analysis, MGC34290 contains a signal peptide and an overlapping SCOP domain (d1qfhal, E set superfamily of immunoglobulin-like beta-sandwich fold proteins). The predicted (ProtComp, Softberry Corp) cell location is ambiguous; three possibilities are suggested: extracellular, plasma membrane, and mitochondrial. MGC34290 is a member of the brush border 61.9 kDa precursor family (ENSEMBL protein family ENSF00000003554).

Targeted or gene trap mutations were generated in strain 129SvEv^{Brd}-derived embryonic stem (ES) cells. The chimeric mice were bred to C57BL/6J albino mice to generate F1 heterozygous animals. These progeny were intercrossed to generate F2 wild type, heterozygous, and homozygous mutant progeny. On rare occasions, for example when very few F1 mice were obtained from the chimera, F1 heterozygous mice were crossed to 129SvEv^{Brd}/C57 hybrid mice to yield additional heterozygous animals for the intercross to generate the F2 mice. Disruption of the target gene was confirmed by Southern hybridization analysis. Level I phenotypic analysis was performed on mice from this generation.

| | wt | het | hom | Total |
|---|---|---|---|---|
| Observed | 21 | 29 | 25 | 75 |
| Expected | 18.75 | 37.5 | 18.75 | 75 |

Chi-Sq. = 4.28
Significance = 0.11765
(hom/n) = 0.33
Avg. Litter Size = 8

Mutation Information: Mutation Type: Homologous Recombination (standard). Coding exons 1 and 2 were targeted (NCBI accession NM_{—}030069.1).

Wild-type expression of the target gene was detected in embryonic stem (ES) cells and, among the 13 adult tissues samples tested by RT-PCR, especially in the colon, and to a much lesser extent in small intestine, spleen, lung, liver, bone, and heart. Disruption of the target gene was confirmed by Southern hybridization analysis.

1. Phenotypic Analysis (for Disrupted Gene: DNA108696-2966 (UNQ3018)

(a) Overall Phenotypic Summary

Mutation of the gene encoding the ortholog of a human hypothetical protein (MGC34290) resulted in an abnormal circadian rhythms response in (−/−) mice. The (−/−) knockout mice demonstrated lower levels of blood neutrophils compared with their (+/+) littermates. In addition, the (−/−) mutants showed an elevated level of IgG2a in response to an Ovalbumin challenge. Both the male and female (−/−) mice exhibited an increased percentage of body fat compared with the wild-type littermates. The (−/−) mice also exhibited a decreased skin proliferation rate. Gene disruption was confirmed by Southern blot.

(b) Phenotypic Analysis: CNS/Neurology

In the area of neurology, analysis focused herein on identifying in vivo validated targets for the treatment of neurological and psychiatric disorders including depression, generalized anxiety disorders, attention deficit hyperactivity disorder, obsessive compulsive disorder, schizophrenia, cognitive disorders, hyperalgesia and sensory disorders. Neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, specific phobia, substance-induced anxiety disorder, acute alcohol withdrawal, obsessive compulsive disorder, agoraphobia, bipolar disorder I or II, bipolar disorder not otherwise specified, cyclothymic disorder, depressive disorder, major depressive disorder, mood disorder, substance-induced mood disorder. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

Procedure:

Behavioral screens were performed on a cohort of 4 wild type, 4 heterozygous and 8 homozygous mutant mice. All behavioral tests were done between 12 and 16 weeks of age unless reduced viability necessitates earlier testing. These tests included open field to measure anxiety, activity levels and exploration.

Circadian Rhythms:

Test Description:

Female mice were individually housed at 4 pm on the first day of testing in 48.2 cm×26.5 cm home cages and administered food and water ad libitum. Animals were exposed to a 12-hour light/dark cycle with lights turning on at 7 am and turning off at 7 pm. The system software records the number of beam interruptions caused by the animal's movements, with beam breaks automatically divided into ambulations. Activity was recorded in 60, one-hour intervals during the three-day test. Data generated were displayed by median activity levels recorded for each hour (circadian rhythm) and median total activity during each light/dark cycle (locomotor activity) over the three-day testing period.

Results:

Some differences were observed during home-cage activity testing. Two out of the four female (−/−) mice exhibited increased ambulatory counts during the day 2 light period when compared with their (+/+) littermates (effect is largely confined to day 2 of testing. In addition, the (−/−) mice exhibited increased light-to-dark and light-to-total activity ratios when compared with their (+/+) littermates, suggesting an abnormal circadian rhythms response in the mutants. Analyzed wt/het/hom: 4/4/4

These results indicate that the mutant mice exhibit abnormal circadian rhythms which are usually associated with sleep disorders and/or anxiety like behavior. Thus, antagonists of PRO9799 polypeptides or its encoded gene would be expected to exhibit similar abnormal behavior. On the other hand, PRO9799 polypeptides or agonists thereof, would be useful in the treatment of such neurological disorders including sleep disorders or other anxiety-like symptoms.

(c) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc.

In the area of immunology, targets were identified herein for the treatment of inflammation and inflammatory disorders. Immune related diseases could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following tests were performed:

Ovalbumin Challenge

Procedure: This assay was carried out on 6 wild types and 14 homozygotes. Chicken ovalbumin (OVA) is a T-cell dependent antigen, which is commonly used as a model protein for studying antigen-specific immune responses in mice. OVA is non-toxic and inert and therefore will not cause harm to the animals even if no immune response is induced. The murine immune response to OVA has been well characterized, to the extent that the immunodominant peptides for eliciting T cell responses have been identified. Anti-OVA antibodies are detectable 8 to 10 days after immunization using enzyme-linked immunosorbent assay (ELIZA), and determination of different isotypes of antibodies gives further information on the complex processes that may lead to a deficient response in genetically engineered mice.

As noted above, this protocol assesses the ability of mice to raise an antigen-specific immune response. Animals were injected IP with 50 mg of chicken ovalbumin emulsified in Complete Feund's Adjuvant and 14 days later the serum titer of anti-ovalbumin antibodies (IgM, IgG1 and IgG2 subclasses) was measured. The amount of OVA-specific antibody in the serum sample is proportional to the Optical Density (OD) value generated by an instrument that scans a 96-well sample plate. Data was collected for a set of serial dilutions of each serum sample.

Results of this challenge: The (−/−) mice exhibited a trend towards an increased mean serum IgG2a response to the ovalbumin challenge when compared with their (+/+) littermates (two of four (−/−) mice). Thus, these knockout mice exhibited an increased ability to elicit an OVA-specific antibody response to the T-cell dependent OVA antigen. Inhibitors (antagonists) of PRO9799 polypeptides would be expected to also stimulate the immune system and would find utility in the cases wherein this effect would be beneficial to the individual such as in the case of leukemia, and other types of cancer, and in immunocompromised patients, such as AIDS sufferers. Accordingly, PRO9799 polypeptides or agonists thereof would be useful in inhibiting the immune response and would be useful candidates for suppressing harmful immune responses, e.g. in the case of graft rejection or graft-versus-host diseases. In addition to the ovalbumin challenge, hematology revealed that the (−/−) knockout mice showed a lower level of circulating neutrophils than the wild-type littermates.

(d) Oncology Phenotypic Analysis

In the area of oncology, targets were identified herein for the treatment of solid tumors, lymphomas and leukemia.

Adult Skin Cell Proliferation:

Procedure: Skin cells were isolated from 16 week old animals (2 wild type and 4 homozygotes). These were developed into primary fibroblast cultures and the fibroblast proliferation rates were measured in a strictly controlled protocol. The ability of this assay to detect hyper-proliferative and hypo-proliferative phenotypes has been demonstrated with p53 and Ku80. Proliferation was measured using Brdu incorporation.

Specifically, in these studies the skin fibroblast proliferation assay was used. An increase in the number of cells in a standardized culture was used as a measure of relative proliferative capacity. Primary fibroblasts were established from skin biopsies taken from wild type and mutant mice. Duplicate or triplicate cultures of 0.05 million cells were plated and allowed to grow for six days. At the end of the culture period, the number of cells present in the culture was determined using a electronic particle counter.

Results:

The female (−/−) mice exhibited a decreased skin fibroblast proliferation rate when compared with their gender-matched (+/+) littermates and the historical mean. [Analyzed wt/het/hom: 2/0/4]

Thus, homozygous mutant mice demonstrated a hypo-proliferative phenotype. As suggested by these observations, antagonists of a PRO9799 polypeptide or its encoding gene would be useful in decreasing abnormal cell proliferation.

O. Generation and Analysis of Mice Comprising DNA 173894-2947 (UNQ3096) Gene Disruptions

In these knockout experiments, the gene encoding PRO21175 polypeptides (designated as DNA173894-2947 (UNQ3096) was disrupted. The gene specific information for these studies is as follows: the mutated mouse gene corresponds to nucleotide reference: NM_{—}145837 or ACCESSION:NM_{—}145837 NID: gi 22003885 ref NM_{—}145837.1 Mus musculus interleukin 17D (IL-17D); protein reference: NP_{—}665836 or ACCESSION:NP_{—}665836 NID: gi 22003886 ref NP 665836.1 (NM_{—}145837) interleukin 17D; interleukin 27A [Mus musculus]; the human gene sequence reference: NM_{—}138284 or ACCESSION:NM_{—}138284 NID: gi 19923714 ref NM_{—}138284.1 Homo sapiens interleukin 17D (IL17D); the human protein sequence corresponds to reference: NP_{—}612141 or ACCESSION:NP_{—}612141 NID: gi 19923715 ref NP_{—}612141.1 (NM_{—}138284) interleukin 17D precursor [Homo sapiens].

The disrupted gene is Il17d (interleukin 17d), which is the ortholog of human IL17D. Aliases include IL27, IL-22, IL-27, and IL27A.

IL17D is a cytokine that is expressed at high levels in skeletal muscle, brain, adipose, heart, lung, and pancreas and at low levels in bone marrow, fetal liver, kidney, leukocytes, liver, lymph node, placenta, spleen, thymus, tonsil, resting CD4 T cells, and resting CD19 B cells. IL17D is likely to play a role in modulating the immune response and tissue homeostasis. IL17D regulates cytokine production in endothelial cells and inhibits myeloid progenitor cell colony formation but not peripheral blood mononuclear cell proliferation (Starnes et al. J Immunol 169(2):642-6 (2002); Moseley et al., Cytokine Growth Factor Rev 14(2):155-74 (2003)).

The nomenclature of cytokines has evolved erratically; apparently IL17D has been called IL-22 in the past but this name is now reserved for IL22. Similarly, some researchers have called the gene IL27A (e.g., NCBI accession AF502584).

Targeted or gene trap mutations were generated in strain 129SvEv^{Brd}-derived embryonic stem (ES) cells. The chimeric mice were bred to C57BL/6J albino mice to generate F1 heterozygous animals. These progeny were intercrossed to generate F2 wild type, heterozygous, and homozygous mutant progeny. On rare occasions, for example when very few F1 mice were obtained from the chimera, F1 heterozygous mice were crossed to 129SvEv^{Brd}/C57 hybrid mice to yield additional heterozygous animals for the intercross to generate the F2 mice. Disruption of the target gene was confirmed by Southern hybridization analysis. Level I phenotypic analysis was performed on mice from this generation.

| | wt | het | hom | Total |
|---|---|---|---|---|
| Observed | 13 | 14 | 10 | 37 |
| Expected | 9.25 | 18.5 | 9.25 | 37 |

Chi-Sq. = 2.68
Significance = 0.26241
(hom/n) = 0.27
Avg. Litter Size = 6

Mutation Information Mutation Type: Homologous Recombination (standard). Coding exon 3 was targeted (NCBI accession AF458063).

Wild-type Expression Panel: Not tested. Disruption of the target gene was confirmed by Southern hybridization analysis.

1. Phenotypic Analysis (for Disrupted Gene: DNA173894-2947 (UNQ3096)

(a) Overall Phenotypic Summary

Mutation of the gene encoding the ortholog of human interleukin 17d (IL17D) resulted in the microscopic observations of multiple renal cortical cysts and hydronephrosis indicative of renal dysfunction. The knockout mice also showed signs of inflammation. In addition, the (−/−) mice demonstrated increased total tissue mass, lean body mass and bone mineral density. All the (−/−) mice were noted no in the tail suspension test, whereas three out of the five (3/5) wild-type and three out of three (3/3) heterozygous (+/−) mice were noted yes—grab ispsilateral leg. Five out of seven (5/7) (−/−) mice, three out of three (3/3) (+/−) and two out of five (2/5) (+/+) mice were noted no for rearing. Gene disruption was confirmed by Southern blot.

(b) Pathology & Radiology Observations

CAT Scan:

Test Description:

The mouse was injected with a CT contrast agent, Omnipaque 300 (Nycomed Amershan, 300 mg of iodine per ml, 0.25 ml per animal, or 2.50-3.75 g iodine/kg of body weight) intraperitoneally. After resting in the cage for ˜10 minutes, the mouse was then sedated by intraperitoneal injection of Avertin (1.25% 2,2,2,-tribromoethanol, 20 ml/kg body weight). A CAT-scan was performed using a MicroCAT scanner (ImTek, Inc.) with the anesthetized animal lying prone on the test bed. Three dimensional images were reconstructed by the Feldkamp algorithm in a cluster of workstations using an ImTek 3D RECON software.

Results:

A hypodense lesion (cyst) in the upper right kidney was observed in one of the (−/−) mice compared to their gender-matched littermates. [Analyzed wt/het/hom: 7/5/9]

Radiology:

Microscopic Observations: The (−/−) mice analyzed exhibited unilateral hydronephrosis, as well as multiple renal cortical cysts. These lesions represent a defect in mesonephric duct development. [Analyzed wt/het/hom: 0/3/5]

In summary, knockout (−/−) mutant mice deficient in the gene encoding PRO21175 polypeptides results in a negative phenotype which is associated with renal dysfunction and/or renal diseases. More specifically, mutant mice deficient in the gene encoding PRO21175 polypeptides exhibit defective mesonephric duct development which results in renal function impairment. As such, PRO21175 polypeptides or agonists thereto would be expected to be important in maintaining normal renal function whereas antagonists (inhibitors) of PRO21175 would mimic renal disease and could serve as a model for studying renal function.

(e) Bone Metabolism: Radiology Phenotypic Analysis

In the area of bone metabolism, targets were identified herein for the treatment of arthritis, osteoporosis, osteopenia and osteopetrosis as well as identifying targets that promote bone healing. Tests included:

DEXA for measurement of bone mineral density on femur and vertebra

MicroCT for very high resolution and very high sensitivity measurements of bone mineral density for both trabecular and cortical bone.

Dexa Analysis—Test Description:

Procedure: A cohort of 4 wild type and 4 heterozygotes were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

DEXA Results:

The (−/−) homozygous mice exhibited an increase in total tissue mass, lean body mass, and bone mineral density compared with their gender-matched (+/+) littermates.

These results demonstrate that the (−/−) mice exhibit a phenotype that is associated with growth abnormalities as well as such bone abnormalities as osteopetrosis. Osteopetrosis is a condition characterized by abnormal thickening and hardening of the bone leading to abnormal fragility. As such, PRO21175 polypeptides or agonists thereof are essential for normal bone development and would be beneficial for the treatment of osteopetrosis. A phenotype associated with an increased bone mineral density suggests that agents which mimic this negative phenotype (e.g. antagonists of PRO21175) would be useful in bone healing.

P. Generation and Analysis of Mice Comprising DNA148009-2889 (UNQ5931) Gene Disruptions

In these knockout experiments, the gene encoding PRO19837 polypeptides (designated as DNA148009-2889 (UNQ5931) was disrupted. The gene specific information for these studies is as follows: the mutated mouse gene corresponds to nucleotide reference: NM_{—}033608 or ACCESSION:NM_{—}033608 NID: gi 18426806 ref NM_{—}033608.1 Mus musculus immunoglobulin superfamily, member 9 (Igsf9); protein reference: NP_{—}291086 or ACCESSION:NP_{—}291086 NID: gi 18426807 ref NP_{—}291086.1 (NM_{—}033608) immunoglobulin superfamily, member 9; KIAA1355 hypothetical protein (human); NCAM-like protein NRT1; neural cell adhesion molecule (Ncam)-like [Mus musculus]; the human gene sequence reference: NM_{—}020789 or ACCESSION:NM_{—}020789 NID: gi 21357326 ref NM_{—}020789.1 Homo sapiens immunoglobulin superfamily, member 9 (IGSF9); the human protein sequence corresponds to reference: NP_{—}065840 or ACCESSION:NP_{—}065840 NID: gi 21357327 ref NP_{—}065840.1 (NM_{—}020789) immunoglobulin superfamily, member 9 [Homo sapiens].

The disrupted mouse gene is Igsf9 (immunoglobulin superfamily, member 9), the ortholog of human IGSF9. Aliases include NRT1, Ncaml, 644ETD8, Kiaa1355-hp, NCAM-like protein NRT1, neural cell adhesion molecule (Ncam)-like IGSF9 is a hypothetical type I membrane protein; it has an extracellular domain with 5 IGg domains, 2 fibronectin domains as well as a C-terminal cytoplasmic domain. The structure of IGSF9 is similar to that of neural cell adhesion molecules. In embryonic mouse, IGSF9 is expressed in dorsal root ganglia, trigeminal ganglia, and olfactory epithelium. In humans, IGSF9 appears to be more widely expressed (Doudney et al., Genomics 79(5):663-70 (2002)). IGSF9 is presumed to be involved in the development of the nervous system.

Targeted or gene trap mutations were generated in strain 129SvEv^{Brd}-derived embryonic stem (ES) cells. The chimeric mice were bred to C57BL/6J albino mice to generate F1 heterozygous animals. These progeny were intercrossed to generate F2 wild type, heterozygous, and homozygous mutant progeny. On rare occasions, for example when very few F1 mice were obtained from the chimera, F1 heterozygous mice were crossed to 129SvEv^{Brd}/C57 hybrid mice to yield additional heterozygous animals for the intercross to generate the F2 mice.

Disruption of the target gene was confirmed by Southern hybridization analysis. Level I phenotypic analysis was performed on mice from this generation.

| | wt | het | hom | Total |
|---|---|---|---|---|
| Observed | 19 | 44 | 21 | 84 |
| Expected | 21 | 42 | 21 | 84 |

Chi-Sq. = 0.29
Significance = 0.86688
(hom/n) = 0.25
Avg. Litter Size = 8

Mutation Information: Mutation Type: Retroviral Insertion (OST). Retroviral insertion occurred in the intron preceeding coding exon 1 (NCBI accession NM_{—}033608.2).

Wild-type expression of the target gene was detected only in eye among the 13 adult tissue samples tested by RT-PCR. RT-PCR analysis revealed that the transcript was absent in the (−/−) mouse analyzed (M-86).

Disruption of the target gene was confirmed by Inverse PCR. UNQ5931 expression in mouse embryos showed wide-spread expression which is excluded from the heart and yolk sac (E10.5).

1. Phenotypic Analysis (for Disrupted Gene: DNA148009-2889 (UNQ5931)

(a) Overall Phenotypic Summary

Mutation of the gene encoding the ortholog of human immunoglobulin superfamily, member 9 (IGSF9) resulted in increased cholesterol levels in (−/−) mice. The (−/−) mice exhibited an enhanced glucose tolerance test as well as increased serum insulin levels compared with their gender-matched (+/+) littermates. Two out of eight (2/8) (−/−) mice showed no rearing behavior. Two out of eight (2/8) (−/−) and one out of four (1/4) (+/−) mice had no defecation. In addition, male (−/−) mutant mice exhibited moderate degeneration of the seminiferous tubules. Transcript was absent by RT-PCR.

(b) Phenotypic Analysis: Cardiology

In the area of cardiovascular biology, targets were identified herein for the treatment of hypertension, atherosclerosis, heart failure, stroke, various coronary artery diseases, dyslipidemias such as high cholesterol (hypercholesterolemia) and elevated serum triglycerides (hypertriglyceridemia), cancer and/or obesity.

The phenotypic tests included the measurement of serum cholesterol and triglycerides.

Blood Lipids

Procedure:

A cohort of 4 wild type, 4 heterozygotes and 8 homozygotes were tested in this assay. High cholesterol levels are recognized risk factors in the development of cardiovascular disease. Measuring blood lipids allowed finding of the biological switches that regulate blood lipid levels and that upon inhibition would lead to a reduction in the risk for cardiovascular disease. Cholesterol measurements were recorded. The COBAS Integra 400 (mfr: Roche) was used for running blood chemistry tests on mice.

Results:

As summarized above, the homozygous (−/−) mutant mice exhibited an increased mean serum cholesterol level (compared to normal levels) when compared with their gender-matched (+/+) littermates and the historical mean. No change in triglycerides was observed. (Analyzed wt/het/hom: 4/4/8)

Thus, mutant mice deficient in the PRO19837 can serve as a model for cardiovascular disease especially those diseases which are associated with an abnormal cholesterol metabolism. PRO19837 polypeptides or its encoding gene would be useful in regulating blood lipids and in particular maintaining normal cholesterol. Thus, PRO19837 polypeptides would be useful in the treatment of such cardiovascular diseases as: hypertension, atherosclerosis, heart failure, stroke, various coronary artery diseases, and/or obesity or diabetes.

Phenotypic Analysis Metabolism-Blood Chemistry—Glucose Tolerance

In the area of metabolism, targets may be identified for the treatment of diabetes. Blood chemistry phenotypic analysis includes glucose tolerance tests to measure insulin sensitivity and changes in glucose metabolism. Abnormal glucose tolerance test results may indicate but may not be limited to the following disorders or conditions: Diabetes Type 1 and Type 2, Syndrome X, various cardiovascular diseases and/or obesity.

Procedure: A cohort of 4 wild type and 8 homozygote mice were used in this assay. The glucose tolerance test is the standard for defining impaired glucose homeostasis in mammals. Glucose tolerance tests were performed using a Lifescan glucometer. Animals were injected IP at 2 g/kg with D-glucose delivered as a 20% solution and blood glucose levels were measured at 0, 30, 60 and 90 minutes after injection.

Results: These studies indicated that (−/−) mice exhibit enhanced glucose tolerance in the presence of normal fasting glucose at ⅔ intervals tested when compared with their gender-matched (+/+) littermates and the historical means. In addition, slightly increased serum insulin levels (or hyperinsulinemia) was apparent in the (−/−) mice.

(c) Pathology

Microscopic observations on the two male (−/−) mice examined exhibited a moderate degeneration of the seminiferous tubules. The lesion was unilateral and the associated epididymus was void of sperm. This negative phenotype suggests that antagonists to PRO19837 would result in male reproductive disorders. In contrast, PRO19837 or agonists thereof would be useful in the prevention or treatment of such disorders.

Q. Generation and Analysis of Mice Comprising DNA175959-2948 (UNQ6427) Gene Disruptions

In these knockout experiments, the gene encoding PRO21331 polypeptides (designated as DNA175959-2948 (UNQ6427) was disrupted. The gene specific information for these studies is as follows: the mutated mouse gene corresponds to nucleotide reference: XM_{—}283647 or Mus musculus RIKEN cDNA A530037CO₄ gene (A530037CO₄Rik); protein reference: XP_{—}283647—similar to VTS20631 [Mus musculus]; the human gene sequence reference: NM_{—}021636 or Homo sapiens leucine-rich repeat-containing G protein-coupled receptor 6 (LGR6); the human protein sequence corresponds to reference: NP-067649 or leucine-rich repeat-containing G protein-coupled receptor 6 [Homo sapiens] gi|37181344|gb|AAQ88486.1|gonadotropin receptor [Homo sapiens].

The disrupted mouse gene Lgr6 (leucine-rich repeat-containing G protein-coupled receptor 6), which is the ortholog of human LGR6.

LGR6 is a member of the leucine-rich repeat subfamily of rhodopsin-like G protein-coupled receptors. The LGR6 gene encodes an 846 amino acid polypeptide and, like glycoprotein hormone receptors, has a large extracellular N-terminal domain that contains leucine-rich repeats. The endogenous ligand and signaling pathways for this receptor are not known. The LGR6 gene is likely to undergo alternative splicing to generate different LGR6 receptor subtypes. LGR6 mRNA is expressed in oviduct, uterus, colon, spleen, kidney, adrenal gland, brain, and heart. High LGR6 mRNA expression is present in small intestine, testis, and ovary (Hsu et al., Mol Endocrinol 14(8):1257-71 (2000)).

Targeted or gene trap mutations were generated in strain 129SvEv^{Brd}-derived embryonic stem (ES) cells. The chimeric mice were bred to C57BL/6J albino mice to generate F1 heterozygous animals. These progeny were intercrossed to generate F2 wild type, heterozygous, and homozygous mutant progeny. On rare occasions, for example when very few F1 mice were obtained from the chimera, F1 heterozygous mice were crossed to 129SvEv^{Brd}/C57 hybrid mice to yield additional heterozygous animals for the intercross to generate the F2 mice. Disruption of the target gene was confirmed by Southern hybridization analysis. Level I phenotypic analysis was performed on mice from this generation.

| | wt | het | hom | Total |
|---|---|---|---|---|
| Observed | 13 | 37 | 18 | 68 |
| Expected | 17 | 34 | 17 | 68 |

Chi-Sq. = 1.26
Significance = 0.53134
(hom/n) = 0.26
Avg. Litter Size = 7

Mutation Information: Mutation Type: Homologous Recombination (standard). Coding exon 18 was targeted (NCBI accession AK085901).

Wild-type Expression Panel: Not tested. Disruption of the target gene was confirmed by Southern hybridization analysis.

1. Phenotypic Analysis (for Disrupted Gene: DNA175959-2948 (UNQ6427)

(a) Overall Phenotypic Summary

Mutation of the gene encoding a homolog of human leucine-rich repeat-containing G protein-coupled receptor 6 (LGR6) resulted in increased triglycerides in homozygous (−/−) mice. In addition, the mutant (−/−) mice showed a trend in lowered bone mineral density measurements as shown by micro CT analysis. Four (4) out of eight (8) or 50% of the (−/−) mice had no defecation and two (2) out of eight (8) of the (−/−) mice had no rearing behavior. Exophthalmus was observed in one of the (−/−) mice and also one of the (+/−) mouse. Gene disruption was confirmed by Southern blot.

(b) Phenotypic Analysis: Cardiology

In the area of cardiovascular biology, targets were identified herein for the treatment of hypertension, atherosclerosis, heart failure, stroke, various coronary artery diseases, dyslipidemias such as high cholesterol (hypercholesterolemia) and elevated serum triglycerides (hypertriglyceridemia), cancer and/or obesity.

The phenotypic tests included the measurement of serum cholesterol and triglycerides.

### Blood Lipids

Procedure:

A cohort of 4 wild type, 4 heterozygotes and 8 homozygotes were tested in this assay. High triglyceride levels are recognized risk factors in the development of cardiovascular disease, diabetes and/or obesity. Measuring blood lipids allowed finding of the biological switches that regulate blood lipid levels and that upon inhibition would lead to a reduction in the risk for cardiovascular disease. The COBAS Integra 400 (mfr: Roche) was used for running blood chemistry tests on mice.

Results:

As summarized above, the homozygous (−/−) mutant mice exhibited an increased mean serum triglyceride level (compared to normal levels) when compared with their gender-matched (+/+) littermates and the historical mean. (188 mg/dL (−/−) vs. 88 mg/dL; p=0.04) (Analyzed wt/het/hom: 4/4/8)

Thus, mutant mice deficient in the PRO21331 can serve as a model for cardiovascular disease, diabetes and/or obesity. PRO21331 polypeptides or its encoding gene would be useful in regulating blood lipids and in particular maintaining normal lipid metabolism. Thus, PRO21331 polypeptides would be useful in the treatment of such cardiovascular diseases as: hypertension, atherosclerosis, heart failure, stroke, various coronary artery diseases, diabetes and/or obesity.

(c) Bone Metabolism: Radiology Phenotypic Analysis

In the area of bone metabolism, targets were identified herein for the treatment of arthritis, osteoporosis, osteopenia and osteopetrosis as well as identifying targets that promote bone healing.

Bone microCT Analysis:

Procedure: MicroCT was used to get very sensitive measurements of BMD. One vertebra and 1 femur were taken from a cohort of 4 wild type and 8 homozygous mice. Measurements were taken of lumbar 5 veterbra traebecular bone volume, traebecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

Micro-CT Analysis Results: The (−/−) mice exhibited notably decreased mean lumbar 5 vertebral trabecular bone volume, number, thickness, and connectivity density when compared with their gender-matched (+/+) littermates and the historical means. These mutants also exhibited notably decreased mean femoral midshaft cross-sectional area. [Analyzed wt/het/hom: 4/4/8]

These results demonstrate that knockout mutant male mice deficient in the gene encoding PRO21331 polypeptides exhibit abnormal bone metabolism with significant bone loss characterized by a decrease in bone mass with decreased density and possibly fragility leading to bone fractures. No hypercalcemia, hyperglycemia, or increased alkaline phosphate was detected in blood chemistry tests to suggest renal, parathyroid, or adrenal dysfunction that might be related to the decrease in bone mineral density. Thus, it appears that PRO21331 or agonists thereof would be useful in maintaining bone homeostasis and would be important in bone healing or for the treatment of arthritis or osteoporosis; whereas antagonists to PRO21331 or its encoding gene would lead to abnormal or pathological bone disorders including inflammatory diseases associated with abnormal bone metabolism such as arthritis, osteoporosis, and osteopenia.

R. Generation and Analysis of Mice Comprising DNA194607 (UNQ8923) Gene Disruptions

In these knockout experiments, the gene encoding PRO23949 polypeptides (designated as DNA194607 (UNQ8923) was disrupted. The gene specific information for these studies is as follows: the mutated mouse gene corresponds to nucleotide reference: NM_{—}015775 or ACCESSION:NM_{—}015775 NID: gi 7657650 ref NM_{—}015775.1 Mus musculus transmembrane protease, serine 2 (Tmprss2); protein reference: □9JIQ8 or ACCESSION:Q9JIQ8 NID: Mus musculus (Mouse). TRANSMEMBRANE PROTEASE, SERINE 2 (EC 3.4.21.-) (EPITHELIASIN) (PLASMIC TRANSMEMBRANE PROTEIN X). MOUSESPTRNRDB; the human gene sequence reference: NM_{—}005656 or ACCESSION:NM_{—}005656 NID: gi 14602458 ref NM_{—}005656.2 Homo sapiens transmembrane protease, serine 2 (TMPRSS2); the human protein sequence corresponds to reference: O15393 or ACCESSION:O15393 NID: Homo sapiens (Human). TRANSMEMBRANE PROTEASE, SERINE 2 PRECURSOR (EC 3.4.21.-). HUMANSPTRNRDB.

The disrupted mouse gene is Tmprss2 (transmembrane protease, serine 2), ortholog of human TMPRSS2. Aliases include epitheliasin, plasma membrane protein X, and PRSS10.

TMPRSS2, a type II plasma membrane protein, is a serine protease primarily found in the apical surfaces of renal tubular and airway epithelia. Prostate, colon, stomach, and salivary gland also express TMPRSS2. The protein contains a signal anchor sequence, a low-density lipoprotein receptor class A domain, a cysteine-rich scavenger receptor-like domain, and a trypsin-like serine protease domain (Paoloni-Giacobino et al., Genomics 44(3):309-20 (1997); Jacquinet et al., FEBS Lett 468(1):93-100 (2000); Vaarala et al., Int J Cancer 94(5):705-10 (2001)). TMPRSS2 is down-regulated in androgen-independent prostate cancer (Afar et al., Cancer Res 61(4):1686-92 (2001)), and a cleaved form is secreted from the prostate. However, TMPRSS2 is overexpressed in the majority of prostate tumors (Vaarala et al., 1999) and is thought to be androgen regulated (Lin et al., Cancer Res 59(17):4180-4 (1999)). Thus, TMPRSS2 may be a target for cancer therapy and diagnosis. TMPRSS2 may also cleave epithelial sodium channel (ENaC), which decreases sodium absorption across airway epithelia. Thus, TMPRSS2 may play a role in airway surface liquid volume regulation and mucociliary clearance efficiency (Donaldson et al., J Biol Chem 277(10):8338-45 (2002)).

Targeted or gene trap mutations were generated in strain 129SvEv^{Brd}-derived embryonic stem (ES) cells. The chimeric mice were bred to C57BL/6J albino mice to generate F1 heterozygous animals. These progeny were intercrossed to generate F2 wild type, heterozygous, and homozygous mutant progeny. On rare occasions, for example when very few F1 mice were obtained from the chimera, F1 heterozygous mice were crossed to 129SvEv^{Brd}/C57 hybrid mice to yield additional heterozygous animals for the intercross to generate the F2 mice. Disruption of the target gene was confirmed by Southern hybridization analysis. Level I phenotypic analysis was performed on mice from this generation.

| | wt | het | hom | Total |
|---|---|---|---|---|
| Observed | 13 | 50 | 21 | 84 |
| Expected | 21 | 42 | 21 | 84 |

Chi-Sq. = 4.57
Significance = 0.10170
(hom/n) = 0.25
Avg. Litter Size = 9

Mutation Information: Mutation Type: Homologous Recombination (standard). Coding exons 1 and 2 were targeted (NCBI accession NM_{—}015775.2).

Wild-type expression of the target gene was detected in embryonic stem (ES) cells and, among the 13 adult tissue samples tested by RT-PCR, in kidney and small intestine and colon. Disruption of the target gene was confirmed by Southern hybridization analysis.

1. Phenotypic Analysis (for Disrupted Gene: DNA194607 (UNQ8923)

(a) Overall Phenotypic Summary

Mutation of the gene encoding the ortholog of human transmembrane protease, serine 2 (TMPRSS2) resulted in decreased cholesterol levels in homozygous mutant (−/−) mice. In addition, male (−/−) mice showed a decreased femoral midshaft cortical thickness and cross-sectional area when compared with their gender matched (+/+) littermates and the historical mean. Mutant (−/−) mice exhibited decreased proliferation in skin fibroblasts in comparison to wild-type controls. Gene disruption was confirmed by Southern blot.

(b) Cardiovascular Phenotypic Analysis/Metabolism-Blood Chemistry

In the area of cardiovascular biology, phenotypic testing was performed to identify potential targets for the treatment of cardiovascular, endothelial or angiogenic disorders such as hypertension, atherosclerosis, heart failure, stroke, various coronary artery diseases, dyslipidemias such as high cholesterol (hypercholesterolemia) and elevated serum triglycerides (hypertriglyceridemia), cancer and/or obesity. The phenotypic tests in this instance included the measurement of serum cholesterol.

Blood Lipids

Procedure: A cohort of 4 wild type and 8 homozygote males were used in these assays. Mean serum cholesterol levels were measured and compared with gender matched (+/+) littermates. The COBAS Integra 400 (mfr: Roche) was used for running blood chemistry tests on mice.

Results: Homozygous mutant mice exhibited a decreased mean serum cholesterol level (˜20 mg/dL) when compared with their gender-matched wild-type littermates and the historical means. In summary, these knockout mutant mice exhibited a positive phenotype with regards to lipid metabolism. Thus, mutant mice deficient in the PRO23949 gene can serve as a model for treatment of cardiovascular disease. Antagonists to PRO23949 or its encoding gene would be useful in regulating blood lipids and in particular in maintaining normal cholesterol metabolism. Such inhibitors or antagonists to PRO23949 polypeptides would be useful in the treatment of such cardiovascular diseases associated with dyslipidemia as: hypertension, atherosclerosis, heart failure, stroke, various coronary artery diseases, obesity and/or diabetes.

(c) Bone Metabolism: Radiology Phenotypic Analysis

In the area of bone metabolism, targets were identified herein for the treatment of arthritis, osteoporosis, osteopenia and osteopetrosis as well as identifying targets that promote bone healing. Tests included:

DEXA for measurement of bone mineral density on femur and vertebra

MicroCT for very high resolution and very high sensitivity measurements of bone mineral density for both trabecular and cortical bone.

Dexa Analysis—Test Description:

Procedure: A cohort of 4 wild type, 4 heterozygotes and 8 homozygotes were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Bone microCT Analysis:

Procedure: MicroCT was also used to get very sensitive measurements of BMD. One vertebra and 1 femur were taken from a cohort of 4 wild type and 8 homozygous mice. Measurements were taken of lumbar 5 veterbra traebecular bone volume, traebecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

Micro-CT Analysis Results: The male (—) mice exhibited notably decreased mean femoral midshaft cortical thickness and cross-sectional area when compared with their gender-matched (+/+) littermates and the historical means. [Analyzed wt/het/hom: 4/4/8]

These results demonstrate that knockout mutant male mice deficient in the gene encoding PRO23949 polypeptides exhibit abnormal bone metabolism with significant bone loss characterized by a decrease in bone cortical thickness and possibly fragility leading to bone fractures. No hypercalcemia, hyperglycemia, or increased alkaline phosphate was detected in blood chemistry tests to suggest renal, parathyroid, or adrenal dysfunction that might be related to the decrease in bone mineral density. As the bone mineral density defect was only seen in male knockout mice, this bone abnormality observation may suggest testosterone deficiency. Thus, it appears that PRO23949 or agonists thereof would be useful in maintaining bone homeostasis mediated by male hormones such as testosterone. In addition, PRO23949 or its encoding gene would be useful in maintaining bone homeostasis and would be important in bone healing or for the treatment of arthritis or osteoporosis; whereas antagonists to PRO23949 or its encoding gene would lead to abnormal or pathological bone disorders including inflammatory diseases associated with abnormal bone metabolism such as arthritis, osteoporosis, and osteopenia.

### (d) Oncology Phenotypic Analysis

In the area of oncology, targets were identified herein for the treatment of solid tumors, lymphomas and leukemia.

Adult Skin Cell Proliferation:

Procedure: Skin cells were isolated from 16 week old animals (2 wild type and 4 homozygotes). These were developed into primary fibroblast cultures and the fibroblast proliferation rates were measured in a strictly controlled protocol. The ability of this assay to detect hyper-proliferative and hypo-proliferative phenotypes has been demonstrated with p53 and Ku80. Proliferation was measured using Brdu incorporation.

Specifically, in these studies the skin fibroblast proliferation assay was used. An increase in the number of cells in a standardized culture was used as a measure of relative proliferative capacity. Primary fibroblasts were established from skin biopsies taken from wild type and mutant mice. Duplicate or triplicate cultures of 0.05 million cells were plated and allowed to grow for six days. At the end of the culture period, the number of cells present in the culture was determined using a electronic particle counter.

Results:

The female (−/−) mice exhibited a decreased skin fibroblast proliferation rate when compared with their gender-matched (+/+) littermates and the historical mean. (Note: only females were tested) [Analyzed wt/het/hom: δ 2/0/4]

Thus, homozygous female mutant mice demonstrated a hypo-proliferative phenotype. As suggested by these observations, antagonists of a PRO23949 polypeptide or its encoding gene would be useful in decreasing abnormal cell proliferation.

S. Generation and Analysis of Mice Comprising DNA50920-1325 (UNQ361) Gene Disruptions

In these knockout experiments, the gene encoding PRO697 polypeptides (designated as DNA50920-1325 (UNQ361) was disrupted. The gene specific information for these studies is as follows: the mutated mouse gene corresponds to nucleotide reference: BC014722 or Mus musculus, secreted frizzled-related sequence protein 2, clone MGC:25299 IMAGE:4487469; protein reference: NP_{—}033170 or secreted frizzled-related sequence protein 2; stromal cell derived factor 5; secreted frizzled-related sequence protein 5 [Mus musculus]; the human gene sequence reference: AK075372 or Homo sapiens cDNA PSEC0060 fis, clone NT2RP2000638, highly similar to Homo sapiens pancreas tumor-related protein (FKSG12); the human protein sequence corresponds to reference: AAH08666—similar to stromal cell derived factor 5 [Homo sapiens].

The gene that is mutated is secreted frizzled-related sequence protein 2 (Sfrp2), which is the ortholog of human SFRP2 (Hs.31386). SFRP2 encodes secreted frizzled-related protein 2. The protein is also known as Sdf5, AI851596, stromal cell derived factor 5, secreted frizzled-related sequence protein 5, FRP-2, SARP1, SDF-5, and secreted apoptosis related protein 1.

Frizzled proteins are membrane-spanning receptors of Wnt-like signaling proteins. Secreted frizzled-related proteins interact with Wnt-type proteins outside the cell, thereby modulating the availability of the Wnt-type proteins to interact with the membrane-bound receptors. Overall, the process is thought to fine tune the impact of the Wnt family proteins upon cellular processes (OMIM 604157; Rattner et al, Proc Natl Acad Sci USA. 94(7):2859-63 (1997)).

Human cell lines transfected with SFRP2 become more sensitive to apoptosis-inducing factors (Melkonyan et al, Proc Natl Acad Sci USA. 94(25):13636-41 (1977)). SFRP2 may be involved in retinal degenerative processes (Jones et al, Invest Opthalmol V is Sci. 41(6):1297-301 (2000)), formation of neural cell progenitors (Aubert et al, Nat. Biotechnol. 20(12): 1240-5 (2002)), or even muscle processes (Levin et al, J Muscle Res Cell Motil. 22(4):361-9 (2001)).

Targeted or gene trap mutations were generated in strain 129SvEv^{Brd}-derived embryonic stem (ES) cells. The chimeric mice were bred to C57BLU6J albino mice to generate F1 heterozygous animals. These progeny were intercrossed to generate F2 wild type, heterozygous, and homozygous mutant progeny. On rare occasions, for example when very few F1 mice were obtained from the chimera, F1 heterozygous mice were crossed to 129SvEv^{Brd}/C57 hybrid mice to yield additional heterozygous animals for the intercross to generate the F2 mice. Disruption of the target gene was confirmed by Southern hybridization analysis. Level I phenotypic analysis was performed on mice from this generation.

| | wt | het | hom | Total |
|---|---|---|---|---|
| Observed | 26 | 46 | 15 | 87 |
| Expected | 21.75 | 43.5 | 21.75 | 87 |

Chi-Sq. = 3.07
Significance = 0.21557
(hom/n) = 0.17
Avg. Litter Size = 9

### Mutation Type: Retroviral Insertion (OST)

Retroviral insertion disrupted the gene prior to the exon encoding amino acid 164 in a protein of 295 amino acids (NCBI accession number NP_{—}033170).
Wild-type expression of the target gene was detected in embryonic stem (ES) cells and, among the 13 adult tissue samples tested by RT-PCR, in brain, lung, kidney, skin fibroblast, heart, and adipose.
RT-PCR analysis revealed that the transcript was absent in the (−/−) mice analyzed.

1. Phenotypic Analysis (for Disrupted Gene: DNA50920-1325 (UNQ361)

(a) Overall Phenotypic Summary

Mutation of the gene encoding the ortholog of human secreted frizzled-related protein 2 (SFRP2) resulted in the observation that mutant (−/−) mice showed a decreased or impaired response to an ovalbumin challenge. Male (−/−) mice appeared to have an increased bone mineral content, lean body mass and bone mineral density. Female (−/−) mice had a decreased total tissue mass and fat. Pathology showed focal hepatic coagulative necrosis with chronic inflammation and diffuse endometrial papillary hyperplasia in one (−/−) mouse. Transcript was absent by RT-PCR.

(b) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders.

In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following test was performed:

Ovalbumin Challenge

Procedure: This assay was carried out on 8 wild types and 16 homozygotes. Chicken ovalbumin (OVA) is a T-cell dependent antigen, which is commonly used as a model protein for studying antigen-specific immune responses in mice. OVA is non-toxic and inert and therefore will not cause harm to the animals even if no immune response is induced. The murine immune response to OVA has been well characterized, to the extent that the immunodominant peptides for eliciting T cell responses have been identified. Anti-OVA antibodies are detectable 8 to 10 days after immunization using enzyme-linked immunosorbent assay (ELIZA), and determination of different isotypes of antibodies gives further information on the complex processes that may lead to a deficient response in genetically engineered mice.

As noted above, this protocol assesses the ability of mice to raise an antigen-specific immune response. Animals were injected IP with 50 mg of chicken ovalbumin emulsified in Complete Feund's Adjuvant and 14 days later the serum titer of anti-ovalbumin antibodies (IgM, IgG1 and IgG2 subclasses) was measured. The amount of OVA-specific antibody in the serum sample is proportional to the Optical Density (OD) value generated by an instrument that scans a 96-well sample plate. Data was collected for a set of serial dilutions of each serum sample.

[Analyzed wt/het/hom: 8/4/16]

Results of this challenge: The (−/−) mice exhibited a decreased mean serum IgG2a response to the ovalbumin challenge when compared with their (+/+) littermates. Thus, these knockout mice exhibited an decreased ability to elicit an OVA-specific antibody response to the T-cell dependent OVA antigen. These results are consistent with an impaired TH1 response. PRO697 polypeptides or agonists thereof would therefore be expected to stimulate the immune system and would find utility in the cases wherein this effect would be beneficial to the individual such as in the case of leukemia, and other types of cancer, and in immunocompromised patients, such as AIDS sufferers. Accordingly, inhibitors or antagonists of PRO697 polypeptides would be useful in inhibiting the immune response and would be useful candidates for suppressing harmful immune responses, e.g. in the case of graft rejection or graft-versus-host diseases.

(c) Bone Metabolism: Radiology Phenotypic Analysis

In the area of bone metabolism, targets were identified herein for the treatment of arthritis, osteoporosis, osteopenia and osteopetrosis as well as identifying targets that promote bone healing. Tests included:

DEXA for measurement of bone mineral density on femur and vertebra

MicroCT for very high resolution and very high sensitivity measurements of bone mineral density for both trabecular and cortical bone.

Dexa Analysis—Test Description:

Procedure: A cohort of 4 wild type, 4 heterozygotes and 8 homozygotes were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

DEXA Results: The male (−/−) mice exhibited an increased mean bone mineral content (BMC), bone mineral density (BMD) and lean body mass (LBM) when compared with their gender-matched (+/+) littermates and the historical means. In addition, the female mutant (−/−) mice demonstrated a decreased total tissue mass (TTM) and fat percentages. These results are consistent with abnormal bone metabolism. These results indicate that the knockout male mutant phenotype is associated with such bone abnormalities as osteopetrosis. Osteopetrosis is a condition characterized by abnormal thickening and hardening of bone and abnormal fragility of the bones. As such, PRO697 polypeptides or agonists thereof would be beneficial for the treatment of osteopetrosis. A phenotype associated with an increased bone mineral content, and total body and femoral bone mineral density suggests that agents which mimic these effects (e.g. antagonists of PRO697 polypeptides) would be useful in bone healing. Female mutant (−/−) mice exhibited decreased total tissue mass and fat which would indicate a tissue wasting condition.

T. Generation and Analysis of Mice Comprising DNA67962-1649 (UNQ749) Gene Disruptions

In these knockout experiments, the gene encoding PRO1480 polypeptides (designated as DNA67962-1649 (UNQ749) was disrupted. The gene specific information for these studies is as follows: the mutated mouse gene corresponds to nucleotide reference: NM_{—}013659 or Mus musculus sema domain, immunoglobulin domain (Ig), transmembrane domain (TM) and short cytoplasmic domain, (semaphorin) 4B (Sema4b); protein reference: IPI00222220—ACCESSION:IPI00222220 NID or Mus musculus (Mouse). SEMAPHORIN 4B. IPI_mouse; the human gene sequence reference: NM_{—}020210 or Homo sapiens sema domain, immunoglobulin domain (Ig), transmembrane domain (TM) and short cytoplasmic domain, (semaphorin) 4B (SEMA4B), transcript variant 1; the human protein sequence corresponds to reference: □9NPR2 or Semaphorin 4B precursor.

The gene of interest is Sema4b (sema domain, immunoglobulin domain (Ig), transmembrane domain (TM) and short cytoplasmic domain, (semaphorin) 4B), ortholog of human SEMA4B. Aliases include SemC and Semac. SEMA4B is a predicted type I plasma membrane protein belonging to the semaphorin family. The protein contains an extracellular segment, which is composed of a sema domain and ah immunoglobulin-like C2-type domain, a transmembrane segment, and a short cytoplasmic segment. Semaphorins exist as both secreted and type I membrane proteins. SEMA4B is likely to function as a receptor that binds with ligands such as plexin domain-containing proteins or neuropilins. The biological role of SEMA4B is not known; however, semaphorins generally play a role in axon guidance, cell migration, cancer, and immune regulation (Puschel et al, Neuron 14(5):941-8 (1995); Williams-Hogarth et al, J Comp Neurol 423(4):565-78 (2000); He et al, Sci STKE (2002)(119):RE1; Trusolino and Comoglio, Nat Rev Cancer 2(4):289-300 (2002); Kumanogoh and Kikutani, Adv Immunol 81:173-98 (2003); Pasterkamp and Kolodkin, Curr Opin Neurobiol 13(1):79-89 (2003)).

Targeted or gene trap mutations were generated in strain 129SvEv^{Brd}-derived embryonic stem (ES) cells. The chimeric mice were bred to C57BL/6J albino mice to generate F1 heterozygous animals. These progeny were intercrossed to generate F2 wild type, heterozygous, and homozygous mutant progeny. On rare occasions, for example when very few F1 mice were obtained from the chimera, F1 heterozygous mice were crossed to 129SvEv^{Brd}/C57 hybrid mice to yield additional heterozygous animals for the intercross to generate the F2 mice. Disruption of the target gene was confirmed by Southern hybridization analysis. Level I phenotypic analysis was performed on mice from this generation.

| | wt | het | hom | Total |
|---|---|---|---|---|
| Observed | 19 | 55 | 14 | 88 |
| Expected | 22 | 44 | 22 | 88 |

Chi-Sq. = 6.07
Significance = 0.04812
(hom/n) = 0.16
Avg. Litter Size = 9

Wild-type expression Panel was not tested.
RT-PCR analysis revealed that the transcript was absent in the homozygous mutant mouse analyzed (M-45).

1. Phenotypic Analysis (for Disrupted Gene: DNA67962-1649 (UNQ749)

(a) Overall Phenotypic Summary

Mutation of the gene encoding the ortholog of human semaphorin 4B (SEMA4B) resulted in the observation that mutant male (−/−) mice showed a decrease in volumetric bone mineral density (vBMD), bone mineral density (BMD), bone mineral content (BMC), total tissue mass (TTM), lean body mass (LBM) and BMC/LBM mice. Two out of the eight (−/−) mice (25%) were noted yes for urination. Three out of the eight (−/−) mice (38%) were noted yes for 20 second tail suspension response—does not grab ipsilateral leg and one out of eight (−/−) mice (13%) in 20 second tail suspension response—does not grab contralateral leg. Transcript was absent by RT-PCR. There was also a reduction in the expected numbers of homozygotes (significance=0.04812) indicating some degree of reduced viability.

(b) Bone Metabolism: Radiology Phenotypic Analysis

In the area of bone metabolism, targets were identified herein for the treatment of arthritis, osteoporosis, osteopenia and osteopetrosis as well as identifying targets that promote bone healing. Tests included:

DEXA for measurement of bone mineral density on femur and vertebra

MicroCT for very high resolution and very high sensitivity measurements of bone mineral density for both trabecular and cortical bone.

Dexa Analysis—Test Description:

Procedure: A cohort of 4 wild type, 4 heterozygotes and 8 homozygotes were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Results:

Male (−/−) mice exhibited bone changes associated with a decrease in volumetric bone mineral density (vBMD), bone mineral density (BMD), total tissue mass (TTM), lean body mass (LBM), bone mineral content (BMC) and BMC/LBM. These results demonstrate that knockout mutant mice exhibit abnormal bone metabolism with significant bone loss similar to osteoporosis characterized by decrease in bone mass with decreased density and possibly fragility leading to bone fractures. Thus, it appears that PRO1480 or its encoding gene plays a vital role maintaining bone homeostasis and would be useful for bone healing or for the treatment of arthritis or osteoporosis, whereas PRO1480 antagonists (or inhibitors of PRO1480 or its encoding gene) would lead to abnormal or pathological bone disorders including inflammatory diseases associated with abnormal bone metabolism including but not limited to arthritis, osteoporosis and osteopenia. In addition to these studies, (−/−) mutant mice showed signs of reduced viability since there was a reduction in the expected numbers of homozygotes (significance=0.04812).

### Example 22

### Use of PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331L PRO23949, PRO697 or PRO1480 as a hybridization probe

The following method describes use of a nucleotide sequence encoding a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide as a hybridization probe.

DNA comprising the coding sequence of full-length or mature PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides as disclosed herein is employed as a probe to screen for homologous DNAs (such as those encoding naturally-occurring variants of PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides) in human tissue cDNA libraries or human tissue genomic libraries.

Hybridization and washing of filters containing either library DNAs is performed under the following high stringency conditions. Hybridization of radiolabeled PRO256-, PRO34421-, PRO334-, PRO770-, PRO983-, PRO1009-, PRO1107-, PRO1158-, PRO1250-, PRO1317-, PRO4334-, PRO4395-, PRO49192-, PRO9799-, PRO21175, PRO19837, PRO21331, PRO23949, PRO697 or PRO1480-derived probe to the filters is performed in a solution of 50% formamide, 5×SSC, 0.1% SDS, 0.1% sodium pyrophosphate,50 mM sodium phosphate, pH 6.8, 2×Denhardt's solution, and 10% dextran sulfate at 42° C. for 20 hours. Washing of the filters is performed in an aqueous solution of 0.1×SSC and 0.1% SDS at 42° C.

DNAs having a desired sequence identity with the DNA encoding full-length native sequence PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides can then be identified using standard techniques known in the art.

### Example 23

### Expression of PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 in E. coli

This example illustrates preparation of an unglycosylated form of PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides by recombinant expression in E. coli.

The DNA sequence encoding a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide is initially amplified using selected PCR primers. The primers should contain restriction enzyme sites which correspond to the restriction enzyme sites on the selected expression vector. A variety of expression vectors may be employed. An example of a suitable vector is pBR322 (derived from E. coli; see Bolivar et al., Gene, 2:95 (1977)) which contains genes for ampicillin and tetracycline resistance. The vector is digested with restriction enzyme and dephosphorylated. The PCR amplified sequences are then ligated into the vector. The vector will preferably include sequences which encode for an antibiotic resistance gene, a trp promoter, a polyhis leader (including the first six STII codons, polyhis sequence, and enterokinase cleavage site), the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 coding region, lambda transcriptional terminator, and an argU gene.

The ligation mixture is then used to transform a selected E. coli strain using the methods described in Sambrook et al., supra. Transformants are identified by their ability to grow on LB plates and antibiotic resistant colonies are then selected. Plasmid DNA can be isolated and confirmed by restriction analysis and DNA sequencing.

Selected clones can be grown overnight in liquid culture medium such as LB broth supplemented with antibiotics. The overnight culture may subsequently be used to inoculate a larger scale culture. The cells are then grown to a desired optical density, during which the expression promoter is turned on.

After culturing the cells for several more hours, the cells can be harvested by centrifugation. The cell pellet obtained by the centrifugation can be solubilized using various agents known in the art, and the solubilized PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 protein can then be purified using a metal chelating column under conditions that allow tight binding of the protein.

PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 may be expressed in E. coli in a poly-His tagged form, using the following procedure. The DNA encoding PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 is initially amplified using selected PCR primers. The primers will contain restriction enzyme sites which correspond to the restriction enzyme sites on the selected expression vector, and other useful sequences providing for efficient and reliable translation initiation, rapid purification on a metal chelation column, and proteolytic removal with enterokinase. The PCR-amplified, poly-His tagged sequences are then ligated into an expression vector, which is used to transform an E. coli host based on strain 52 (W3110 fuhA(tonA) 1 on galE rpoHts(htpRts) clpP(lacIq). Transformants are first grown in LB containing 50 mg/ml carbenicillin at 30° C. with shaking until an O.D.600 of 3-5 is reached. Cultures are then diluted 50-100 fold into CRAP media (prepared by mixing 3.57 g (NH₄)₂SO₄, 0.71 g sodium citrate.2H2O, 1.07 g KCl, 5.36 g Difco yeast extract, 5.36 g Sheffield hycase SF in 500 mL water, as well as 110 mM MPOS, pH 7.3, 0.55% (w/v) glucose and 7 mM MgSO₄) and grown for approximately 20-30 hours at 30° C. with shaking. Samples are removed to verify expression by SDS-PAGE analysis, and the bulk culture is centrifuged to pellet the cells. Cell pellets are frozen until purification and refolding.

E. coli paste from 0.5 to 1 L fermentations (6-10 g pellets) is resuspended in 10 volumes (w/v) in 7 M guanidine, 20 mM Tris, pH 8 buffer. Solid sodium sulfite and sodium tetrathionate is added to make final concentrations of 0.1M and 0.02 M, respectively, and the solution is stirred overnight at 4° C. This step results in a denatured protein with all cysteine residues blocked by sulfitolization. The solution is centrifuged at 40,000 rpm in a Beckman Ultracentifuge for 30 min. The supernatant is diluted with 3-5 volumes of metal chelate column buffer (6 M guanidine, 20 mM Tris, pH 7.4) and filtered through 0.22 micron filters to clarify. The clarified extract is loaded onto a 5 ml Qiagen Ni-NTA metal chelate column equilibrated in the metal chelate column buffer. The column is washed with additional buffer containing 50 mM imidazole (Calbiochem, Utrol grade), pH 7.4. The protein is eluted with buffer containing 250 mM imidazole. Fractions containing the desired protein are pooled and stored at 4° C. Protein concentration is estimated by its absorbance at 280 nm using the calculated extinction coefficient based on its amino acid sequence.

The proteins are refolded by diluting the sample slowly into freshly prepared refolding buffer consisting of: 20 mM Tris, pH 8.6, 0.3 M NaCl, 2.5 M urea, 5 mM cysteine, 20 mM glycine and 1 mM EDTA. Refolding volumes are chosen so that the final protein concentration is between 50 to 100 micrograms/ml. The refolding solution is stirred gently at 4° C. for 12-36 hours. The refolding reaction is quenched by the addition of TFA to a final concentration of 0.4% (pH of approximately 3). Before further purification of the protein, the solution is filtered through a 0.22 micron filter and acetonitrile is added to 2-10% final concentration. The refolded protein is chromatographed on a Poros R1/H reversed phase column using a mobile buffer of 0.1% TFA with elution with a gradient of acetonitrile from 10 to 80%. Aliquots of fractions with A280 absorbance are analyzed on SDS polyacrylamide gels and fractions containing homogeneous refolded protein are pooled. Generally, the properly refolded species of most proteins are eluted at the lowest concentrations of acetonitrile since those species are the most compact with their hydrophobic interiors shielded from interaction with the reversed phase resin. Aggregated species are usually eluted at higher acetonitrile concentrations. In addition to resolving misfolded forms of proteins from the desired form, the reversed phase step also removes endotoxin from the samples.

Fractions containing the desired folded PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide are pooled and the acetonitrile removed using a gentle stream of nitrogen directed at the solution. Proteins are formulated into 20 mM Hepes, pH 6.8 with 0.14 M sodium chloride and 4% mannitol by dialysis or by gel filtration using G25 Superfine (Pharmacia) resins equilibrated in the formulation buffer and sterile filtered.

### Example 24

### Expression of PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 in mammalian cells

This example illustrates preparation of a potentially glycosylated form of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide by recombinant expression in mammalian cells.

The vector, pRK5 (see EP 307,247, published Mar. 15, 1989), is employed as the expression vector. Optionally, the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 DNA is ligated into pRK5 with selected restriction enzymes to allow insertion of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 DNA using ligation methods such as described in Sambrook et al., supra. The resulting vector is called pRK5-PRO256, pRK5-PRO34421, pRK5-PRO334, pRK5-PRO770, pRK5-PRO983, pRK5-PRO1009, pRK5-PRO1107, pRK5-PRO1158, pRK5-PRO1250, pRK5-PRO1317, pRK5-PRO4334, pRK5-PRO4395, pRK5-PRO49192, pRK5-PRO9799, pRK5-PRO21175, pRK5-PRO19837, pRK5-PRO21331, pRK5-PRO23949, PRO697 or pRK5-PRO1480.

The selected host cells may be 293 cells. Human 293 cells (ATCC CCL 1573) are grown to confluence in tissue culture plates in medium such as DMEM supplemented with fetal calf serum and optionally, nutrient components and/or antibiotics. About 10 μg pRK5-PRO256, pRK5-PRO34421, pRK5-PRO334, pRK5-PRO770, pRK5-PRO983, pRK5-PRO1009, pRK5-PRO1107, pRK5-PRO1158, pRK5-PRO1250, pRK5-PRO1317, pRK5-PRO4334, pRK5-PRO4395, pRK5-PRO49192, pRK5-PRO9799, pRK5-PRO21175, pRK5-PRO19837, pRK5-PRO21331, pRK5-PRO23949, PRO697 or pRK5-PRO1480 DNA is mixed with about 1 μg DNA encoding the VA RNA gene [Thimmappaya et al., Cell, 31:543 (1982)] and dissolved in 500 μl of 1 mM Tris-HCl, 0.1 mM EDTA, 0.227 M CaCl₂. To this mixture is added, dropwise, 500 μl of 50 mM HEPES (pH 7.35),280 mM NaCl, 1.5 mM NaPO₄, and a precipitate is allowed to form for 10 minutes at 25° C. The precipitate is suspended and added to the 293 cells and allowed to settle for about four hours at 37° C. The culture medium is aspirated off and 2 ml of 20% glycerol in PBS is added for 30 seconds. The 293 cells are then washed with serum free medium, fresh medium is added and the cells are incubated for about 5 days.

Approximately 24 hours after the transfections, the culture medium is removed and replaced with culture medium (alone) or culture medium containing 200 μCi/ml ³⁵S-cysteine and 200 μCi/ml ³⁵S-methionine. After a 12 hour incubation, the conditioned medium is collected, concentrated on a spin filter, and loaded onto a 15% SDS gel. The processed gel may be dried and exposed to film for a selected period of time to reveal the presence of PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides. The cultures containing transfected cells may undergo further incubation (in serum free medium) and the medium is tested in selected bioassays.

In an alternative technique, PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 may be introduced into 293 cells transiently using the dextran sulfate method described by Somparyrac et al., Proc. Natl. Acad. Sci., 12:7575 (1981). 293 cells are grown to maximal density in a spinner flask and 700 μg pRK5-PRO256, pRK5-PRO34421, pRK5-PRO334, pRK5-PRO770, pRK5-PRO983, pRK5-PRO1009, pRK5-PRO1107, pRK5-PRO1158, pRK5-PRO1250, pRK5-PRO1317, pRK5-PRO4334, pRK5-PRO4395, pRK5-PRO49192, pRK5-PRO9799, pRK5-PRO21175, pRK5-PRO19837, pRK5-PRO21331, pRK5-PRO23949, PRO697 or pRK5-PRO1480DNA is added. The cells are first concentrated from the spinner flask by centrifugation and washed with PBS. The DNA-dextran precipitate is incubated on the cell pellet for four hours. The cells are treated with 20% glycerol for 90 seconds, washed with tissue culture medium, and re-introduced into the spinner flask containing tissue culture medium, 5 μg/ml bovine insulin and 0.1 μg/ml bovine transferrin. After about four days, the conditioned media is centrifuged and filtered to remove cells and debris. The sample containing expressed PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 can then be concentrated and purified by any selected method, such as dialysis and/or column chromatography.

PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 can be expressed in CHO cells. The pRK5-PRO256, pRK5-PRO34421, pRK5-PRO334, pRK5-PRO770, pRK5-PRO983, pRK5-PRO1009, pRK5-PRO1107, pRK5-PRO1158, pRK5-PRO1250, pRK5-PRO1317, pRK5-PRO4334, pRK5-PRO4395, pRK5-PRO49192, pRK5-PRO9799, pRK5-PRO21175, pRK5-PRO19837, pRK5-PRO21331, pRK5-PRO23949, PRO697 or pRK5-PRO1480 can be transfected into CHO cells using known reagents such as CaPO₄ or DEAE-dextran. As described above, the cell cultures can be incubated, and the medium replaced with culture medium (alone) or medium containing a radiolabel such as ³⁵S-methionine. After determining the presence of PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the culture medium may be replaced with serum free medium. Preferably, the cultures are incubated for about 6 days, and then the conditioned medium is harvested. The medium containing the expressed PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 can then be concentrated and purified by any selected method.

Epitope-tagged PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 may also be expressed in host CHO cells. The PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 may be subcloned out of the pRK5 vector. The subclone insert can undergo PCR to fuse in frame with a selected epitope tag such as a poly-his tag into a Baculovirus expression vector. The poly-his tagged PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 insertcan then be subcloned into a SV40 driven vector containing a selection marker such as DHFR for selection of stable clones. Finally, the CHO cells can be transfected (as described above) with the SV40 driven vector. Labeling may be performed, as described above, to verify expression. The culture medium containing the expressed poly-His tagged PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 can then be concentrated and purified by any selected method, such as by Ni²⁺-chelate affinity chromatography.

PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 may also be expressed in CHO and/or COS cells by a transient expression procedure or in CHO cells by another stable expression procedure.

Stable expression in CHO cells is performed using the following procedure. The proteins are expressed as an IgG construct (immunoadhesin), in which the coding sequences for the soluble forms (e.g. extracellular domains) of the respective proteins are fused to an IgG1 constant region sequence containing the hinge, CH2 and CH2 domains and/or is a poly-His tagged form.

Following PCR amplification, the respective DNAs are subcloned in a CHO expression vector using standard techniques as described in Ausubel et al., Current Protocols of Molecular Biology, Unit 3.16, John Wiley and Sons (1997). CHO expression vectors are constructed to have compatible restriction sites 5′ and 3′ of the DNA of interest to allow the convenient shuttling of cDNA's. The vector used expression in CHO cells is as described in Lucas et al., Nucl. Acids Res. 24:9 (1774-1779 (1996), and uses the SV40 early promoter/enhancer to drive expression of the cDNA of interest and dihydrofolate reductase (DHFR). DHFR expression permits selection for stable maintenance of the plasmid following transfection.

Twelve micrograms of the desired plasmid DNA is introduced into approximately 10 million CHO cells using commercially available transfection reagents Superfect® (Qiagen), Dosper® or Fugene® (Boehringer Mannheim). The cells are grown as described in Lucas et al., supra. Approximately 3×10⁷ cells are frozen in an ampule for further growth and production as described below.

The ampules containing the plasmid DNA are thawed by placement into water bath and mixed by vortexing. The contents are pipetted into a centrifuge tube containing 10 mLs of media and centrifuged at 1000 rpm for 5 minutes. The supernatant is aspirated and the cells are resuspended in 10 mL of selective media (0.2 μm filtered PS20 with 5% 0.2 μm diafiltered fetal bovine serum). The cells are then aliquoted into a 100 mL spinner containing 90 mL of selective media. After 1-2 days, the cells are transferred into a 250 mL spinner filled with 150 mL selective growth medium and incubated at 37° C. After another 2-3 days, 250 mL, 500 mL and 2000 mL spinners are seeded with 3×10⁵ cells/mL. The cell media is exchanged with fresh media by centrifugation and resuspension in production medium. Although any suitable CHO media may be employed, a production medium described in U.S. Pat. No. 5,122,469, issued Jun. 16, 1992 may actually be used. A 3 L production spinner is seeded at 1.2×10⁶ cells/mL. On day 0, the cell number pH i.e. determined. On day 1, the spinner is sampled and sparging with filtered air is commenced. On day 2, the spinner is sampled, the temperature shifted to 33° C., and 30 mL of 500 g/L glucose and 0.6 mL of 10% antifoam (e.g., 35% polydimethylsiloxane emulsion, Dow Corning 365 Medical Grade Emulsion) taken. Throughout the production, the pH is adjusted as necessary to keep it at around 7.2. After 10 days, or until the viability dropped below 70%, the cell culture is harvested by centrifugation and filtering through a 0.22 μm filter. The filtrate was either stored at 4° C. or immediately loaded onto columns for purification.

For the poly-His tagged constructs, the proteins are purified using a Ni-NTA column (Qiagen). Before purification, imidazole is added to the conditioned media to a concentration of 5 mM. The conditioned media is pumped onto a 6 ml Ni-NTA column equilibrated in 20 mM Hepes, pH 7.4, buffer containing 0.3 M NaCl and 5 mM imidazole at a flow rate of 4-5 ml/min. at 4° C. After loading, the column is washed with additional equilibration buffer and the protein eluted with equilibration buffer containing 0.25 M imidazole. The highly purified protein is subsequently desalted into a storage buffer containing 10 mM Hepes, 0.14 M NaCl and 4% mannitol, pH 6.8, with a 25 ml G25 Superfine (Pharmacia) column and stored at −80° C.

Immunoadhesin (Fc-containing) constructs are purified from the conditioned media as follows. The conditioned medium is pumped onto a 5 ml Protein A column (Pharmacia) which-had been equilibrated in 20 mM Na phosphate buffer, pH 6.8. After loading, the column is washed extensively with equilibration buffer before elution with 100 mM citric acid, pH 3.5. The eluted protein is immediately neutralized by collecting 1 ml fractions into tubes containing 275 μL of 1 M Tris buffer, pH 9. The highly purified protein is subsequently desalted into storage buffer as described above for the poly-His tagged proteins. The homogeneity is assessed by SDS polyacrylamide gels and by N-terminal amino acid sequencing by Edman degradation.

### Example 25

### Expression of PRO256. PRO34421, PRO334, PRO770. PRO983, PRO1009, PRO1107, PRO1158, PRO1250. PRO1317, PRO4334, PRO4395, PRO49192, PRO9799, PRO21175, PRO19837, PRO21331, PRO23949, PRO697 or PRO1480 in Yeast

The following method describes recombinant expression of PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 in yeast.

First, yeast expression vectors are constructed for intracellular production or secretion of PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 from the ADH2/GAPDH promoter. DNA encoding PRO256, PRO34421, PRO334, PRO770, PRO983, PRO1009, PRO107, PRO1158, PRO1250, PRO1317, PRO4334, PRO4395, PRO49192, PRO9799, PRO21175, PRO19837, PRO21331, PRO23949, PRO697 or PRO1480 and the promoter is inserted into suitable restriction enzyme sites in the selected plasmid to direct intracellular expression of PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480. For secretion, DNA encoding PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 can be cloned into the selected plasmid, together with DNA encoding the ADH2/GAPDH promoter, a native PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 signal peptide or other mammalian signal peptide, or, for example, a yeast alpha-factor or invertase secretory signal/leader sequence, and linker sequences (if needed) for expression of PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480.

Yeast cells, such as yeast strain AB 110, can then be transformed with the expression plasmids described above and cultured in selected fermentation media. The transformed yeast supernatants can be analyzed by precipitation with 10% trichloroacetic acid and separation by SDS-PAGE, followed by staining of the gels with Coomassie Blue stain.

Recombinant PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 can subsequently be isolated and purified by removing the yeast cells from the fermentation medium by centrifugation and then concentrating the medium using selected cartridge filters. The concentrate containing PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 may further be purified using selected column chromatography resins.

### Example 26

### Expression of PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331L PRO23949, PRO697 or PRO1480 in Baculovirus-Infected Insect Cells

The following method describes recombinant expression of PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 in Baculovirus-infected insect cells.

The sequence coding for PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 is fused upstream of an epitope tag contained within a baculovirus expression vector. Such epitope tags include poly-his tags and immunoglobulin tags (like Fc regions of IgG). A variety of plasmids may be employed, including plasmids derived from commercially available plasmids such as pVL1393 (Novagen). Briefly, the sequence encoding PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 or the desired portion of the coding sequence of PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 such as the sequence encoding the extracellular domain of a transmembrane protein or the sequence encoding the mature protein if the protein is extracellular is amplified by PCR with primers complementary to the 5′ and 3′ regions. The 5′ primer may incorporate flanking (selected) restriction enzyme sites. The product is then digested with those selected restriction enzymes and subcloned into the expression vector.

Recombinant baculovirus is generated by co-transfecting the above plasmid and BaculoGold™ virus DNA (Pharmingen) into Spodoptera frugiperda (“Sf9”) cells (ATCC CRL 1711) using lipofectin (commercially available from GIBCO-BRL). After 4-5 days of incubation at 28° C., the released viruses are harvested and used for further amplifications. Viral infection and protein expression are performed as described by O'Reilley et al., Baculovirus expression vectors: A Laboratory Manual, Oxford: Oxford University Press (1994).

Expressed poly-his tagged PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 can then be purified, for example, by Ni²⁺-chelate affinity chromatography as follows. Extracts are prepared from recombinant virus-infected Sf9 cells as described by Rupert et al., Nature, 362:175-179 (1993). Briefly, Sf9 cells are washed, resuspended in sonication buffer (25 mL Hepes, pH 7.9; 12.5 mM MgCl₂; 0.1 mM EDTA; 10% glycerol; 0.1% NP-40; 0.4 M KCl), and sonicated twice for 20 seconds on ice. The sonicates are cleared by centrifugation, and the supernatant is diluted 50-fold in loading buffer (50 mM phosphate, 300 mM NaCl, 10% glycerol, pH 7.8) and filtered through a 0.45 μm filter. A Ni²⁺-NTA agarose column (commercially available from Qiagen) is prepared with a bed volume of 5 mL, washed with 25 mL of water and equilibrated with 25 mL of loading buffer. The filtered cell extract is loaded onto the column at 0.5 mL per minute. The column is washed to baseline A₂₈₀ with loading buffer, at which point fraction collection is started. Next, the column is washed with a secondary wash buffer (50 mM phosphate; 300 mM NaCl, 10% glycerol, pH 6.0), which elutes nonspecifically bound protein. After reaching A₂₈₀ baseline again, the column is developed with a 0 to 500 mM Imidazole gradient in the secondary wash buffer. One mL fractions are collected and analyzed by SDS-PAGE and silver staining or Western blot with Ni²⁺-NTA-conjugated to alkaline phosphatase (Qiagen). Fractions containing the eluted His₁₀-tagged PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 are pooled and dialyzed against loading buffer.

Alternatively, purification of the IgG tagged (or Fc tagged) PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 can be performed using known chromatography techniques, including for instance, Protein A or protein G column chromatography.

### Example 27

### Preparation of Antibodies that Bind PRO256, PRO34421, PRO334, PRO770, PRO983, PRO1009, PRO107, PRO1158, PRO1250, PRO1317, PRO4334, PRO4395, PRO49192, PRO9799, PRO21175, PRO19837, PRO21331, PRO23949, PRO697 or PRO1480

This example illustrates preparation of monoclonal antibodies which can specifically bind PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480.

Techniques for producing the monoclonal antibodies are known in the art and are described, for instance, in Goding, supra. Immunogens that may be employed include purified PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides, fusion proteins containing PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides, and cells expressing recombinant PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides on the cell surface. Selection of the immunogen can be made by the skilled artisan without undue experimentation.

Mice, such as Balb/c, are immunized with the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 immunogen emulsified in complete Freund's adjuvant and injected subcutaneously or intraperitoneally in an amount from 1-100 micrograms. Alternatively, the immunogen is emulsified in MPL-TDM adjuvant (Ribi Immunochemical Research, Hamilton, Mont.) and injected into the animal's hind foot pads. The immunized mice are then boosted 10 to 12 days later with additional immunogen emulsified in the selected adjuvant. Thereafter, for several weeks, the mice may also be boosted with additional immunization injections. Serum samples may be periodically obtained from the mice by retro-orbital bleeding for testing in ELISA assays to detect anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibodies.

After a suitable antibody titer has been detected, the animals “positive” for antibodies can be injected with a final intravenous injection of PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480. Three to four days later, the mice are sacrificed and the spleen cells are harvested. The spleen cells are then fused (using 35% polyethylene glycol) to a selected murine myeloma cell line such as P3X63AgU.1, available from ATCC, No. CRL 1597. The fusions generate hybridoma cells which can then be plated in 96 well tissue culture plates containing HAT (hypoxanthine, aminopterin, and thymidine) medium to inhibit proliferation of non-fused cells, myeloma hybrids, and spleen cell hybrids.

The hybridoma cells will be screened in an ELISA for reactivity against PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480. Determination of “positive” hybridoma cells secreting the desired monoclonal antibodies against PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 is within the skill in the art.

The positive hybridoma cells can be injected intraperitoneally into syngeneic Balb/c mice to produce ascites containing the anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 monoclonal antibodies. Alternatively, the hybridoma cells can be grown in tissue culture flasks or roller bottles. Purification of the monoclonal antibodies produced in the ascites can be accomplished using ammonium sulfate precipitation, followed by gel exclusion chromatography. Alternatively, affinity chromatography based upon binding of antibody to protein A or protein G can be employed.

### Example 28

### Purification of PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 Polypeptides Using Specific Antibodies

Native or recombinant PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides may be purified by a variety of standard techniques in the art of protein purification. For example, pro-PRO256, pro-PRO34421, pro-PRO334, pro-PRO770, pro-PRO983, pro-PRO1009, pro-PRO1107, pro-PRO1158, pro-PRO1250, pro-PRO1317, pro-PRO4334, pro-PRO4395, pro-PRO49192,pro-PRO9799, pro-PRO21175,pro-PRO19837, pro-PRO21331, pro-PRO23949, pro-PRO697 or pro-PRO1480 polypeptide, mature PRO256, mature PRO34421, mature PRO334, mature PRO770, mature PRO983, mature PRO1009, mature PRO1107, mature PRO1158, mature PRO1250, mature PRO1317, mature PRO4334, mature PRO4395, mature PRO49192, mature PRO9799, mature PRO21175, mature PRO19837, mature PRO21331, mature PRO23949, mature PRO697 or mature PRO1480 polypeptide, or pre-PRO256, pre-PRO34421, pre-PRO334, pre-PRO770, pre-PRO983, pre-PRO1009, pre-PRO1107, pre-PRO1158, pre-PRO1250, pre-PRO1317, pre-PRO4334, pre-PRO4395, pre-PRO49192, pre-PRO9799, pre-PRO21175, pre-PRO19837, pre-PRO21331, pre-PRO23949, pre-PRO697 or pre-PRO1480 polypeptide is purified by immunoaffinity chromatography using antibodies specific for the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide of interest. In general, an immunoaffinity column is constructed by covalently coupling the anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody to an activated chromatographic resin.

Polyclonal immunoglobulins are prepared from immune sera either by precipitation with ammonium sulfate or by purification on immobilized Protein A (Pharmacia LKB Biotechnology, Piscataway, N.J.). Likewise, monoclonal antibodies are prepared from mouse ascites fluid by ammonium sulfate precipitation or chromatography on immobilized Protein A. Partially purified immunoglobulin is covalently attached to a chromatographic resin such as CnBr-activated SEPHAROSE™ (Pharmacia LKB Biotechnology). The antibody is coupled to the resin, the resin is blocked, and the derivative resin is washed according to the manufacturer's instructions.

Such an immunoaffinity column is utilized in the purification of PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide by preparing a fraction from cells containing PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide in a soluble form. This preparation is derived by solubilization of the whole cell or of a subcellular fraction obtained via differential centrifugation by the addition of detergent or by other methods well known in the art. Alternatively, soluble polypeptide containing a signal sequence may be secreted in useful quantity into the medium in which the cells are grown.

A soluble PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide-containing preparation is passed over the immunoaffinity column, and the column is washed under conditions that allow the preferential absorbance of PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide (e.g., high ionic strength buffers in the presence of detergent). Then, the column is eluted under conditions that disrupt antibody/PRO256, antibody/PRO34421, antibody/PRO334, antibody/PRO770, antibody/PRO983, antibody/PRO1009, antibody/PRO1107, antibody/PRO1158, antibody/PRO1250, antibody/PRO1317, antibody/PRO4334, antibody/PRO4395, antibody/PRO49192, antibody/PRO9799, antibody/PRO21175, antibody/PRO19837, antibody/PRO21331, antibody/PRO23949, antibody/PRO697 or antibody/PRO1480 polypeptide binding (e.g., a low pH buffer such as approximately pH 2-3, or a high concentration of a chaotrope such as urea or thiocyanate ion), and PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide is collected.

### Example 29

### Drug Screening

This invention is particularly useful for screening compounds by using PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptides or binding fragment thereof in any of a variety of drug screening techniques. The PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide or fragment employed in such a test may either be free in solution, affixed to a solid support, borne on a cell surface, or located intracellularly. One method of drug screening utilizes eukaryotic or prokaryotic host cells which are stably transformed with recombinant nucleic acids expressing the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide or fragment. Drugs are screened against such transformed cells in competitive binding assays. Such cells, either in viable or fixed form, can be used for standard binding assays. One may measure, for example, the formation of complexes between PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide or a fragment and the agent being tested. Alternatively, one can examine the diminution in complex formation between the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide and its target cell or target receptors caused by the agent being tested.

Thus, the present invention provides methods of screening for drugs or any other agents which can affect a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide-associated disease or disorder. These methods comprise contacting such an agent with an PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide or fragment thereof and assaying (I) for the presence of a complex between the agent and the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide or fragment, or (ii) for the presence of a complex between the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide or fragment and the cell, by methods well known in the art. In such competitive binding assays, the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide or fragment is typically labeled. After suitable incubation, free PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide or fragment is separated from that present in bound form, and the amount of free or uncomplexed label is a measure of the ability of the particular agent to bind to PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide or to interfere with the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide/cell complex.

Another technique for drug screening provides high throughput screening for compounds having suitable binding affinity to a polypeptide and is described in detail in WO 84/03564, published on Sep. 13, 1984. Briefly stated, large numbers of different small peptide test compounds are synthesized on a solid substrate, such as plastic pins or some other surface. As applied to a PRO256, PRO34421, PRO334, PRO770, PRO983, PRO1009, PRO1107, PRO1158, PRO1250, PRO11317, PRO4334, PRO4395, PRO49192, PRO9799, PRO21175, PRO19837, PRO21331, PRO23949, PRO697 or PRO1480 polypeptide, the peptide test compounds are reacted with PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide and washed. Bound PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide is detected by methods well known in the art. Purified PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide can also be coated directly onto plates for use in the aforementioned drug screening techniques. In addition, non-neutralizing antibodies can be used to capture the peptide and immobilize it on the solid support.

This invention also contemplates the use of competitive drug screening assays in which neutralizing antibodies capable of binding PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide specifically compete with a test compound for binding to PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide or fragments thereof. In this manner, the antibodies can be used to detect the presence of any peptide which shares one or more antigenic determinants with PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide.

### Example 30

### Rational Drug Design

The goal of rational drug design is to produce structural analogs of biologically active polypeptide of interest (i.e., a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide) or of small molecules with which they interact, e.g., agonists, antagonists, or inhibitors. Any of these examples can be used to fashion drugs which are more active or stable forms of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide or which enhance or interfere with the function of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide in vivo (c.f., Hodgson, Bio/Technology, 2: 19-21 (1991)).

In one approach, the three-dimensional structure of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, or of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide-inhibitor complex, is determined by x-ray crystallography, by computer modeling or, most typically, by a combination of the two approaches. Both the shape and charges of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide must be ascertained to elucidate the structure and to determine active site(s) of the molecule. Less often, useful information regarding the structure of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide may be gained by modeling based on the structure of homologous proteins. In both cases, relevant structural information is used to design analogous PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide-like molecules or to identify efficient inhibitors. Useful examples of rational drug design may include molecules which have improved activity or stability as shown by Braxton and Wells, Biochemistry 31:7796-7801 (1992) or which act as inhibitors, agonists, or antagonists of native peptides as shown by Athauda et al., J. Biochem., 113:742-746 (1993).

It is also possible to isolate a target-specific antibody, selected by functional assay, as described above, and then to solve its crystal structure. This approach, in principle, yields a pharmacore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original receptor. The anti-id could then be used to identify and isolate peptides from banks of chemically or biologically produced peptides. The isolated peptides would then act as the pharmacore.

By virtue of the present invention, sufficient amounts of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide may be made available to perform such analytical studies as X-ray crystallography. In addition, knowledge of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide amino acid sequence provided herein will provide guidance to those employing computer modeling techniques in place of or in addition to x-ray crystallography.

1-149. (canceled)

150. A method of identifying a phenotype associated with a disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising:
(a) providing a non-human transgenic animal whose genome comprises a disruption of a gene which is an ortholog of a human gene that encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide;
(b) measuring a physiological characteristic of the non-human transgenic animal; and
(c) comparing the measured physiological characteristic with that of a gender matched wild-type animal, wherein the physiological characteristic of the non-human transgenic animal that differs from the physiological characteristic of the wild-type animal is identified as a phenotype resulting from the gene disruption in the non-human transgenic animal.

151. The method of claim 150, wherein the non-human transgenic animal is heterozygous for the disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide.

152. The method of claim 150, wherein the phenotype exhibited by the non-human transgenic animal as compared with gender matched wild-type littermates is at least one of the following: a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality.

153. The method of claim 152, wherein the neurological disorder is an increased anxiety-like response during open field activity testing.

154. The method of claim 152, wherein the neurological disorder is a decreased anxiety-like response during open field activity testing.

155. The method of claim 152, wherein the neurological disorder is an abnormal circadian rhythm during home-cage activity testing.

156. The method of claim 152, wherein the neurological disorder is an enhanced motor coordination during inverted screen testing.

157. The method of claim 152, wherein the neurological disorder is an impaired motor coordination during inverted screen testing.

158. The method of claim 152, wherein the neurological disorder is depression, generalized anxiety disorders, attention deficit disorder, sleep disorder, hyperactivity disorder, obsessive compulsive disorder, schizophrenia, cognitive disorders, hyperalgesia or sensory disorders.

159. The method of claim 152, wherein the eye abnormality is a retinal abnormality.

160. The method of claim 152, wherein the eye abnormality is consistent with vision problems or blindness.

161. The method of claim 159, wherein the retinal abnormality is consistent with retinitis pigmentosa.

162. The method of claim 159, wherein the retinal abnormality is characterized by retinal degeneration or retinal dysplasia.

163. The method of claim 159, wherein the retinal abnormality is consistent with retinal dysplasia, various retinopathies, including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstrom's syndrome, Cockayne's syndrome, dysplasia spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis.

164. The method of claim 152, wherein the eye abnormality is a cataract.

165. The method of claim 164, wherein the cataract is consistent with systemic diseases such as human Down's syndrome, Hallerman-Streiff syndrome, Lowe syndrome, galactosemia, Marfan syndrome, Trismoy 13-15, Alport syndrome, myotonic dystrophy, Fabry disease, hypoparathyroidism or Conradi syndrome.

166. The method of claim 152, wherein the developmental abnormality comprises embryonic lethality or reduced viability.

167. The method of claim 152, wherein the cardiovascular, endothelial or angiogenic disorders are arterial diseases, such as diabetes mellitus; papilledema; optic atrophy; atherosclerosis; angina; myocardial infarctions such as acute myocardial infarctions, cardiac hypertrophy, and heart failure such as congestive heart failure; hypertension; inflammatory vasculitides; Reynaud's disease and Reynaud's phenomenon; aneurysms and arterial restenosis; venous and lymphatic disorders such as thrombophlebitis, lymphangitis, and lymphedema; peripheral vascular disease; cancer such as vascular tumors, e.g., hemangioma (capillary and cavernous), glomus tumors, telangiectasia, bacillary angiomatosis, hemangioendothelioma, angiosarcoma, hemangiopericytoma, Kaposi's sarcoma, lymphangioma, and lymphangiosarcoma; tumor angiogenesis; trauma such as wounds, burns, and other injured tissue, implant fixation, scarring; ischemia reperfusion injury; rheumatoid arthritis; cerebrovascular disease; renal diseases such as acute renal failure, or osteoporosis.

168. The method of claim 152, wherein the immunological disorders are systemic lupus erythematosis; rheumatoid arthritis; juvenile chronic arthritis; spondyloarthropathies; systemic sclerosis (scleroderma); idiopathic inflammatory myopathies (dermatomyositis, polymyositis); Sjögren's syndrome; systemic vasculitis; sarcoidosis; autoimmune hemolytic anemia (immune pancytopenia, paroxysmal nocturnal hemoglobinuria); autoimmune thrombocytopenia (idiopathic thrombocytopenic purpura, immune-mediated thrombocytopenia); thyroiditis (Grave's disease, Hashimoto's thyroiditis, juvenile lymphocytic thyroiditis, atrophic thyroiditis); diabetes mellitus; immune-mediated renal disease (glomerulonephritis, tubulointerstitial nephritis); demyelinating diseases of the central and peripheral nervous systems such as multiple sclerosis, idiopathic demyelinating polyneuropathy or Guillain-Barré syndrome, and chronic inflammatory demyelinating polyneuropathy; hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis; inflammatory bowel disease (ulcerative colitis: Crohn's disease); gluten-sensitive enteropathy, and Whipple's disease; autoimmune or immune-mediated skin diseases including bullous skin diseases, erythema multiform and contact dermatitis, psoriasis; allergic diseases such as asthma, allergic rhinitis, atopic dermatitis, food hypersensitivity and urticaria; immunologic diseases of the lung such as eosinophilic pneumonias, idiopathic pulmonary fibrosis and hypersensitivity pneumonitis; or transplantation associated diseases including graft rejection and graft-versus-host disease.

169. The method of claim 152, wherein the bone metabolic abnormality or disorder is arthritis, osteoporosis or osteopetrosis.

170. The method of claim 150, wherein the non-human transgenic animal exhibits at least one of the following physiological characteristics compared with gender matched wild-type littermates: a decreased anxiety-like response during open field activity testing; an abnormal circadian rhythm during home-cage activity testing; an enhanced motor coordination during inverted screen testing; exophthalamus in functional observation testing; severe retinal degeneration marked by attenuated retinal vessels; retinal microaneurisms; decreased mean artery-to-vein ratio; decreased lens size; mature cataracts; an increased mean serum cholesterol level; an increased mean serum triglyceride level; a decreased mean serum cholesterol level; an enhanced glucose tolerance; a decreased glucose tolerance; an increased mean serum insulin level; a decreased mean serum insulin level; a decreased mean serum IgG1 and IgG2a responses to an ovalbumin challenge; an increased mean serum IgG2a response to an ovalbumin challenge; an impaired IgG2a response to an ovalbumin challenge; a decreased mean absolute blood neutrophil count; an increased mean serum levels of IgG1, IgG3, IgA, IgG2a and IgG2b; an increased mean serum TNF-alpha and IL6 response to a LPS challenge; a decreased mean platelet count; a reduced level of RBC's, platelets, hemoglobin and hematocrit; an increased mean percent body fat; a decreased skin fibroblast proliferation; an increased skin fibroblast proliferation; an increased total tissue mass (TTM); an increased lean body mass (LBM); an increased bone mineral density (BMD); an increased bone mineral content (BMC), an increased bone mineral content index (BMC/LBM); an increased midshaft femur total area; a decrease in trabecular bone volume and connectivity density; a decreased volumetric bone mineral density; a decreased bone mineral content index (BMC/LBM); a decreased mean bone mineral density in total body, femur and vertebrate; a decreased mean bone mineral density (BMD), a decreased mean trabecular bone volume, decreased thickness, and decreased connectivity density; a decreased body weight and length; a decreased total tissue mass (TTM); a decreased lean body mass (LBM); a decreased total fat mass; a decreased bone mineral content (BMC); a decreased mean volumetric bone mineral density (vBMD) in total body and femur; a decreased femoral midshaft cross-sectional area and thickness; growth retardation with decreased mean body weight and length, decreased mean percent of total body fat, decreased total tissue mass and decreased bone mineral density; a decreased femoral midshaft cortical thickness; cardiomegaly; an impaired renal function; renal mesonephric duct development abnormalities; seminiferous tubular degeneration; greatly reduced viability [only three (−/−) mutant mice survived showing severe growth retardation as compared to the expected 14 (−/−) mutants]; a significant reduction in expected numbers of homozygotes; and embryonic lethality.

171. An isolated cell derived from a non-human transgenic animal whose genome comprises disruption of a gene which is an ortholog of a human gene that encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide.

172. The isolated cell of claim 171 which is a murine cell.

173. The isolated cell of claim 172, wherein the murine cell is an embryonic stem cell.

174. The isolated cell of claim 171, wherein the non-human transgenic animal exhibits at least one of the following phenotypes compared with gender matched wild-type littermates: a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality.

175. A method of identifying an agent that modulates a phenotype associated with a disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising:
(a) providing a non-human transgenic animal whose genome comprises a disruption of a gene which is an ortholog of a human gene that encodes for the PRO256, PRO34421, PRO334, PRO770, PRO983, PRO100 9, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide;
(b) measuring a physiological characteristic of the non-human transgenic animal of (a);
(c) comparing the measured physiological characteristic of (b) with that of a gender matched wild-type animal, wherein the physiological characteristic of the non-human transgenic animal that differs from the physiological characteristic of the wild-type animal is identified as a phenotype resulting from the gene disruption in the non-human transgenic animal;
(d) administering a test agent to the non-human transgenic animal of (a); and
(e) determining whether the test agent modulates the identified phenotype associated with gene disruption in the non-human transgenic animal.

176. The method of claim 175, wherein the phenotype associated with the gene disruption comprises a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality.

177. The method of claim 176, wherein the neurological disorder is an increased anxiety-like response during open field activity testing.

178. The method of claim 176, wherein the neurological disorder is a decreased anxiety-like response during open field activity testing.

179. The method of claim 176, wherein the neurological disorder is an abnormal circadian rhythm during home-cage activity testing.

180. The method of claim 176, wherein the neurological disorder is an enhanced motor coordination during inverted screen testing.

181. The method of claim 176, wherein the neurological disorder is an impaired motor coordination during inverted screen testing.

182. The method of claim 176, wherein the neurological disorder is depression, generalized anxiety disorders, attention deficit disorder, sleep disorder, hyperactivity disorder, obsessive compulsive disorder, schizophrenia, cognitive disorders, hyperalgesia or sensory disorders.

183. The method of claim 176, wherein the eye abnormality is a retinal abnormality.

184. The method of claim 176, wherein the eye abnormality is consistent with vision problems or blindness.

185. The method of claim 183, wherein the retinal abnormality is consistent with retinitis pigmentosa.

186. The method of claim 183, wherein the retinal abnormality is characterized by retinal degeneration or retinal dysplasia.

187. The method of claim 183, wherein the retinal abnormality is consistent with retinal dysplasia, various retinopathies, including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstrom's syndrome, Cockayne's syndrome, dysplasia spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis.

188. The method of claim 176, wherein the eye abnormality is a cataract.

189. The method of claim 188, wherein the cataract is consistent with systemic diseases such as human Down's syndrome, Hallerman-Streiff syndrome, Lowe syndrome, galactosemia, Marfan syndrome, Trismoy 13-15, Alport syndrome, myotonic dystrophy, Fabry disease, hypoparathyroidism or Conradi syndrome.

190. The method of claim 176, wherein the developmental abnormality comprises embryonic lethality or reduced viability.

191. The method of claim 176, wherein the cardiovascular, endothelial or angiogenic disorders are arterial diseases, such as diabetes mellitus; papilledema; optic atrophy; atherosclerosis; angina; myocardial infarctions such as acute myocardial infarctions, cardiac hypertrophy, and heart failure such as congestive heart failure; hypertension; inflammatory vasculitides; Reynaud's disease and Reynaud's phenomenon; aneurysms and arterial restenosis; venous and lymphatic disorders such as thrombophlebitis, lymphangitis, and lymphedema; peripheral vascular disease; cancer such as vascular tumors, e.g., hemangioma (capillary and cavernous), glomus tumors, telangiectasia, bacillary angiomatosis, hemangioendothelioma, angiosarcoma, hemangiopericytoma, Kaposi's sarcoma, lymphangioma, and lymphangiosarcoma; tumor angiogenesis; trauma such as wounds, burns, and other injured tissue, implant fixation, scarring; ischemia reperfusion injury; rheumatoid arthritis; cerebrovascular disease; renal diseases such as acute renal failure, or osteoporosis.

192. The method of claim 176, wherein the immunological disorders are systemic lupus erythematosis; rheumatoid arthritis; juvenile chronic arthritis spondyloarthropathies; systemic sclerosis (scleroderma); idiopathic inflammatory myopathies (dermatomyositis, polymyositis); Sjögren's syndrome; systemic vasculitis; sarcoidosis; autoimmune hemolytic anemia (immune pancytopenia, paroxysmal nocturnal hemoglobinuria); autoimmune thrombocytopenia (idiopathic thrombocytopenic purpura, immune-mediated thrombocytopenia); thyroiditis (Grave's disease, Hashimoto's thyroiditis, juvenile lymphocytic thyroiditis, atrophic thyroiditis); diabetes mellitus; immune-mediated renal disease (glomerulonephritis, tubulointerstitial nephritis); demyelinating diseases of the central and peripheral nervous systems such as multiple sclerosis, idiopathic demyelinating polyneuropathy or Guillain-Barré syndrome, and chronic inflammatory demyelinating polyneuropathy; hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis; inflammatory bowel disease (ulcerative colitis: Crohn's disease); gluten-sensitive enteropathy, and Whipple's disease; autoimmune or immune-mediated skin diseases including bullous skin diseases, erythema multiform and contact dermatitis, psoriasis; allergic diseases such as asthma, allergic rhinitis, atopic dermatitis, food hypersensitivity and urticaria; immunologic diseases of the lung such as eosinophilic pneumonia, idiopathic pulmonary fibrosis and hypersensitivity pneumonitis; or transplantation-associated diseases including graft rejection and graft-versus-host disease.

193. The method of claim 176, wherein said bone metabolic abnormality or disorder is arthritis, osteoporosis or osteopetrosis.

194. The method of claim 175, wherein the non-human transgenic animal exhibits at least one of the following physiological characteristics compared with gender matched wild-type littermates: a decreased anxiety-like response during open field activity testing; an abnormal circadian rhythm during home-cage activity testing; an enhanced motor coordination during inverted screen testing; exophthalamus in functional observation testing; severe retinal degeneration marked by attenuated retinal vessels; retinal microaneurisms; decreased mean artery-to-vein ratio; decreased lens size; mature cataracts; an increased mean serum cholesterol level; an increased mean serum triglyceride level; a decreased mean serum cholesterol level; an enhanced glucose tolerance; a decreased glucose tolerance; an increased mean serum insulin level; a decreased mean serum insulin level; a decreased mean serum IgG1 and IgG2a responses to an ovalbumin challenge; an increased mean serum IgG2a response to an ovalbumin challenge; an impaired IgG2a response to an ovalbumin challenge; a decreased mean absolute blood neutrophil count; an increased mean serum levels of IgG1, IgG3, IgA, IgG2a and IgG2b; an increased mean serum TNF-alpha and IL6 response to a LPS challenge; a decreased mean platelet count; a reduced level of RBC's, platelets, hemoglobin and hematocrit; an increased mean percent body fat; a decreased skin fibroblast proliferation; an increased skin fibroblast proliferation; an increased total tissue mass (TTM); an increased lean body mass (LBM); an increased bone mineral density (BMD); an increased bone mineral content (BMC), an increased bone mineral content index (BMC/LBM); an increased midshaft femur total area; a decrease in trabecular bone volume and connectivity density; a decreased volumetric bone mineral density; a decreased bone mineral content index (BMC/LBM); a decreased mean bone mineral density in total body, femur and vertebrate; a decreased mean bone mineral density (BMD), a decreased mean trabecular bone volume, decreased thickness, and decreased connectivity density; a decreased body weight and length; a decreased total tissue mass (TTM); a decreased lean body mass (LBM); a decreased total fat mass; a decreased bone mineral content (BMC); a decreased mean volumetric bone mineral density (vBMD) in total body and femur; a decreased femoral midshaft cross-sectional area and thickness; growth retardation with decreased mean body weight and length, decreased mean percent of total body fat, decreased total tissue mass and decreased bone mineral density; a decreased femoral midshaft cortical thickness; cardiomegaly; an impaired renal function; renal mesonephric duct development abnormalities; seminiferous tubular degeneration; greatly reduced viability [only three (−/−) mutant mice survived showing severe growth retardation as compared to the expected 14 (−/−) mutants]; a significant reduction in expected numbers of homozygotes; and embryonic lethality.

195. An agent identified by the method of claim 175.

196. The agent of claim 195 which is an agonist or antagonist of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide.

197. The agent of claim 196, wherein the agonist is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.

198. The agent of claim 196, wherein the antagonist is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.

199. A method of identifying an agent that modulates a physiological characteristic associated with a disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising:
(a) providing a non-human transgenic animal whose genome comprises a disruption of a gene which is an ortholog of a human gene that encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide;
(b) measuring a physiological characteristic exhibited by the non-human transgenic animal of (a);
(c) comparing the measured physiological characteristic of (b) with that of a gender matched wild-type animal, wherein the physiological characteristic exhibited by the non-human transgenic animal that differs from the physiological characteristic exhibited by the wild-type animal is identified as a physiological characteristic associated with gene disruption;
(d) administering a test agent to the non-human transgenic animal of (a); and
(e) determining whether the physiological characteristic associated with gene disruption is modulated.

200. The method of claim 199, wherein the non-human transgenic animal exhibits at least one of the following physiological characteristics compared with gender matched wild-type littermates: a decreased anxiety-like response during open field activity testing; an abnormal circadian rhythm during home-cage activity testing; an enhanced motor coordination during inverted screen testing; exophthalamus in functional observation testing; severe retinal degeneration marked by attenuated retinal vessels; retinal microaneurisms; decreased mean artery-to-vein ratio; decreased lens size; mature cataracts; an increased mean serum cholesterol level; an increased mean serum triglyceride level; a decreased mean serum cholesterol level; an enhanced glucose tolerance; a decreased glucose tolerance; an increased mean serum insulin level; a decreased mean serum insulin level; a decreased mean serum IgG1 and IgG2a responses to an ovalbumin challenge; an increased mean serum IgG2a response to an ovalbumin challenge; an impaired IgG2a response to an ovalbumin challenge; a decreased mean absolute blood neutrophil count; an increased mean serum levels of IgG1, IgG3, IgA, IgG2a and IgG2b; an increased mean serum TNF-alpha and IL6 response to a LPS challenge; a decreased mean platelet count; a reduced level of RBC's, platelets, hemoglobin and hematocrit; an increased mean percent body fat; a decreased skin fibroblast proliferation; an increased skin fibroblast proliferation; an increased total tissue mass (TTM); an increased lean body mass (LBM); an increased bone mineral density (BMD); an increased bone mineral content (BMC), an increased bone mineral content index (BMC/LBM); an increased midshaft femur total area; a decrease in trabecular bone volume and connectivity density; a decreased volumetric bone mineral density; a decreased bone mineral content index (BMC/LBM); a decreased mean bone mineral density in total body, femur and vertebrate; a decreased mean bone mineral density (BMD), a decreased mean trabecular bone volume, decreased thickness, and decreased connectivity density; a decreased body weight and length; a decreased total tissue mass (TTM); a decreased lean body mass (LBM); a decreased total fat mass; a decreased bone mineral content (BMC); a decreased mean volumetric bone mineral density (vBMD) in total body and femur; a decreased femoral midshaft cross-sectional area and thickness; growth retardation with decreased mean body weight and length, decreased mean percent of total body fat, decreased total tissue mass and decreased bone mineral density; a decreased femoral midshaft cortical thickness; cardiomegaly; an impaired renal function; renal mesonephric duct development abnormalities; seminiferous tubular degeneration; greatly reduced viability [only three (−/−) mutant mice survived showing severe growth retardation as compared to the expected 14 (−/−) mutants]; a significant reduction in expected numbers of homozygotes; and embryonic lethality.

201. An agent identified by the method of claim 199.

202. The agent of claim 201 which is an agonist or antagonist of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide.

203. The agent of claim 202, wherein the agonist is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.

204. The agent of claim 202, wherein the antagonist is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti.—PRO49192, anti-PRO9799, anti-PRO21175, anti-PRO19837, anti-PRO21331, anti-PRO23949, anti-PRO697 or anti-PRO1480 antibody.

205. A method of identifying an agent which modulates a behavior associated with a disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising:
(a) providing a non-human transgenic animal whose genome comprises a disruption of a gene which is an ortholog of a human gene that encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide;
(b) observing the behavior exhibited by the non-human transgenic animal of (a);
(c) comparing the observed behavior of (b) with that of a gender matched wild-type animal, wherein the observed behavior exhibited by the non-human transgenic animal that differs from the observed behavior exhibited by the wild-type animal is identified as a behavior associated with gene disruption;
(d) administering a test agent to the non-human transgenic animal of (a); and
(e) determining whether the agent modulates the behavior associated with gene disruption.

206. The method of claim 205, wherein the behavior is an increased anxiety-like response during open field activity testing.

207. The method of claim 205, wherein the behavior is a decreased anxiety-like response during open field activity testing.

208. The method of claim 205, wherein the behavior is an abnormal circadian rhythm during home-cage activity testing.

209. The method of claim 205, wherein the behavior is an enhanced motor coordination during inverted screen testing.

210. The method of claim 205, wherein the behavior is an impaired motor coordination during inverted screen testing.

211. The method of claim 205, wherein the behavior is depression, generalized anxiety disorders, attention deficit disorder, sleep disorder, hyperactivity disorder, obsessive compulsive disorder, schizophrenia, cognitive disorders, hyperalgesia or sensory disorders.

212. An agent identified by the method of claim 205.

213. The agent of claim 212 which is an agonist or antagonist of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide.

214. The agent of claim 213, wherein the agonist is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.

215. The agent of claim 213, wherein the antagonist is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.

216. A method of identifying an agent that ameliorates or modulates a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality associated with a disruption in a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising:
(a) providing a non-human transgenic animal whose genome comprises a disruption of a gene which is an ortholog of a human gene that encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide;
(b) administering a test agent to said non-human transgenic animal; and
(c) determining whether said test agent ameliorates or modulates the neurological disorder; cardiovascular, endothelial or angiogenic disorder; eye abnormality; immunological disorder; oncological disorder; bone metabolic abnormality or disorder; lipid metabolic disorder; or developmental abnormality in the non-human transgenic animal.

217. The method of claim 216, wherein the neurological disorder is an increased anxiety-like response during open field activity testing.

218. The method of claim 216, wherein the neurological disorder is a decreased anxiety-like response during open field activity testing.

219. The method of claim 216, wherein the neurological disorder is an abnormal circadian rhythm during home-cage activity testing.

220. The method of claim 216, wherein the neurological disorder is an enhanced motor coordination during inverted screen testing.

221. The method of claim 216, wherein the neurological disorder is an impaired motor coordination during inverted screen testing.

222. The method of claim 216, wherein the neurological disorder is depression, generalized anxiety disorders, attention deficit disorder, sleep disorder, hyperactivity disorder, obsessive compulsive disorder, schizophrenia, cognitive disorders, hyperalgesia or sensory disorders.

223. The method of claim 216, wherein the eye abnormality is a retinal abnormality.

224. The method of claim 216, wherein the eye abnormality is consistent with vision problems or blindness.

225. The method of claim 223, wherein the retinal abnormality is consistent with retinitis pigmentosa.

226. The method of claim 223, wherein the retinal abnormality is characterized by retinal degeneration or retinal dysplasia.

227. The method of claim 223, wherein the retinal abnormality is consistent with retinal dysplasia, various retinopathies, including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstrom's syndrome, Cockayne's syndrome, dysplasia spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis.

228. The method of claim 216, wherein the eye abnormality is a cataract.

229. The method of claim 228, wherein the cataract is a systemic disease such as human Down's syndrome, Hallerman-Streiff syndrome, Lowe syndrome, galactosemia, Marfan syndrome, Trismoy 13-15, Alport syndrome, myotonic dystrophy, Fabry disease, hypoparathyroidism or Conradi syndrome.

230. The method of claim 216, wherein the developmental abnormality comprises embryonic lethality or reduced viability.

231. The method of claim 216, wherein the cardiovascular, endothelial or angiogenic disorders are arterial diseases, such as diabetes mellitus; papilledema; optic atrophy; atherosclerosis; angina; myocardial infarctions such as acute myocardial infarctions, cardiac hypertrophy, and heart failure such as congestive heart failure; hypertension; inflammatory vasculitides; Reynaud's disease and Reynaud's phenomenon; aneurysms and arterial restenosis; venous and lymphatic disorders such as thrombophlebitis, lymphangitis, and lymphedema; peripheral vascular disease; cancer such as vascular tumors, e.g., hemangioma (capillary and cavernous), glomus tumors, telangiectasia, bacillary angiomatosis, hemangioendothelioma, angiosarcoma, hemangiopericytoma, Kaposi's sarcoma, lymphangioma, and lymphangiosarcoma; tumor angiogenesis; trauma such as wounds, burns, and other injured tissue, implant fixation, scarring; ischemia reperfusion injury; rheumatoid arthritis; cerebrovascular disease; renal diseases such as acute renal failure, or osteoporosis.

232. The method of claim 216, wherein the immunological disorders are systemic lupus erythematosis; rheumatoid arthritis; juvenile chronic arthritis; spondyloarthropathies; systemic sclerosis (scleroderma); idiopathic inflammatory myopathies (dermatomyositis, polymyositis); Sjögren's syndrome; systemic vasculitis; sarcoidosis; autoimmune hemolytic anemia (immune pancytopenia, paroxysmal nocturnal hemoglobinuria); autoimmune thrombocytopenia (idiopathic thrombocytopenic purpura, immune-mediated thrombocytopenia); thyroiditis (Grave's disease, Hashimoto's thyroiditis, juvenile lymphocytic thyroiditis, atrophic thyroiditis); diabetes mellitus; immune-mediated renal disease (glomerulonephritis, tubulointerstitial nephritis); demyelinating diseases of the central and peripheral nervous systems such as multiple sclerosis, idiopathic demyelinating polyneuropathy or Guillain-Barré syndrome, and chronic inflammatory demyelinating polyneuropathy; hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis; inflammatory bowel disease (ulcerative colitis: Crohn's disease); gluten-sensitive enteropathy, and Whipple's disease; autoimmune or immune-mediated skin diseases including bullous skin diseases, erythema multiform and contact dermatitis, psoriasis; allergic diseases such as asthma, allergic rhinitis, atopic dermatitis, food hypersensitivity and urticaria; immunologic diseases of the lung such as eosinophilic pneumonia, idiopathic pulmonary fibrosis and hypersensitivity pneumonitis; or transplantation associated diseases including graft rejection and graft-versus-host disease.

233. The method of claim 216, wherein said bone metabolic abnormality or disorder is arthritis, osteoporosis or osteopetrosis.

234. The method of claim 216, wherein the non-human transgenic animal exhibits at least one of the following physiological characteristics compared with gender matched wild-type littermates: a decreased anxiety-like response during open field activity testing; an abnormal circadian rhythm during home-cage activity testing; an enhanced motor coordination during inverted screen testing; exophthalamus in functional observation testing; severe retinal degeneration marked by attenuated retinal vessels; retinal microaneurisms; decreased mean artery-to-vein ratio; decreased lens size; mature cataracts; an increased mean serum cholesterol level; an increased mean serum triglyceride level; a decreased mean serum cholesterol level; an enhanced glucose tolerance; a decreased glucose tolerance; an increased mean serum insulin level; a decreased mean serum insulin level; a decreased mean serum IgG1 and IgG2a responses to an ovalbumin challenge; an increased mean serum IgG2a response to an ovalbumin challenge; an impaired IgG2a response to an ovalbumin challenge; a decreased mean absolute blood neutrophil count; an increased mean serum levels of IgG1, IgG3, IgA, IgG2a and IgG2b; an increased mean serum TNF-alpha and IL6 response to a LPS challenge; a decreased mean platelet count; a reduced level of RBC's, platelets, hemoglobin and hematocrit; an increased mean percent body fat; a decreased skin fibroblast proliferation; an increased skin fibroblast proliferation; an increased total tissue mass (TTM); an increased lean body mass (LBM); an increased bone mineral density (BMD); an increased bone mineral content (BMC), an increased bone mineral content index (BMC/LBM); an increased midshaft femur total area; a decrease in trabecular bone volume and connectivity density; a decreased volumetric bone mineral density; a decreased bone mineral content index (BMC/LBM); a decreased mean bone mineral density in total body, femur and vertebrate; a decreased mean bone mineral density (BMD), a decreased mean trabecular bone volume, decreased thickness, and decreased connectivity density; a decreased body weight and length; a decreased total tissue mass (TTM); a decreased lean body mass (LBM); a decreased total fat mass; a decreased bone mineral content (BMC); a decreased mean volumetric bone mineral density (vBMD) in total body and femur; a decreased femoral midshaft cross-sectional area and thickness; growth retardation with decreased mean body weight and length, decreased mean percent of total body fat, decreased total tissue mass and decreased bone mineral density; a decreased femoral midshaft cortical thickness; cardiomegaly; an impaired renal function; renal mesonephric duct development abnormalities; seminiferous tubular degeneration; greatly reduced viability [only three (−/−) mutant mice survived showing severe growth retardation as compared to the expected 14 (−/−) mutants]; a significant reduction in expected numbers of homozygotes; and embryonic lethality.

235. An agent identified by the method of claim 216.

236. The agent of claim 235 which is an agonist or antagonist of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide.

237. The agent of claim 236, wherein the agonist is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.

238. The agent of claim 236, wherein the antagonist is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.

239. A therapeutic agent identified by the method of claim 216.

240. A method of identifying an agent that modulates the expression of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising:
(a) contacting a test agent with a host cell expressing a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide; and
(b) determining whether the test agent modulates the expression of the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide by the host cell.

241. An agent identified by the method of claim 240.

242. The agent of claim 241 which is an agonist or antagonist of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide.

243. The agent of claim 242, wherein the agonist is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.

244. The agent of claim 242, wherein the antagonist is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.

245. A method of evaluating a therapeutic agent capable of affecting a condition associated with a disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising:
(a) providing a non-human transgenic animal whose genome comprises a disruption of a gene which is an ortholog of a human gene that encodes for the PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide;
(b) measuring a physiological characteristic of the non-human transgenic animal of (a);
(c) comparing the measured physiological characteristic of (b) with that of a gender matched wild-type animal, wherein the physiological characteristic of the non-human transgenic animal that differs from the physiological characteristic of the wild-type animal is identified as a condition resulting from the gene disruption in the non-human transgenic animal;
(d) administering a test agent to the non-human transgenic animal of (a); and
(e) evaluating the effects of the test agent on the identified condition associated with gene disruption in the non-human transgenic animal.

246. The method of claim 245, wherein the condition is a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality.

247. A therapeutic agent identified by the method of claim 245.

248. The therapeutic agent of claim 247 which is an agonist or antagonist of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide.

249. The therapeutic agent of claim 248, wherein the agonist is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.

250. The therapeutic agent of claim 248, wherein the antagonist is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.

251. A pharmaceutical composition comprising the therapeutic agent of claim 247.

252. A method of treating or preventing or ameliorating a neurological disorder; cardiovascular, endothelial or angiogenic disorder; immunological disorder; oncological disorder; bone metabolic abnormality or disorder, or embryonic lethality associated with the disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising administering to a subject in need of such treatment whom may already have the disorder, or may be prone to have the disorder or may be in whom the disorder is to be prevented, a therapeutically effective amount of the therapeutic agent of claim 239, or agonists or antagonists thereof, thereby effectively treating or preventing or ameliorating said disorder.

253. The method of claim 252, wherein the neurological disorder is an increased anxiety-like response during open field activity testing.

254. The method of claim 252, wherein the neurological disorder is a decreased anxiety-like response during open field activity testing.

255. The method of claim 252, wherein the neurological disorder is an abnormal circadian rhythm during home-cage activity testing.

256. The method of claim 252, wherein the neurological disorder is an enhanced motor coordination during inverted screen testing.

257. The method of claim 252, wherein the neurological disorder is an impaired motor coordination during inverted screen testing.

258. The method of claim 252, wherein the neurological disorder is depression, generalized anxiety disorders, attention deficit disorder, sleep disorder, hyperactivity disorder, obsessive compulsive disorder, schizophrenia, cognitive disorders, hyperalgesia or sensory disorders.

259. The method of claim 252, wherein the eye abnormality is a retinal abnormality.

260. The method of claim 252, wherein the eye abnormality is consistent with vision problems or blindness.

261. The method of claim 259, wherein the retinal abnormality is consistent with retinitis pigmentosa.

262. The method of claim 259, wherein the retinal abnormality is characterized by retinal degeneration or retinal dysplasia.

263. The method of claim 259, wherein the retinal abnormality is consistent with retinal dysplasia, various retinopathies, including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstrom's syndrome, Cockayne's syndrome, dysplasia spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis.

264. The method of claim 252, wherein the eye abnormality is a cataract.

265. The method of claim 264, wherein the cataract is a systemic disease such as human Down's syndrome, Hallerman-Streiff syndrome, Lowe syndrome, galactosemia, Marfan syndrome, Trismoy 13-15, Alport syndrome, myotonic dystrophy, Fabry disease, hypoparathyroidism or Conradi syndrome.

266. The method of claim 252, wherein the developmental abnormality comprises embryonic lethality or reduced viability.

267. The method of claim 252, wherein the cardiovascular, endothelial or angiogenic disorders are arterial diseases, such as diabetes mellitus; papilledema; optic atrophy; atherosclerosis; angina; myocardial infarctions such as acute myocardial infarctions, cardiac hypertrophy, and heart failure such as congestive heart failure; hypertension; inflammatory vasculitides; Reynaud's disease and Reynaud's phenomenon; aneurysms and arterial restenosis; venous and lymphatic disorders such as thrombophlebitis, lymphangitis, and lymphedema; peripheral vascular disease; cancer such as vascular tumors, e.g., hemangioma (capillary and cavernous), glomus tumors, telangiectasia, bacillary angiomatosis, hemangioendothelioma, angiosarcoma, hemangiopericytoma, Kaposi's sarcoma, lymphangioma, and lymphangiosarcoma; tumor angiogenesis; trauma such as wounds, burns, and other injured tissue, implant fixation, scarring; ischemia reperfusion injury; rheumatoid arthritis; cerebrovascular disease; renal diseases such as acute renal failure, or osteoporosis.

268. The method of claim 252, wherein the immunological disorders are systemic lupus erythematosis; rheumatoid arthritis; juvenile chronic arthritis spondyloarthropathies; systemic sclerosis (scleroderma); idiopathic inflammatory myopathies (dermatomyositis, polymyositis); Sjögren's syndrome; systemic vasculitis; sarcoidosis; autoimmune hemolytic anemia (immune pancytopenia, paroxysmal nocturnal hemoglobinuria); autoimmune thrombocytopenia (idiopathic thrombocytopenic purpura, immune-mediated thrombocytopenia); thyroiditis (Grave's disease, Hashimoto's thyroiditis, juvenile lymphocytic thyroiditis, atrophic thyroiditis); diabetes mellitus; immune-mediated renal disease (glomerulonephritis, tubulointerstitial nephritis); demyelinating diseases of the central and peripheral nervous systems such as multiple sclerosis, idiopathic demyelinating polyneuropathy or Guillain-Barré syndrome, and chronic inflammatory demyelinating polyneuropathy; hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis; inflammatory bowel disease (ulcerative colitis: Crohn's disease); gluten-sensitive enteropathy, and Whipple's disease; autoimmune or immune-mediated skin diseases including bullous skin diseases, erythema multiform and contact dermatitis, psoriasis; allergic diseases such as asthma, allergic rhinitis, atopic dermatitis, food hypersensitivity and urticaria; immunologic diseases of the lung such as eosinophilic pneumonia, idiopathic pulmonary fibrosis and hypersensitivity pneumonitis; or transplantation associated diseases including graft rejection and graft-versus-host disease.

269. The method of claim 252, wherein said bone metabolic abnormality or disorder is arthritis, osteoporosis or osteopetrosis.

270. A method of modulating a phenotype associated with a disruption of a gene which encodes for a PRO256, PRO34421, PRO334, PRO770, PRO983, PRO1009, PRO107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising administering to a subject whom may already have the phenotype, or may be prone to have the phenotype or may be in whom the phenotype is to be prevented, an effective amount of the agent of claim 195, or agonists or antagonists thereof, thereby effectively modulating the phenotype.

271. A method of modulating a physiological characteristic associated with a disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising administering to a subject whom may already exhibit the physiological characteristic, or may be prone to exhibit the physiological characteristic or may be in whom the physiological characteristic is to be prevented, an effective amount of the agent of claim 201, or agonists or antagonists thereof, thereby effectively modulating the physiological characteristic.

272. A method of modulating a behavior associated with a disruption of a gene which encodes for a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising administering to a subject whom may already exhibit the behavior, or may be prone to exhibit the behavior or may be in whom the exhibited behavior is to be prevented, an effective amount of the agent of claim 212, or agonists or antagonists thereof, thereby effectively modulating the behavior.

273. A method of modulating the expression of a PRO256, PRO34421, PRO334, PRO770, PRO983, PRO1019, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising administering to a host cell expressing said PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, an effective amount of the agent of claim 241, or agonists or antagonists thereof, thereby effectively modulating the expression of said polypeptide.

274. A method of modulating a condition associated with a disruption of a gene which encodes for a PRO256, PRO34421, PRO334, PRO770, PRO983, PRO1009, PRO107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising administering to a subject whom may have the condition, or may be prone to have the condition or may be in whom the condition is to be prevented, a therapeutically effective amount of the therapeutic agent of claim 247, or agonists or antagonists thereof, thereby effectively modulating the condition.

275. A method of identifying an agent that mimics a condition or phenotype associated with a disruption in a gene which encodes a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising:
(a) providing a non-human transgenic animal whose genome comprises a disruption of a gene which is an ortholog of a human gene that encodes a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide;
(b) measuring a physiological characteristic of the non-human transgenic animal of (a);
(c) comparing the measured physiological characteristic of (b) with that of a gender matched wild-type animal, wherein the physiological characteristic of the non-human transgenic animal that differs from the physiological characteristic of the gender matched wild-type animal is identified as a condition or phenotype resulting from the gene disruption in the non-human transgenic animal;
(d) administering a test agent to said gender matched wild-type animal; and
(e) determining whether said test agent mimics the condition or phenotype initially observed in the non-human transgenic animal.

276. The method of claim 275, wherein the condition or phenotype associated with the disruption of the gene which is an ortholog of a human gene that encodes a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide is enhanced glucose tolerance.

277. The method of claim 275, wherein the condition or phenotype associated with the disruption of the gene which is an ortholog of a human gene that encodes a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide is increased insulin sensitivity.

278. An agent identified by the method of claim 275.

279. The agent of claim 278 which is an antagonist of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide.

280. The agent of claim 279, wherein the antagonist is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.

281. A method of mimicking a condition or phenotype associated with a disruption of a gene which encodes a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising administering to a subject in whom the condition or phenotype is to be mimicked, an effective amount of the agent of claim 278 or an antagonist of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, thereby effectively mimicking the condition or phenotype.

282. The method of claim 281, wherein the condition or phenotype associated with the disruption of the gene which is an ortholog of a human gene that encodes a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide is enhanced glucose tolerance.

283. The method of claim 281, wherein the condition or phenotype associated with the disruption of the gene which is an ortholog of a human gene that encodes a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide is increased insulin sensitivity.

284. A method of evaluating a therapeutic agent capable of mimicking a condition or phenotype associated with a disruption of a gene which encodes a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising:
(a) providing a non-human transgenic animal whose genome comprises a disruption of a gene which is an ortholog of a human gene that encodes a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide;
(b) measuring a physiological characteristic of the non-human transgenic animal of (a);
(c) comparing the measured physiological characteristic of (b) with that of a gender matched wild-type animal, wherein the physiological characteristic of the non-human transgenic animal that differs from the physiological characteristic of the gender matched wild-type animal is identified as a condition or phenotype resulting from the gene disruption in the non-human transgenic animal;
(d) administering a test agent to said gender matched wild-type animal of (c); and
(e) evaluating the ability of the test agent to mimic the condition or phenotype associated with gene disruption in the non-human transgenic animal.

285. A therapeutic agent identified by the method of claim 284.

286. The therapeutic agent of claim 285 which is an antagonist of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide.

287. The therapeutic agent of claim 286, wherein the antagonist is an anti-PRO256, anti-PRO34421, anti-PRO334, anti-PRO770, anti-PRO983, anti-PRO1009, anti-PRO1107, anti-PRO1158, anti-PRO1250, anti-PRO1317, anti-PRO4334, anti-PRO4395, anti-PRO49192, anti-PRO9799, anti-PRO21175, anti-PRO19837, anti-PRO21331, anti-PRO23949, anti-PRO697 or anti-PRO1480 antibody.

288. A pharmaceutical composition comprising the therapeutic agent of claim 285.

289. A method of mimicking a condition or phenotype associated with a disruption of a gene which encodes a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, the method comprising administering to a subject in whom the condition or phenotype disorder is to be mimicked, a therapeutically effective amount of the therapeutic agent of claim 285, or an antagonist of a PRO256, PRO34421, PRO334, PRO770, PRO983, PRO1009, PRO1107, PRO1158, PRO1250, PRO1317, PRO4334, PRO4395, PRO49192, PRO9799, PRO21175, PRO19837, PRO21331, PRO23949, PRO697 or PRO1480 polypeptide, thereby effectively mimicking the condition or phenotype.


SEQUENCE LISTING (75 sequences)

SEQ ID NO: 1 - DNA, 2482 bp, Homo sapiens
gggggagaaggcggccgagccccagctctccgagcaccgggtcggaagccgcgacccgagccgcgcaggaagctgggaccggaacctcggcggacccggccccacccaactcacctgcgcaggtcaccagcaccctcggaacccagaggcccgcgctctgaaggtgacccccctggggaggaaggcgatggcccctgcgaggacgatggcccgcgcccgcctcgccccggccggcatccctgccgtcgccttgtggcttctgtgcacgctcggcctccagggcacccaggccgggccaccgcccgcgccccctgggctgcccgcgggagccgactgcctgaacagctttaccgccggggtgcctggcttcgtgctggacaccaacgcctcggtcagcaacggagctaccttcctggagtcccccaccgtgcgccggggctgggactgcgtgcgcgcctgctgcaccacccagaactgcaacttggcgctagtggagctgcagcccgaccgcggggaggacgccatcgccgcctgcttcctcatcaactgcctctacgagcagaacttcgtgtgcaagttcgcgcccagggagggcttcatcaactacctcacgagggaagtgtaccgctcctaccgccagctgcggacccagggctttggagggtctgggatccccaaggcctgggcaggcatagacttgaaggtacaaccccaggaacccctggtgctgaaggatgtggaaaacacagattggcgcctactgcggggtgacacggatgtcagggtagagaggaaagacccaaaccaggtggaactgtggggactcaaggaaggcacctacctgttccagctgacagtgactagctcagaccacccagaggacacggccaacgtcacagtcactgtgctgtccaccaagcagacagaagactactgcctcgcatccaacaaggtgggtcgctgccggggctctttcccacgctggtactatgaccccacggagcagatctgcaagagtttcgtttatggaggctgcttgggcaacaagaacaactaccttcgggaagaagagtgcattctagcctgtcggggtgtgcaaggtgggcctttgagaggcagctctggggctcaggcgactttcccccagggcccctccatggaaaggcgccatccagtgtgctctggcacctgtcagcccacccagttccgctgcagcaatggctgctgcatcgacagtttcctggagtgtgacgacacccccaactgccccgacgcctccgacgaggctgcctgtgaaaaatacacgagtggctttgacgagctccagcgcatccatttccccagtgacaaagggcactgcgtggacctgccagacacaggactctgcaaggagagcatcccgcgctggtactacaaccccttcagcgaacactgcgcccgctttacctatggtggttgttatggcaacaagaacaactttgaggaagagcagcagtgcctcgagtcttgtcgcggcatctccaagaaggatgtgtttggcctgaggcgggaaatccccattcccagcacaggctctgtggagatggctgtcacagtgttcctggtcatctgcattgtggtggtggtagccatcttgggttactgcttcttcaagaaccagagaaaggacttccacggacaccaccaccacccaccacccacccctgccagctccactgtctccactaccgaggacacggagcacctggtctataaccacaccacccggcccctctgagcctgggtctcaccggctctcacctggccctgcttcctgcttgccaaggcagaggcctgggctgggaaaaactttggaaccagactcttgcctgtttcccaggcccactgtgcctcagagaccagggctccagcccctcttggagaagtctcagctaagctcacgtcctgagaaagctcaaaggtttggaaggagcagaaaacccttgggccagaagtaccagactagatggacctgcctgcataggagtttggaggaagttggagttttgtttcctctgttcaaagctgcctgtccctaccccatggtgctaggaagaggagtggggtggtgtcagaccctggaggccccaaccctgtcctcccgagctcctcttccatgctgtgcgcccagggctgggaggaaggacttccctgtgtagtttgtgctgtaaagagttgctttttgtttatttaatgctgtggcatgggtgaagaggaggggaagaggcctgtttggcctctctgtcctctcttcctcttcccccaagattgagctctctgcccttgatcagccccaccctggcctagaccagcagacagagccaggagaggctcagctgcattccgcagcccccacccccaaggttctccaacatcacagcccagcccacccactgggtaataaaagtggtttgtggaaaaaaaaaaaaaaaaaaaaaaaaaa

SEQ ID NO: 2 - protein, 529 aa, Homo sapiens [sequence omitted]

SEQ ID NO: 3 - DNA, 2306 bp, Homo sapiens
ctgcagggacagcacccggtaactgcgagtggagcggaggacccgagcggctgaggagagaggaggcggcggcttagctgctacggggtccggccggcgccctcccgaggggggctcaggaggaggaaggaggacccgtgcgagaatgcctctgccctggagccttgcgctcccgctgctgctctcctgggtggcaggtggtttcgggaacgcggccagtgcaaggcatcacgggttgttagcatcggcacgtcagcctggggtctgtcactatggaactaaactggcctgctgctacggctggagaagaaacagcaagggagtctgtgaagctacatgcgaacctggatgtaagtttggtgagtgcgtgggaccaaacaaatgcagatgctttccaggatacaccgggaaaacctgcagtcaagatgtgaatgagtgtggaatgaaaccccggccatgccaacacagatgtgtgaatacacacggaagctacaagtgcttttgcctcagtggccacatgctcatgccagatgctacgtgtgtgaactctaggacatgtgccatgataaactgtcagtatagctgtgaagacacagaagaagggccacagtgcctgtgtccatcctcaggactccgcctggccccaaatggaagagactgtctagatattgatgaatgtgcctctggtaaagtcatctgtccctacaatcgaagatgtgtgaacacatttggaagctactactgcaaatgtcacattggtttcgaactgcaatatatcagtggacgatatgactgtatagatataaatgaatgtactatggatagccatacgtgcagccaccatgccaattgcttcaatacccaagggtccttcaagtgtaaatgcaagcagggatataaaggcaatggacttcggtgttctgctatccctgaaaattctgtgaaggaagtcctcagagcacctggtaccatcaaagacagaatcaagaagttgcttgctcacaaaaacagcatgaaaaagaaggcaaaaattaaaaatgttaccccagaacccaccaggactcctacccctaaggtgaacttgcagcccttcaactatgaagagatagtttccagaggcgggaactctcatggaggtaaaaaagggaatgaagagaaaatgaaagaggggcttgaggatgagaaaagagaagagaaagccctgaagaatgacwtagaggagcgaagcctgcgaggagatgtgtttttccctaaggtgaatgaagcaggtgaattcggcctgattctggtccaaaggaaagcgctaacttccaaactggaacataaagatttaaatatctcggttgactgcagcttcaatcatgggatctgtgactggaaacaggatagagaagatgattttgactggaatcctgctgatcgagataatgctattggcttctatatggcagttccggccttggcaggtcacaagaaagacattggccgattgaaacttctcctacctgacctgcaaccccaaagcaacttctgtttgctctttgattaccggctggccggagacaaagtcgggaaacttcgagtgtttgtgaaaaacagtaacaatgccctggcatgggagaagaccacgagtgaggatgaaaagtggaagacagggaaaattcagttgtatcaaggaactgatgctaccaaaagcatcatttttgaagcagaacgtggcaagggcaaaaccggcgaaatcgcagtggatggcgtcttgcttgtttcaggcttatgtccagatagccttttatctgtggatgactgaatgttactatctttatatttgactttgtatgtcagttccctggtttttttgatattgsatcataggacctctggcattttaaaattactaagctgaaaaattgtaatgtaccaacagaaattattattgtaagatgcctttmttgtataagatatgccaatatttgctttaaatatcatatcactgtatcttctcagtcatttctgaatctttccacattatattataaaatatggaaatgtcaggtttatctcccctcctcagtatatctgatttgtataagtaagttgatgagcttctctctgcaacatttctagaaaatagahaaaaaagcacagagaaatgtttaactgtttgactcttatgatagtttttggaaactatgacatcaaagatagacttttgcctaagtggcttagctgggtctttcatagccaaacttgtatatttaaattctttgtaataataatatccaaatcatcaaaaaaaaaaaaaaaaa

SEQ ID NO: 4 - protein, 554 aa, Homo sapiens
MPLPWSLALPLLLSWVAGGFGNAASARHHGLLASARQPGVCHYGTKLACCYGWRRNSKGVCEATCEPGCKFGECVGPNKCRCFPGYTGKTCSQDVNECGMKPRPCQHRCVNTHGSYKCFCLSGHMLMPDATCVNSRTCAMINCQYSCEDTEEGPQCLCPSSGLRLAPNGRDCLDIDECASGKVICPYNRRCVNTFGSYYCKCHIGFELQYISGRYDCIDINECTMDSHTCSHHANCFNTQGSFKCKCKQGYKGNGLRCSAIPENSVKEVLRAPGTIKDRIKKLLAHKNSMKKKAKIKNVTPEPTRTPTPKVNLQPFNYEEIVSRGGNSHGGKKGNEEKMKEGLEDEKREEKALKNDIEERSLRGDVFFPKVNEAGEFGLILVQRKALTSKLEHKADLNISVDCSFNHGICDWKQDREDDFDWNPADRDNAIGFYMAVPALAGHKKDIGRLKLLLPDLQPQSNFCLLFDYRLAGDKVGKLRVFVKNSNNALAWEKTTSEDEKWKTGKIQLYQGTDATKSIIFEAERGKGKTGEIAVDGVLLVSGLCPDSLLSVDD

SEQ ID NO: 5 - DNA, 3003 bp, Homo sapiens
gggagggggctccgggcgccgcgcagcagacctgctccggccgcgcgcctcgccgctgtcctccgggagcggcagcagtagcccgggcggcgagggctgggggttcctcgagactctcagaggggcgcctcccatcggcgcccaccaccccaacctgttcctcgcgcgccactgcgctgcgccccaggacccgctgcccaacatggattttctcctggcgctggtgctggtatcctcgctctacctgcaggcggccgccgagttcgacgggaggtggcccaggcaaatagtgtcatcgattggcctatgtcgttatggtgggaggattgactgctgctggggctgggctcgccagtcttggggacagtgtcagcctgtgtgccaaccacgatgcaaacatggtgaatgtatcgggccaaacaagtgcaagtgtcatcctggttatgctggaaaaacctgtaatcaagatctaaatgagtgtggcctgaagccccggccctgtaagcacaggtgcatgaacacttacggcagctacaagtgctactgtctcaacggatatatgctcatgccggatggttcctgctcaagtgccctgacctgctccatggcaaactgtcagtatggctgtgatgttgttaaaggacaaatacggtgccagtgcccatcccctggcctgcacctggctcctgatgggaggacctgtgtagatgttgatgaatgtgctacaggaagagcctcctgccctagatttaggcaatgtgtcaacacttttgggagctacatctgcaagtgtcataaaggcttcgatctcatgtatattggaggcaaatatcaatgtcatgacatagacgaatgctcacttggtcagtatcagtgcagcagctttgctcgatgttataacgtacgtgggtcctacaagtgcaaatgtaaagaaggataccagggtgatggactgacttgtgtgtatatcccaaaagttatgattgaaccttcaggtccaattcatgtaccaaagggaaatggtaccattttaaagggtgacacaggaaataataattggattcctgatgttggaagtacttggtggcctccgaagacaccatatattcctcctatcattaccaacaggcctacttctaagccaacaacaagacctacaccaaagccaacaccaattcctactccaccaccaccaccacccctgccaacagagctcagaacacctctaccacctacaaccccagaaaggccaaccaccggactgacaactatagcaccagctgccagtacacctccaggagggattacagttgacaacagggtacagacagaccctcagaaacccagaggagatgtgttcagtgttctggtacacagttgtaattttgaccatggactttgtggatggatcagggagaaagacaatgacttgcactgggaaccaatcagggacccagcaggtggacaatatctgacagtgtcggcagccaaagccccagggggaaaagctgcacgcttggtgctacctctcggccgcctcatgcattcaggggacctgtgcctgtcattcaggcacaaggtgacggggctgcactctggcacactccaggtgtttgtgagaaaacacggtgcccacggagcagccctgtggggaagaaatggtggccatggctggaggcaaacacagatcaccttgcgaggggctgacatcaagagcgaatcacaaagatgattaaagggttggaaaaaaagatctatgatggaaaattaaaggaactgggattattgagcctggagaagagaagactgaggggcaaaccattgatggttttcaagtatatgaagggttggcacagagagggtggcgaccagctgttctccatatgcactaagaatagaacaagaggaaactggcttagactagagtataagggagcatttcttggcaggggccattgttagaatacttcataaaaaaagaagtgtgaaaatctcagtatctctctctctttctaaaaaattagataaaaatttgtctatttaagatggttaaagatgttcttacccaaggaaaagtaacaaattatagaatttcccaaaagatgttttgatcctactagtagtatgcagtgaaaatctttagaactaaataatttggacaaggcttaatttaggcatttccctcttgacctcctaatggagagggattgaaaggggaagagcccaccaaatgctgagctcactgaaatatctctcccttatggcaatcctagcagtattaaagaaaaaaggaaactatttattccaaatgagagtatgatggacagatattttagtatctcagtaatgtcctagtgtggcggtggttttcaatgtttcttcatggtaaaggtataagcctttcatttgttcaatggatgatgtttcagatttttttttttttaagagatccttcaaggaacacagttcagagagattttcatcgggtgcattctctctgcttcgtgtgtgacaagttatcttggctgctgagaaagagtgccctgccccacaccggcagacctttccttcacctcatcagtatgattcagtttctcttatcaattggactctcccaggttccacagaacagtaatattttttgaacaataggtacaatagaaggtcttctgtcatttaacctggtaaaggcagggctggagggggaaaataaatcattaagcctttgagtaacggcagaatatatggctgtagatccatttttaatggttcatttcctttatggtcatataactgcacagctgaagatgaaaggggaaaataaatgaaaattttacttttcgatgccaatgatacattgcactaaactgatggaagaagttatccaaagtactgtataacatcttgtttattatttaatgttttctaaaataaaaaatgttagtggttttccaaatggcctaataaaaacaattatttgtaaataaaaacactgttagtaat

SEQ ID NO: 6 - protein, 509 aa, Homo sapiens
MDFLLALVLVSSLYLQAAAEFDGRWPRQIVSSIGLCRYGGRIDCCWGWARQSWGQCQPVCQPRCKHGECIGPNKCKCHPGYAGKTCNQDLNECGLKPRPCKHRCMNTYGSYKCYCLNGYMLMPDGSCSSALTCSMANCQYGCDVVKGQIRCQCPSPGLHLAPDGRTCVDVDECATGRASCPRFRQCVNTFGSYICKCHKGFDLMYIGGKYQCHDIDECSLGQYQCSSFARCYNVRGSYKCKCKEGYQGDGLTCVYIPKVMIEPSGPIHVPKGNGTILKGDTGNNNWIPDVGSTWWPPKTPYIPPIITNRPTSKPTTRPTPKPTPIPTPPPPPPLPTELRTPLPPTTPERPTTGLTTIAPAASTPPGGITVDNRVQTDPQKPRGDVFSVLVHSCNFDHGLCGWIREKDNDLHWEPIRDPAGGQYLTVSAAKAPGGKAARLVLPLGRLMHSGDLCLSFRHKVTGLHSGTLQVFVRKHGAHGAALWGRNGGHGWRQTQITLRGADIKSESQR

SEQ ID NO: 7 - DNA, 595 bp, Homo sapiens
gccacgttgtcttctttccttcaccaccacccaggagctcagagatctaagctgctttccatcttttctcccagccccaggacactgactctgtacaggatggggccgtcctcttgcctccttctcatcctaatcccccttctccagctgatcaacccggggagtactcagtgttccttagactccgttatggataagaagatcaaggatgttctcaacagtctagagtacagtccctctcctataagcaagaagctctcgtgtgctagtgtcaaaagccaaggcagaccgtcctcctgccctgctgggatggctgtcactggctgtgcttgtggctatggctgtggttcgtgggatgttcagctggaaaccacctgccactgccagtgcagtgtggtggactggaccactgcccgctgctgccacctgacctgacagggaggaggctgagaactcagttttgtgaccatgacagtaatgaaaccagggtcccaaccaagaaatctaactcaaacgtcccacttcatttgttccattcctgattcttgggtaataaagacaaactttgtacctcaaaaaaaaaaaaaaaaaaaaa

SEQ ID NO: 8 - protein, 111 aa, Homo sapiens
MGPSSCLLLILIPLLQLINPGSTQCSLDSVMDKKIKDVLNSLEYSPSPISKKLSCASVKSQGRPSSCPAGMAVTGCACGYGCGSWDVQLETTCHCQCSVVDWTTARCCHLT

SEQ ID NO: 9 - DNA, 2285 bp, Homo sapiens
gcgtgccgtcagctcgccgggcaccgcggcctcgccctcgccctccgcccctgcgcctgcaccgcgtagaccgacccccccctccagcgcgcccacccggtagaggacccccgcccgtgccccgaccggtccccgcctttttgtaaaacttaaagcgggcgcagcattaacgcttcccgccccggtgacctctcaggggtctccccgccaaaggtgctccgccgctaaggaacatggcgaaggtggagcaggtcctgagcctcgagccgcagcacgagctcaaattccgaggtcccttcaccgatgttgtcaccaccaacctaaagcttggcaacccgacagaccgaaatgtgtgttttaaggtgaagactacagcaccacgtaggtactgtgtgaggcccaacagcggaatcatcgatgcaggggcctcaattaatgtatctgtgatgttacagcctttcgattatgatcccaatgagaaaagtaaacacaagtttatggttcagtctatgtttgctccaactgacacttcagatatggaagcagtatggaaggaggcaaaaccggaagaccttatggattcaaaacttagatgtgtgtttgaattgccagcagagaatgataaaccacatgatgtagaaataaataaaattatatccacaactgcatcaaagacagaaacaccaatagtgtctaagtctctgagttcttctttggatgacaccgaagttaagaaggttatggaagaatgtaagaggctgcaaggtgaagttcagaggctacgggaggagaacaagcagttcaaggaagaagatggactgcggatgaggaagacagtgcagagcaacagccccatttcagcattagccccaactgggaaggaagaaggccttagcacccggctcttggctctggtggttttgttctttatcgttggtgtaattattgggaagattgccttgtagaggtagcatgcacaggatggtaaattggattggtggatccaccatatcatgggatttaaatttatcataaccatgtgtaaaaagaaattaatgtatgatgacatctcacaggtcttgcctttaaattacccctccctgcacacacatacacagatacacacacacaaatataatgtaacgatcttttagaaagttaaaaatgtatagtaactgattgagggggaaaaagaatgatctttattaatgacaagggaaaccatgagtaatgccacaatggcatattgtaaatgtcattttaaacattggtaggccttggtacatgatgctggattacctctcttaaaatgacacccttcctcgcctgttggtgctggcccttggggagctggagcccagcatgctggggagtgcggtcagctccacacagtagtccccacgtggcccactcccggcccaggctgctttccgtgtcttcagttctgtccaagccatcagctccttgggactgatgaacagagtcagaagcccaaaggaattgcactgtggcagcatcagacgtactcgtcataagtgagaggcgtgtgttgactgattgacccagcgctttggaaataaatggcagtgctttgttcacttaaagggaccaagctaaatttgtattggttcatgtagtgaagtcaaactgttattcagagatgtttaatgcatatttaacttatttaatgtatttcatctcatgttttcttattgtcacaagagtacagttaatgctgcgtgctgctgaactctgttgggtgaactggtattgctgctggagggctgtgggctcctctgtctctggagagtctggtcatgtggaggtggggtttattgggatgctggagaagagctgccaggaagtgttttttctgggtcagtaaataacaactgtcatagggagggaaattctcagtagtgacagtcaactctaggttaccttttttaatgaagagtagtcagtcttctagattgttcttataccacctctcaaccattactcacacttccagcgcccaggtccaagtctgagcctgacctccccttggggacctagcctggagtcaggacaaatggatcgggctgcagagggttagaagcgagggcaccagcagttgtgggtggggagcaagggaagagagaaactcttcagcgaatccttctagtactagttgagagtttgactgtgaattaattttatgccataaaagaccaacccagttctgtttgactatgtagcatcttgaaaagaaaaattataataaagccccaaaattaagaaaa

SEQ ID NO: 10 - protein, 243 aa, Homo sapiens
MAKVEQVLSLEPQHELKFRGPFTDVVTTNLKLGNPTDRNVCFKVKTTAPRRYCVRPNSGIIDAGASINVSVMLQPFDYDPNEKSKHKFMVQSMFAPTDTSDMEAVWKEAKPEDLMDSKLRCVFELPAENDKPHDVEINKIISTTASKTETPIVSKSLSSSLDDTEVKKVMEECKRLQGEVQRLREENKQFKEEDGLRMRKTVQSNSPISALAPTGKEEGLSTRLLALVVLFFIVGVIIGKIAL

SEQ ID NO: 11 - DNA, 2187 bp, Homo sapiens
cggacgcgtgggcgggccgggacgcagggcaaagcgagccatggctgtctacgtcgggatgctgcgcctggggaggctgtgcgccgggagctcgggggtgctgggggcccgggccgccctctctcggagttggcaggaagccaggttgcagggtgtccgcttcctcagttccagagaggtggatcgcatggtctccacgcccatcggaggcctcagctacgttcaggggtgcaccaaaaagcatcttaacagcaagactgtgggccagtgcctggagaccacagcacagagggtcccagaacgagaggccttggtcgtcctccatgaagacgtcaggttgacctttgcccaactcaaggaggaggtggacaaagctgcttctggcctcctgagcattggcctctgcaaaggtgaccggctgggcatgtggggacctaactcctatgcatgggtgctcatgcagttggccaccgcccaggcgggcatcattctggtgtctgtgaacccagcctaccaggctatggaactggagtatgtcctcaagaaggtgggctgcaaggcccttgtgttccccaagcaattcaagacccagcaatactacaacgtcctgaagcagatctgtccagaagtggagaatgcccagccaggggccttgaagagtcagaggctcccagatctgaccacagtcatctcggtggatgcccctttgccggggaccctgctcctggatgaagtggtggcggctggcagcacacggcagcatctggaccagctccaatacaaccagcagttcctgtcctgccatgaccccatcaacatccagttcacctcggggacaacaggcagccccaagggggccaccctctcccactacaacattgtcaacaactccaacattttaggagagcgcctgaaactgcatgagaagacaccagagcagttgcggatgatcctgcccaaccccctgtaccattgcctgggttccgtggcaggcacaatgatgtgtctgatgtacggtgccaccctcatcctggcctctcccatcttcaatggcaagaaggcactggaggccatcagcagagagagaggcaccttcctgtatggtacccccacgatgttcgtggacattctgaaccagccagacttctccagttatgacatctcgaccatgtgtggaggtgtcattgctgggtcccctgcacctccagagttgatccgagccatcatcaacaagataaatatgaaggacctggtggttgcttatggaaccacagagaacagtcccgtgacattcgcgcacttccctgaggacactgtggagcagaaggcagaaagcgtgggcagaattatgcctcacacggaggcccggatcatgaacatggaggcagggacgctggcaaagctgaacacgcccggggagctgtgcatccgagggtactgcgtcatgctgggctactggggtgagcctcagaagacagaggaagcagtggatcaggacaagtggtattggacaggagatgtcgccacaatgaatgagcagggcttctgcaagatcgtgggccgctctaaggatatgatcatccggggtggtgagaacatctaccccgcagagctcgaggacttctttcacacacacccgaaggtgcaggaagtgcaggtggtgggagtgaaggacgatcggatgggggaagagatttgtgcctgcattcggctgaaggacggggaggagaccacggtggaggagataaaagctttctgcaaagggaagatctctcacttcaagattccgaagtacatcgtgtttgtcacaaactaccccctcaccatttcaggaaagatccagaaattcaaacttcgagagcagatggaacgacatctaaatctgtgaataaagcagcaggcctgtcctggccggttggcttgactctctcctgtcagaatgcaacctggctttatgcacctagatgtccccagcacccagttctgagccaggcacatcaaatgtcaaggaattgactgaacgaactaagagctcctggatgggtccgggaactcgcctgggcacaaggtgccaaaaggcaggcagcctgcccaggccctccctcctgtccatcccccacattcccctgtctgtccttgtgatttggcataaagagcttctgttttctttgaaaaaaaaaaaaaaaa

SEQ ID NO: 12 - protein, 615 aa, Homo sapiens
MAVYVGMLRLGRLCAGSSGVLGARAALSRSWQEARLQGVRFLSSREVDRMVSTPIGGLSYVQGCTKKHLNSKTVGQCLETTAQRVPEREALVVLHEDVRLTFAQLKEEVDKAASGLLSIGLCKGDRLGMWGPNSYAWVLMQLATAQAGIILVSVNPAYQAMELEYVLKKVGCKALVFPKQFKTQQYYNVLKQICPEVENAQPGALKSQRLPDLTTVISVDAPLPGTLLLDEVVAAGSTRQHLDQLQYNQQFLSCHDPINIQFTSGTTGSPKGATLSHYNIVNNSNILGERLKLHEKTPEQLRMILPNPLYHCLGSVAGTMMCLMYGATLILASPIFNGKKALEAISRERGTFLYGTPTMFVDILNQPDFSSYDISTMCGGVIAGSPAPPELIRAIINKINMKDLVVAYGTTENSPVTFAHFPEDTVEQKAESVGRIMPHTEARIMNMEAGTLAKLNTPGELCIRGYCVMLGYWGEPQKTEEAVDQDKWYWTGDVATMNEQGFCKIVGRSKDMIIRGGENIYPAELEDFFHTHPKVQEVQVVGVKDDRMGEEICACIRLKDGEETTVEEIKAFCKGKISHFKIPKYIVFVTNYPLTISGKIQKFKLREQMERHLNL

SEQ ID NO: 13 - DNA, 2623 bp, Homo sapiens
ttgagcgcaggtgagctcctgcgcgttccgggggcgttcctccagtcaccctcccgccgttacccgcggcgcgcccgagggagtctcctccagaccctccctcccgttgctccaaactaatacggactgaacggatcgctgcgagggtgggagagaaaattagggggagaaaggacagagagagcaactaccatccatagccagatagattatcttacactgaactgatcaagtactttgaaaatgacttcgaaatttatcttggtgtccttcatacttgctgcactgagtctttcaaccaccttttctctccaactagaccagcaaaaggttctactagtttcttttgatggattccgttgggattacttatataaagttccaacgccccattttcattatattatgaaatatggtgttcacgtgaagcaagttactaatgtttttattacaaaaacctaccctaaccattatactttggtaactggcctctttgcagagaatcatgggattgttgcaaatgatatgtttgatcctattcggaacaaatctttctccttggatcacatgaatatttatgattccaagttttgggaagaagcgacaccaatatggatcacaaaccagagggcaggacatactagtggtgcagccatgtggcccggaacagatgtaaaaatacataagcgctttcctactcattacatgccttacaatgagtcagtttcatttgaagatagagttgccaaaattgttgaatggtttacgtcaaaagagcccataaatcttggtcttctctattgggaagaccctgatgacatgggccaccatttgggacctgacagtccgctcatggggcctgtcatttcagatattgacaagaagttaggatatctcatacaaatgctgaaaaaggcaaagttgtggaacactctgaacctaatcatcacaagtgatcatggaatgacgcagtgctctgaggaaaggttaatagaacttgaccagtacctggataaagaccactataccctgattgatcaatctccagtagcagccatcttgccaaaagaaggtaaatttgatgaagtctatgaagcactaactcacgctcatcctaatcttactgtttacaaaaaagaagacgttccagaaaggtggcattacaaatacaacagtcgaattcaaccaatcatagcagtggctgatgaagggtggcacattttacagaataagtcagatgactttctgttaggcaaccacggttacgataatgcgttagcagatatgcatccaatatttttagcccatggtcctgccttcagaaagaatttctcaaaagaagccatgaactccacagatttgtacccactactatgccacctcctcaatatcactgccatgccacacaatggatcattctggaatgtccaggatctgctcaattcagcaatgccaagggtggtcccttatacacagagtactatactcctccctggtagtgttaaaccagcagaatatgaccaagaggggtcatacccttatttcataggggtctctcttggcagcattatagtgattgtattttttgtaattttcattaagcatttaattcacagtcaaatacctgccttacaagatatgcatgctgaaatagctcaaccattattacaagcctaatgttactttgaagtggatttgcatattgaagtggagattccataattatgtcagtgtttaaaggtttcaaattctgggaaaccagttccaaacatctgcagaaaccattaagcagttacatatttaggtatacacacacacacacacacacacatacacacacacggaccaaaatacttacacctgcaaaggaataaagatgtgagagtatgtctccattgttcactgtagcatagggatagataagatcctgctttatttggacttggcgcagataatgtatatatttagcaactttgcactatgtaaagtaccttatatattgcactttaaatttctctcctgatgggtactttaatttgaaatgcactttatggacagttatgtcttataacttgattgaaaatgacaactttttgcacccatgtcacagaatacttgttacgcattgttcaaactgaaggaaatttctaataatcccgaataatgaacatagaaatctatctccataaattgagagaagaagaaggtgataagtgttgaaaattaaatgtgataacctttgaaccttgaattttggagatgtattcccaacagcagaatgcaactgtgggcatttcttgtcttatttctttccagagaacgtggttttcatttatttttccctcaaaagagagtcaaatactgacagattcgttctaaatatattgtttctgtcataaaattattgtgatttcctgatgagtcatattactgtgattttcataataatgaagacaccatgaatatacttttcttctatatagttcagcaatggcctgaatagaagcaaccaggcaccatctcagcaatgttttctcttgtttgtaattatttgctcctttgaaaattaaatcactattaattacattaaaaatcaaattggataaaaaaaaaaaaaaaaaaa

SEQ ID NO: 14 - protein, 477 aa, Homo sapiens [sequence omitted]

SEQ ID NO: 15 - DNA, 744 bp, Homo sapiens
acggaccgagggttcgagggagggacacggaccaggaacctgagctaggtcaaagacgcccgggccaggtgccccgtcgcaggtgcccctggccggagatgcggtaggaggggcgagcgcgagaagccccttcctcggcgctgccaacccgccacccagcccatggcgaaccccgggctggggctgcttctggcgctgggcctgccgttcctgctggcccgctggggccgagcctgggggcaaatacagaccacttctgcaaatgagaatagcactgttttgccttcatccaccagctccagctccgatggcaacctgcgtccggaagccatcactgctatcatcgtggtcttctccctcttggctgccttgctcctggctgtggggctggcactgttggtgcggaagcttcgggagaagcggcagacggagggcacctaccggcccagtagcgaggagcagttctcccatgcagccgaggcccgggcccctcaggactccaaggagacggtgcagggctgcctgcccatctaggtcccctctcctgcatctgtctcccttcattgctgtgtgaccttggggaaaggcagtgccctctctgggcagtcagatccacccagtgcttaatagcagggaagaaggtacttcaaagactctgcccctgaggtcaagagaggatggggctattcacttttatatatttatataaaattagtagtgagatgtaaaaaaaaaaaaaaaaaa

SEQ ID NO: 16 - protein, 123 aa, Homo sapiens
MANPGLGLLLALGLPFLLARWGRAWGQIQTTSANENSTVLPSSTSSSSDGNLRPEAITAIIVVFSLLAALLLAVGLALLVRKLREKRQTEGTYRPSSEEQFSHAAEARAPQDSKETVQGCLPI

SEQ ID NO: 17 - DNA, 3316 bp, Homo sapiens
ctgacatggcctgactcgggacagctcagagcagggcagaactggggacactctgggccggccttctgcctgcatggacgctctgaagccaccctgtctctggaggaaccacgagcgagggaagaaggacagggactcgtgtggcaggaagaactcagagccgggaagcccccattcactagaagcactgagagatgcggccccctcgcagggtctgaatttcctgctgctgttcacaaagatgctttttatctttaactttttgttttccccacttccgaccccggcgttgatctgcatcctgacatttggagctgccatcttcttgtggctgatcaccagacctcaacccgtcttacctcttcttgacctgaacaatcagtctgtgggaattgagggaggagcacggaagggggtttcccagaagaacaatgacctaacaagttgctgcttctcagatgccaagactatgtatgaggttttccaaagaggactcgctgtgtctgacaatgggccctgcttgggatatagaaaaccaaaccagccctacagatggctatcttacaaacaggtgtctgatagagcagagtacctgggttcctgtctcttgcataaaggttataaatcatcaccagaccagtttgtcggcatctttgctcagaataggccagagtggatcatctccgaattggcttgttacacgtactctatggtagctgtacctctgtatgacaccttgggaccagaagccatcgtacatattgtcaacaaggctgatatcgccatggtgatctgtgacacaccccaaaaggcattggtgctgatagggaatgtagagaaaggcttcaccccgagcctgaaggtgatcatccttatggacccctttgatgatgacctgaagcaaagaggggagaagagtggaattgagatcttatccctatatgatgctgagaacctaggcaaagagcacttcagaaaacctgtgcctcctagcccagaagacctgagcgtcatctgcttcaccagtgggaccacaggtgaccccaaaggagccatgataacccatcaaaatattgtttcaaatgctgctgcctttctcaaatgtgtggagcatgcttatgagcccactcctgatgatgtggccatatcctacctccctctggctcatatgtttgagaggattgtacaggctgttgtgtacagctgtggagccagagttggattcttccaaggggatattcggttgctggctgacgacatgaagactttgaagcccacattgtttcccgcggtgcctcgactccttaacaggatctacgataaggtacaaaatgaggccaagacacccttgaagaagttcttgttgaagctggctgtttccagtaaattcaaagagcttcaaaagggtatcatcaggcatgatagtttctgggacaagctcatctttgcaaagatccaggacagcctgggcggaagggttcgtgtaattgtcactggagctgcccccatgtccacttcagtcatgacattcttccgggcagcaatgggatgtcaggtgtatgaagcttatggtcaaacagaatgcacaggtggctgtacatttacattacctggggactggacatcaggtcacgttggggtgcccctggcttgcaattacgtgaagctggaagatgtggctgacatgaactactttacagtgaataatgaaggagaggtctgcatcaagggtacaaacgtgttcaaaggatacctgaaggaccctgagaagacacaggaagccctggacagtgatggctggcttcacacaggagacattggtcgctggctcccgaatggaactctgaagatcatcgaccgtaaaaagaacattttcaagctggcccaaggagaatacattgcaccagagaagatagaaaatatctacaacaggagtcaaccagtgttacaaatttttgtacacggggagagcttacggtcatccttagtaggagtggtggttcctgacacagatgtacttccctcatttgcagccaagcttggggtgaagggctcctttgaggaactgtgccaaaaccaagttgtaagggaagccattttagaagacttgcagaaaattgggaaagaaagtggccttaaaacttttgaacaggtcaaagccatttttcttcatccagagccattttccattgaaaatgggctcttgacaccaacattgaaagcaaagcgaggagagctttccaaatactttcggacccaaattgacagcctgtatgagcacatccaggattaggataaggtacttaagtacctgccggcccactgtgcactgcttgtgagaaaatggattaaaaactattcttacatttgttttgcctttcctcctatttttttttaacctgttaaactctaaagccatagcttttgttttatattgagacatataatgtgtaaacttagttcccaaataaatcaatcctgtctttcccatcttcgatgttgctaatattaaggcttcagggctacttttatcaacatgcctgtcttcaagatcccagtttatgttctgtgtccttcctcatgatttccaaccttaatactattagtaaccacaagttcaagggtcaaagggaccctctgtgccttcttctttgttttgtgataaacataacttgccaacagtctctatgcttatttacatcttctactgttcaaactaagagatttttaaattctgaaaaactgcttacaattcatgttttctagccactccacaaaccactaaaattttagttttagcctatcactcatgtcaatcatatctatgagacaaatgtctccgatgctcttctgcgtaaattaaattgtgtactgaagggaaaagtttgatcataccaaacatttcctaaactctctagttagatatctgacttgggagtattaaaaattgggtctatgacatactgtccaaaaggaatgctgttcttaaagcattatttacagtaggaactggggagtaaatctgttccctacagtttgctgctgagctggaagctgtgggggaaggagttgacaggtgggcccagtgaacttttccagtaaatgaagcaagcactgaataaaaacctcctgaactgggaacaaagatctacaggcaagcaagatgcccacacaacaggcttattttctgtgaaggaaccaactgatctcccccacccttggattagagttcctgctctaccttacccacagataacacatgttgtttctacttgtaaatgtaaagtctttaaaataaactattacagataaaaaa

SEQ ID NO: 18 - protein, 739 aa, Homo sapiens
MDALKPPCLWRNHERGKKDRDSCGRKNSEPGSPHSLEALRDAAPSQGLNFLLLFTKMLFIFNFLFSPLPTPALICILTFGAAIFLWLITRPQPVLPLLDLNNQSVGIEGGARKGVSQKNNDLTSCCFSDAKTMYEVFQRGLAVSDNGPCLGYRKPNQPYRWLSYKQVSDRAEYLGSCLLHKGYKSSPDQFVGIFAQNRPEWIISELACYTYSMVAVPLYDTLGPEAIVHIVNKADIAMVICDTPQKALVLIGNVEKGFTPSLKVIILMDPFDDDLKQRGEKSGIEILSLYDAENLGKEHFRKPVPPSPEDLSVICFTSGTTGDPKGAMITHQNIVSNAAAFLKCVEHAYEPTPDDVAISYLPLAHMFERIVQAVVYSCGARVGFFQGDIRLLADDMKTLKPTLFPAVPRLLNRIYDKVQNEAKTPLKKFLLKLAVSSKFKELQKGIIRHDSFWDKLIFAKIQDSLGGRVRVIVTGAAPMSTSVMTFFRAAMGCQVYEAYGQTECTGGCTFTLPGDWTSGHVGVPLACNYVKLEDVADMNYFTVNNEGEVCIKGTNVFKGYLKDPEKTQEALDSDGWLHTGDIGRWLPNGTLKIIDRKKNIFKLAQGEYIAPEKIENIYNRSQPVLQIFVHGESLRSSLVGVVVPDTDVLPSFAAKLGVKGSFEELCQNQVVREAILEDLQKIGKESGLKTFEQVKAIFLHPEPFSIENGLLTPTLKAKRGELSKYFRTQIDSLYEHIQD

SEQ ID NO: 19 - DNA, 3143 bp, Homo sapiens
gggctgaggcactgagagaccggaaagcctggcattccagagggagggaaacgcagcggcatccccaggctccagagctccctggtgacagtctgtggctgagcatggccctcccagccctgggcctggacccctggagcctcctgggccttttcctcttccaactgcttcagctgctgctgccgacgacgaccgcggggggaggcgggcaggggcccatgcccagggtcagatactatgcaggggatgaacgtagggcacttagcttcttccaccagaagggcctccaggattttgacactctgctcctgagtggtgatggaaatactctctacgtgggggctcgagaagccattctggccttggatatccaggatccaggggtccccaggctaaagaacatgataccgtggccagccagtgacagaaaaaagagtgaatgtgcctttaagaagaagagcaatgagacacagtgtttcaacttcatccgtgtcctggtttcttacaatgtcacccatctctacacctgcggcaccttcgccttcagccctgcttgtaccttcattgaacttcaagattcctacctgttgcccatctcggaggacaaggtcatggagggaaaaggccaaagcccctttgaccccgctcacaagcatacggctgtcttggtggatgggatgctctattctggtactatgaacaacttcctgggcagtgagcccatcctgatgcgcacactgggatcccagcctgtcctcaagaccgacaacttcctccgctggctgcatcatgacgcctcctttgtggcagccatcccttcgacccaggtcgtctacttcttcttcgaggagacagccagcgagtttgacttctttgagaggctccacacatcgcgggtggctagagtctgcaagaatgacgtgggcggcgaaaagctgctgcagaagaagtggaccaccttcctgaaggcccagctgctctgcacccagccggggcagctgcccttcaacgtcatccgccacgcggtcctgctccccgccgattctcccacagctccccacatctacgcagtcttcacctcccagtggcaggttggcgggaccaggagctctgcggtttgtgccttctctctcttggacattgaacgtgtctttaaggggaaatacaaagagttgaacaaagaaacttcacgctggactacttataggggccctgagaccaacccccggccaggcagttgctcagtgggcccctcctctgataaggccctgaccttcatgaaggaccatttcctgatggatgagcaagtggtggggacgcccctgctggtgaaatctggcgtggagtatacacggcttgcagtggagacagcccagggccttgatgggcacagccatcttgtcatgtacctgggaaccaccacagggtcgctccacaaggctgtggtaagtggggacagcagtgctcatctggtggaagagattcagctgttccctgaccctgaacctgttcgcaacctgcagctggcccccacccagggtgcagtgtttgtaggcttctcaggaggtgtctggagggtgccccgagccaactgtagtgtctatgagagctgtgtggactgtgtccttgcccgggacccccactgtgcctgggaccctgagtcccgaacctgttgcctcctgtctgcccccaacctgaactcctggaagcaggacatggagcgggggaacccagagtgggcatgtgccagtggccccatgagcaggagccttcggcctcagagccgcccgcaaatcattaaagaagtcctggctgtccccaactccatcctggagctcccctgcccccacctgtcagccttggcctcttattattggagtcatggcccagcagcagtcccagaagcctcttccactgtctacaatggctccctcttgctgatagtgcaggatggagttgggggtctctaccagtgctgggcaactgagaatggcttttcataccctgtgatctcctactgggtggacagccaggaccagaccctggccctggatcctgaactggcaggcatcccccgggagcatgtgaaggtcccgttgaccagggtcagtggtggggccgccctggctgcccagcagtcctactggccccactttgtcactgtcactgtcctctttgccttagtgctttcaggagccctcatcatcctcgtggcctccccattgagagcactccgggctcggggcaaggttcagggctgtgagaccctgcgccctggggagaaggccccgttaagcagagagcaacacctccagtctcccaaggaatgcaggacctctgccagtgatgtggacgctgacaacaactgcctaggcactgaggtagcttaaactctaggcacaggccggggctgcggtgcaggcacctggccatgctggctgggcggcccaagcacagccctgactaggatgacagcagcacaaaagaccacctttctcccctgagaggagcttctgctactctgcatcactgatgacactcagcagggtgatgcacagcagtctgcctcccctatgggactcccttctaccaagcacatgagctctctaacagggtgggggctacccccagacctgctcctacactgatattgaagaacctggagaggatccttcagttctggccattccagggaccctccagaaacacagtgtttcaagagaccctaaaaaacctgcctgtcccaggaccctatggtaatgaacaccaaacatctaaacaatcatatgctaacatgccactcctggaaactccactctgaagctgccgctttggacaccaacactcccttctcccagggtcatgcagggatctgctccctcctgcttcccttaccagtcgtgcaccgctgactcccaggaagtctttcctgaagtctgaccacctttcttcttgcttcagttggggcagactctgatcccttctgccctggcagaatggcaggggtaatctgagccttcttcactcctttaccctagctgaccccttcacctctccccctcccttttcctttgttttgggattcagaaaactgcttgtcagagactgtttattttttattaaaaatataaggcttaaaaaaa

SEQ ID NO: 20 - protein, 761 aa, Homo sapiens
MALPALGLDPWSLLGLFLFQLLQLLLPTTTAGGGGQGPMPRVRYYAGDERRALSFFHQKGLQDFDTLLLSGDGNTLYVGAREAILALDIQDPGVPRLKNMIPWPASDRKKSECAFKKKSNETQCFNFIRVLVSYNVTHLYTCGTFAFSPACTFIELQDSYLLPISEDKVMEGKGQSPFDPAHKHTAVLVDGMLYSGTMNNFLGSEPILMRTLGSQPVLKTDNFLRWLHHDASFVAAIPSTQVVYFFFEETASEFDFFERLHTSRVARVCKNDVGGEKLLQKKWTTFLKAQLLCTQPGQLPFNVIRHAVLLPADSPTAPHIYAVFTSQWQVGGTRSSAVCAFSLLDIERVFKGKYKELNKETSRWTTYRGPETNPRPGSCSVGPSSDKALTFMKDHFLMDEQVVGTPLLVKSGVEYTRLAVETAQGLDGHSHLVMYLGTTTGSLHKAVVSGDSSAHLVEEIQLFPDPEPVRNLQLAPTQGAVFVGFSGGVWRVPRANCSVYESCVDCVLARDPHCAWDPESRTCCLLSAPNLNSWKQDMERGNPEWACASGPMSRSLRPQSRPQIIKEVLAVPNSILELPCPHLSALASYYWSHGPAAVPEASSTVYNGSLLLIVQDGVGGLYQCWATENGFSYPVISYWVDSQDQTLALDPELAGIPREHVKVPLTRVSGGAALAAQQSYWPHFVTVTVLFALVLSGALIILVASPLRALRARGKVQGCETLRPGEKAPLSREQHLQSPKECRTSASDVDADNNCLGTEVA

SEQ ID NO: 21 - DNA, 1579 bp, Homo sapiens
gagagaatagctacagattctccatcctcagtctttgcaaggcgacagctgtgccagccgggctctggcaggctcctggcagcatggcagtgaagcttgggaccctcctgctggcccttgccctgggcctggcccagccagcctctgcccgccggaagctgctggtgtttctgctggatggttttcgctcagactacatcagtgatgaggcgctggagtcattgcctggtttcaaagagattgtgagcaggggagtaaaagtggattacttgactccagacttccctagtctctcgtatcccaattattataccctaatgactggccgccattgtgaagtccatcagatgatcgggaactacatgtgggaccccaccaccaacaagtcctttgacattggcgtcaacaaagacagcctaatgcctctctggtggaatggatcagaacctctgtgggtcactctgaccaaggccaaaaggaaggtctacatgtactactggccaggctgtgaggttgagattctgggtgtcagacccacctactgcctagaatataaaaatgtcccaacggatatcaattttgccaatgcagtcagcgatgctcttgactccttcaagagtggccgggccgacctggcagccatataccatgagcgcattgacgtggaaggccaccactacgggcctgcatctccgcagaggaaagatgccctcaaggctgtagacactgtcctgaagtacatgaccaagtggatccaggagcggggcctgcaggaccgcctgaacgtcattattttctcggatcacggaatgaccgacattttctggatggacaaagtgattgagctgaataagtacatcagcctgaatgacctgcagcaagtgaaggaccgcgggcctgttgtgagcctttggccggcccctgggaaacactctgagatatataacaaactgagcacagtggaacacatgactgtctacgagaaagaagccatcccaagcaggttctattacaagaaaggaaagtttgtctctcctttgactttagtggctgatgaaggctggttcataactgagaatcgagagatgcttccgttttggatgaacagcaccggcaggcgggaaggttggcagcgtggatggcacggctacgacaacgagctcatggacatgcggggcatcttcctggccttcggacctgatttcaaatccaacttcagagctgctcctatcaggtcggtggacgtctacaatgtcatgtgcaatgtggtgggcatcaccccgctgcccaacaacggatcctggtccagggtgatgtgcatgctgaagggccgcgccggcactgccccgcctgtctggcccagccactgtgccctggcactgattcttctcttcctgcttgcataactgatcatattgcttgtctcagaaaaaaacaccatcagcaaagtgggcctccaaagccagatgattttcattttatgtgtgaataatagcttcattaacacaatcaagaccatgcacattgtaaatacattattcttggataattctatacataaaagttcctacttgttaaa

SEQ ID NO: 22 - protein, 440 aa, Homo sapiens [sequence omitted]

SEQ ID NO: 23 - DNA, 3233 bp, Homo sapiens
gccgggagcttccctgatggtgccgccgcctccgagccggggaggagctgccaggggccagctgggcaggagcctgggtccgctgctgctgctcctggcgttgggacacacgtggacctacagagaggagccggaggacggcgacagagaaatctgctcagagagcaaaatcgcgacgactaaatacccgtgtctgaagtcttcaggcgagctcaccacatgctacaggaaaaagtgctgcaaaggatataaatttgttcttggacaatgcatcccagaagattacgacgtttgtgccgaggctccctgtgaacagcagtgcacggacaactttggccgagtgctgtgtacttgttatccgggataccgatatgaccgggagagacaccggaagcgggagaagccatactgtctggatattgatgagtgtgccagcagcaatgggacgctgtgtgcccacatctgcatcaataccttgggcagctaccgctgcgagtgccgggaaggctacatccgggaagatgatgggaagacatgtaccaggggagacaaatatcccaatgacactggccatgagaagtctgagaacatggtgaaagccggaacttgctgtgccacatgcaaggagttctaccagatgaagcagaccgtgctgcagctgaagcaaaagattgctctgctccccaacaatgcagctgacctgggcaagtatatcactggtgacaaggtgctggcctcaaacacctaccttccaggacctcctggcctgcctgggggccagggccctcccggctcaccaggaccaaagggaagcccaggcttccccggtatgccaggccctcctgggcagcccggcccacggggctcaatgggacccatgggaccatctcctgatctgtcccacattaagcaaggccggaggggccctgtgggtccaccaggggcaccaggaagagatggttctaagggggagagaggagcgcctgggcccagagggtctccaggaccccctggttctttcgacttcctgctacttatgctggctgacatccgcaatgacatcactgagctgcaggaaaaggtgttcgggcaccggactcactcttcagcagaggagttccctttacctcaggaatttcccagctacccagaagccatggacctgggctctggagatgaccatccaagaagaactgagacaagagacttgagagcccccagagacttctacccatagcacatcccaacaccgtcacgccaaaggaagagaaagatcaactcacctgcagttaaaccatctaaagagaagaaagaccactggagacctagaaaacatacatttttctcttctcttctcctgacgtctctccactcctcttcttccaaatacgatgctattttcagagtcccctcctaggcctgcagacatgagggagtgaatgattgatttacctgcttctcactaagagtccattggggtggtttgcattgtaacttttcttttacatcctatttttccaggaactttggatttaagtactctcacagtgtcttaaatcataaattcttgaagttaaatttggcagagtatcaaaagggggaaaatgacaaagtgagctctaagaaaatgtgaggctacttctaagatgtgtgttcacaatagaccataactcctctagtatcaaaattggggctcttcagttaaaaaggggtggggaggacaaacgtgtcgatgtgctttggtggagaattttttccttgtgcttctagtagactttaaatattgtatccctttgtcaaaccttgtttcccaaattcaattaaagagaggagagaattgaatggcgtttagagaagatagaaaagaatcacagtcatatatttactgttatatagattgccacattctaaaattcaaatacggtgcttaaggtttcatgccatgcttatctgtaagtatcctatttagggaagaagattaaactctcttttcaaaaaaacaaagtgaaatgcctggattcacattaaaacaatgggctctcgtttgctataatattttaaagctgtttaatcaacagtggagtctgctctataaatatagattatttgttcaataaactggctgagcttagagagaggtgcagaattcctggttctgagcaggtgcccagaaggtaccattaggtgccatgatccaggctgaaccaatatacagtggggctgaagtctgcaaggaggttgctggcttgggctgacctcactaatgccatcagcagcggtaggtaaattttttctccttgggtattacaagtttttgtctggagccaaccaagcttgccaccaacatattgagagtaatacactattgaaagttatcttggatggggagaaaaaaaaatagtggttttccttgtttgcaaaaacttccttcctattctcattttttcttaattttctttaatttagtccaagttccagttcttttaggccttctctttgatttattttcccctgcatgtgagaagcagttcagaaaaaggtctatatctccacctcctagtgagttagagtgttttctcagagcacctctgggtggcaaagggaagcatgttcctgccaaggtttgctgtggattcagaagcaccaggagcaagagaccagaaggatgatctgctcctttgtaacgttgttgagggccctcttgtttccaatgagcagcttataggttactcacagtccactttctcactggacacacaaagtggctctttatctacctttgcgggagattttcactctcctgcaaatgatcgttctcacactcatattagctcatgttggaatttcccatcctgccatgtcctttcccatttctttttggcttttttgcctccaccttttagcccacatcatttaactccactactgtgaaagcttgcttaaagaaaatccctcttggccgggtgtggtagcccacgcctctaatcccagcactttgggaggctgaggcggggagatcacaaggtcaggagatcgagaccagcctgaccaacatggtgaaaccctgtctctactaaaaatacaaaaattagctgggcgtgttggcacacacctgtaatcccagctactcaggaggctgaggcaggagaattactttaacctgcggggggagcctagattgcgctactgcactccagcctaggcaacagagggagactctgtctcattaaaaa

SEQ ID NO: 24 - protein, 406 aa, Homo sapiens
MVPPPPSRGGAARGQLGRSLGPLLLLLALGHTWTYREEPEDGDREICSESKIATTKYPCLKSSGELTTCYRKKCCKGYKFVLGQCIPEDYDVCAEAPCEQQCTDNFGRVLCTCYPGYRYDRERHRKREKPYCLDIDECASSNGTLCAHICINTLGSYRCECREGYIREDDGKTCTRGDKYPNDTGHEKSENMVKAGTCCATCKEFYQMKQTVLQLKQKIALLPNNAADLGKYITGDKVLASNTYLPGPPGLPGGQGPPGSPGPKGSPGFPGMPGPPGQPGPRGSMGPMGPSPDLSHIKQGRRGPVGPPGAPGRDGSKGERGAPGPRGSPGPPGSFDFLLLMLADIRNDITELQEKVFGHRTHSSAEEFPLPQEFPSYPEAMDLGSGDDHPRRTETRDLRAPRDFYP

SEQ ID NO: 25 - DNA, 1621 bp, Homo sapiens
cggaaggtgcctgtgacgaggattggccggtaagcagagcatggcgggtgcgggcccgaagcggcgggcgctagcggccccggtggccgaggagaaggaagaggcgcgggagaagatgctggcctccaagcgcgcggacggcgcggcgccggcaggcgagggcgagggcgtgaccctgcagcggaacatcacgctactcaacggcgtggccatcatcgtgggcgccatcatcggctcgggcatcttcgtgacgcccacgggcgtgcttaaggaggcaggctcgccggggctggcgctggtgatgtgggccgcgtgcggcgtcttctccatcgtgggcgcgctctgctacgcggagctcggcaccaccatctccaaatcgggcggcgactacgcctacatgctggaggtctacggctcgctgcccgccttcctcaagctctggatcgagctgctcatcatccggccttcatcgcagtacatcgtggccctggtcttcgccgcctacctgctcaagccgctcttccccacctgcccggtgcccgaggaggcagccaagctcgtggcctgcctctgcgtgctgctgctcacggccgtgaactgctacagcgtgaaggccgccacccgggtccaggatgcctttgccgccgccaagctcctggccctggccctgatcatcctgctgggcttcgtccagatcgggaagggtgatgtgtccaatctagatcccaacttctcatttgaaggcaccaaactggatgtggggaacattgtgctggcattatacagcggcctctttgcctatggaggatggaattacttgaatttcgtcacagaggaaatgatcaacccctacagaaacctgcccctggccatcatcatctccctgcccatcgtgacgctggtgtacgtgctgaccaacctggcctacttcaccaccctgtccaccgagcagatgctgtcgtccgaggccgtggccgtggacttcgggaactatcacctgggcgtcatgtcctggatcatccccgtcttcgtgggcctgtcctgcttcggctccgtcaatgggtccctgttcacatcctccaggctcttcttcgtggggtcccgggaaggccacctgccctccatcctctccatgatccacccacagctcctcacccccgtgccgtccctcgtgttcacgtgtgtgatgacgctgctctacgccttctccaaggacatcttctccgtcatcaacttcttcagcttcttcaactggctctgcgtggccctggccatcatcggcatgatctggctgcgccacagaaagcctgagcttgagcggcccatcaaggtgaacctggccctgcctgtgttcttcatcctggcctgcctcttcctgatcgccgtctccttctggaagacacccgtggagtgtggcatcggcttcaccatcatcctcagcgggctgcccgtctacttcttcggggtctggtggaaaaacaagcccaagtggctcctccagggcatcttctccacgaccgtcctgtgtcagaagctcatgcaggtggtcccccaggagacatagccaggaggccgagtggctgccggaggagcatgcgcagaggccagttaaagtaagggc

SEQ ID NO: 26 - protein, 507 aa, Homo sapiens
MAGAGPKRRALAAPAAEEKEEAREKMLAAKSADGSAPAGEGEGVTLQRNITLLNGVAIIVGTIIGSGIFVTPTGVLKEAGSPGLALVVWAACGVFSIVGALCYAELGTTISKSGGDYAYMLEVYGSLPAFLKLWIELLIIRPSSQYIVALVFATYLLKPLFPTCPVPEEAAKLVACLCVLLLTAVNCYSVKAATRVQDAFAAAKLLALALIILLGFVQIGKGDVSNLDPNFSFEGTKLDVGNIVLALYSGLFAYGGWNYLNFVTEEMINPYRNLPLAIIISLPIVTLVYVLTNLAYFTTLSTEQMLSSEAVAVDFGNYHLGVMSWIIPVFVGLSCFGSVNGSLFTSSRLFFVGSREGHLPSILSMIHPQLLTPVPSLVFTCVMTLLYAFSKDIFSVINFFSFFNWLCVALAIIGMIWLRHRKPELERPIKVNLALPVFFILACLFLIAVSFWKTPVECGIGFTIILSGLPVYFFGVWWKNKPKWLLQGIFSTTVLCQKLMQVVPQET

SEQ ID NO: 27 - DNA, 1739 bp, Homo sapiens
tcgcacactggtggcttcagaagaaattctcaacacctagctcgccagagagtctatgtatgggattgaacaatctgtaaactaaaggatcctaatcatgaaaataagtatgataaattataagtcactattggcactgttgtttatattagcctcctggatcatttttacagttttccagaactccacaaaggtttggtctgctctaaacttatccatctccctccattactggaacaactccacaaagtccttattccctaaaacaccactgatatcattaaagccactaacagagactgaactcagaataaaggaaatcatagagaaactagatcagcagatcccacccagacctttcacccacgtgaacaccaccaccagcgccacacatagcacagccaccatcctcaaccctcgagatacgtactgcaggggagaccagctgcacatcctgctggaggtgagggaccacttgggacgcaggaagcaatatggcggggatttcctgagggccaggatgtcttccccagcgctgatggcaggtgcttcaggaaaggtgactgacttcaacaacggcacctacctggtcagcttcactctgttctgggagggccaggtctctctgtctctgctgctcatccaccccagtgaaggggtgtcagctctctggagtgcaaggaaccaaggctatgacagggtgatcttcactggccagtttgtcaatggcacttcccaagtccactctgaatgtggcctgatcctaaacacaaatgctgaattgtgccagtacctggacaacagagaccaagaaggcttctactgtgtgaggcctcaacacatgccctgtgctgcactcactcacatgtattctaagaacaagaaagtttcttatcttagcaaacaagaaaagagcctctttgaaaggtcaaatgtgggtgtagagattatggaaaaattcaatacaattagtgtctccaaatgcaacaaagaaacagttgcaatgaaagagaaatgcaagtttggaatgacatccacaatccccagtgggcatgtctggagaaacacatggaatcctgtctcctgtagtttggctacagtcaaaatgaaggaatgcctgagaggaaaactcatatacctaatgggagattccacgatccgccagtggatggaatacttcaaagccagtatcaacacactgaagtcagtggatctgcatgaatctggaaaattgcaacaccagcttgctgtggatttggataggaacatcaacatccagtggcaaaaatattgttatcccttgataggatcaatgacctattcagtcaaagagatggagtacctcacccgggccattgacagaactggaggagaaaaaaatactgtcattgttatttccctgggccagcatttcagaccctttcccattgatgtttttatccgaagggccctcaatgtccacaaagccattcagcatcttcttctgagaagcccagacactatggttatcatcaaaacagaaaacatcagggagatgtacaatgatgcagaaagatttagtgactttcatggttacattcaatatctcatcataaaggacattttccaggatctcagtgtgagtatcattgatgcctgggatataacaattgcatatggcacaaataatgtacacccacctcaacatgtagtcggaaatcagattaatatattattaaactatatttgttaaataacaa

SEQ ID NO: 28 - protein, 544 aa, Homo sapiens
MKISMINYKSLLALLFILASWIIFTVFQNSTKVWSALNLSISLHYWNNSTKSLFPKTPLISLKPLTETELRIKEIIEKLDQQIPPRPFTHVNTTTSATHSTATILNPRDTYCRGDQLHILLEVRDHLGRRKQYGGDFLRARMSSPALMAGASGKVTDFNNGTYLVSFTLFWEGQVSLSLLLIHPSEGVSALWSARNQGYDRVIFTGQFVNGTSQVHSECGLILNTNAELCQYLDNRDQEGFYCVRPQHMPCAALTHMYSKNKKVSYLSKQEKSLFERSNVGVEIMEKFNTISVSKCNKETVAMKEKCKFGMTSTIPSGHVWRNTWNPVSCSLATVKMKECLRGKLIYLMGDSTIRQWMEYFKASINTLKSVDLHESGKLQHQLAVDLDRNINIQWQKYCYPLIGSMTYSVKEMEYLTRAIDRTGGEKNTVIVISLGQHFRPFPIDVFIRRALNVHKAIQHLLLRSPDTMVIIKTENIREMYNDAERFSDFHGYIQYLIIKDIFQDLSVSIIDAWDITIAYGTNNVHPPQHVVGNQINILLNYIC

SEQ ID NO: 29 - DNA, 1754 bp, Homo sapiens
atgctggtagccggcttcctgctggcgctgccgccgagctgggccgcgggcgcccccagggcgggcaggcgccccgcgcggccgcggggctgcgcggaccggccggaggagctactggagcagctgtacgggcgcctggcggccggcgtgctcagtgccttccaccacacgctgcagctggggccgcgtgagcaggcgcgcaacgcgagctgcccggcagggggcaggcccggcgaccgccgcttccggccgcccaccaacctgcgcagcgtgtcgccctgggcctacagaatctcctacgacccggcgaggtaccccaggtacctgcctgaagcctactgcctgtgccggggctgcctgaccgggctgttcggcgaggaggacgtgcgcttccgcagcgcccctgtctacatgcccaccgtcgtcctgcgccgcacccccgcctgcgccggcggccgttccgtctacaccgaggcctacgtcaccatccccgtgggctgcacctgcgtccccgagccggagaaggacgcagacagcatcaactccagcatcgacaaacagggcgccaagctcctgctgggccccaacgacgcgcccgctggcccctgaggccggtcctgccccgggaggtctccccggcccgcatcccgaggcgcccaagctggagccgcctggagggctcggtcggcgacctctgaagagagtgcaccgagcaaaccaagtgccggagcaccagcgccgcctttccatggagactcgtaagcagcttcatctgacacgggcatccctggcttgcttttagctacaagcaagcagcgtggctggaagctgatgggaaacgacccggcacgggcatcctgtgtgcggcccgcatggagggtttggaaaagttcacggaggctccctgaggagcctctcagatcggctgctgcgggtgcagggcgtgactcaccgctgggtgcttgccaaagagatagggacgcatatgctttttaaagcaatctaaaaataataataagtatagcgactatatacctacttttaaaatcaactgttttgaatagaggcagagctattttatattatcaaatgagagctactctgttacatttcttaacatataaacatcgttttttacttcttctggtagaattttttaaagcataattggaatccttggataaattttgtagctggtacactctggcctgggtctctgaattcagcctgtcaccgatggctgactgatgaaatggacacgtctcatctgacccactcttccttccactgaaggtcttcacgggcctccaggtggaccaaagggatgcacaggcggctcgcatgccccagggccagctaagagttccaaagatctcagatttggttttagtcatgaatacataaacagtctcaaactcgcacaattttttcccccttttgaaagccactggggccaatttgtggttaagaggtggtgagataagaagtggaacgtgacatctttgccagttgtcagaagaatccaagcaggtattggcttagttgtaagggctttaggatcaggctgaatatgaggacaaagtgggccacgttagcatctgcagagatcaatctggaggcttctgtttctgcattctgccacgagagctaggtccttgatcttttctttagattgaaagtctgtctctgaacacaattatttgtaaaagttagtagttcttttttaaatcattaaaagaggcttgctgaaggat

SEQ ID NO: 30 - protein, 202 aa, Homo sapiens [sequence omitted]

SEQ ID NO: 31 - DNA, 2692 bp, Homo sapiens
cccacgcgtccggcgagagtggagccgagcggtgcggagcagatctggtggttctccggagagcagcttcctcgggtgttacatgagccaagccctcactgtacagaagagtgagagctgaaacctgttccctgagctgatcagaaggacatcccttggcccctccatctgggctcctgtggataggaggggctgggtgagcaggccagctgggctatggtgtggtgcctcggcctggccgtcctcagcctggtcatcagccagggggctgacggtcgagggaagcctgaggtggtatcggtggtgggccgggctggggagagtgtggtgctgggctgtgacctgctgcccccggccggccggccccccctgcatgtcatcgagtggctgcgctttggattcctgcttcccatcttcatccagttcggcctctactctccccgaattgaccctgattacgtgggacgagtccggctgcagaagggggcctctctccagattgagggtctccgggtggaagaccagggctggtacgagtgccgcgtgttcttcctggaccagcacatccctgaagacgattttgctaacggctcctgggtgcatctgacagtcaattcaccccctcaattccaggagacacctcctgctgtgttggaagtgcaggaactggagcctgtgaccctgcgttgtgtggcccgtggcagccccctgcctcatgtgacgtggaagctccgaggaaaggaccttggccagggccagggccaggtgcaagtgcagaacgggacgctgcggatccgccgggtagagcgaggcagctctggggtctacacctgccaagcctccagcactgagggcagcgccacccacgccacccagctgctagtgctaggacccccagtcatcgtggtgccccccaagaacagcacagtcaatgcctcccaggatgcttcattggcctgccacgctgaggcataccctgctaacctcacctacagctggttccaggacaacatcaatgtcttccacattagccgcctgcagccccgggtgcggatcctggtggacgggagcctgcggctgctggccacccagcctgatgatgccggctgctacacctgtgtgcccagcaatggcctcctgcatccaccctcagcctctgcctacctcactgtgctctacccagcccaggtgacagctatgcctcctgagacacccctgcccataggcatgccgggggtgatccgctgcccggttcgtgccaaccccccactgctctttgtcagctggaccaaggatggaaaggccctgcagctggacaagttccctggctggtcccagggcacagaaggctcactgatcatcgccctggggaacgaggatgccctgggagaatactcctgcaccccctacaacagtcttggtaccgccgggccctctcctgtgacccgcgtgctgctcaaggctcccccagcttttatagagcggcccaaggaagaatatttccaagaagtagggcgggagctgctcatcccctgctccgcccaaggggaccctcctccagcagccccacccagtcccttgccaggtcctggacccctcctccagtacctgagcctgcccttcttccgagagatgaatgtggatggggactggcccccgcttgaggagcccagccctgctgcacccccagattacatggatacccggcgctgtcccacctcatctttccttcgttctccagaaacccctcctgtatcccccagggaatcacttcctggggctgtggtaggggctggggccactgcagagcccccttacacagccctggctgactggacactgagggagcggctgctgccaggccttctccctgctgcccctcgaggcagcctcaccagccagagcagtgggcgaggcagcgcttcgttcctgcggcccccctccacagccccctctgcaggaggcagctacctcagccctgctccaggagacaccagcagctgggccagtggccctgagagatggccccgaagggagcatgtggtgacagtcagcaagaggaggaacacatctgtggacgagaactatgagtgggactcagaattccctggggacatggaattgctggagactttgcacctgggcttggccagctcccggctcagacctgaagctgagccagagctaggtgtgaagactccagaggagggctgcctcctgaacactgcccatgttactggccctgaggcccgctgtgctgcccttcgggaggaattcctggccttccgccgccgccgagatgctactagggctcggctaccagcctatcgacagccagtcccccaccccgaacaggccactctgctgtgaacgtccctaatgtgaggctgtgaaaaggcatatggacctgcaaaggaggcccccaaccagacagacctagtttcaaacgagggcactgcccctgcctgcccctttggtgcccaggcacagaccctgatagtgggtttgggtcaccttggtatggaatgtatgtgctgaccccctaggtgagtctggggattggaacagggatcttaggtctgcctctctctctctctctctctctctctctctctgtgtgtgtgtgtgtgtgtgtgtgtgtgtgaagttttttacaggtgaataaacaaagtttgaaagataaaaaaaaaaa

SEQ ID NO: 32 - protein, 717 aa, Homo sapiens [sequence omitted]

SEQ ID NO: 33 - DNA, 3325 bp, Homo sapiens
gaacgcttgtgtctaactgatgctcctaatgcggaagcccctgaaaggcggttgtggtgcaaaggaaaacccacaggccaaggaatgggaagaccaaggttgacacttgtttgtcaagtgtcaataatcatctctgcccgggacctcagcatgaacaacctcacagagcttcagcctggcctcttccaccacctgcgcttcttggaggagctgcgtctctctgggaaccatctctcacacatcccaggacaagcattctctggtctctacagcctgaaaatcctgatgctgcagaacaatcagctgggaggaatccccgcagaggcgctgtgggagctgccgagcctgcagtcgctgcgcctagatgccaacctcatctccctggtcccggagaggagctttgaggggctgtcctccctccgccacctctggctggacgacaatgcactcacggagatccctgtcagggccctcaacaacctccctgccctgcaggccatgaccctggccctcaaccgcatcagccacatccccgactacgcgttccagaatctcaccagccttgtggtgctgcatttgcataacaaccgcatccagcatctggggacccacagcttcgaggggctgcacaatctggagacactagacctgaattataacaagctgcaggagttccctgtggccatccggaccctgggcagactgcaggaactggggttccataacaacaacatcaaggccatcccagaaaaggccttcatggggaaccctctgctacagacgatacacttttatgataacccaatccagtttgtgggaagatcggcattccagtacctgcctaaactccacacactatctctgaatggtgccatggacatccaggagtttccagatctcaaaggcaccaccagcctggagatcctgaccctgacccgcgcaggcatccggctgctcccatcggggatgtgccaacagctgcccaggctccgagtcctggaactgtctcacaatcaaattgaggagctgcccagcctgcacaggtgtcagaaattggaggaaatcggcctccaacacaaccgcatctgggaaattggagctgacaccttcagccagctgagctccctgcaagccctggatcttagctggaacgccatccggtccatccaccctgaggccttctccaccctgcactccctggtcaagctggacctgacagacaaccagctgaccacactgcccctggctggacttgggggcttgatgcatctgaagctcaaagggaaccttgctctctcccaggccttctccaaggacagtttcccaaaactgaggatcctggaggtgccttatgcctaccagtgctgtccctatgggatgtgtgccagcttcttcaaggcctctgggcagtgggaggctgaagaccttcaccttgatgatgaggagtcttcaaaaaggcccctgggcctccttgccagacaagcagagaaccactatgaccaggacctggatgagctccagctggagatggaggactcaaagccacaccccagtgtccagtgtagccctactccaggccccttcaagccctgtgagtacctctttgaaagctggggcatccgcctggccgtgtgggccatcgtgttgctctccgtgctctgcaatggactggtgctgctgaccgtgttcgctggcgggcctgcccccctgcccccggtcaagtttgtggtaggtgcgattgcaggcgccaacaccttgactggcatttcctgtggccttctagcctcagtcgatgccctgacctttggtcagttctctgagtacggagcccgctgggagacggggctaggctgccgggccactggcttcctggcagtacttgggtcggaggcatcggtgctgctgctcactctggccgcagtgcagtgcagcgtctccgtctcctgtgtccgggcctatgggaagtccccctccctgggcagcgttcgagcaggggtcctaggctgcctggcactggcagggctggccgccgcactgcccctggcctcagtgggagaatacggggcctccccactctgcctgccctacgcgccacctgagggtcagccagcagccctgggcttcaccgtggccctggtgatgatgaactccttctgtttcctggtcgtggccggtgcctacatcaaactgtactgtgacctgccgcggggcgactttgaggccgtgtgggactgcgccatggtgaggcacgtggcctggctcatcttcgcagacgggctcctctactgtcccgtggccttcctcagcttcgcctccatgctgggcctcttccctgtcacgcccgaggccgtcaagtctgtcctgctggtggtgctgcccctgcctgcctgcctcaacccactgctgtacctgctcttcaacccccacttccgggatgaccttcggcggcttcggccccgcgcaggggactcagggcccctagcctatgctgcggccggggagctggagaagagctcctgtgattctacccaggccctggtagccttctctgatgtggatctcattctggaagcttctgaagctgggcggccccctgggctggagacctatggcttcccctcagtgaccctcatctcctgtcagcagccaggggcccccaggctggagggcagccattgtgtagagccagaggggaaccactttgggaacccccaaccctccatggatggagaactgctgctgagggcagagggatctacgccagcaggtggaggcttgtcagggggtggcggctttcagccctctggcttggcctttgcttcacacgtgtaaatatccctccccattcttctcttcccctctcttccctttcctctctccccctcggtgaatgatggctgcttctaaaacaaatacaaccaaaactcagcagtgtgatctatagcaggatggcccagtacctggctccactgatcacctctctcctgtgaccatcaccaacgggtgcctcttggcctggctttcccttggccttcctcagcttcaccttgatactgggcctcttccttgtcatgtctgaagctgtggaccagagacctggacttttgtctgcttaagggaaatgagggaagtaaagacagtgaaggggtggagggttgatcagggcacagtggacagggagacctcacagagaaaggcctggaaggtgatttcccgtgtgactcatggataggatacaaaatgtgttccatgtaccattaatcttgacatatgccatgcataaagacttcctattaaaataagctttggaagagaaaaaaaaaaa

SEQ ID NO: 34 - protein, 915 aa, Homo sapiens
MGRPRLTLVCQVSIIISARDLSMNNLTELQPGLFHHLRFLEELRLSGNHLSHIPGQAFSGLYSLKILMLQNNQLGGIPAEALWELPSLQSLRLDANLISLVPERSFEGLSSLRHLWLDDNALTEIPVRALNNLPALQAMTLALNRISHIPDYAFQNLTSLVVLHLHNNRIQHLGTHSFEGLHNLETLDLNYNKLQEFPVAIRTLGRLQELGFHNNNIKAIPEKAFMGNPLLQTIHFYDNPIQFVGRSAFQYLPKLHTLSLNGAMDIQEFPDLKGTTSLEILTLTRAGIRLLPSGMCQQLPRLRVLELSHNQIEELPSLHRCQKLEEIGLQHNRIWEIGADTFSQLSSLQALDLSWNAIRSIHPEAFSTLHSLVKLDLTDNQLTTLPLAGLGGLMHLKLKGNLALSQAFSKDSFPKLRILEVPYAYQCCPYGMCASFFKASGQWEAEDLHLDDEESSKRPLGLLARQAENHYDQDLDELQLEMEDSKPHPSVQCSPTPGPFKPCEYLFESWGIRLAVWAIVLLSVLCNGLVLLTVFAGGPAPLPPVKFVVGAIAGANTLTGISCGLLASVDALTFGQFSEYGARWETGLGCRATGFLAVLGSEASVLLLTLAAVQCSVSVSCVRAYGKSPSLGSVRAGVLGCLALAGLAAALPLASVGEYGASPLCLPYAPPEGQPAALGFTVALVMMNSFCFLVVAGAYIKLYCDLPRGDFEAVWDCAMVRHVAWLIFADGLLYCPVAFLSFASMLGLFPVTPEAVKSVLLVVLPLPACLNPLLYLLFNPHFRDDLRRLRPRAGDSGPLAYAAAGELEKSSCDSTQALVAFSDVDLILEASEAGRPPGLETYGFPSVTLISCQQPGAPRLEGSHCVEPEGNHFGNPQPSMDGELLLRAEGSTPAGGGLSGGGGFQPSGLAFASHV

SEQ ID NO: 35 - DNA, 2479 bp, Homo sapiens
gtcatattgaacattccagatacctatcattactcgatgctgttgataacagcaagatggctttgaactcagggtcaccaccagctattggaccttactatgaaaaccatggataccaaccggaaaacccctatcccgcacagcccactgtggtccccactgtctacgaggtgcatccggctcagtactacccgtcccccgtgccccagtacgccccgagggtcctgacgcaggcttccaaccccgtcgtctgcacgcagcccaaatccccatccgggacagtgtgcacctcaaagactaagaaagcactgtgcatcaccttgaccctggggaccttcctcgtgggagctgcgctggccgctggcctactctggaagttcatgggcagcaagtgctccaactctgggatagagtgcgactcctcaggtacctgcatcaacccctctaactggtgtgatggcgtgtcacactgccccggcggggaggacgagaatcggtgtgttcgcctctacggaccaaacttcatccttcagatgtactcatctcagaggaagtcctggcaccctgtgtgccaagacgactggaacgagaactacgggcgggcggcctgcagggacatgggctataagaataatttttactctagccaaggaatagtggatgacagcggatccaccagctttatgaaactgaacacaagtgccggcaatgtcgatatctataaaaaactgtaccacagtgatgcctgttcttcaaaagcagtggtttctttacgctgtttagcctgcggggtcaacttgaactcaagccgccagagcaggatcgtgggcggtgagagcgcgctcccgggggcctggccctggcaggtcagcctgcacgtccagaacgtccacgtgtgcggaggctccatcatcacccccgagtggatcgtgacagccgcccactgcgtggaaaaacctcttaacaatccatggcattggacggcatttgcggggattttgagacaatctttcatgttctatggagccggataccaagtacaaaaagtgatttctcatccaaattatgactccaagaccaagaacaatgacattgcgctgatgaagctgcagaagcctctgactttcaacgacctagtgaaaccagtgtgtctgcccaacccaggcatgatgctgcagccagaacagctctgctggatttccgggtggggggccaccgaggagaaagggaagacctcagaagtgctgaacgctgccaaggtgcttctcattgagacacagagatgcaacagcagatatgtctatgacaacctgatcacaccagccatgatctgtgccggcttcctgcaggggaacgtcgattcttgccagggtgacagtggagggcctctggtcacttcgaacaacaatatctggtggctgataggggatacaagctggggttctggctgtgccaaagcttacagaccaggagtgtacgggaatgtgatggtattcacggactggatttatcgacaaatgaaggcaaacggctaatccacatggtcttcgtccttgacgtcgttttacaagaaaacaatggggctggttttgcttccccgtgcatgatttactcttagagatgattcagaggtcacttcatttttattaaacagtgaacttgtctggctttggcactctctgccatactgtgcaggctgcagtggctcccctgcccagcctgctctccctaaccccttgtccgcaaggggtgatggccggctggttgtgggcactggcggtcaattgtggaaggaagagggttggaggctgcccccattgagatcttcctgctgagtcctttccaggggccaattttggatgagcatggagctgtcacttctcagctgctggatgacttgagatgaaaaaggagagacatggaaagggagacagccaggtggcacctgcagcggctgccctctggggccacttggtagtgtccccagcctacttcacaaggggattttgctgatgggttcttagagccttagcagccctggatggtggccagaaataaagggaccagcccttcatgggtggtgacgtggtagtcacttgtaaggggaacagaaacatttttgttcttatggggtgagaatatagacagtgcccttggtgcgagggaagcaattgaaaaggaacttgccctgagcactcctggtgcaggtctccacctgcacattgggtggggctcctgggagggagactcagccttcctcctcatcctccctgaccctgctcctagcaccctggagagtgaatgccccttggtccctggcagggcgccaagtttggcaccatgtcggcctcttcaggcctgatagtcattggaaattgaggtccatgggggaaatcaaggatgctcagtttaaggtacactgtttccatgttatgtttctacacattgatggtggtgaccctgagttcaaagccatctt

SEQ ID NO: 36 - protein, 492 aa, Homo sapiens
MALNSGSPPAIGPYYENHGYQPENPYPAQPTVVPTVYEVHPAQYYPSPVPQYAPRVLTQASNPVVCTQPKSPSGTVCTSKTKKALCITLTLGTFLVGAALAAGLLWKFMGSKCSNSGIECDSSGTCINPSNWCDGVSHCPGGEDENRCVRLYGPNFILQMYSSQRKSWHPVCQDDWNENYGRAACRDMGYKNNFYSSQGIVDDSGSTSFMKLNTSAGNVDIYKKLYHSDACSSKAVVSLRCLACGVNLNSSRQSRIVGGESALPGAWPWQVSLHVQNVHVCGGSIITPEWIVTAAHCVEKPLNNPWHWTAFAGILRQSFMFYGAGYQVQKVISHPNYDSKTKNNDIALMKLQKPLTFNDLVKPVCLPNPGMMLQPEQLCWISGWGATEEKGKTSEVLNAAKVLLIETQRCNSRYVYDNLITPAMICAGFLQGNVDSCQGDSGGPLVTSNNNIWWLIGDTSWGSGCAKAYRPGVYGNVMVFTDWIYRQMKANG

SEQ ID NO: 37 - DNA, 1196 bp, Homo sapiens
cccggctccgctccctctgccccctcggggtcgcgcgcccacgatgctgcagggccctggctcgctgctgctgctcttcctcgcctcgcactgctgcctgggctcggcgcgcgggctcttcctctttggccagcccgacttctcctacaagcgcagcaattgcaagcccatcccggtcaacctgcagctgtgccacggcatcgaataccagaacatgcggctgcccaacctgctgggccacgagaccatgaaggaggtgctggagcaggccggcgcttggatcccgctggtcatgaagcagtgccacccggacaccaagaagttcctgtgctcgctcttcgcccccgtctgcctcgatgacctagacgagaccatccagccatgccactcgctctgcgtgcaggtgaaggaccgctgcgccccggtcatgtccgccttcggcttcccctggcccgacatgcttgagtgcgaccgtttcccccaggacaacgacctttgcatccccctcgctagcagcgaccacctcctgccagccaccgaggaagctccaaaggtatgtgaagcctgcaaaaataaaaatgatgatgacaacgacataatggaaacgctttgtaaaaatgattttgcactgaaaataaaagtgaaggagataacctacatcaaccgagataccaaaatcatcctggagaccaagagcaagaccatttacaagctgaacggtgtgtccgaaagggacctgaagaaatcggtgctgtggctcaaagacagcttgcagtgcacctgtgaggagatgaacgacatcaacgcgccctatctggtcatgggacagaaacagggtggggagctggtgatcacctcggtgaagcggtggcagaaggggcagagagagttcaagcgcatctcccgcagcatccgcaagctgcagtgctagtcccggcatcctgatggctccgacaggcctgctccagagcacggctgaccatttctgctccgggatctcagctcccgttccccaagcacactcctagctgctccagtctcagcctgggcagcttccccctgccttttgcacgtttgcatccccagcatttcctgagttataaggccacaggagtggatagctgttttcacctaaaggaaaagcccacccgaatcttgtagaaatattcaaactaataaaatcatgaatattttaa

SEQ ID NO: 38 - protein, 295 aa, Homo sapiens [sequence omitted]

SEQ ID NO: 39 - DNA, 3781 bp, Homo sapiens
ctccgggtccccaggggctgcgccgggccggcctggcaagggggacgagtcagtggacactccaggaagagcggccccgcggggggcgatgaccgtgcgctgaccctgactcactccaggtccggaggcgggggcccccggggcgactcgggggcggaccgcggggcggagctgccgcccgtgagtccggccgagccacctgagcccgagccgcgggacaccgtcgctcctgctctccgaatgctgcgcaccgcgatgggcctgaggagctggctcgccgccccatggggcgcgctgccgcctcggccaccgctgctgctgctcctgctgctgctgctcctgctgcagccgccgcctccgacctgggcgctcagcccccggatcagcctgcctctgggctctgaagagcggccattcctcagattcgaagctgaacacatctccaactacacagcccttctgctgagcagggatggcaggaccctgtacgtgggtgctcgagaggccctctttgcactcagtagcaacctcagcttcctgccaggcggggagtaccaggagctgctttggggtgcagacgcagagaagaaacagcagtgcagcttcaagggcaaggacccacagcgcgactgtcaaaactacatcaagatcctcctgccgctcagcggcagtcacctgttcacctgtggcacagcagccttcagccccatgtgtacctacatcaacatggagaacttcaccctggcaagggacgagaaggggaatgtcctcctggaagatggcaagggccgttgtcccttcgacccgaatttcaagtccactgccctggtggttgatggcgagctctacactggaacagtcagcagcttccaagggaatgacccggccatctcgcggagccaaagccttcgccccaccaagaccgagagctccctcaactggctgcaagacccagcttttgtggcctcagcctacattcctgagagcctgggcagcttgcaaggcgatgatgacaagatctactttttcttcagcgagactggccaggaatttgagttctttgagaacaccattgtgtcccgcattgcccgcatctgcaagggcgatgagggtggagagcgggtgctacagcagcgctggacctccttcctcaaggcccagctgctgtgctcacggcccgacgatggcttccccttcaacgtgctgcaggatgtcttcacgctgagccccagcccccaggactggcgtgacacccttttctatggggtcttcacttcccagtggcacaggggaactacagaaggctctgccgtctgtgtcttcacaatgaaggatgtgcagagagtcttcagcggcctctacaaggaggtgaaccgtgagacacagcagtggtacaccgtgacccacccggtgcccacaccccggcctggagcgtgcatcaccaacagtgcccgggaaaggaagatcaactcatccctgcagctcccagaccgcgtgctgaacttcctcaaggaccacttcctgatggacgggcaggtccgaagccgcatgctgctgctgcagccccaggctcgctaccagcgcgtggctgtacaccgcgtccctggcctgcaccacacctacgatgtcctcttcctgggcactggtgacggccggctccacaaggcagtgagcgtgggcccccgggtgcacatcattgaggagctgcagatcttctcatcgggacagcccgtgcagaatctgctcctggacacccacagggggctgctgtatgcggcctcacactcgggcgtagtccaggtgcccatggccaactgcagcctgtaccggagctgtggggactgcctcctcgcccgggacccctactgtgcttggagcggctccagctgcaagcacgtcagcctctaccagcctcagctggccaccaggccgtggatccaggacatcgagggagccagcgccaaggacctttgcagcgcgtcttcggttgtgtccccgtcttttgtaccaacaggggagaagccatgtgagcaagtccagttccagcccaacacagtgaacactttggcctgcccgctcctctccaacctggcgacccgactctggctacgcaacggggcccccgtcaatgcctcggcctcctgccacgtgctacccactggggacctgctgctggtgggcacccaacagctgggggagttccagtgctggtcactagaggagggcttccagcagctggtagccagctactgcccagaggtggtggaggacggggtggcagaccaaacagatgagggtggcagtgtacccgtcattatcagcacatcgcgtgtgagtgcaccagctggtggcaaggccagctggggtgcagacaggtcctactggaaggagttcctggtgatgtgcacgctctttgtgctggccgtgctgctcccagttttattcttgctctaccggcaccggaacagcatgaaagtcttcctgaagcagggggaatgtgccagcgtgcaccccaagacctgccctgtggtgctgccccctgagacccgcccactcaacggcctagggccccctagcaccccgctcgatcaccgagggtaccagtccctgtcagacagccccccgggggcccgagtcttcactgagtcagagaagaggccactcagcatccaagacagcttcgtggaggtatccccagtgtgcccccggccccgggtccgccttggctcggagatccgtgactctgtggtgtgagagctgacttccagaggacgctgccctggcttcaggggctgtgaatgctcggagagggtcaactggacctcccctccgctctgctcttcgtggaacacgaccgtggtgcccggcccttgggagccttggagccagctggcctgctgctctccagtcaagtagcgaagctcctaccacccagacacccaaacagccgtggccccagaggtcctggccaaatatgggggcctgcctaggttggtggaacagtgctccttatgtaaactgagccctttgtttaaaaaacaattccaaatgtgaaactagaatgagagggaagagatagcatggcatgcagcacacacggctgctccagttcatggcctcccaggggtgctggggatgcatccaaagtggttgtctgagacagagttggaaaccctcaccaactggcctcttcaccttccacattatcccgctgccaccggctgccctgtctcactgcagattcaggaccagcttgggctgcgtgcgttctgccttgccagtcagccgaggatgtagttgttgctgccgtcgtcccaccacctcagggaccagagggctaggttggcactgcggccctcaccaggtcctgggctcggacccaactcctggacctttccagcctgtatcaggctgtggccacacgagaggacagcgcgagctcaggagagatttcgtgacaatgtacgcctttccctcagaattcagggaagagactgtcgcctgccttcctccgttgttgcgtgagaacccgtgtgccccttcccaccatatccaccctcgctccatctttgaactcaaacacgaggaactaactgcaccctggtcctctccccagtccccagttcaccctccatccctcaccttcctccactctaagggatatcaacactgcccagcacaggggccctgaatttatgtggtttttatacattttttaataagatgcactttatgtcattttttaataaagtctgaagaattactgtttaaaaaaaaaaaa

SEQ ID NO: 40 - protein, 837 aa, Homo sapiens [sequence omitted]

SEQ ID NO: 41 - DNA, 43 bp, Artificial sequence
  oligonucleotide probe
tgtaaaacgacggccagttaaatagacctgcaattattaatct

SEQ ID NO: 42 - DNA, 41 bp, Artificial sequence
  sequence is synthesized
caggaaacagctatgaccacctgcacacctgcaaatccatt

SEQ ID NO: 43 - DNA, 21 bp, Artificial sequence
  oligonucleotide probe
tgtccaccaagcagacagaag

SEQ ID NO: 44 - DNA, 21 bp, Artificial sequence
  oligonucleotide probe
actggatggcgcctttccatg

SEQ ID NO: 45 - DNA, 50 bp, Artificial sequence
  oligonucleotide probe
ctgacagtgactagctcagaccacccagaggacacggccaacgtcacagt

SEQ ID NO: 46 - DNA, 45 bp, Artificial sequence
  oligonucleotide probe
gggctctttcccacgctggtactatgaccccacggagcagatctg

SEQ ID NO: 47 - DNA, 24 bp, Artificial sequence
  oligonucleotide probe
gatggttcctgctcaagtgccctg

SEQ ID NO: 48 - DNA, 24 bp, Artificial sequence
  oligonucleotide probe
ttgcacttgtaggacccacgtacg

SEQ ID NO: 49 - DNA, 50 bp, Artificial sequence
  oligonucleotide probe
ctgatgggaggacctgtgtagatgttgatgaatgtgctacaggaagagcc

SEQ ID NO: 50 - DNA, 27 bp, Artificial sequence
  oligonucleotide probe
gcaccaccgtaggtacttgtgtgaggc

SEQ ID NO: 51 - DNA, 23 bp, Artificial sequence
  oligonucleotide probe
aaccaccagagccaagagccggg

SEQ ID NO: 52 - DNA, 50 bp, Artificial sequence
  oligonucleotide probe
cagcggaatcatcgatgcaggggcctcaattaatgtatctgtgatgttac

SEQ ID NO: 53 - DNA, 24 bp, Artificial sequence
  oligonucleotide probe
ctgctggtgaaatctggcgtggag

SEQ ID NO: 54 - DNA, 24 bp, Artificial sequence
  oligonucleotide probe
gtctggtcctggctgtccacccag

SEQ ID NO: 55 - DNA, 45 bp, Artificial sequence
  oligonucleotide probe
catcttgtcatgtacctgggaaccaccacagggtcgctccacaag

SEQ ID NO: 56 - DNA, 23 bp, Artificial sequence
  oligonucleotide probe
cccgtgtctgaagtcttcaggcg

SEQ ID NO: 57 - DNA, 24 bp, Artificial sequence
  oligonucleotide probe
tccagacagtatggcttctcccgc

SEQ ID NO: 58 - DNA, 45 bp, Artificial sequence
  oligonucleotide probe
attacgacgtttgtgccgaggctccctgtgaacagcagtgcacgg

SEQ ID NO: 59 - DNA, 23 bp, Artificial sequence
  oligonucleotide probe
gctcagtgccttccaccacacgc

SEQ ID NO: 60 - DNA, 21 bp, Artificial sequence
  oligonucleotide probe
ctgcgtccttctccggctcgg

SEQ ID NO: 61 - DNA, 45 bp, Artificial sequence
  oligonucleotide probe
cgttccgtctacaccgaggcctacgtcaccatccccgtgggctgc

SEQ ID NO: 62 - DNA, 20 bp, Artificial sequence
  oligonucleotide probe
ccaccttcctccccttgtgc

SEQ ID NO: 63 - DNA, 20 bp, Artificial sequence
  oligonucleotide probe
caggtgtagcagccggcatc

SEQ ID NO: 64 - DNA, 40 bp, Artificial sequence
  oligonucleotide probe
cagtcatcgtggtgccccccaagaacagcacagtcaatgc

SEQ ID NO: 65 - DNA, 23 bp, Artificial sequence
  oligonucleotide probe
gcatctgggaaattggagctgac

SEQ ID NO: 66 - DNA, 22 bp, Artificial sequence
  oligonucleotide probe
gcacacatcccatagggacagc

SEQ ID NO: 67 - DNA, 39 bp, Artificial sequence
  oligonucleotide probe
gctgagctccctgcaagccctggatcttagctggaacgc

SEQ ID NO: 68 - DNA, 21 bp, Artificial sequence
  oligonucleotide probe
cctggctcgctgctgctgctc

SEQ ID NO: 69 - DNA, 25 bp, Artificial sequence
  oligonucleotide probe
cctcacaggtgcactgcaagctgtc

SEQ ID NO: 70 - DNA, 47 bp, Artificial sequence
  oligonucleotide probe
ctcttcctctttggccagcccgacttctcctacaagcgcagaattgc

SEQ ID NO: 71 - DNA, 24 bp, Artificial sequence
  oligonucleotide probe
agcccgtgcagaatctgctcctgg

SEQ ID NO: 72 - DNA, 24 bp, Artificial sequence
  oligonucleotide probe
tgaagccagggcagcgtcctctgg

SEQ ID NO: 73 - DNA, 18 bp, Artificial sequence
  oligonucleotide probe
gtacaggctgcagttggc

SEQ ID NO: 74 - DNA, 41 bp, Artificial sequence
  oligonucleotide probe
agaagccatgtgagcaagtccagttccagcccaacacagtg

SEQ ID NO: 75 - DNA, 45 bp, Artificial sequence
  oligonucleotide probe
gagctgcagatcttctcatcgggacagcccgtgcagaatctgctc